(19) United States
(12) Patent Application Publication
(10) Pub. No.: US 2008/0275085 A1
(43) Pub. Date: Nov. 6, 2008

(54) Piperidine derivative and use thereof

(76) Inventors: Junya Shirai, Osaka (JP); Shinji Morimoto, Osaka (JP); Hideyuki Sugiyama, Osaka (JP); Nobuki Sakauchi, Osaka (JP); Takeshi Yoshikawa, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 12/081,926

(22) Filed: Apr. 23, 2008

(30) Foreign Application Priority Data
Apr. 24, 2007 (JP) 114858/2007

(51) Int. Cl. A61K 31/4545; C07D 401/04; A61P 1/00; A61K 31/445

(52) U.S. Cl. 514/318; 546/208; 514/326

(57) ABSTRACT

The present invention provides a novel piperidine derivative and a tachykinin receptor antagonist containing same, as well as a compound represented by the formula:

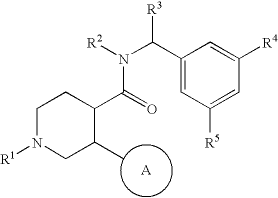

wherein R¹ is carbamoylmethyl, methylsulfonylethylcarbonyl and the like; R² is methyl or cyclopropyl; R³ is a hydrogen atom or methyl; R⁴ is a chlorine atom or trifluoromethyl; R⁵ is a chlorine atom or trifluoromethyl; and a group represented by the formula:

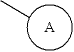

is a group represented by the formula:

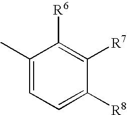

wherein R⁶ is a hydrogen atom, methyl, ethyl or isopropyl; R⁷ is a hydrogen atom, methyl or a chlorine atom; and R⁸ is a hydrogen atom, a fluorine atom, a chlorine atom or methyl; or 3-methylthiophen-2-yl, and a salt thereof.

### TECHNICAL FIELD

The present invention relates to a novel piperidine derivative having a superior antagonistic action for a tachykinin receptor, and use thereof.

### BACKGROUND OF THE INVENTION

Tachykinin is a generic term for a group of neuropeptides. Substance P (SP), neurokinin-A and neurokinin-B are known in mammals, and these peptides are known to bind to the corresponding receptors (neurokinin-1, neurokinin-2 and neurokinin-3) that exist in a living body and thereby to exhibit various biological activities.

Of such neuropeptides, SP has the longest history and has been studied in detail. In 1931, the existence of SP in the extract from equine intestines was confirmed, and in 1971, its structure was determined. SP is a peptide consisting of 11 amino acids.

SP is broadly distributed over the central and peripheral nervous systems, and has various physiological activities such as vasodilation, enhancement of vascular extravasation, contraction of smooth muscles, excitation of neurons, salivation, enhancement of diuresis, immunological enhancement and the like, in addition to the function as a transmitter substance for primary sensory neurons. In particular, it is known that SP released from the terminal in the spinal (dorsal) horn due to a pain impulse transmits the information of pain to secondary neurons, and that SP released from the peripheral terminal induces an inflammatory response in the receptor thereof. Thus, it is considered that SP is involved in various disorders (e.g., pain, headache, particularly migraine, Alzheimer's disease, multiple sclerosis, cardiovascular modulation, chronic inflammatory diseases such as chronic rheumatic arthritis, respiratory diseases including asthma or allergic rhinitis, intestinal inflammatory diseases including ulcerative colitis and Crohn's disease, ocular damage and ocular inflammatory diseases, proliferative vitreous retinopathy, irritable bowel syndrome, urinary frequency, psychosis, vomiting etc.) (e.g., non-patent reference 1 and non-patent reference 2).

At present, the following compounds have been known as compounds having antagonistic actions for SP receptors. In patent reference 1, disclosed are the compound of the formula:

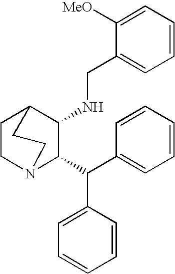

, and the like;
in patent reference 2, disclosed are the compound of the formula:

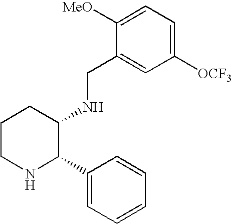

, and the like;
in patent reference 3, disclosed are the compound of the formula:

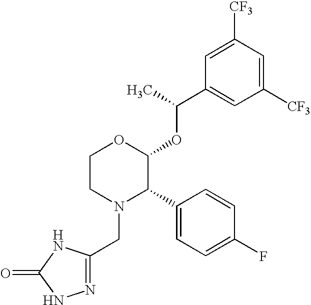

, and the like; and
in patent reference 4, disclosed are the heterocyclic compound of the formula:

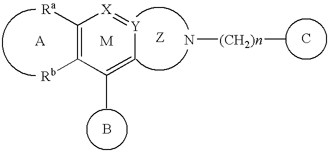

wherein Ring M is a heterocycle having —N═C<, —CO—N< or —CS—N< as a partial structure of

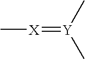

R^{a} and R^{b} are bonded to each other to form Ring A, or they are the same or different and represent a hydrogen atom or a substituent in Ring M; Ring A and Ring B are homocyclic or heterocyclic rings optionally having substituent(s), respectively and at least one of them is a heterocyclic ring optionally having substituent(s); Ring C is a homocyclic or heterocyclic ring optionally having substituent(s); Ring Z is a nitrogen-containing heterocyclic ring optionally having substituent(s); and n is an integer of 1 to 6, or salts thereof, and the like.

Patent reference 5 describes a compound having a tachykinin receptor antagonistic action, which is represented by the formula:

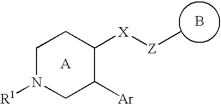

wherein Ar is an aryl group, an aralkyl group or an aromatic heterocyclic group, each of which optionally having substituent(s), R¹ is a hydrogen atom, a hydrocarbon group optionally having substituent(s), an acyl group or a heterocyclic group optionally having substituent(s), X is an oxygen atom or an imino group optionally having a substituent, Z is a methylene group optionally having substituent(s), ring A is a piperidine ring optionally further having substituent(s), ring B is an aromatic ring optionally having substituent(s), provided when Z is a methylene group substituted by an oxo group, then R¹ is not a methyl group and when Z is a methylene group substituted by a methyl group, then ring B is an aromatic ring having substituent(s)] or a salt thereof.

Patent reference 6 describes a compound having a tachykinin receptor antagonistic action, which is represented by the formula:

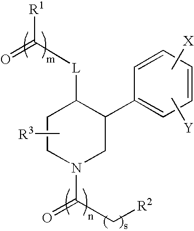

wherein m is 0 or 1; n is 0 or 1; s is 0 or 1; L is —O— or —N(R⁴)—; R¹ and R² are each independently hydrogen atom, aryl, heteroaryl, C₁₋₆ alkyl, heterocycloalkyl, C₁₋₆ alkylheterocycloalkyl, C₁₋₆ alkylheteroaryl, C₁₋₆ alkyl-O-aryl, C₁₋₆ alkylaryl, or —CH₂N(R⁴)(R⁵), wherein each of said heterocyloalkyl, C₁₋₆ alkylheterocycloalkyl, C₁₋₆ alkylheteroaryl, C₁₋₆ alkyl-O-aryl, aryl, C₁₋₆ alkylaryl, heteroaryl, and —CH₂N(R⁴)(R⁵), is optionally substituted with 1 to 3 substituents independently selected from X′, Y′ or Z′; R³ is hydrogen atom, CF₃, OH, or C₁₋₆ alkyl; R⁴ and R⁵ are each independently selected from hydrogen atom, C₁₋₆ alkyl or C₁₋₆ (C═O)R⁷; R⁷ is C₁₋₆ alkyl, OH, —CH₂N(R⁴)(R⁵) or —OR⁴; R⁸ and R⁹ are each independently C₁₋₆ alkyl; X, Y, X′, Y′ and Z′ are each independently selected from hydrogen atom, C₁₋₆ alkyl, C₁₋₆ alkyl-NR⁴R⁵, CF₃, OH, —O—C₁₋₆ alkyl, C₁₋₆ alkyl-C(═O)R⁷, aryl, heteroaryl, cycloalkyl, NO₂, C₁₋₆ alkylaryl, —O-aryl, halogen, CN, —CH₃N(R⁴)(R⁵), —C(═O)R⁷, —R⁶C(═O)R⁷ or —R⁶C(═O)N(R⁴)(R⁵); and R⁶ is a bond, —CH₂—, —O— or —NR⁴—, or a salt thereof.

Patent reference 7 describes a compound having a tachykinin receptor antagonistic action, which is represented by the formula:

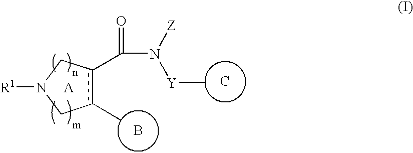

wherein ring A is a nitrogen-containing heterocycle optionally further having substituent(s), ring B and ring C are each an aromatic ring optionally having substituent(s), R¹ is a hydrogen atom, a hydrocarbon group optionally having substituent(s), an acyl group or a heterocyclic group optionally having substituent(s), Z is an optionally halogenated C₁₋₆ alkyl group, Y is a methylene group optionally having substituent(s), m and n are each an integer of 0 to 5, m+n is an integer of 2 to 5, and ______ is a single bond or a double bond, or a salt thereof.

Patent reference 8 describes a piperidine compound having a tachykinin receptor antagonistic action, which is represented by the formula:

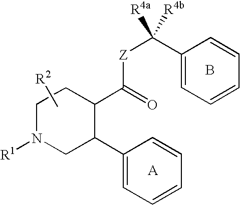

wherein ring A is a benzene ring optionally having substituent(s),
ring B is a benzene ring optionally having substituent(s),
R¹ is a hydrogen atom or a substituent of an amino group,
R² is a hydrogen atom, a hydroxyl group optionally having substituent(s), an amino group optionally having substituent(s), an alkyl group optionally having substituent(s), a carbonyl group having substituent(s) or a halogen atom,
Z is an oxygen atom or a group represented by —N(R³)—,
R³ is a hydrogen atom or an alkyl group optionally having substituent(s),
R^{4a} and R^{4b} are the same or different and each is a hydrogen atom or an alkyl group optionally having substituent(s), or groups bonded to each other at the terminal to form an alkylene group, or a pharmacologically acceptable salt thereof.
patent reference 1: EP 436,334 A
patent reference 2: WO 92/17449
patent reference 3: WO 95/16679
patent reference 4: JP-A-9-263585
patent reference 5: WO 03/101964
patent reference 6: US 2005/0256164 A
patent reference 7: WO 2005/068427
patent reference 8: WO 2006/004195
non-patent reference 1: Physiological Reviews, vol. 73, p. 229-308 (1993)
non-patent reference 2: Journal of Autonomic Pharmacology, vol. 13, p. 23-93 (1993)

### DISCLOSURE OF THE INVENTION

### Problems to be Solved by the Invention

An object of the present invention is to provide a piperidine derivative having antagonistic action for a tachykinin receptor etc. with a different chemical structure from the known compounds including the above-mentioned compounds, an agent for the prophylaxis or treatment of lower urinary tract diseases comprising the derivative, and the like.

The present inventors have made extensive studies in consideration of the above-mentioned situation and, as a result, have found unexpectedly that piperidine derivatives represented by the formula (I) below or a salt thereof have excellent antagonistic action for a tachykinin receptor (particularly antagonistic action for a SP receptor) and the like as based on their peculiar chemical structures and are sufficiently satisfactory as pharmaceutical compositions. On the basis of these findings, the present inventors have completed the present invention.

Specifically, the present invention provides:
[1] A compound represented by the formula:

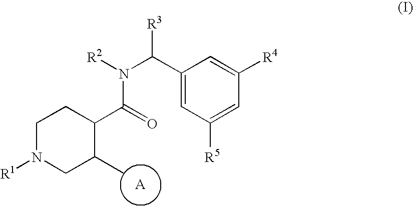

wherein
R¹ is
(1) carbamoylmethyl,
(2) methylsulfonylethylcarbonyl,
(3) aminosulfonylpropylcarbonyl,
(4) phenylsulfonylethylcarbonyl,
(5) pyridin-2-ylcarbonyl,
(6) 5-methoxycarbonylpyridin-2-ylcarbonyl,
(7) 5-hydroxypyridin-2-ylcarbonyl,
(8) 6-hydroxypyridin-2-ylcarbonyl,
(9) 5-bromopyridin-2-ylcarbonyl,
(10) 6-methylpyridin-2-ylcarbonyl,
(11) 5-carbamoylpyridin-2-ylcarbonyl,
(12) 2-arminopyridin-5-ylcarbonyl,
(13) 2-acetylaminopyridin-5-ylcarbonyl,
(14) pyridin-3-ylcarbonyl,
(15) pyrazin-2-ylcarbonyl,
(16) pyrimidin-5-ylcarbonyl,
(17) 2-aminopyrimidin-5-ylcarbonyl,
(18) 2-acetylaminopyrimidin-5-ylcarbonyl,
(19) 2-methoxycarbonylaminopyrimidin-5-ylcarbonyl,
(20) azetidin-3-ylcarbonyl,
(21) 1-tert-butoxycarbonylazetidin-3-ylcarbonyl,
(22) 1-(1-hydroxy-1-methylethylcarbonyl)azetidin-3-ylcarbonyl,
(23) 1-hydroxymethylcarbonylazetidin-3-ylcarbonyl,
(24) adamantan-1-ylcarbonyl,
(25) 1-carbamoyl-1-methylethylcarbonyl,
(26) dimethylaminomethylcarbonyl,
(27) 1,2,3,4-tetrahydropyrimidine-2,4-dion-1-ylmethylcarbonyl,
(28) 2,3,5,6-tetrahydropyrimidine-2,6-dion-4-ylmethylcarbonyl,
(29) 1-hydroxymethylcarbonylpiperidin-4-ylcarbonyl,
(30) 1-ethoxycarbonylpiperidin-4-ylcarbonyl,
(31) 1-isopropoxycarbonylpiperidin-4-ylcarbonyl,
(32) 1-cyclopropylsulfonylpiperidin-4-ylcarbonyl,
(33) 1-(1-hydroxyethylcarbonyl)piperidin-4-ylcarbonyl,
(34) 5,5-dimethyloxazolidine-2,4-dion-3-ylmethylcarbonyl,
(35) 5,5-dimethyloxazolidine-2,4-dion-3-ylethylcarbonyl,
(36) oxazolidine-2,4-dion-3-ylmethylcarbonyl,
(37) 1-(2-hydroxy-2-methylpropylcarbonyl)piperidin-4-ylcarbonyl,
(38) 3-methylimidazolidine-4,4-dimethyl-2,5-dion-1-ylmethylcarbonyl,
(39) 4,4-dimethylpiperidine-2,6-dion-1-ylmethylcarbonyl,
(40) morpholine-2,6-dion-1-ylmethylcarbonyl,
(41) imidazol-2-ylcarbonyl,
(42) imidazol-4-ylcarbonyl,
(43) 5,5-dimethyloxazolidine-2,4-dion-3-ylethyl,
(44) 5,5-dimethyloxazolidine-2,4-dion-3-ylpropyl,
(45) 6-chloropyridazin-3-yl,
(46) 6-acetylaminopyridazin-3-yl,
(47) 5-methyl-1,3,4-thiadiazol-2-yl,
(48) 5-acetylaminopyridin-2-yl,
(49) 4-nitrothiazol-2-yl,
(50) 2,2,2-trichloroethoxycarbonyl,
(51) ethylsulfonyl,
(52) cyclopropylsulfonyl,
(53) isobutylsulfonyl,
(54) 1-benzyloxycarbonylpiperidin-4-ylsulfonyl,
(55) 1-hydroxymethylcarbonylpiperidin-4-ylsulfonyl,
(56) ethoxyethylsulfonyl,
(57) hydroxyethylsulfonyl,
(58) morpholin-1-ylethylsulfonyl,
(59) 5,5-dimethyloxazolidine-2,4-dion-3-ylethylsulfonyl,
(60) indol-6-ylcarbonyl,
(61) benzimidazol-6-ylcarbonyl,
(62) benzthiazol-2-ylcarbonyl,
(63) thiazol-2-ylcarbonyl,
(64) cyclopropylaminocarbonylmethyl,
(65) 4-hydroxypiperidin-1-ylcarbonylmethyl,
(66) 1-tert-butoxycarbonylpiperidin-4-yl,
(67) piperidin-4-yl,
(68) 1-isopropoxycarbonylpiperidin-4-yl,
(69) 1-aminocarbonylcarbonylpiperidin-4-yl,
(70) 1-carbamoylpiperidin-4-yl,
(71) 1-hydroxymethylcarbonylpiperidin-4-yl,
(72) 1-(1-hydroxy-1-methylethylcarbonyl)piperidin-4-yl,
(73) 1-carbamoyl-1-methylethyl,
(74) 1-acetylpiperidin-4-ylmethyl,
(75) 4-carbamoylpiperidin-1-ylcarbonylmethyl,
(76) 3-oxopiperazin-1-ylcarbonylmethyl,
(77) methoxycarbonylethyl,
(78) carbamoylethyl,
(79) cyclopenten-3-on-1-yl,
(80) 4,4-dimethylcyclohexen-3-on-1-yl,
(81) 2-hydroxy-1-methylethyl,
(82) 1-carbamoylethyl,
(83) 2-methylsulfonyl-1-methylethyl,
(84) 3-hydroxy-3-methylbutyl,
(85) 4-acetylaminobenzoyl,
(86) 4-cyanobenzoyl,
(87) 4-carbamoylbenzoyl,
(88) 10-camphorsulfonyl,
(89) 6-chloropyridazin-3-yl,
(90) pyridin-2-yl,
(91) 1-(1-hydroxy-1-methylethylcarbonyl)piperidin-4-ylcarbonyl,
(92) 1-methoxymethylcarbonylpiperidin-4-ylcarbonyl,
(93) 1-aminocarbonylpiperidin-4-ylcarbonyl,
(94) 1-aminocarbonylcarbonylpiperidin-4-ylcarbonyl,
(95) 1-isopropylpiperidin-4-ylcarbonyl,
(96) 1-(1-hydroxycyclopropylcarbonyl)piperidin-4-ylcarbonyl,
(97) tetrazol-5-ylmethylcarbonyl,
(98) 1-(tetrazol-1-ylmethylcarbonyl)piperidin-4-ylcarbonyl,
(99) 1-(acetylaminomethylcarbonyl)piperidin-4-ylcarbonyl,
(100) 2-hydroxyethylcarbonyl,
(101) thiomorpholine-1,1-dioxido-4-ylmethylcarbonyl,
(102) dimethylaminosulfonyl,
(103) 4-acetylaminophenylsulfonyl,
(104) pyridin-2-ylsulfonyl,
(105) pyridin-3-ylsulfonyl,
(106) 6-chloropyridin-3-ylsulfonyl,
(107) 6-(morpholin-1-yl)pyridin-3-ylsulfonyl,
(108) piperidin-3-ylcarbonyl,
(109) 1-tert-butoxycarbonylpiperidin-3-ylcarbonyl,
(110) 1-methoxycarbonylpiperidin-3-ylcarbonyl,
(111) 1-acetylpiperidin-3-ylcarbonyl,
(112) 1-hydroxymethylcarbonylpiperidin-3-ylcarbonyl,
(113) 1-(1-hydroxy-1-methylethylcarbonyl)piperidin-3-ylcarbonyl,
(114) 1-methylsulfonylmethylcarbonylpiperidin-3-ylcarbonyl,
(115) 1-methylsulfonylpiperidin-3-ylcarbonyl,
(116) 1-aminocarbonylcarbonylpiperidin-3-ylcarbonyl,
(117) 4-hydroxymethylcarbonylaminocyclohexan-1-ylcarbonyl,
(118) 4-methoxymethylcarbonylaminocyclohexan-1-ylcarbonyl,
(119) 4-aminocarbonylaminocyclohexan-1-ylcarbonyl,
(120) 4-aminocarbonylcarbonylaminocyclohexan-1-ylcarbonyl,
(121) 4-methylsulfonylaminocyclohexan-1-ylcarbonyl,
(122) tetrahydrofuran-3-ylcarbonyl,
(123) thiazolidin-2-on-4-ylcarbonyl,
(124) 1-tert-butoxycarbonylpyrrolidin-3-ylcarbonyl,
(125) 1-methoxycarbonylpyrrolidin-3-ylcarbonyl, or
(126) 1-hydroxymethylcarbonylpyrrolidin-3-ylcarbonyl;
R² is methyl or cyclopropyl;
R³ is a hydrogen atom or methyl;
R⁴ is a chlorine atom or trifluoromethyl;
R⁵ is a chlorine atom or trifluoromethyl; and a group represented by

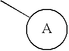

is a group represented by the formula:

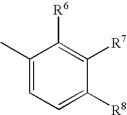

wherein
R⁶ is a hydrogen atom, methyl, ethyl or isopropyl;
R⁷ is a hydrogen atom, methyl or chlorine atom; and
R⁸ is a hydrogen atom, a fluorine atom, a chlorine atom or methyl; or
3-methylthiophen-2-yl;
(hereinafter sometimes to be abbreviated as compound (I)) or a salt thereof.
[2] The compound of the above-mentioned [1], wherein the partial structure:

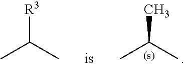

[3] A compound represented by the formula:

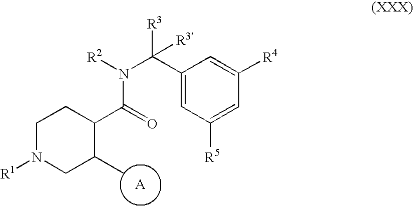

wherein
R¹ is a hydrogen atom, a hydrocarbon group optionally having substituent(s), an acyl group or a heterocyclic group optionally having substituent(s);
R² is an optionally halogenated C₁₋₆ alkyl group;
R³ and R^{3′} are each independently a hydrogen atom or methyl, or
R³ and R^{3′} are optionally bonded to each other to form a ring together with a carbon atom bonded thereto;
R⁴ is a chlorine atom or trifluoromethyl;
R⁵ is a chlorine atom or trifluoromethyl; and a group represented by the formula:

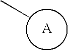

is a heterocyclic group optionally having substituent(s); (hereinafter sometimes to be abbreviated as compound (XXX)), or a salt thereof.
[4] A compound represented by the formula:

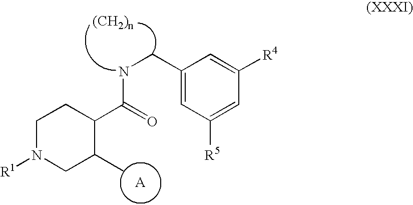

wherein
R¹ is a hydrogen atom, a hydrocarbon group optionally having substituent(s), an acyl group or a heterocyclic group optionally having substituent(s);
R⁴ is a chlorine atom or trifluoromethyl;
R⁵ is a chlorine atom or trifluoromethyl; a group represented by the formula:

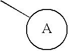

is a group represented by the formula:

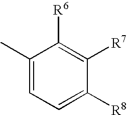

wherein
R⁶ is a hydrogen atom, methyl, ethyl or isopropyl;
R⁷ is a hydrogen atom, methyl or a chlorine atom; and
R⁸ is a hydrogen atom, a fluorine atom, a chlorine atom or methyl; and
n is an integer of 3 to 6;
(hereinafter sometimes to be abbreviated as compound (XXXI)) or a salt thereof.
[5] An optically active compound represented by the formula:

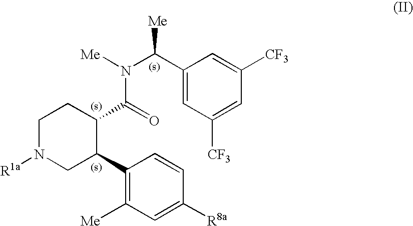

wherein
R^{1a} is
(1) 5,5-dimethyloxazolidine-2,4-dion-3-ylmethylcarbonyl,
(2) 1-hydroxymethylcarbonylpiperidin-4-ylcarbonyl,
(3) carbamoylmethyl,
(4) pyrimidin-5-ylcarbonyl,
(5) methylsulfonylethylcarbonyl,
(6) cyclopropylsulfonyl,
(7) aminocarbonylcarbonyl,
(8) methylsulfonyl, or
(9) methylsulfonylethyl;
R^{8a} is
(1) a hydrogen atom, or
(2) a fluorine atom; and
(s) shows that the steric configuration of an asymmetric carbon is an S-configuration;
(hereinafter sometimes to be abbreviated as compound (II)), or a salt thereof.
[6] (3R,4R)—N-[3,5-bis(Trifluoromethyl)benzyl]-1-{[1-(hydroxyacetyl)piperidin-4-yl]carbonyl}-N-methyl-3-(2-methylphenyl)piperidine-4-carboxamide or a salt thereof.
[7] (3S,4S)—N-[3,5-bis(trifluoromethyl)benzyl]-1-{[1-(hydroxyacetyl)piperidin-4-yl]carbonyl}-N-methyl-3-(2-methylphenyl)piperidine-4-carboxamide or a salt thereof.
[8] (3S,4S)—N-[3,5-bis(trifluoromethyl)benzyl]-1-({1-[(2S)-2-hydroxypropanoyl]piperidin-4-yl}carbonyl)-N-methyl-3-(2-methylphenyl)piperidine-4-carboxamide or a salt thereof.
[9] (3S,4S)—N-[3,5-bis(trifluoromethyl)benzyl]-1-({1-[(2R)-2-hydroxypropanoyl]piperidin-4-yl}carbonyl)-N-methyl-3-(2-methylphenyl)piperidine-4-carboxamide or a salt thereof.
[10] (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-N-methyl-3-(2-methylphenyl)piperidine-4-carboxamide or a salt thereof.
[11] (3S,4S)—N-[3,5-bis(trifluoromethyl)benzyl]-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.
[12] (3R*,4R*)—N-[3,5-bis(trifluoromethyl)benzyl]-1-[2-(cyclopropylamino)-2-oxoethyl]-3-(4-fluoro-2-methylphenyl)-N-methylpiperidine-4-carboxamide or a salt thereof.
[13] (3R*,4R*)—N-[3,5-bis(trifluoromethyl)benzyl]-3-(4-fluoro-2-methylphenyl)-1-[2-(4-hydroxypiperidin-1-yl)-2-oxoethyl]-N-methylpiperidine-4-carboxamide or a salt thereof.
[14] (3R*,4R*)-1′-[amino(oxo)acetyl)-N-[3,5-bis(trifluoromethyl)benzyl]-3-(4-fluoro-2-methylphenyl)-N-methyl-1,4′-bipiperidine-4-carboxamide or a salt thereof.
[15] (3S,4S)—N⁴-[3,5-bis(trifluoromethyl)benzyl]-3-(4-fluoro-2-methylphenyl)-N⁴-methyl-1,4′-bipiperidine-1′,4-dicarboxamide or a salt thereof.
[16] (3R*,4R*)-1-(2-amino-2-oxoethyl)-N-[3,5-bis(trifluoromethyl)benzyl]-3-(4-fluoro-2-methylphenyl)-N-methylpiperidine-4-carboxamide or a salt thereof.
[17] (3R*,4R*)-1-(2-amino-1,1-dimethyl-2-oxoethyl)-N-[3,5-bis(trifluoromethyl)benzyl]-3-(4-fluoro-2-methylphenyl)-N-methylpiperidine-4-carboxamide or a salt thereof.
[18] (3R*,4R*)—N-[3,5-bis(trifluoromethyl)benzyl]-3-(4-fluoro-2-methylphenyl)-N-methyl-1-(3-oxocyclopent-1-en-1-yl)piperidine-4-carboxamide or a salt thereof.
[19] (3R*,4R*)—N-[3,5-bis(trifluoromethyl)benzyl]-1-(4,4-dimethyl-3-oxocyclohex-1-en-1-yl)-3-(4-fluoro-2-methylphenyl)-N-methylpiperidine-4-carboxamide or a salt thereof.
[20] (3R*,4R*)—N-[3,5-bis(trifluoromethyl)benzyl]-3-(4-fluoro-2-methylphenyl)-1-(2-hydroxy-1-methylethyl)-N-methylpiperidine-4-carboxamide or a salt thereof.
[21] (3R*,4R*)—N-[3,5-bis(trifluoromethyl)benzyl]-3-(4-fluoro-2-methylphenyl)-1-(3-hydroxy-3-methylbutyl)-N-methylpiperidine-4-carboxamide or a salt thereof.
[22] (3R*,4R*)-1-[5-(acetylamino)pyridin-2-yl]-N-[3,5-bis(trifluoromethyl)benzyl]-3-(4-fluoro-2-methylphenyl)-N-methylpiperidine-4-carboxamide or a salt thereof.
[23] (3R*,4R*)—N-[3,5-bis(trifluoromethyl)benzyl]-1-(ethylsulfonyl)-3-(4-fluoro-2-methylphenyl)-N-methylpiperidine-4-carboxamide or a salt thereof.
[24] (3R*,4R*)—N-[3,5-bis(trifluoromethyl)benzyl]-1-[(2-ethoxyethyl)sulfonyl]-3-(4-fluoro-2-methylphenyl)-N-methylpiperidine-4-carboxamide or a salt thereof.
[25] (3R*,4R*)—N-[3,5-bis(trifluoromethyl)benzyl]-3-(4-fluoro-2-methylphenyl)-N-methyl-1-[(2-morpholin-4-ylethyl)sulfonyl]piperidine-4-carboxamide or a salt thereof.
[26] (3R*,4R*)—N-[3,5-bis(trifluoromethyl)benzyl]-1-(dimethylsulfamoyl)-3-(4-fluoro-2-methylphenyl)-N-methylpiperidine-4-carboxamide or a salt thereof.
[27] (3R*,4R*)—N-[3,5-bis(trifluoromethyl)benzyl]-3-(4-fluoro-2-methylphenyl)-N-methyl-1-(pyridin-2-ylsulfonyl)piperidine-4-carboxamide or a salt thereof.
[28] (3S,4S)—N-[3,5-bis(trifluoromethyl)benzyl]-3-(4-fluoro-2-methylphenyl)-1-{[1-(hydroxyacetyl)piperidin-4-yl]carbonyl}-N-methylpiperidine-4-carboxamide or a salt thereof.
[29] (3S,4S)—N-[3,5-bis(trifluoromethyl)benzyl]-3-(4-fluoro-2-methylphenyl)-1-({1-[(2S)-2-hydroxypropanoyl]piperidin-4-yl}carbonyl)-N-methylpiperidine-4-carboxamide or a salt thereof.
[30] (3S,4S)—N-[3,5-bis(trifluoromethyl)benzyl]-3-(4-fluoro-2-methylphenyl)-1-({1-[(2R)-2-hydroxypropanoyl]piperidin-4-yl}carbonyl)-N-methylpiperidine-4-carboxamide or a salt thereof.
[31] (3S,4S)—N-{(1S)-1-[3,5-bis(trifluoromethyl)phenyl]ethyl}-3-(4-fluoro-2-methylphenyl)-1-{[1-(hydroxyacetyl)piperidin-4-yl]carbonyl}-N-methylpiperidine-4-carboxamide or a salt thereof.
[32] (3S,4S)—N-{(1R)-1-[3,5-bis(trifluoromethyl)phenyl]ethyl}-3-(4-fluoro-2-methylphenyl)-1-{[1-(hydroxyacetyl)piperidin-4-yl]carbonyl}-N-methylpiperidine-4-carboxamide or a salt thereof.
[33] (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.
[34] (3S,4S)—N-{(1R)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.
[35] (3S,4S)-1-(2-amino-2-oxoethyl)-N-{(1S)-1-[3,5-bis(trifluoromethyl)phenyl]ethyl}-3-(4-fluoro-2-methylphenyl)-N-methylpiperidine-4-carboxamide or a salt thereof.
[36] (3S,4S)-1-(2-amino-2-oxoethyl)-N-{(1R)-1-[3,5-bis(trifluoromethyl)phenyl]ethyl}-3-(4-fluoro-2-methylphenyl)-N-methylpiperidine-4-carboxamide or a salt thereof.
[37] (3S,4S)—N-{(1S)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide or a salt thereof.
[38] (3S,4S)—N-{(1R)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide or a salt thereof.
[39] (3S,4S)—N-{(1S)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide or a salt thereof.
[40] (3S,4S)—N-{(1R)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide or a salt thereof.
[41] (3S,4S)—N-{(1S)-1-[3,5-bis(trifluoromethyl)phenyl]ethyl}-1-(cyclopropylsulfonyl)-3-(4-fluoro-2-methylphenyl)-N-methylpiperidine-4-carboxamide or a salt thereof.
[42] (3R*,4R*)-1-(2-amino-2-oxoethyl)-N-[3,5-bis(trifluoromethyl)benzyl]-N-cyclopropyl-3-(4-fluoro-2-methylphenyl)piperidine-4-carboxamide or a salt thereof.
[43] N-{(1S)-1-[3,5-bis(trifluoromethyl)phenyl]ethyl}-1-{[1-(hydroxyacetyl)piperidin-4-yl]carbonyl}-N-methyl-3-(3-methylthiophen-2-yl)piperidine-4-carboxamide or a salt thereof.
[44] N-{(1S)-1-[3,5-bis(trifluoromethyl)phenyl]ethyl}-1-{[1-(hydroxyacetyl)piperidin-4-yl]carbonyl}-N-methyl-3-(3-methylthiophen-2-yl)piperidine-4-carboxamide or a salt thereof.
[45] (3S,4S)-1-[amino(oxo)acetyl]-N-{(1S)-1-[3,5-bis(trifluoromethyl)phenyl]ethyl}-3-(4-fluoro-2-methylphenyl)-N-methylpiperidine-4-carboxamide or a salt thereof.
[46] (3S,4S)—N-{(1S)-1-[3,5-bis(trifluoromethyl)phenyl]ethyl}-3-(4-fluoro-2-methylphenyl)-N-methyl-1-(methylsulfonyl)piperidine-4-carboxamide or a salt thereof.
[47] (3S,4S)—N-{(1S)-1-[3,5-bis(trifluoromethyl)phenyl]ethyl}-3-(4-fluoro-2-methylphenyl)-N-methyl-1-[2-(methylsulfonyl)ethyl]piperidine-4-carboxamide or a salt thereof.
[48] A prodrug of the compound of any one of the above-mentioned [1] to [47].
[49] A pharmaceutical composition comprising the compound of any one of the above-mentioned [1] to [47], or a prodrug thereof.
[50] The pharmaceutical composition of the above-mentioned [49], which is a tachykinin receptor antagonist.
[51] The pharmaceutical composition of the above-mentioned [49], which is an agent for the prophylaxis or treatment of a lower urinary tract disease, a gastrointestinal disease or a central nervous system disease.
[52] The pharmaceutical composition of the above-mentioned [49], which is an agent for the prophylaxis or treatment of overactive bladder, irritable bowel syndrome, inflammatory bowel disease, vomiting, nausea, depression, anxiety neurosis, anxiety, pelvic visceral pain or interstitial cystitis.
[53] A method for the prophylaxis or treatment of a lower urinary tract disease, a gastrointestinal disease or a central nervous system disease in a mammal, which comprises administering an effective amount of the compound of any one of the above-mentioned [1] to [47], a salt thereof, or a prodrug thereof.
[54] Use of the compound of any one of the above-mentioned [1] to [47], a salt thereof or a prodrug thereof for the production of an agent for the prophylaxis or treatment of a lower urinary tract disease, a gastrointestinal disease or a central nervous system disease, and the like.

### Effect of the Invention

Compound (I), compound (II), compound (XXX) and compound (XXXI), salts thereof and prodrugs thereof have a high tachykinin receptor antagonistic action, particularly, a high Substance P receptor antagonistic action, superior drug efficacy sustainability (metabolic stability), and low toxicity (e.g. vascular toxicity), are safe as pharmaceutical agents, and least impact on other agents. Accordingly, compound (I), compound (II), compound (XXX) and compound (XXXI), salts thereof and prodrugs thereof are useful as pharmaceutical agents, for example, tachykinin receptor antagonists, agents for the prophylaxis or treatment of lower urinary tract diseases and the like.

### DETAILED DESCRIPTION OF THE INVENTION

The substituents of compound (I) are explained in the following.

R¹ is (1) carbamoylmethyl, (2) methylsulfonylethylcarbonyl, (3) aminosulfonylpropylcarbonyl, (4) phenylsulfonylethylcarbonyl, (5) pyridin-2-ylcarbonyl, (6) 5-methoxycarbonylpyridin-2-ylcarbonyl, (7) 5-hydroxypyridin-2-ylcarbonyl, (8) 6-hydroxypyridin-2-ylcarbonyl, (9) 5-bromopyridin-2-ylcarbonyl, (10) 6-methylpyridin-2-ylcarbonyl, (11) 5-carbamoylpyridin-2-ylcarbonyl, (12) 2-aminopyridin-5-ylcarbonyl, (13) 2-acetylaminopyridin-5-ylcarbonyl, (14) pyridin-3-ylcarbonyl, (15) pyrazin-2-ylcarbonyl, (16) pyrimidin-5-ylcarbonyl, (17) 2-aminopyrimidin-5-ylcarbonyl, (18) 2-acetylaminopyrimidin-5-ylcarbonyl, (19) 2-methoxycarbonylaminopyrimidin-5-ylcarbonyl, (20) azetidin-3-ylcarbonyl, (21) 1-tert-butoxycarbonylazetidin-3-ylcarbonyl, (22) 1-(1-hydroxy-1-methylethylcarbonyl)azetidin-3-ylcarbonyl, (23) 1-hydroxymethylcarbonylazetidin-3-ylcarbonyl, (24) adamantan-1-ylcarbonyl, (25) 1-carbamoyl-1-methylethylcarbonyl, (26) dimethylaminomethylcarbonyl, (27) 1,2,3,4-tetrahydropyrimidine-2,4-dion-1-ylmethylcarbonyl, (28) 2,3,5,6-tetrahydropyrimidine-2,6-dion-4-ylmethylcarbonyl, (29) 1-hydroxymethylcarbonylpiperidin-4-ylcarbonyl, (30) 1-ethoxycarbonylpiperidin-4-ylcarbonyl, (31) 1-isopropoxycarbonylpiperidin-4-ylcarbonyl, (32) 1-cyclopropylsulfonylpiperidin-4-ylcarbonyl, (33) 1-(1-hydroxyethylcarbonyl)piperidin-4-ylcarbonyl, (34) 5,5-dimethyloxazolidine-2,4-dion-3-ylmethylcarbonyl, (35) 5,5-dimethyloxazolidine-2,4-dion-3-ylethylcarbonyl, (36) oxazolidine-2,4-dion-3-ylmethylcarbonyl, (37) 1-(2-hydroxy-2-methylpropylcarbonyl)piperidin-4-ylcarbonyl, (38) 3-methylimidazolidine-4,4-dimethyl-2,5-dion-1-ylmethylcarbonyl, (39) 4,4-dimethylpiperidine-2,6-dion-1-ylmethylcarbonyl, (40) morpholine-2,6-dion-1-ylmethylcarbonyl, (41) imidazol-2-ylcarbonyl, (42) imidazol-4-ylcarbonyl, (43) 5,5-dimethyloxazolidine-2,4-dion-3-ylethyl, (44) 5,5-dimethyloxazolidine-2,4-dion-3-ylpropyl, (45) 6-chloropyridazin-3-yl, (46) 6-acetylaminopyridazin-3-yl, (47) 5-methyl-1,3,4-thiadiazol-2-yl, (48) 5-acetylaminopyridin-2-yl, (49) 4-nitrothiazol-2-yl, (50) 2,2,2-trichloroethoxycarbonyl, (51) ethylsulfonyl, (52)cyclopropylsulfonyl, (53) isobutylsulfonyl, (54) 1-benzyloxycarbonylpiperidin-4-ylsulfonyl, (55) 1-hydroxymethylcarbonylpiperidin-4-ylsulfonyl, (56) ethoxyethylsulfonyl, (57) hydroxyethylsulfonyl, (58) morpholin-1-ylethylsulfonyl, (59) 5,5-dimethyloxazolidine-2,4-dion-3-ylethylsulfonyl, (60) indol-6-ylcarbonyl, (61) benzimidazol-6-ylcarbonyl, (62) benzthiazol-2-ylcarbonyl, (63) thiazol-2-ylcarbonyl, (64) cyclopropylaminocarbonylmethyl, (65) 4-hydroxypiperidin-1-ylcarbonylmethyl, (66) 1-tert-butoxycarbonylpiperidin-4-yl, (67) piperidin-4-yl, (68) 1-isopropoxycarbonylpiperidin-4-yl, (69) 1-aminocarbonylcarbonylpiperidin-4-yl, (70) 1-carbamoylpiperidin-4-yl, (71) 1-hydroxymethylcarbonylpiperidin-4-yl, (72) 1-(1-hydroxy-1-methylethylcarbonyl)piperidin-4-yl, (73) 1-carbamoyl-1-methylethyl, (74) 1-acetylpiperidin-4-ylmethyl, (75) 4-carbamoylpiperidin-1-ylcarbonylmethyl, (76) 3-oxopiperazin-1-ylcarbonylmethyl, (77) methoxycarbonylethyl, (78) carbamoylethyl, (79) cyclopenten-3-on-1-yl, (80) 4,4-dimethylcyclohexen-3-on-1-yl, (81) 2-hydroxy-1-methylethyl, (82) 1-carbamoylethyl, (83) 2-methylsulfonyl-1-methylethyl, (84) 3-hydroxy-3-methylbutyl, (85) 4-acetylaminobenzoyl, (86) 4-cyanobenzoyl, (87) 4-carbamoylbenzoyl, (88) 10-camphorsulfonyl, (89) 6-chloropyridazin-3-yl, (90) pyridin-2-yl, (91) 1-(1-hydroxy-1-methylethylcarbonyl)piperidin-4-ylcarbonyl, (92) 1-methoxymethylcarbonylpiperidin-4-ylcarbonyl, (93) 1-aminocarbonylpiperidin-4-ylcarbonyl, (94) 1-aminocarbonylcarbonylpiperidin-4-ylcarbonyl, (95) 1-isopropylpiperidin-4-ylcarbonyl, (96) 1-(1-hydroxycyclopropylcarbonyl)piperidin-4-ylcarbonyl, (97) tetrazol-5-ylmethylcarbonyl, (98) 1-(tetrazol-1-ylmethylcarbonyl)piperidin-4-ylcarbonyl, (99) 1-(acetylaminomethylcarbonyl)piperidin-4-ylcarbonyl, (100) 2-hydroxyethylcarbonyl, (101) thiomorpholine-1,1-dioxido-4-ylmethylcarbonyl, (102) dimethylaminosulfonyl, (103) 4-acetylaminophenylsulfonyl, (104) pyridin-2-ylsulfonyl, (105) pyridin-3-ylsulfonyl, (106) 6-chloropyridin-3-ylsulfonyl, (107) 6-(morpholin-1-yl)pyridin-3-ylsulfonyl, (108) piperidin-3-ylcarbonyl, (109) 1-tert-butoxycarbonylpiperidin-3-ylcarbonyl, (110) 1-methoxycarbonylpiperidin-3-ylcarbonyl, (111) 1-acetylpiperidin-3-ylcarbonyl, (112) 1-hydroxymethylcarbonylpiperidin-3-ylcarbonyl, (113) 1-(1-hydroxy-1-methylethylcarbonyl)piperidin-3-ylcarbonyl, (114) 1-methylsulfonylmethylcarbonylpiperidin-3-ylcarbonyl, (115) 1-methylsulfonylpiperidin-3-ylcarbonyl, (116) 1-aminocarbonylcarbonylpiperidin-3-ylcarbonyl, (117) 4-hydroxymethylcarbonylaminocyclohexan-1-ylcarbonyl, (118) 4-methoxymethylcarbonylaminocyclohexan-1-ylcarbonyl, (119) 4-aminocarbonylaminocyclohexan-1-ylcarbonyl, (120) 4-aminocarbonylcarbonylaminocyclohexan-1-ylcarbonyl, (121) 4-methylsulfonylaminocyclohexan-1-ylcarbonyl, (122) tetrahydrofuran-3-ylcarbonyl, (123) thiazolidin-2-on-4-ylcarbonyl, (124) 1-tert-butoxycarbonylpyrrolidin-3-ylcarbonyl, (125) 1-methoxycarbonylpyrrolidin-3-ylcarbonyl, or (126) 1-hydroxymethylcarbonylpyrrolidin-3-ylcarbonyl.

Of these, R¹ is preferably (1) 1-hydroxymethylcarbonylpiperidin-4-ylcarbonyl, (2) 1-(1-hydroxyethylcarbonyl)piperidin-4-ylcarbonyl, (3) 5,5-dimethyloxazolidine-2,4-dion-3-ylmethylcarbonyl, (4) carbamoylmethyl, (5) pyrimidin-5-ylcarbonyl, (6) methylsulfonylethylcarbonyl or (7) cyclopropylsulfonyl.

In compound (I), R² is methyl or cyclopropyl. Of these, methyl is preferable.

In compound (I), R³ is a hydrogen atom or methyl.

In compound (I), R⁴ is a chlorine atom or trifluoromethyl.

In compound (I), R⁵ is a chlorine atom or trifluoromethyl.

In compound (I), the group represented by the formula

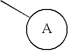

is a group represented by the formula:

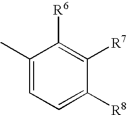

wherein R⁶ is a hydrogen atom, methyl, ethyl or isopropyl, R⁷ is a hydrogen atom, methyl or a chlorine atom, and R⁸ is a hydrogen atom, a fluorine atom, a chlorine atom or methyl, or 3-methylthiophen-2-yl.

In compound (I), the partial structure:

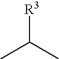

is preferably

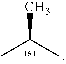

That is, the absolute configuration of the asymmetric carbon to which R³ (or CH₃) is bonded is preferably an S-configuration.

Specifically, compound (I) is preferably

(3S,4S)—N-[3,5-bis(trifluoromethyl)benzyl]-1-[[1-glycoloylpiperidin-4-yl]carbonyl]-N-methyl-3-(2-methylphenyl)piperidine-4-carboxamide,

(3S,4S)—N-[3,5-bis(trifluoromethyl)benzyl]-1-({1-[(2S)-2-hydroxypropanoyl]piperidin-4-yl}carbonyl)-N-methyl-3-(2-methylphenyl)piperidine-4-carboxamide,

(3S,4S)—N-[3,5-bis(trifluoromethyl)benzyl]-1-({1-[(2R)-2-hydroxypropanoyl]piperidin-4-yl}carbonyl)-N-methyl-3-(2-methylphenyl)piperidine-4-carboxamide,

(3S,4S)—N-[3,5-bis(trifluoromethyl)benzyl]-1-[(5,5-dimethyl-2,4-dioxo-1,3-oxazolidin-3-yl)acetyl]-3-(4-fluoro-2-methylphenyl)-N-methylpiperidine-4-carboxamide,

(3S,4S)—N-[3,5-bis(trifluoromethyl)benzyl]-3-(4-fluoro-2-methylphenyl)-1-[(1-glycoloylpiperidin-4-yl)carbonyl]-N-methylpiperidine-4-carboxamide,

(3S,4S)—N-[3,5-bis(trifluoromethyl)benzyl]-3-(4-fluoro-2-methylphenyl)-1-({1-[(2S)-2-hydroxypropanoyl]piperidin-4-yl}carbonyl)-N-methylpiperidine-4-carboxamide,

(3S,4S)—N-[3,5-bis(trifluoromethyl)benzyl]-3-(4-fluoro-2-methylphenyl)-1-({1-[(2R)-2-hydroxypropanoyl]piperidin-4-yl}carbonyl)-N-methylpiperidine-4-carboxamide and salts thereof and the like.

Specifically, the following compounds and salts thereof are preferable.

(3S,4S)—N-[3,5-bis(trifluoromethyl)benzyl]-1-[[1-glycoloylpiperidin-4-yl]carbonyl]-N-methyl-3-(2-methylphenyl)piperidine-4-carboxamide,

(3S,4S)—N-[3,5-bis(trifluoromethyl)benzyl]-1-({1-[(2S)-2-hydroxypropanoyl]piperidin-4-yl}carbonyl)-N-methyl-3-(2-methylphenyl)piperidine-4-carboxamide, and

(3S,4S)—N-[3,5-bis(trifluoromethyl)benzyl]-1-({1-[(2R)-2-hydroxypropanoyl]piperidin-4-yl}carbonyl)-N-methyl-3-(2-methylphenyl)piperidine-4-carboxamide.

The substituents of compound (II) are explained in the following.

R^{1a} is (1) 5,5-dimethyloxazolidine-2,4-dion-3-ylmethylcarbonyl, (2) 1-hydroxymethylcarbonylpiperidin-4-ylcarbonyl, (3) carbamoylmethyl, (4) pyrimidin-5-ylcarbonyl, (5) methylsulfonylethylcarbonyl, (6) cyclopropylsulfonyl, (7) aminocarbonylcarbonyl, (8) methylsulfonyl, or (9) methylsulfonylethyl.

Specifically, R^{1a} is preferably (1) 5,5-dimethyloxazolidine-2,4-dion-3-ylmethylcarbonyl, (2) 1-hydroxymethylcarbonylpiperidin-4-ylcarbonyl, or (3) aminocarbonylcarbonyl.

R^{8a} is a hydrogen atom or a fluorine atom.

Specifically, as compound (II), the following compounds and salts thereof are preferable.

(3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-N-methyl-3-(2-methylphenyl)piperidine-4-carboxamide,

(3S,4S)—N-{(1S)-1-[3,5-bis(trifluoromethyl)phenyl]ethyl}-3-(4-fluoro-2-methylphenyl)-1-[(1-glycoloylpiperidin-4-yl)carbonyl]-N-methylpiperidine-4-carboxamide,

(3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide,

(3S,4S)-1-(2-amino-2-oxoethyl)-N-{(1S)-1-[3,5-bis(trifluoromethyl)phenyl]ethyl}-3-(4-fluoro-2-methylphenyl)-N-methylpiperidine-4-carboxamide,

(3S,4S)—N-{(1S)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide,

(3S,4S)—N-{(1S)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide,

(3S,4S)—N-{(1S)-1-[3,5-bis(trifluoromethyl)phenyl]ethyl}-1-(cyclopropylsulfonyl)-3-(4-fluoro-2-methylphenyl)-N-methylpiperidine-4-carboxamide,

(3R*,4S*)—N-{(1S)-1-[3,5-bis(trifluoromethyl)phenyl]ethyl}-1-[(1-glycoloylpiperidin-4-yl)carbonyl]-N-methyl-3-(3-methyl-2-thienyl)piperidine-4-carboxamide (high polarity),

(3S,4S)-1-[amino(oxo)acetyl]-N-{(1S)-1-[3,5-bis(trifluoromethyl)phenyl]ethyl}-3-(4-fluoro-2-methylphenyl)-N-methylpiperidine-4-carboxamide,

(3S,4S)—N-{(1S)-1-[3,5-bis(trifluoromethyl)phenyl]ethyl}-3-(4-fluoro-2-methylphenyl)-N-methyl-1-(methylsulfonyl)piperidine-4-carboxamide, and

(3S,4S)—N-{(1S)-1-[3,5-bis(trifluoromethyl)phenyl]ethyl}-3-(4-fluoro-2-methylphenyl)-N-methyl-1-[2-(methylsulfonyl)ethyl]piperidine-4-carboxamide.

The substituents of compound (XXX) are explained in the following.

In compound (XXX), R¹ is a hydrogen atom, a hydrocarbon group optionally having substituent(s), an acyl group or a heterocyclic group optionally having substituent(s).

The “hydrocarbon group” of the “hydrocarbon group optionally having substituent(s)” for R¹ is, for example, an aliphatic hydrocarbon group, a monocyclic saturated hydrocarbon group, an aromatic hydrocarbon group and the like, with preference given to one having 1 to 16 carbon atoms. Specifically, for example, alkyl, alkenyl, alkynyl, cycloalkyl, aryl and aralkyl and the like are used.

As the “alkyl”, for example, C₁₋₆ alkyl (e.g., methyl, ethyl, propyl, isopropyl, butyl, isobutyl, sec-butyl, tert-butyl, pentyl, hexyl etc.) and the like are preferable, and C₁₋₄ alkyl is more preferable.

As the “alkenyl”, for example, C₂₋₆ alkenyl (e.g., vinyl, allyl, isopropenyl, 1-butenyl, 2-butenyl, 3-butenyl, 2-methyl-2-propenyl, 1-methyl-2-propenyl, 2-methyl-1-propenyl etc.) and the like are preferable.

As the “alkynyl”, for example, C₂₋₆ alkynyl (e.g., ethynyl, propargyl, 1-butynyl, 2-butynyl, 3-butynyl, 1-hexynyl etc.) and the like are preferable.

As the “cycloalkyl”, for example, C₃₋₈ cycloalkyl (e.g., cyclopropyl, cyclobutyl, cyclopentyl, cyclohexyl etc.) and the like are preferable, and C₃₋₆ cycloalkyl is more preferable.

As the “aryl”, for example, C₆₋₁₄ aryl (e.g., phenyl, 1-naphthyl, 2-naphthyl, 2-biphenylyl, 3-biphenylyl, 4-biphenylyl, 2-anthryl etc.) and the like are preferable.

As the “aralkyl”, for example, C₇₋₁₆ aralkyl (e.g., benzyl, phenethyl, diphenylmethyl, 1-naphthylmethyl, 2-naphthylmethyl, 2,2-diphenylethyl, 3-phenylpropyl, 4-phenylbutyl, 5-phenylpentyl etc.) and the like are preferable.

Examples of the “heterocyclic group” of the “heterocyclic group optionally having substituent(s)” for R¹ include a 5- to 14-membered (preferably 5- to 10-membered) monocyclic to tricyclic (preferably monocyclic or bicyclic) aromatic or nonaromatic heterocyclic group containing, besides carbon atom, one or two kinds of 1 to 4 (preferably 1 to 3) hetero atoms selected from nitrogen atom, oxygen atom and sulfur atom, and the like. A 5-membered ring group containing, besides carbon atom, 1 to 4 hetero atoms selected from oxygen atom, sulfur atom and nitrogen atom, for example, 2- or 3-thienyl, 2- or 3-furyl, 1-, 2- or 3-pyrrolyl, 1-, 2- or 3-pyrrolidinyl, 2-, 4- or 5-oxazolyl, 3-, 4- or 5-isoxazolyl, 2-, 4- or 5-thiazolyl, 3-, 4- or 5-isothiazolyl, 3-, 4- or 5-pyrazolyl, 2-, 3- or 4-pyrazolidinyl, 2-, 4- or 5-imidazolyl, 1-, 2- or 4-imidazolidinyl, 1,2,3-triazolyl, 1,2,4-triazolyl, 1H- or 2H-tetrazolyl and the like, a 6-membered ring group containing, besides carbon atom, 1 to 4 hetero atoms selected from oxygen atom, sulfur atom and nitrogen atom, for example, 2-, 3- or 4-pyridyl, N-oxido-2-, 3- or 4-pyridyl, 2-, 4- or 5-pyrimidinyl, N-oxido-2-, 4- or 5-pyrimidinyl, thiomorpholinyl, morpholinyl, piperidino, 2-, 3- or 4-piperidyl, thiopyranyl, 1,4-oxazinyl, 1,4-thiazinyl, 1,3-thiazinyl, piperazinyl, triazinyl, 3- or 4-pyridazinyl, pyrazinyl, N-oxido-3- or 4-pyridazinyl and the like, a bicyclic or tricyclic condensed ring group containing, besides carbon atom, 1 to 4 hetero atoms selected from oxygen atom, sulfur atom and nitrogen atom (preferably, a group formed by condensation of the above-mentioned 5- or 6-membered ring with one or two 5- or 6-membered ring groups containing, besides carbon atom, 1 to 4 hetero atoms selected from oxygen atom, sulfur atom and nitrogen atom), for example, indolyl, benzofuryl, benzothiazolyl, benzoxazolyl, benzimidazolyl, quinolyl, isoquinolyl, phthalazinyl, quinazolinyl, quinoxalinyl, indolizinyl, quinolizinyl, 1,8-naphthyridinyl, dibenzofuranyl, carbazolyl, acrydinyl, phenanthridinyl, chromanyl, phenothiazinyl, phenoxazinyl and the like, and the like are used. Of these, 5- to 7-membered (preferably 5- or 6-membered) aromatic or nonaromatic heterocyclic group containing, besides carbon atom, 1 to 4 hetero atoms selected from oxygen atom, sulfur atom and nitrogen atom is preferable.

The “substituent” of the “hydrocarbon group optionally having substituent(s)” and “heterocyclic group optionally having substituent(s)” for R¹ is, for example, 1 to 3 selected from (1) halogen atom (e.g., fluorine, chlorine, bromine, iodine etc.), (2) nitro, (3) cyano, (4) C₁₋₆ alkyl optionally having 1 to 5 (preferably 1 to 3) halogen atoms (e.g., methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl etc.), (5) C₂₋₆ alkenyl optionally having 1 to 3 halogen atoms (e.g., vinyl, allyl, isopropenyl, butenyl, isobutenyl, sec-butenyl, 3,3,3-trifluoro-1-propenyl, 4,4,4-trifluoro-1-butenyl etc.), (6) C₂₋₆ alkynyl optionally having 1 to 3 halogen atoms (e.g., ethynyl, propargyl, butynyl, 1-hexynyl, 3,3,3-trifluoro-1-propynyl, 4,4,4-trifluoro-1-butynyl etc.), (7) C₃₋₆ cycloalkyl optionally having 1 to 5 (preferably 1 to 3) halogen atoms (e.g., cyclopropyl, cyclobutyl, cyclopentyl, cyclohexyl, 4,4-dichlorocyclohexyl, 2,2,3,3-tetrafluorocyclopentyl, 4-chlorocyclohexyl etc.), (8) C₆₋₁₄ aryl (e.g., phenyl, 1-naphthyl, 2-naphthyl, biphenylyl, 2-anthryl etc.), (9) C₇₋₁₆ aralkyl (e.g., benzyl, phenethyl, diphenylmethyl, 1-naphthylmethyl, 2-naphthylmethyl, 2,2-diphenylethyl, 3-phenylpropyl, 4-phenylbutyl, 5-phenylpentyl etc.), (10) hydroxy, (11) C₁₋₆ alkoxy optionally having 1 to 3 halogen atoms (e.g., methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy, hexyloxy etc.), (12) C₆₋₁₄ aryloxy (e.g., phenyloxy, naphthyloxy etc.), (13) mercapto, (14) C₁₋₆ alkylthio optionally having 1 to 3 halogen atoms (e.g., methylthio, difluoromethylthio, trifluoromethylthio, ethylthio, propylthio, isopropylthio, butylthio, 4,4,4-trifluorobutylthio, pentylthio, hexylthio etc.), (15) C₆₋₁₄ arylthio (e.g., phenylthio, naphthylthio etc.), (16) amino, (17) mono-C₁₋₆ alkylamino (e.g., methylamino, ethylamino etc.), (18) mono-C₆₋₁₄ arylamino (e.g., phenylamino, 1-naphthylamino, 2-naphthylamino etc.), (19) di-C₁₋₆ alkylamino (e.g., dimethylamino, diethylamino etc.), (20) di-C₆₋₁₄ arylamino (e.g., diphenylamino etc.), (21) formyl, (22) C₁₋₆ alkyl-carbonyl (e.g., acetyl, propionyl etc.), (23) C₆₋₁₄ aryl-carbonyl (e.g., benzoyl, 1-naphthoyl, 2-naphthoyl etc.), (24) carboxy, (25) C₁₋₆ alkoxy-carbonyl (e.g., methoxycarbonyl, ethoxycarbonyl, propoxycarbonyl, tert-butoxycarbonyl etc.), (26) C₆₋₁₄ aryloxy-carbonyl (e.g., phenoxycarbonyl etc.), (27) carbamoyl, (28) thiocarbamoyl, (29) mono-C₁₋₆ alkyl-carbamoyl (e.g., methylcarbamoyl, ethylcarbamoyl etc.), (30) di-C₁₋₆ alkyl-carbamoyl (e.g., dimethylcarbamoyl, diethylcarbamoyl, ethylmethylcarbamoyl etc.), (31) C₆₋₁₄ aryl-carbamoyl (e.g., phenylcarbamoyl, 1-naphthylcarbamoyl, 2-naphthylcarbamoyl etc.), (32) C₁₋₆ alkylsulfonyl (e.g., methylsulfonyl, ethylsulfonyl etc.), (33) C₆₋₁₄ arylsulfonyl (e.g., phenylsulfonyl, 1-naphthylsulfonyl, 2-naphthylsulfonyl etc.), (34) C₁₋₆ alkylsulfinyl (e.g., methylsulfinyl, ethylsulfinyl etc.), (35) C₆₋₁₄ arylsulfinyl (e.g., phenylsulfinyl, 1-naphthylsulfinyl, 2-naphthylsulfinyl etc.), (36) formylamino, (37) C₁₋₆ alkyl-carbonylamino (e.g., acetylamino etc.), (38) C₆₋₁₄ aryl-carbonylamino (e.g., benzoylamino, naphthoylamino etc.), (39) C₁₋₆ alkoxy-carbonylamino (e.g., methoxycarbonylamino, ethoxycarbonylamino, propoxycarbonylamino, butoxycarbonylamino etc.), (40) C₁₋₆ alkylsulfonylamino (e.g., methylsulfonylamino, ethylsulfonylamino etc.), (41) C₆₋₁₄ arylsulfonylamino (e.g., phenylsulfonylamino, 2-naphthylsulfonylamino, 1-naphthylsulfonylamino etc.), (42) C₁₋₆ alkyl-carbonyloxy (e.g., acetoxy, propionyloxy etc.), (43) C₆₋₁₄ aryl-carbonyloxy (e.g., benzoyloxy, naphthylcarbonyloxy etc.), (44) C₁₋₆ alkoxy-carbonyloxy (e.g., methoxycarbonyloxy, ethoxycarbonyloxy, propoxycarbonyloxy, butoxycarbonyloxy etc.), (45) mono-C₁₋₆ alkyl-carbamoyloxy (e.g., methylcarbamoyloxy, ethylcarbamoyloxy etc.), (46) di-C₁₋₆ alkyl-carbamoyloxy (e.g., dimethylcarbamoyloxy, diethylcarbamoyloxy etc.), (47) C₆₋₁₄ aryl-carbamoyloxy (e.g., phenylcarbamoyloxy, naphthylcarbamoyloxy etc.), (48) 5 to 7-membered saturated cyclic amino containing, besides carbon atom and one nitrogen atom, one or two kinds of 1 to 4 hetero atoms selected from nitrogen atom, oxygen atom and sulfur atom (e.g., pyrrolidin-1-yl, piperidino, piperazin-1-yl, morpholino, thiomorpholino, hexahydroazepin-1-yl etc.), (49), 5- to 10-membered aromatic heterocyclic group containing, besides carbon atom, one or two kinds of 1 to 4 hetero atoms selected from nitrogen atom, sulfur atom and oxygen atom (e.g., 2-thienyl, 3-thienyl, 2-pyridyl, 3-pyridyl, 4-pyridyl, 2-quinolyl, 3-quinolyl, 4-quinolyl, 5-quinolyl, 8-quinolyl, 1-isoquinolyl, 3-isoquinolyl, 4-isoquinolyl, 5-isoquinolyl, 1-indolyl, 2-indolyl, 3-indolyl, 2-benzothiazolyl, 2-benzo[b]thienyl, 3-benzo[b]thienyl, 2-benzo[b]furanyl, 3-benzo[b]furanyl etc.), (50) C₁₋₃ alkylenedioxy (e.g., methylenedioxy, ethylenedioxy etc.), (51) oxo and the like.

As the “acyl group” for R¹, for example, an acyl group represented by the formula: —(C═O)—R¹¹, —(C═O)—OR¹¹, —(C═O)—NR¹¹R¹², —(C═S)—NHR¹¹ or —SO₂—R¹³ wherein R¹¹ is a hydrogen atom, a hydrocarbon group optionally having substituent(s), a heterocyclic group optionally having substituent(s), a C₁₋₆ alkoxy group, a carbamoyl group, a C₁₋₆ alkoxy-carbonyl group or a C₁₋₆ alkyl-carbamoyl group, R¹² is a hydrogen atom or a C₁₋₆ alkyl group, and R¹³ is a hydrocarbon group optionally having substituent(s) or a heterocyclic group optionally having substituent(s) can be mentioned.

As the “hydrocarbon group optionally having substituent(s)” and “heterocyclic group optionally having substituent(s)” for R¹¹ or R¹³, those similar to the “hydrocarbon group optionally having substituent(s)” and “heterocyclic group optionally having substituent(s)” for R¹ can be used.

As the “C₁₋₆ alkoxy group” for R¹¹, for example, methoxy, ethoxy, propoxy, isopropoxy, butoxy, isobutoxy, sec-butoxy, tert-butoxy, pentyloxy, hexyloxy and the like can be mentioned.

As the “C₁₋₆ alkoxy-carbonyl group” for R¹¹, methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl and the like can be mentioned.

As the “C₁₋₆ alkyl-carbamoyl group” for R¹¹, methylcarbamoyl, ethylcarbamoyl, propylcarbamoyl, isopropylcarbamoyl, butylcarbamoyl, isobutylcarbamoyl, sec-butylcarbamoyl, tert-butylcarbamoyl, pentylcarbamoyl, hexylcarbamoyl and the like can be mentioned.

As the “C₁₋₆ alkyl group” for R¹², for example, methyl, ethyl, propyl, isopropyl, butyl, isobutyl, sec-butyl, tert-butyl, pentyl, hexyl and the like can be mentioned.

As R¹, (1) a hydrogen atom, (2) a C₁₋₆ alkyl-carbonyl group optionally substituted by 1 to 3 hydroxy, (3) a C₁₋₆ alkoxy-carbonyl group, and (4) an aminocarbonylcarbonyl (carbamoylcarbonyl) group are preferable, and a hydrogen atom, glycoloyl, t-butoxycarbonyl, acetyl and aminocarbonylcarbonyl (carbamoylcarbonyl) are more preferable.

In compound (XXX), R² is an optionally halogenated C₁₋₆ alkyl group. As R², a C₁₋₃ alkyl group is preferable, and a methyl group is more preferable.

In compound (XXX), R³ and R^{3′} are each independently a hydrogen atom or methyl, or R³ and R^{3′} are optionally bonded to each other to form a ring together with the carbon atom bonded thereto.

The “ring formed by R³ and R^{3′} bonded to each other, together with the carbon atom bonded thereto” is, for example, cyclopropane ring, cyclobutane ring, cyclopentane ring, cyclohexane ring and the like.

R³ and R^{3′} are preferably a hydrogen atom and a methyl group, respectively, and a combination of one of them being a hydrogen atom and the other being a methyl group is more preferable.

In compound (XXX), R⁴ is a chlorine atom or trifluoromethyl. As R⁴, trifluoromethyl is preferable.

In compound (XXX), R⁵ is a chlorine atom or trifluoromethyl. As R⁵, trifluoromethyl is preferable.

In compound (XXX), the group represented by the formula:

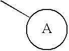

is a heterocyclic group optionally having substituent(s).

As the “heterocyclic group optionally having substituent(s)”, those similar to the “heterocyclic group optionally having substituent(s)” for R¹ explained above can be mentioned.

As the “heterocyclic group optionally having substituent(s)”, a 5- or 6-membered aromatic heterocyclic group containing, besides carbon atom, one or two kinds of 1 to 4 hetero atoms selected from nitrogen atom, oxygen atom and sulfur atom (e.g., furyl, thienyl, pyridyl, imidazolyl, thiazolyl, oxazolyl, thiadiazolyl, triazolyl, tetrazolyl etc.) and the like (said heterocyclic group is optionally having substituent(s)) are preferable, a 5- or 6-membered aromatic heterocyclic group optionally substituted by 1 to 3 C₁₋₆ alkyl is preferable, and a 3-methylthiophen-2-yl group is particularly preferable.

As compound (XXX), the following compound or a salt thereof is specifically preferable.

tert-butyl (3R*,4S*)-4-{[{(1S)-1-[3,5-bis(trifluoromethyl)phenyl]ethyl}[methyl]amino]carbonyl}-3-(3-methyl-2-thienyl)piperidine-1-carboxylate,

N-{(3R*,4S*)-(1S)-1-[3,5-bis(trifluoromethyl)phenyl]ethyl}-N-methyl-3-(3-methyl-2-thienyl)piperidine-4-carboxamide and

(3R*,4S*)-1-[amino(oxo)acetyl]-N-{(1S)-1-[3,5-bis(trifluoromethyl)phenyl]ethyl}-N-methyl-3-(3-methyl-2-thienyl)piperidine-4-carboxamide.

The substituents of compound (XXXI) are explained below.

In compound (XXXI), R¹ is a hydrogen atom, a hydrocarbon group optionally having substituent(s), an acyl group or a heterocyclic group optionally having substituent(s).

As the “hydrocarbon group optionally having substituent(s)”, “acyl group” and “heterocyclic group optionally having substituent(s)” for R¹, those similar to the “hydrocarbon group optionally having substituent(s)”, “acyl group” and “heterocyclic group optionally having substituent(s)” for R¹ in compound (XXX) explained above can be mentioned.

As R¹, (1) a hydrogen atom, (2) a C₁₋₆ alkoxy-carbonyl group, or (3) a C₁₋₆ alkyl-carbonyl group optionally substituted by 1 or 2 C₁₋₆ alkyl-carbonylamino is preferable, and a hydrogen atom, a t-butoxycarbonyl group and an acetylaminomethylcarbonyl group are more preferable.

In compound (XXXI), R⁴ is a chlorine atom or trifluoromethyl. As R⁴, a chlorine atom is preferable.

In compound (XXXI), R⁵ is a chlorine atom or trifluoromethyl. As R⁵, a chlorine atom is preferable.

In compound (XXXI), a group represented by the formula:

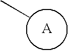

is a group represented by the formula:

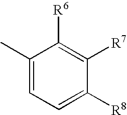

wherein R⁶ is a hydrogen atom, methyl, ethyl or isopropyl, R⁷ is a hydrogen atom, methyl or a chlorine atom, and R⁸ is a hydrogen atom, a fluorine atom, a chlorine atom or methyl. As this group, a 4-fluoro-2-methylphenyl group is preferable.

In compound (XXXI), n is an integer of 3 to 6 and n is preferably 3.

As compound (XXXI), the following compound or a salt thereof is specifically preferable.

tert-butyl (3R*,4R*)-4-{[2-(3,5-dichlorophenyl)pyrrolidin-1-yl]carbonyl}-3-(4-fluoro-2-methylphenyl)piperidine-1-carboxylate,

(3R*,4R*)-4-{[2-(3,5-dichlorophenyl)pyrrolidin-1-yl]carbonyl}-3-(4-fluoro-2-methylphenyl)piperidine, and

N-{2-[(3R*,4R*)-4-{[2-(3,5-dichlorophenyl)pyrrolidin-1-yl]carbonyl}-3-(4-fluoro-2-methylphenyl)piperidin-1-yl]-2-oxoethyl}acetamide.

The salts of compound (I), compound (II), compound (XXX) and compound (XXXI) include, for example, a metal salt, an ammonium salt, a salt with an organic base, a salt with an inorganic acid, a salt with an organic acid, a salt with basic or acidic amino acid etc. Suitable examples of the metal salt include an alkali metal salt such as a sodium salt, a potassium salt etc.; an alkaline earth metal salt such as a calcium salt, a magnesium salt, a barium salt etc.; an aluminum salt etc. Suitable examples of the salts with an organic base include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine etc. Suitable examples of the salts with an inorganic acid include salts with hydrochloric acid, hydrobromic acid, nitric acid, sulfuric acid, phosphoric acid etc. Suitable examples of the salts with an organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid etc. Suitable examples of the salts with basic amino acid include salts with arginine, lysine, ornithine etc. Suitable examples of the-salts with acidic amino acid include salts with asparaginic acid and glutamic acid etc.

Of these, pharmaceutically acceptable salts are preferred. For example, if the compound has an acidic functional group, preferred are inorganic salts such as an alkali metal salt (e.g., sodium salt, potassium salt etc.), an alkaline earth metal salt (e.g., calcium salt, magnesium salt, barium salt etc.), an ammonium salt etc. If the compound has a basic functional group, preferred are salts with an inorganic acid such as hydrochloric acid, hydrobromic acid, nitric acid, sulfuric acid, phosphoric acid etc., or salts with an organic acid such as acetic acid, phthalic acid, fumaric acid, oxalic acid, tartaric acid, maleic acid, citric acid, succinic acid, methanesulfonic acid, p-toluenesulfonic acid etc.

The prodrug of compound (I), compound (II), compound (XXX) or compound (XXXI) of the present invention means a compound which is converted to the compound (I), compound (II), compound (XXX) or compound (XXXI) of the present invention under the physiological condition in the living body by a reaction with an enzyme, a gastric acid, or the like, that is, by enzymatic oxidation, reduction, hydrolysis etc. or by hydrolysis with gastric acid etc.

The prodrug of compound (I), compound (II), compound (XXX) or compound (XXXI) of the present invention includes a compound wherein the amino group of compound (I), compound (II), compound (XXX) or compound (XXXI) of the present invention is modified with acyl, alkyl or phosphoryl (e.g., a compound wherein the amino group of compound (I), compound (II), compound (XXX) or compound (XXXI) of the present invention is modified with eicosanoyl, alanyl, pentylaminocarbonyl, (5-methyl-2-oxo-1,3-dioxolen-4-yl)methoxycarbonyl, tetrahydrofuranyl, pyrrolidylmethyl, pivaloyloxymethyl, tert-butyl etc.), and the like; a compound wherein the hydroxy group of compound (I), compound (II), compound (XXX) or compound (XXXI) of the present invention is modified with acyl, alkyl, phosphoryl or boryl (e.g., a compound wherein the hydroxy group of compound (I), compound (II), compound (XXX) or compound (XXXI) of the present invention is modified with acetyl, palmitoyl, propanoyl, pivaloyl, succinyl, fumaryl, alanyl or dimethylaminomethylcarbonyl etc.) and the like; a compound wherein a carboxyl group of compound (I), compound (II), compound (XXX) or compound (XXXI) of the present invention is modified with ester or amide (e.g., a compound wherein a carboxyl group of compound (I), compound (II), compound (XXX) or compound (XXXI) of the present invention is modified with ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester or methylamide etc.) and the like; etc. These compounds can be prepared by a method known per se from compound (I), compound (II), compound (XXX) or compound (XXXI) of the present invention etc.

In addition, the prodrug of compound (I), compound (II), compound (XXX) or compound (XXXI) of the present invention may be a compound, which is converted into compound (I), compound (II), compound (XXX) or compound (XXXI) of the present invention under the physiological conditions, as described in “Pharmaceutical Research and Development”, Vol. 7 (Drug Design), pp. 163-198 (1990), published by Hirokawa Publishing Co.

Solvate, for example, hydrates of the compound (I), compound (II), compound (XXX) or compound (XXXI) of the present invention and a salt thereof are all included in the scope of the present invention. The compound (I), compound (II), compound (XXX) or compound (XXXI) of the present invention may be labeled with an isotope (e.g., ³H, ¹⁴C, ³⁵S, ¹²⁵I etc.) and the like. In addition, the compound (I), compound (II), compound (XXX) or compound (XXXI) may be a deuterated compound.

If the compound (I), etc. of the present invention has chiral center, isomers such as an enantiomer or a diastereomer may exist. Such isomers and a mixture thereof are all included in the scope of-the present invention. In addition, there can be instances where the conformational isomers are generated in cases, but such isomers or a mixture thereof are also included in compound (I) or a salt thereof of the present invention. Compound (I), etc. is preferably a trans-isomer in view of activity.

The production methods of compound (I), compound (II), compound (XXX) or compound (XXXI) and salts thereof of the present invention are explained in the following.

Compound (I), compound (II), compound (XXX) or compound (XXXI) and salts thereof of the present invention can be produced according to the method described in WO2005/068427 or WO2006/004195.

### [Method A]

Compound (I), compound (II) and salts thereof of the present invention can be produced by subjecting a compound represented by the formula:

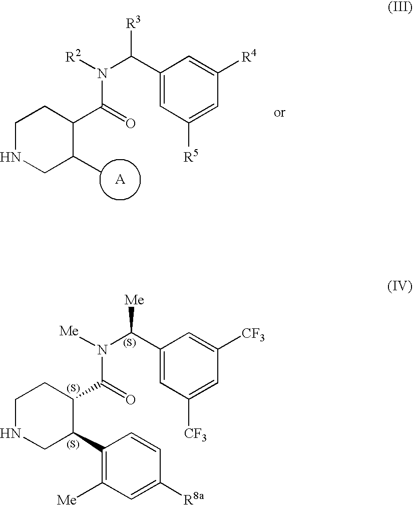

wherein each symbol is as defined above (hereinafter to be referred to as compound (III) or compound (IV)), or a salt thereof to alkylation reaction or acylation reaction. This reaction can be carried out according to a method known per se, for example, by reacting the compound with a compound represented by the formula:

R¹—OH (V)

or

R^{1a}—OH (Va)

wherein R¹ and R^{1a} are as defined above (hereinafter to be referred to as compound (V) or compound (Va)), which is an alkylating agent or an acylating agent, or a salt thereof or a reactive derivative thereof.

As a reactive derivative of compound (V) or compound (Va) or a salt thereof, for example, a compound represented by the formula:

R¹-L¹ (VI)

or

R^{1a}-L¹ (VIa)

wherein L¹ is a leaving group and other symbols are as defined above (hereinafter to be simply referred to as a reactive derivative) or a salt thereof can be used.

As the leaving group for L¹, for example, a halogen atom (a chlorine atom, a bromine atom, an iodine atom etc.), a substituted sulfonyloxy group (a C₁₋₆ alkylsulfonyloxy group such as methanesulfonyloxy, ethanesulfonyloxy and the like; a C₆₋₁₄ arylsulfonyloxy group such as benzenesulfonyloxy, p-toluenesulfonyloxy and the like; a C₇₋₁₆ aralkylsulfonyloxy group such as a benzylsulfonyloxy group etc. and the like), acyloxy (acetoxy, benzoyloxy etc.), an oxy group substituted with a heterocycle or an aryl group (succinic acid imide, benzotriazole, quinoline, 4-nitrophenyl etc.), a heterocycle (imidazole etc.) and the like can be used.

The reaction using the above-mentioned reactive derivative as an alkylating agent can be generally carried out by reacting the reactive derivative in a solvent in the presence of a base. Examples of the solvent include alcohols (methanol, ethanol, propanol etc.), ethers (dimethoxyethane, dioxane, tetrahydrofuran etc.), ketones (acetone etc.), nitriles (acetonitrile etc.), amides (N,N-dimethylformamide etc.), sulfoxides (dimethyl sulfoxide etc.), water and the like, which may be used in a suitable mixture. The base includes, for example, an organic base (trimethylamine, triethylamine, N-methylmorpholine, pyridine, picoline, N,N-dimethylaniline etc.), an inorganic base (potassium carbonate, sodium carbonate, potassium hydroxide, sodium hydroxide etc.), and a metal hydride (sodium hydride etc.). The amount of the base to be used is, for example, about 1 to about 100 molar equivalents, preferably about 1 to about 10 molar equivalents, per 1 mol of the substrate.

The reactive derivative includes, for example, halides (chloride, bromide, iodide etc.), sulfuric acid esters, or sulfonic acid esters (methanesulfonate, p-toluenesulfonate, benzenesulfonate etc.) and the like, and particularly halides. The amount of the reactive derivative to be used is, for example, 1 to 5 molar equivalents, preferably 1 to 3 molar equivalents, per 1 mol of the substrate.

If necessary, the reaction can be facilitated by adding an additive. Such an additive includes, for example, iodide salt (sodium iodide, potassium iodide, etc.) and the like, and the amount to be used is about 0.1 to 10 molar equivalents, preferably about 0.1 to 5 molar equivalents, per 1 mol of the substrate.

The reaction temperature is generally −10° C. to 200° C., preferably about 0° C. to 110° C., and the reaction time is generally 0.5 hr to 48 hr, preferably 0.5 hr to 16 hr.

The reaction using the above-mentioned reactive derivative as an acylating agent depends on the kind of reactive derivative or substrate, but it is generally carried out in a solvent. If necessary, a suitable base may be added to promote the reaction. The solvent includes, for example, hydrocarbons (benzene, toluene, etc.), ethers (diethyl ether, dioxane, tetrahydrofuran, etc.), esters (ethyl acetate, etc.), halogenated hydrocarbons (chloroform, dichloromethane, etc.), esters (ethyl acetate, etc.), amides (N,N-dimethylformamide, etc.), aromatic amines (pyridine, etc.), water and the like, which may be used in a suitable mixture. In addition, the base includes, for example, alkali metal hydroxides (sodium hydroxide, potassium hydroxide, etc.), carbonates (hydrogen carbonates such as sodium hydrogen carbonate, potassium hydrogen carbonate, etc.; sodium carbonate; potassium carbonate, etc.), acetates (sodium acetate, etc.), tertiary amines (trimethylamine, triethylamine, N-methylmorpholine, etc.), aromatic amines (pyridine, picoline, N,N-dimethylaniline, etc.) and the like. The amount of the base to be used is, for example, about 1 to 100 molar equivalents, preferably about 1 to 10 molar equivalents, relative to 1 mol of the substrate.

The acylating agent includes, for example, carboxylic acid, sulfonic acid, phosphoric acid, carbonic acid or a reactive derivative thereof (e.g., acid halide, acid anhydride, mixed acid anhydride, active ester, etc.), isocyanic acid ester, isothiocyanic acid ester and the like.

The amount of such acylating agent to be used is generally about 1 to 10 molar equivalents, preferably about 1 to 3 molar equivalents, relative to 1 mol of the substrate. The reaction temperature is generally about −10° C. to 150° C., preferably about 0° C. to 100° C., and the reaction time is generally about 15 min to 24 hr, preferably about 30 min to 16 hr.

In addition, compound (I) or compound (II) or a salt thereof can be also produced by reacting compound (III) or (IV) or a salt thereof with aldehydes and ketones, and reducing the produced imine or iminium ion.

The reaction to produce imine or iminium ion is generally carried out in a solvent that does not adversely affect the reaction. Such solvent includes, for example, aromatic hydrocarbons (toluene, xylene, etc.), aliphatic hydrocarbons (heptane, hexane, etc.), halogenated hydrocarbons (chloroform, dichloromethane, etc.), ethers (diethyl ether, tetrahydrofuran, dioxane, etc.), alcohols (methanol, ethanol, 2-propanol, butanol, benzyl alcohol, etc.), nitriles (acetonitrile, etc.), amides (N,N-dimethylformamide, etc,), sulfoxides (dimethyl sulfoxide, etc.) and the like. Such solvent may be used in a mixture at a suitable ratio. The aldehyde includes, for example, formalin, optionally substituted C₁₋₅ alkyl-aldehyde (e.g., acetaldehyde, etc.), optionally substituted aromatic aldehyde (e.g., benzaldehyde, etc.) and the like, and the amount to be used is, for example, about 1 to 100 molar equivalents, preferably about 1 to 5 molar equivalents, relative to 1 mol of the substrate.

If necessary, the reaction can advantageously proceed by adding a catalyst. Such catalyst includes, for example, mineral acids (hydrochloric acid, hydrobromic acid, sulfuric acid, etc.), carboxylic acids (formic acid, acetic acid, propionic acid, trifluoroacetic acid, etc.), sulfonic acids (methanesulfonic acid, p-toluenesulfonic acid, etc.), Lewis acids (aluminum chloride, zinc chloride, zinc bromide, boron trifluoride, titanium chloride, etc.), acetates (sodium acetate, potassium acetate, etc.) and molecular sieves (molecular sieves 3A, 4A, 5A, etc.). The amount of the catalyst to be used is, for example, about 0.01 to 50 molar equivalents, preferably about 0.1 to 10 molar equivalents, relative to 1 mol of the substrate.

The reaction temperature is generally about 0° C. to 200° C., preferably about 20° C. to 150° C., and the reaction time is generally 0.5 to 48 hr, preferably 0.5 to 24 hr.

The reduction of imine or iminium ion can be carried out by a method known per se, for example, a method using metal hydride or a method by catalytic hydrogenation.

The metal hydride as the reducing agent includes, for example, metal hydrides (sodium borohydride, lithium borohydride, zinc borohydride, sodium cyanoborohydride, sodium triacetoxyborohydride, lithium cyanoborohydride, dibutylaluminum hydride, aluminum hydride, lithium aluminum hydride, etc.), a borane complex (a borane-tetrahydrofuran complex, catechol borane, etc.) and the like. The metal hydride includes preferably sodium borohydride, sodium cyanoborohydride, sodium triacetoxyborohydride, etc. The amount of the reducing agent to be used is, for example, about 1 to 50 molar equivalents, preferably about 1 to 10 molar equivalents, relative to 1 mol of the substrate. In addition, the reaction solvent includes, for example, aromatic hydrocarbons (toluene, xylene, etc.), aliphatic hydrocarbons (heptane, hexane, etc.), halogenated hydrocarbons (chloroform, dichloromethane, etc.), ethers (diethyl ether, tetrahydrofuran, dioxane, etc.), alcohols (methanol, ethanol, 2-propanol, butanol, benzyl alcohol, etc.), nitriles (acetonitrile, etc.), amides (dimethylformamide, etc.), sulfoxides (dimethyl sulfoxide, etc.) and the like. Such solvent may be used in a mixture at a suitable ratio. The reaction temperature is generally about −80° C. to 80° C., preferably about −40° C. to 40° C., and the reaction time is generally about 5 min to 48 hr, preferably about 1 to 24 hr.

The catalytic hydrogenation can be carried out under hydrogen atmosphere and in the presence of a catalyst. The catalyst to be used is preferably palladium (palladium-carbon, palladium hydroxide, palladium oxide, etc.), nickel (Raney-nickel, etc.), platinum (platinum oxide, platinum carbon, etc.), rhodium (rhodium acetate, etc.) and the like, and the amount to be used is, relative to 1 mol of substrate, for example, about 0.001 to 1 equivalent, preferably about 0.01 to 0.5 equivalent, relative to 1 mol of the substrate.

The catalytic hydrogenation is generally carried out in a solvent inert to the reaction. As such solvent, for example, alcohols (methanol, ethanol, propanol, butanol etc.), hydrocarbons (benzene, toluene, xylene etc.), halogenated hydrocarbons (dichloromethane, chloroform etc.), ethers (diethyl ether, dioxane, tetrahydrofuran etc.), esters (ethyl acetate etc.), amides (N,N-dimethylformamide etc.), carboxylic acids (acetic acid etc.), water or a mixture thereof can be used. The hydrogen pressure under which the reaction proceeds is generally about 1 to 50 atm, preferably about 1 to 10 atm. The reaction temperature is generally about 0° C. to 150° C., preferably 20° C. to 100° C., and the reaction time is generally about 5 min to 72 hr, preferably 0.5 to 40 hr.

Compound (I) or compound (II) can be also produced directly from compound (III) or (IV) in the present process, while carrying out the reaction of producing and of reducing imine or iminium ion at the same time, without isolating the intermediate imine or iminium ion. In this case, pH of the reaction mixture is preferably about 4 to 5.

Compound (III) to be used as a starting compound in Method A can be produced by subjecting compound (VII) obtained by Method B mentioned below or a salt thereof to deacylation or dealkylation. Compound (IV) to be used as a starting compound in Method A can be produced according to a known method (e.g., WO2006/004195).

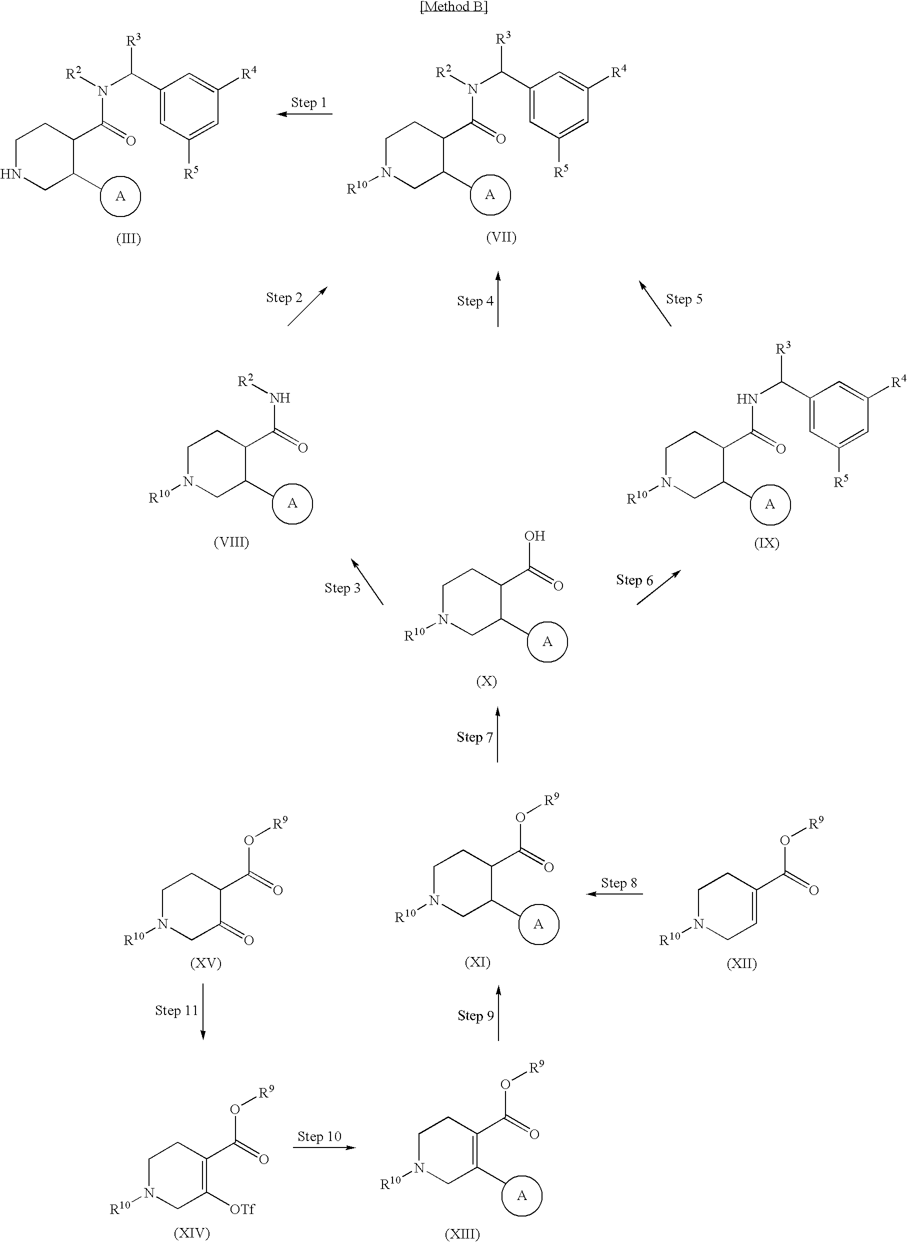

wherein R⁹ is a hydrocarbon group optionally having substituent(s), R¹⁰ is a hydrocarbon group optionally having substituent(s) or an acyl group optionally having substituent(s), and other symbols are as defined above.

The hydrocarbon group optionally having substituent(s) for R⁹ is a carboxyl-protecting group mentioned below (e.g., methyl, ethyl, n-propyl, isopropyl, benzyl etc.). The hydrocarbon group optionally having substituent(s) and the acyl group optionally having substituent(s) for R¹⁰ are the amino-protecting groups mentioned below (e.g., methyl, ethyl, n-propyl, isopropyl, benzyl, t-butyloxycarbonyl, acyl, propionyl, benzoyl etc.).

### (Step 1)

In this step, compound (III) is produced by subjecting compound (VII) or a salt thereof to deacylation reaction or dealkylation reaction.

Such deacylation reaction can be carried out according to a known method. For example, the reaction is generally carried out in the presence of an acid or a base, if necessary, in a solvent that does not adversely affect the-reaction, though subject to change depending on the kind of the substrate.

The acid is preferably a mineral acid (hydrochloric acid, hydrobromic acid, sulfuric acid etc.), carboxylic acid (acetic acid, trifluoroacetic acid, trichloroacetic acid etc.), sulfonic acid (methanesulfonic acid, toluenesulfonic acid etc.), Lewis acid (aluminum chloride, tin chloride, zinc bromide etc.) and the like. If necessary, the acid may be a mixture of two or more acids. The amount of the acid to be used varies depending on the kind of the solvent and other reaction conditions, but it is generally about 0.1 molar equivalents or more, per 1 mol of compound (VII), and the acid can be used as a solvent.

The base is, for example, preferably an inorganic base (alkali metal hydroxides such as sodium hydroxide, potassium hydroxide etc., alkali metal hydrogen carbonates such as sodium hydrogen carbonate, potassium hydrogen carbonate etc., alkali metal carbonates such as sodium carbonate, potassium carbonate etc., alkoxides such as sodium methoxide, sodium ethoxide etc. etc.), or an organic base (amines such as trimethylamine, triethylamine, diisopropylethylamine etc., cyclic amines such as pyridine, 4-dimethylaminopyridine etc.) and the like, and preferably, sodium hydroxide, potassium hydroxide, sodium ethoxide and the like.

The amount of the base to be used varies depending on the kind of the solvent and other reaction conditions, but is generally about 0.1 to 10 molar equivalents, preferably about 0.1 to 5 molar equivalents, per 1 mol of compound (VII).

The solvent that does not adversely affect the reaction includes, for example, alcohols (methanol, ethanol, propanol, 2-propanol, butanol, isobutanol, t-butanol, etc.), aromatic hydrocarbons (benzene, toluene, xylene, etc.), aliphatic hydrocarbons (hexane, heptane, etc.), halogenated hydrocarbons (dichloromethane, chloroform, etc.), ethers (diethyl ether, diisopropyl ether, t-butyl methyl ether, tetrahydrofuran, dioxane, dimethoxyethane, etc.), nitriles (acetonitrile, etc.), esters (ethyl acetate, etc.), carboxylic acids (acetic acid, etc.), amides (dimethylformamide, etc.), sulfoxides (dimethyl sulfoxide, etc.), water and the like. Such solvent may be used in a mixture of two or more at a suitable ratio.

The reaction temperature is for example, about −50° C. to 200° C., preferably about 0° C. to 100° C., and the reaction time varies depending on the kind of compound (VII) or a salt thereof, the reaction temperature and the like, and it is for example, about 0.5 hr to 100 hr, preferably about 0.5 hr to 24 hr.

Dealkylation can be carried out by a known method, for example, the method described in Theodara W. Greene, Peter G. M. Wuts, “Protective Groups in Organic Synthesis, 3^{rd} Ed.,” (1999) Wiley-Interscience, and the like, or an analogous method thereto. For example, the dealkylation can be carried out by treatment with an acid, a base, ultraviolet radiation, a transition metal catalyst and the like, or by oxidation, reduction or acylation followed by hydrolysis etc., or a combination thereof can be used.

### (Step 2)

In this step, compound (VII) is produced by reacting compound (VIII) with a compound represented by the formula:

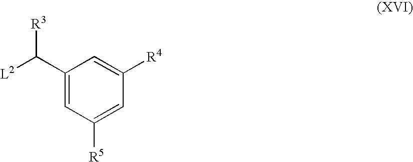

wherein L² is a leaving group and other symbols are as defined above, or a salt thereof.

As the leaving group for L², for example, a halogen atom (a chlorine atom, a bromine atom, an iodine atom etc.), a substituted sulfonyloxy group (a methanesulfonyloxy group, an ethanesulfonyloxy group, a benzenesulfonyloxy group, a toluenesulfonyloxy group, a benzylsulfonyloxy group etc.), an acyloxy group (an acetoxy group, a benzoyloxy group etc.), an oxy group substituted by a heterocycle or an aryl group (succinic acid imide, benzotriazole, quinoline, 4-nitrophenyl etc.), a heterocycle (imidazole etc.) and the like can be used, and particularly, a halogen atom is preferable. The amount of compound (XVI) to be used is, for example, about 1 to 5 molar equivalents, preferably about 1 to 3 molar equivalents, per 1 mol of compound (VIII).

This reaction can be generally carried out by reacting compound (XVI) in a solvent in the presence of a base. Examples of the solvent include alcohols (methanol, ethanol, propanol etc.), ethers (dimethoxyethane, dioxane, tetrahydrofuran etc.), ketones (acetone etc.), nitriles (acetonitrile etc.), amides (N,N-dimethylformamide etc.), sulfoxides (dimethyl sulfoxide etc.), water and the like, which may be used in a suitable mixture. Examples of the base include organic bases (trimethylamine, triethylamine, N-methylmorpholine, pyridine, picoline, N,N-dimethylaniline etc.), inorganic bases (potassium carbonate, sodium carbonate, potassium hydroxide, sodium hydroxide etc.) and the like. The amount of the base to be used is, for example, about 1 to 100 molar equivalents, preferably about 1 to 10 molar equivalents, per 1 mol of the substrate.

If necessary, the reaction can be facilitated by adding an additive. Examples of such additive include iodide salt (sodium iodide, potassium iodide, etc.) and the like, and the amount to be used is about 0.1 to 10 molar equivalents, preferably about 0.1 to 5 molar equivalents, per 1 mol of compound (VIII).

The reaction temperature is generally −10° C. to 200° C., preferably about 0° C. to 110° C., and the reaction time is generally 0.5 hr to 48 hr, preferably 0.5 hr to 16 hr.

### (Step 3)

In this step, compound (VIII) is produced by subjecting compound (X) or a salt thereof and a compound represented by the formula:

R²—NH₂ (XVII)

wherein each symbol is as defined above, or a salt thereof to dehydrative condensation.

Compound (XVII) and a salt thereof are commercially available, or can be produced according to a known method. The amount thereof to be used is about 1 to 10 molar equivalents, preferably about 1 to 2 molar equivalents, per 1 mol of compound (X).

As the method for dehydrative condensation, a method known per se, for example, the method described in “4th Ed. Jikken Kagaku Koza (Courses in Experimental Chemistry) 22, Organic Synthesis IV”, The Chemical Society of Japan Ed. 1991 and the like, or a method analogous thereto can be employed. As such method, for example, a method using a condensing agent, a method via a reactive derivative and the like can be mentioned.

Examples of the condensing agent to be used for “a method using a condensing agent” include dicyclohexylcarbodiimide, diisopropylcarbodiimide, N-ethyl-N′-3-dimethylaminopropylcarbodiimide and its hydrochloride, benzotriazol-1-yl-tris(dimethylamino)phosphonium hexafluorophosphate, diphenylphosphoryl azido and the like. They may be used alone or in combination with an additive (e.g., N-hydroxysuccinimide, 1-hydroxybenzotriazole, 3-hydroxy-4-oxo-3,4-dihydro-1,2,3-benzotriazine, etc.). The amount of the condensing agent to be used is about 1 to 10 molar equivalents, preferably about 1 to 2 molar equivalents, per 1 mol of compound (X). The amount of the additive to be used is about 1 to 10 molar equivalents, preferably about 1 to 2 molar equivalents, per 1 mol of compound (X).

The above-mentioned reaction is generally carried out in a solvent that does not adversely affect the reaction, and a suitable base may be added to promote the reaction. As the solvent, for example, hydrocarbons (benzene, toluene, etc.), ethers (diethyl ether, dioxane, tetrahydrofuran, etc.), esters (ethyl acetate, etc.), halogenated hydrocarbons (chloroform, dichloromethane, etc.), amides (N,N-dimethylformamide, etc.), aromatic amines (pyridine, etc.), water and the like can be mentioned, which may be appropriately mixed. As the base, for example, alkali metal hydroxides (sodium hydroxide, potassium hydroxide, etc.), hydrogen carbonates (sodium hydrogen carbonate, potassium hydrogen carbonate, etc.), carbonates (sodium carbonate, potassium carbonate, etc.), acetates (sodium acetate, etc.), tertiary amines (trimethylamine, triethylamine, N-methylmorpholine, etc.), aromatic amines (pyridine, picoline, N,N-dimethylaniline, etc.) and the like can be mentioned. The amount of the base to be used is generally about 1 to 100 molar equivalents, preferably about 1 to 5 molar equivalents, per 1 mol of the substrate. The reaction temperature is generally about −80° C. to 150° C., preferably about 0° C. to 50° C., and the reaction time is generally about 0.5 to 48 hr, preferably 0.5 to 16 hr.

As the reactive derivative of the “method via a reactive derivative”, for example, acid halide, acid anhydride, mixed acid anhydride, active ester and the like can be mentioned. Conversion to a reactive derivative can be carried out according to a method known per se. For example, for conversion to an acid halide, a method using an acid halide (e.g., thionyl chloride, oxalyl chloride, etc.), a method using a halide of phosphorus and phosphoric acid (e.g., phosphorus trichloride, phosphorus pentachloride, etc.) and the like can be mentioned. The above-mentioned reaction using a reactive derivative is generally carried out in a solvent that does not adversely affect the reaction and a base suitable for promoting the reaction can be added, though subject to change depending on the kind of the reactive derivative or a substrate. The kind and the amount of the solvent and base to be used for the reaction, the reaction temperature and reaction time are the same as those described for the above-mentioned “method using a condensing agent”.

### (Step 4)

In this step, compound (VII) is produced by subjecting compound (X) or a salt thereof and a compound represented by the formula:

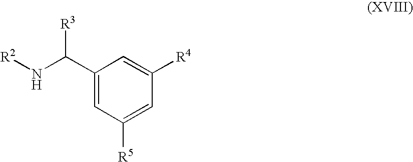

wherein each symbol is as defined above, or a salt thereof to dehydrative condensation. This step can be carried out in the same manner as in step 3 of method B.

Compound (XVIII) or a salt thereof may be a commercially available product, or can be produced according to a known method. The amount thereof to be used is about 1 to 10 molar equivalents, preferably about 1 to 2 molar equivalents, per 1 mol of compound (X).

### (Step 5)

In this step, compound (VII) is produced by reacting compound (IX) with a compound represented by the formula:

R²-L³ (XIX)

wherein L³ is a leaving group, and other symbols are as defined above, or a salt thereof. This step can be carried out in the same manner as in step 2 of method B.

As the leaving group for L³, those similar to L² in step 2 of method B can be mentioned.

Compound (XIX) or a salt thereof may be a commercially available product, or can be produced according to a known method. The amount thereof to be used is about 1 to 10 molar equivalents, preferably about 1 to 2 molar equivalents, per 1 mol of compound (IX).

### (Step 6)

In this step, compound (IX) is produced by subjecting compound (X) or a salt thereof and a compound represented by the formula:

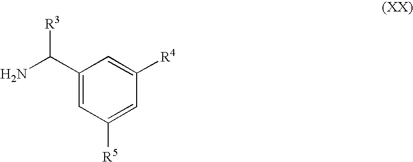

wherein each symbol is as defined above, or a salt thereof to dehydrative condensation. This step can be performed in the same manner as in step 3 of method B.

Compound (XX) or a salt thereof may be a commercially available product, or can be produced according to a known method. The amount thereof to be used is about 1 to 10 molar equivalents, preferably about 1 to 2 molar equivalents, per 1 mol of compound (X).

### (Step 7)

In this step, compound (X) is converted to compound (XI) by subjecting the compound to hydrolysis. This reaction can be performed according to a method known per se, generally in the presence of an acid or a base in, if necessary, a solvent that does not adversely affect the reaction.

As the acid, for example, mineral acids (hydrochloric acid, hydrobromic acid, sulfuric acid etc.), carboxylic acids (acetic acid, trifluoroacetic acid, trichloroacetic acid etc.), sulfonic acids (methanesulfonic acid, toluenesulfonic acid etc.), Lewis acid (aluminum chloride, tin chloride, zinc bromide etc.) and the like can be used. If necessary, the acid may be a mixture of two or more acids. The amount of the acid to be used varies depending on the kind of the solvent and other reaction conditions, but it is generally about 0.1 molar equivalents or more, per 1 mol of compound (XI), and the acid can be used as a solvent.

As the base, for example, inorganic base (alkali metal hydroxide such as lithium hydroxide, sodium hydroxide, potassium hydroxide etc., alkali metal hydrogen carbonate such as sodium hydrogen carbonate, potassium hydrogen carbonate etc., alkali metal carbonate such as sodium carbonate, potassium carbonate etc., alkoxide such as sodium methoxide, sodium ethoxide etc. and the like) or organic base (amines such as trimethylamine, triethylamine, diisopropylethylamine etc., cyclic amines such as pyridine, 4-dimethylaminopyridine etc. and the like) and the like can be used. Of these, lithium hydroxide, sodium hydroxide, potassium hydroxide, sodium ethoxide and the like are preferable. While the amount of the base to be used varies depending on the kind of the solvent and other reaction conditions, it is generally about 0.1 to 10 molar equivalents, preferably about 0.1 to 5 molar equivalents, per 1 mol of compound (XI).

The solvent that does not adversely affect the reaction includes, for example, alcohols (methanol, ethanol, propanol, 2-propanol, butanol, isobutanol, t-butanol etc.), hydrocarbons (benzene, toluene, xylene, hexane, heptane etc.), halogenated hydrocarbons (dichloromethane, chloroform etc.), ethers (diethyl ether, diisopropyl ether, t-butyl methyl ether, tetrahydrofuran, dioxane, dimethoxyethane etc.), nitriles (acetonitrile etc.), carboxylic acids (acetic acid etc.), amides (dimethylformamide, dimethylacetamide etc.), sulfoxides (dimethyl sulfoxide etc.), water and the like. These solvents may be used in a mixture of two or more kinds thereof at a suitable ratio.

The reaction temperature is, for example, about −50° C. to 200° C., preferably about 0° C. to 100° C., and the reaction time varies depending on the kind of compound (XI) or a salt thereof, the reaction temperature and the like. It is, for example, about 0.5 hr to 100 hr, preferably about 0.5 hr to 24 hr.

### (Step 8)

In this step, compound (XI) is produced by adding a compound represented by the formula:

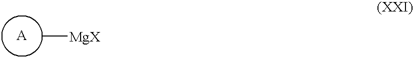

wherein X is a halogen atom and other symbols are as defined above, or a salt thereof to compound (XII) or a salt thereof.

Compound (XII) or a salt thereof, which is a starting material, may be a commercially available product, or can be produced according to a method known per se (e.g., Journal of the Chemical Society, Perkin Transactions 1: Organic and Bio-Organic Chemistry (1972-1999), 1981, vol. 6, pages 1754-1762).

A Grignard reagent represented by the formula (XXI) may be a commercially available product, or can be prepared according to a method known per se, for example, the method described in “4th Ed. Jikken Kagaku Koza (Courses in Experimental Chemistry) 24, Organic Synthesis VI”, The Chemical Society of Japan Ed. 1991, or an analogous method thereto.

In this step, the reaction proceeds advantageously by adding an additive as necessary. Such additive includes, for example, copper salt (e.g., copper chloride, copper bromide, copper iodide, copper cyanide etc.), lithium salt (e.g., lithium chloride, lithium bromide, lithium iodide etc.), Lewis acid (e.g., boron trifluoride, trimethylsilyl chloride, aluminum chloride etc.), Lewis base (e.g., tributylphosphine, triphenylphosphine, dimethylethylenediamine etc.), a mixture thereof and the like. Of these, copper bromide, copper iodide, copper cyanide and the like are preferable. The amount of the additive to be used is about 0.001 to 10 molar equivalents, preferably about 0.1 to 2 molar equivalents, per 1 mol of the Grignard reagent represented by the formula (XXI).

The step is carried out in a solvent inert to the reaction. As such solvent, for example, hydrocarbons (hexane, benzene, toluene, xylene etc.), halogenated hydrocarbons (dichloromethane, chloroform etc.), ethers (diethyl ether, dioxane, tetrahydrofuran etc.) or a mixture thereof can be used. The reaction temperature is generally about −80° C. to 50° C., preferably about −35° C. to 0° C., and the reaction time is generally 5 min to 48 hr, preferably 1 hr to 24 hr.

### (Step 9)

In this step, compound (XIII) is converted to compound (XI) by subjecting the compound to reduction reaction. This step can be carried out according to a method known per se and, for example, compound (XI) can be produced by reducing compound (XIII) with a metal or a metal salt, reducing compound (XIII) by catalytic hydrogenation using a transition metal catalyst.

The metal and metal salt to be used for the “reduction by metal or metal salt” are preferably, for example, alkali metal (lithium, sodium, potassium etc.), alkaline earth metal (magnesium, calcium etc.), other metals (zinc, chrome, titanium, iron, samarium, selenium etc.), metal salt (zinc-amalgam, zinc-copper alloy, aluminum-amalgam, sodium hydrosulfite etc.) and the like. The amount of the reducing agent to be used is about 1 to 50 molar equivalents, preferably about 1 to 5 molar equivalents, per 1 mol of the substrate.

The solvent to be used for the reaction includes, for example, alcohols (methanol, ethanol, 2-propanol, t-butanol, benzyl alcohol etc.), amines (liquid ammonia, methylamine, ethylamine, ethylenediamine etc.), ethers (diethyl ether, tetrahydrofuran, dioxane, dimethoxyethane etc.), mineral acids (hydrochloric acid, hydrobromic acid, sulfuric acid etc.), carboxylic acids (acetic acid etc.), amides (hexamethylphosphoamide), water and the like. These solvents can be used alone or in a mixture.

The reaction temperature is generally about −80° C. to 150° C., preferably about −80° C. to 100° C., and the reaction time is generally 5 min to 48 hr, preferably 1 hr to 24 hr.

The transition metal catalyst to be used for the “reduction by catalytic hydrogenation using a transition metal catalyst” is preferably, for example, palladium (palladium-carbon, palladium hydroxide, palladium oxide etc.), nickel (Raney-nickel etc.), platinum (platinum oxide, platinum carbon etc.), rhodium (rhodium acetate, rhodium carbon etc.) and the like, and the amount thereof to be used is, relative to 1 mol of the substrate, for example, about 0.001 to 1 equivalents, preferably about 0.01 to 0.5 equivalent, relative to 1 mol of the substrate. The catalytic hydrogenation reaction is generally carried out in a solvent inert to the reaction. As such solvent, for example, alcohols (methanol, ethanol, propanol, butanol etc.), hydrocarbons (benzene, toluene, xylene etc.), halogenated hydrocarbons (dichloromethane, chloroform etc.), ethers (diethyl ether, dioxane, tetrahydrofuran etc.), esters (ethyl acetate etc.), amides (N,N-dimethylformamide etc.), carboxylic acids (acetic acid etc.), water or a mixture thereof can be used. The hydrogen pressure, under which the reaction is carried out, is generally about 1 to 500 atm, preferably about 1 to 100 atm. The reaction temperature is generally about 0° C. to 150° C., preferably about 20° C. to 100° C., and the reaction time is generally 5 min to 72 hr, preferably 0.5 hr to 40 hr.

### (Step 10)

In this step, compound (XIII) is produced by subjecting compound (XIV) or a salt thereof, and a compound represented by the formula:

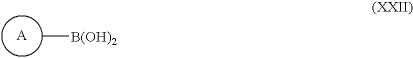

wherein each symbol is as defined above, or a salt thereof to coupling reaction.

This step can be carried out by a method known per se [e.g., Chemical Reviews, Vol. 95, p. 2457 (1995) and the like] and, for example, carried out in the presence of a transition metal catalyst and a base in a solvent that does not adversely affect the reaction.

As the transition metal catalyst to be used, for example, palladium catalysts (palladium acetate, palladium chloride, tetrakis(triphenylphosphine)palladium, etc.), nickel catalysts (nickel chloride, etc.) and the like are used. Where necessary, ligands (triphenylphosphine, tri-t-butylphosphine, etc.) may be added or metal oxides (copper oxide, silver oxide, etc.) and the like may be used as cocatalysts. While the amount of the catalyst to be used varies depending on the kind of the catalyst, it is generally about 0.0001 to 1 molar equivalent, preferably about 0.01 to 0.5 molar equivalents, per 1 mol of compound (XIV). The amount of the ligand to be used is generally about 0.0001 to 4 molar equivalents, preferably about 0.01 to 2 molar equivalents, per 1 mol of compound (XIV), and the amount of the cocatalyst to be used is about 0.0001 to 4 molar equivalents, preferably about 0.01 to 2 molar equivalents, per 1 mol of compound (XIV).

As the base to be used, for example, organic amines (trimethylamine, triethylamine, diisopropylamine, N-methylmorpholine, 1,8-diazabicyclo[5.4.0]undec-7-ene, pyridine, N,N-dimethylaniline, etc.), alkali metal salts (sodium hydrogen carbonate, potassium hydrogen carbonate, sodium carbonate, potassium carbonate, cesium carbonate, sodium phosphate, potassium phosphate, sodium hydroxide, potassium hydroxide, etc.), metal hydrides (potassium hydride, sodium hydride, etc.), alkali metal alkoxides (sodium methoxide, sodium ethoxide, sodium t-butoxide, potassium t-butoxide, etc.), alkali disilazides (lithium disilazide, sodium disilazide, potassium disilazide, etc.) and the like can be mentioned. Of these, alkali metal salts such as potassium carbonate, cesium carbonate, sodium phosphate, potassium phosphate and the like; alkali metal alkoxides such as sodium t-butoxide, potassium t-butoxide and the like; organic amines such as triethylamine, diisopropylamine and the like; and the like are preferable. The amount of the base to be used is about 0.1 to 10 molar equivalents, preferably about 1 to 5 molar equivalents, per 1 mol of compound (XIV).

The solvent to be used may be any as long as it does not adversely affect the reaction and, for example, hydrocarbons (benzene, toluene, xylene etc.), halogenated hydrocarbons (chloroform, 1,2-dichloroethane etc.), nitriles (acetonitrile etc.), ethers (dimethoxyethane, tetrahydrofuran), alcohols (methanol, ethanol etc.), aprotic polar solvent (dimethylformamide, dimethyl sulfoxide, hexamethylphosphoroamide etc.), water or a mixture thereof can be used. The reaction temperature is generally about −10° C. to 200° C., preferably about 0° C. to 150° C., and the reaction time is generally 0.5 hr to 48 hr, preferably 0.5 hr to 16 hr.

### (Step 11)

In this step, compound (XIV) or a salt thereof is produced by subjecting compound (XV) or a salt thereof to triflatation. In the above-mentioned scheme, Tf is a trifluoromethanesulfonyl group and other symbols are as defined above.

Compound (XV) or a salt thereof, which is a starting material, may be a commercially available product, or can be produced according to a known method (e.g., Heterocycles, 1978, vol. 11, pages 267-273 etc.).

This step can be carried out according to a method known per se, for example, a method described in “4th Ed. Jikken Kagaku Koza (Courses in Experimental Chemistry) 24, Organic Synthesis VI”, The Chemical Society of Japan Ed. 1991 and the like, or an analogous method thereto. For example, the step can be performed by reacting a triflating agent in the presence of a base in a solvent that does not adversely affect the reaction.

The base to be used includes, for example, organic amines (trimethylamine, triethylamine, diisopropylamine, N-methylmorpholine, 1,8-diazabicyclo[5,4,0]undec-7-ene, pyridine, N,N-dimethylaniline etc.), alkali metal salt (sodium hydrogen carbonate, potassium hydrogen carbonate, sodium carbonate, potassium carbonate, cesium carbonate, sodium hydroxide, potassium hydroxide etc.), metal hydride (potassium hydride, sodium hydride etc.) and the like, preferably, organic amines such as triethylamine, diisopropylamine and the like, metal hydride such as sodium hydride, etc. and the like. The amount of the base to be used is about 0.1 to 10 molar equivalents, preferably about 1 to 5 molar equivalents, per 1 mol of compound (XV).

The solvent to be used may be any as long as it does not adversely affect the reaction and, for example, hydrocarbons (benzene, toluene, xylene etc.), halogenated hydrocarbons (chloroform, 1,2-dichloroethane etc.), esters(ethyl acetate etc.), nitriles(acetonitrile etc.), ethers(dimethoxyethane, tetrahydrofuran), aprotic polar solvent (dimethylformamide, dimethyl sulfoxide, hexamethylphosphoroamide etc.) or a mixture thereof can be used.

The triflating agent includes, for example, sulfonic acid anhydride (e.g., trifluoromethanesulfonic acid anhydride etc.), halogenated sulfonyls (e.g., trifluoromethanesulfonyl chloride etc.), sulfonimides (e.g., N-phenylbis(trifluoromethanesulfonimide) etc.), sulfonate esters (e.g., ethyl trifluoromethanesulfonate etc.) and the like, preferably, sulfonic acid anhydride such as trifluoromethanesulfonic acid anhydride and the like, sulfonimides such as N-phenylbis(trifluoromethanesulfonimide) and the like. The amount of the triflating agent to be used is about 0.1 to 10 molar equivalents, preferably about 1 to 5 molar equivalents, per 1 mol of compound (XV).

The reaction temperature is generally about −80° C. to 100° C., preferably about −80° C. to 20° C., and the reaction time is generally 5 min to 48 hr, preferably 5 min to 8 hr.

Compound (XXX) and salts thereof of the present invention can be produced by Method C mentioned below.

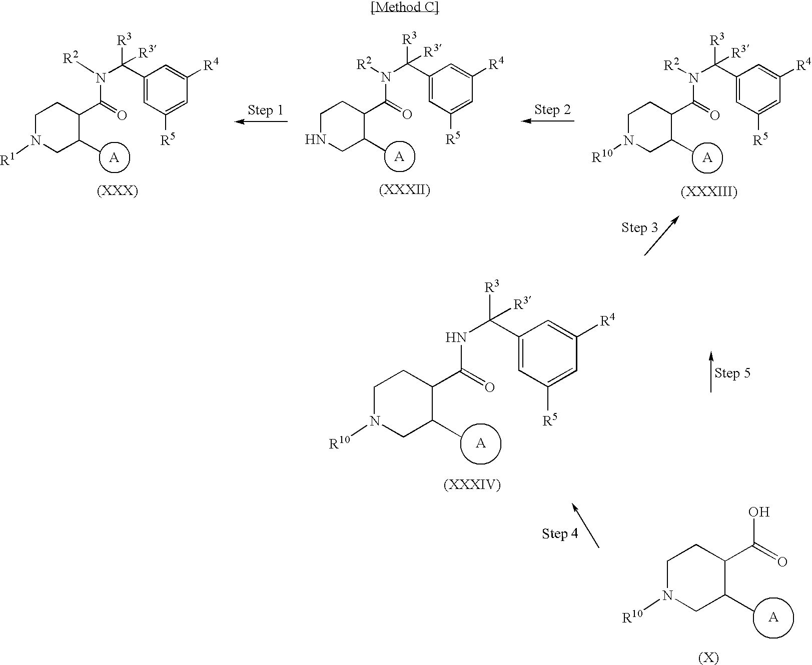

wherein each symbol is as defined above.

### (Step 1)

In this step, compound (XXX) or a salt thereof is produced by subjecting compound (XXXII) or a salt thereof to alkylation reaction or acylation reaction. This step can be performed in the same manner as in Method A.

### (Step 2)

In this step, compound (XXXII) or a salt thereof is produced by subjecting compound (XXXIII) or a salt thereof to dealkylation reaction or deacylation reaction. This step can be performed in the same manner as in step 1 of Method B.

### (Step 3)

In this step, compound (XXXIII) is produced by reacting compound (XXXIV) or a salt thereof with compound (XIX) or a salt thereof. This step can be performed in the same manner as in step 5 of Method B.

### (Step 4)

In this step, compound (XXXIV) is produced by subjecting compound (X) or a salt thereof and a compound represented by the formula (XXXV)

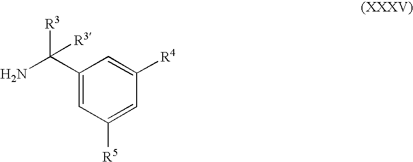

wherein each symbol is as defined above (hereinafter to be abbreviated as compound (XXXV)), or a salt thereof to dehydrative condensation. This step can be performed in the same manner as in step 3 of Method B.

### (Step 5)

In this step, compound (XXXIII) is produced by subjecting compound (X) or a salt thereof and a compound represented by the formula (XXXVI)

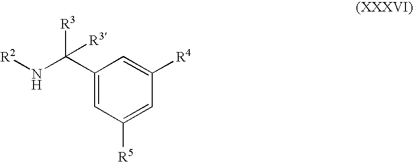

wherein each symbol is as defined above (hereinafter to be abbreviated as compound (XXXVI)), or a salt thereof to dehydrative condensation. This step can be performed in the same manner as in step 3 of Method B.

Compound (XXXI) and salts thereof of the present invention can be produced by Method D mentioned below.

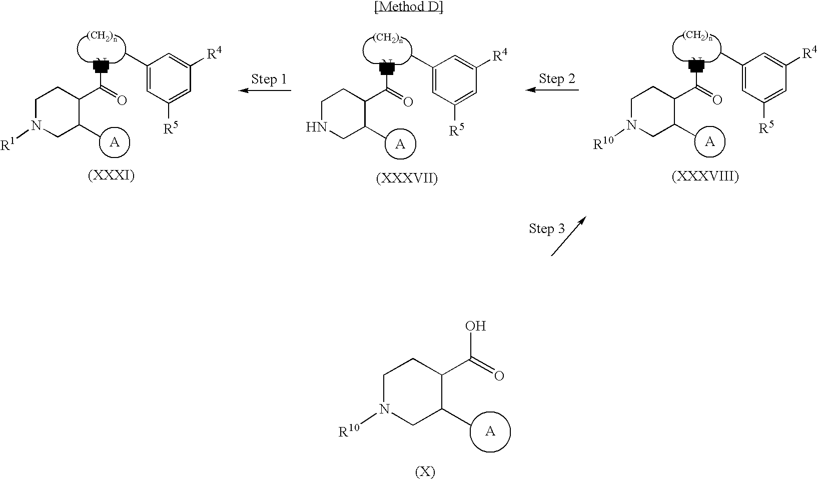

wherein each symbol is as defined above.

### (Step 1)

In this step, compound (XXXI) or a salt thereof is produced by subjecting compound (XXXVII) or a salt thereof to alkylation reaction or acylation reaction. This step can be performed in the same manner as in Method A.

### (Step 2)

In this step, compound (XXXVII) or a salt thereof is produced by subjecting compound (XXXVIII) or a salt thereof to dealkylation reaction or deacylation reaction. This step can be performed in the same manner as in step 1 of Method B.

### (Step 3)

In this step, compound (XXXVIII) is produced by subjecting compound (X) or a salt thereof and a compound represented by the formula (XXXIX)

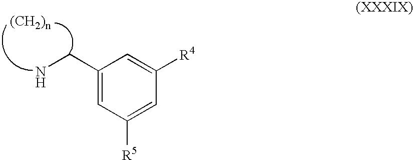

wherein each symbol is as defined above (hereinafter to be abbreviated as compound (XXXIX)), or a salt thereof to dehydrative condensation. This step can be performed in the same manner as in step 3 of Method B.

Compound (XXXIX) or a salt thereof may be a commercially available product, or can be produced according to a known method (ex. WO2006/015150).

In each of the reactions for the synthesis of the objective compounds and the starting materials, when the starting compounds have an amino group, a carboxyl group or a hydroxyl group as a substituent, such groups may be protected with the protecting groups which are generally used in peptide chemistry etc. In such a case, if necessary, such protecting groups can be removed to obtain the objective compounds after the reactions.

Such a protecting group includes, for example, protecting groups described in “Protective Groups in Organic Synthesis, 3^{rd} Ed. (1999)”, edited by Theodara W. Greene, Peter G. M. Wuts, published by Wiley-Interscience.

Examples of the protecting group for the amino group include a formyl group, a C₁₋₆ alkyl-carbonyl group (an acetyl group, a propionyl group etc.), a phenylcarbonyl group, a C₁₋₆ alkyl-oxycarbonyl group (methoxycarbonyl group, an ethoxycarbonyl group etc.), an aryloxycarbonyl group (a phenyloxycarbonyl group etc.), a C₇₋₁₀ aralkyl-carbonyl group (a benzyloxycarbonyl group etc.), a benzyl group, a benzhydryl group, a trityl group, a phthaloyl etc., each of which may have substituent(s). Examples of such substituent include a halogen atom (a fluorine atom, a chlorine atom, a bromine atom, an iodine atom etc.), a C₁₋₆ alkyl-carbonyl group (an acetyl group, a propionyl group, a butylcarbonyl group etc.), a nitro group and the like. The number of substituent(s) is 1 to 3.

Examples of the protecting group for the carboxyl group include a C₁₋₆ alkyl group (a methyl group, an ethyl group, a n-propyl group, an isopropyl group, a n-butyl group, a t-butyl group etc.), a phenyl group, a trityl group, a silyl group and the like can be mentioned, each of which may have substituent(s). Examples of these substituents include a halogen atom (a fluorine atom, a chlorine atom, a bromine atom, an iodine atom etc.), a formyl group, a C₁₋₆ alkyl-carbonyl group (an acetyl group, a propionyl group, a butylcarbonyl group etc.), a nitro group and the like. The number of substituent(s) is 1 to 3.

Examples of the hydroxyl-protecting group include a C₁₋₆ alkyl group (a methyl group, an ethyl group, a n-propyl group, an isopropyl group, a n-butyl group, a t-butyl group etc.), a phenyl group, a C₇₋₁₀ aralkyl group (a benzyl group etc.), a formyl group, C₁₋₆ alkyl-carbonyl group (an acetyl group, a propionyl group etc.), an aryloxycarbonyl group (a phenyloxycarbonyl group etc.), a C₇₋₁₀ aralkyl-carbonyl group (a benzyloxycarbonyl group etc.), a pyranyl group, a furanyl group, a silyl group and the like, each of which may have substituent(s). Examples of these substituents include a halogen atom (a fluorine atom, a chlorine atom, a bromine atom, an iodine atom etc.), a C₁₋₆ alkyl group, a phenyl group, a C₇₋₁₀ aralkyl group, a nitro group and the like. The number of substituent(s) is 1 to 4.

Such protecting groups can be removed by a known deprotection method or the method described in “Protective Groups in Organic Synthesis, 3^{rd} Ed. (1999)”, edited by Theodora W. Greene, Peter G. M. Wuts, published by Wiley-Interscience, or the like, or an analogous method thereto. For example, treatment with an acid, a base, a reducing agent, ultraviolet radiation, hydrazine, phenylhydrazine, sodium N-methyldithiocarbamate, tetrabutylammonium fluoride, palladium acetate or the like, can be used.

In addition, when the starting compound may form a salt in each of the above-mentioned reactions, the compound may be used as a salt. Such salt includes, for example, those exemplified as the salts of compound (I), compound (II), compound (XXX) and compound (XXXI).

Compound (I), compound (II), compound (XXX) and compound (XXXI) thus prepared by such methods, can be isolated and purified by a typical separation means such as recrystallization, distillation, chromatography and the like.

When compound (I), compound (II), compound (XXX) and compound (XXXI) include an optical isomer, a stereoisomer, a regioisomer and a rotamer, these are also included in the scope of the compounds, and can be obtained as single products according to synthesis and separation methods known per se (for example, concentration, solvent extraction, column chromatography, recrystallization etc.). For example, when compound (I) has an optical isomer, the optical isomer resolved from this compound is also encompassed in compound (I).

The optical isomer can be prepared by a method known per se. To be specific, an optically active synthetic intermediate is used, or the final racemate product is subjected to optical resolution according to a conventional method to give an optical isomer.

The method of optical resolution may be a method known per se, such as a fractional recrystallization method, a chiral column method, a diastereomer method etc.

### 1) Fractional Recrystallization Method

A method wherein a salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine etc.) is formed, which is separated by a fractional recrystallization method, and if desired, a free optical isomer is obtained by a neutralization step.

### 2) Chiral Column Method

A method wherein a racemate or a salt thereof is applied to a column for separation of an optical isomer (a chiral column) to allow separation. In the case of a liquid chromatography, for example, a mixture of the optical isomers is applied to a chiral column such as ENANTIO-OVM (manufactured by Tosoh Corporation), CHIRAL series (manufactured by Daicel Chemical Industries, Ltd.) and the like, and developed with water, various buffers (e.g., phosphate buffer) and organic solvents (e.g., ethanol, methanol, isopropanol, acetonitrile, trifluoroacetic acid, diethylamine etc.) solely or in admixture to separate the optical isomer. In the case of a gas chromatography, for example, a chiral column such as CP-Chirasil-DeX CB (manufactured by GL Sciences Inc.) and the like is used to allow separation.

### 3) Diastereomer Method

A method wherein a racemic mixture is prepared into a diastereomeric mixture by chemical reaction with an optically active reagent, which is made into a single substance by a typical separation means (e.g., a fractional recrystallization method, a chromatography method etc.) and the like, and is subjected to a chemical treatment such as hydrolysis and the like to separate an optically active reagent moiety, whereby an optical isomer is obtained. For example, when compound (I) contains hydroxy, or primary or secondary amino group within a molecule, the compound and an optically active organic acid (e.g., MTPA [α-methoxy-α-(trifluoromethyl)phenylacetic acid], (−)-menthoxyacetic acid etc.) and the like are subjected to condensation reaction to give diastereomers of the ester compound or the amide compound, respectively. When compound (I) has a carboxylic acid group, this compound and an optically active amine or an alcohol reagent are subjected to condensation reaction to give diastereomers of the amide compound or the ester compound, respectively. The separated diastereomer is converted to an optical isomer of the original compound by acid hydrolysis or base hydrolysis.

Compound (I), compound (II), compound (XXX) and compound (XXXI) may be in the form of crystals.

The crystal of compound (I), compound (II), compound (XXX) and compound (XXXI) can be prepared by crystallization of compound (I), compound (II), compound (XXX) and compound (XXXI) by a crystallization method known per se.

Examples of the crystallization method include a method of crystallization from a solution, a method of crystallization from vapor, a method of crystallization from the melts and the like.

The “crystallization from a solution” is typically a method of shifting a non-saturated state to supersaturated state by varying factors involved in solubility of compounds (solvent composition, pH, temperature, ionic strength, redox state etc.) or the amount of solvent. To be specific, for example, a concentration method, a cold removing method, a reaction method (a diffusion method, an electrolysis method), a hydrothermal growth method, a flux method and the like can be mentioned. Examples of the solvent to be used include aromatic hydrocarbons (e.g., benzene, toluene, xylene etc.), halogenated hydrocarbons (e.g.,. dichloromethane, chloroform etc.), saturated hydrocarbons (e.g., hexane, heptane, cyclohexane etc.), ethers (e.g., diethyl ether, diisopropyl ether, tetrahydrofuran, dioxane etc.), nitrites (e.g., acetonitrile etc.), ketones (e.g., acetone etc.), sulfoxides (e.g., dimethyl sulfoxide etc.), acid amides (e.g., N,N-dimethylformamide etc.), esters (e.g., ethyl acetate etc.), alcohols (e.g., methanol, ethanol, isopropyl alcohol etc.), water and the like. These solvents are used alone or in a combination of two or more at a suitable ratio (e.g., 1:1 to 1:100 (a volume ratio)). Where necessary, a seed crystal can also be used.

The “crystallization from vapor” is, for example, a vaporization method (a sealed tube method, a gas stream method), a gas phase reaction method, a chemical transportation method and the like.

The “crystallization from the melts” is, for example, a normal freezing method (a Czochralski method, a temperature gradient method and a Bridgman method), a zone melting method (a zone leveling method and a floating zone method), a special growth method (a VLS method and a liquid phase epitaxy method) and the like.

Preferable examples of the crystallization method include a method of dissolving compound (I), compound (II), compound (XXX) or compound (XXXI) in a suitable solvent (e.g., alcohols such as methanol, ethanol etc. and the like) at a temperature of 20 to 120° C., and cooling the resulting solution to a temperature not higher than the temperature of dissolution (e.g., 0 to 50° C., preferably 0 to 20° C.) and the like.

The thus obtained crystals of the present invention can be isolated, for example, by filtration and the like.

As an analysis method of the obtained crystal, crystal analysis by powder X-ray diffraction is generally employed. Moreover, as a method for determining the crystal orientation, a mechanical method, an optical method and the like can also be mentioned.

The crystals of compound (I), compound (II), compound (XXX) or compound (XXXI) obtained in the above-mentioned production method (hereinafter to be abbreviated as “crystal of the present invention”) has high purity, high quality and low hygroscopicity, is free of denaturation even after a long-term preservation under normal conditions, and is extremely superior in stability. The crystal is also superior in biological properties (e.g., in vivo kinetics (absorbability, distribution, metabolism, excretion), efficacy expression etc.), and is extremely useful as a pharmaceutical agent.

In the present specification, the specific rotation ([α]D) means that measured using, for example, polarimeter (JASCO Corporation (JASCO), P-1030 polarimeter (No. AP-2)) and the like.

In the present specification, the melting point means that measured using, for example, a micromelting point apparatus (Yanako, MP-500D) or a DSC (differential scanning calorimetry) device (SEIKO, EXSTAR 6000) and the like.

In the present specification, the peak by powder X-ray diffraction means that measured using, for example, RINT Ultima⁺ 2100 (Rigaku Corporation) and the like with a Cu-Kα ray and the like as a ray source.

In general, the melting points and the peak by powder X-ray diffraction may vary depending on the measurement apparatuses, the measurement conditions and the like. The crystal in the present specification may show different values from the melting point or the peak by powder X-ray diffraction described in the present specification, as long as it is within each of a general error range.

The compound of the present invention has excellent antagonistic action for a tachykinin receptor, particularly Substance P receptor antagonistic action, neurokinin A receptor antagonistic action, in addition to inhibitory action for the increased permeability of blood vessel of a trachea induced by capsaicin. The compound of the present invention has low toxicity and thus it is safe.

Accordingly, the compound of the present invention having a superior antagonistic action for Substance P receptors and neurokinin A receptors etc. can be used as a safe pharmaceutical composition for preventing or treating the following Substance P-related diseases in mammals (e.g., mouse, rat, hamster, rabbit, cat, dog, bovine, sheep, monkey, human etc.).
(1) Lower urinary tract diseases [for example, abnormal urination such as overactive bladder, stress urinary incontinence, mixed urinary incontinence, lower urinary tract symptoms due to prostatomegaly, pelvic visceral pain, lower urinary tract symptoms due to chronic prostatitis, lower urinary tract symptoms due to interstitial cystitis and the like etc.],
(2) Gastrointestinal diseases [for example, irritable bowel syndrome, inflammatory bowel disease, ulcerative colitis, Crohn's disease, abnormality (e.g., gastritis, gastric ulcer etc.) caused by urease positive herical gram negative bacteria (e.g., Helicobacter pylori etc.), gastric cancer, postgastrostomy disorder, dyspepsia, esophageal ulcer, pancreatitis, colon polyp, cholelithiasis, hemorrhoids, peptic ulcer, situational ileitis, vomiting, nausea, motion disease, anorexia, gluttony, constipation, diarrhea, borborygmus etc.],
(3) Inflammatory or allergic diseases [for example, allergic rhinitis, conjunctivitis, gastrointestinal allergy, pollinosis, anaphylaxis, dermatitis, herpes, psoriasis, bronchitis, expectoration, retinopathy, postoperative and posttraumatic inflammation, regression of puffiness, pharyngitis, cystitis, meningitidis, inflammatory ophthalmic diseases etc.],
(4) Osteoarthropathy diseases [for example, rheumatoid arthritis (chronic rheumatoid arthritis), arthritis deformans, rheumatoid myelitis, osteoporosis, abnormal growth of cells, bone fracture, bone refracture, osteomalacia, osteopenia, osseous Behcet's disease, rigid myelitis, articular tissue destruction by gonarthrosis deformans and similar diseases thereto etc.],
(5) Respiratory diseases [for example, cold syndrome, pneumonia, asthma, pulmonary hypertension, pulmonary thrombi/pulmonary obliteration, pulmonary sarcoidosis, pulmonary tuberculosis, interstitial pneumonia, silicosis, adult tachypnea syndrome, chronic obliterative pulmonary diseases, cough etc.],
(6) Infectious diseases [HIV infectious diseases, virus infectious diseases due to cytomegalo virus, influenza virus, herpes virus and the like, rickettsia infectious diseases, bacterial infectious diseases, sexually-transmitted diseases, carinii pneumonia, helicobacter pylori infectious disease, systemic fungal infectious diseases, tuberculosis, invasive staphylococcal infectious diseases, acute viral encephalitis, acute bacterial meningitidis, AIDS encephalitis, septicemia, sepsis, sepsis gravis, septic shock, endotoxin shock, toxic shock syndromes etc.],
(7) Cancers [for example, primary, metastatic or recurrent breast cancer, prostatic cancer, pancreatic cancer, gastric cancer, lung cancer, colorectal cancer (colon cancer, rectal cancer, anal cancer), esophagus cancer, duodenal cancer, head and neck cancer (tongue cancer, pharynx cancer, larynx cancer), brain tumor, neurinoma, non-small cell lung cancer, small cell lung cancer, hepatic cancer, renal cancer, colic cancer, uterine cancer (cancer of the uterine body, uterine cervical cancer), ovarian cancer, bladder cancer, skin cancer, hemangioma, malignant lymphoma, malignant melanoma, thyroid cancer, bone tumor, hemangioma, angiofibroma, retinosarcoma, penis cancer, pediatric solid cancer, Kaposi's sarcoma, Kaposi's sarcoma caused by AIDS, tumor of the maxillary sinus, fibrous histiocytoma, smooth muscle sarcoma, rhabdomyosarcoma, liposarcoma, fibroid tumors of the uterus, osteoblastoma, osteosarcoma, chondrosarcoma, carcinomatous mesothelial tumor, tumors such as leukemia, Hodgkin's disease etc.],
(8) central neurological disease [for example, neurodegenerative disease (e.g., Alzheimer's disease, Down's disease, Parkinson's disease, Creutzfeldt-Jakob's disease, amyotrophic lateral sclerosis (ALS), Huntington chorea, diabetic neuropathy, multiple sclerosis etc.), mental diseases (e.g., schizophrenia, depression, mania, anxiety neurosis, obsessive-compulsive neurosis, panic disorder, epilepsy, alcohol dependence, anxiety, anxious mental state, emotional abnormality, cyclothymic temperament, nervous erethism, autism, faint, addiction, low sex drive etc.), central nervous system and peripheral nerve disorders (e.g., head trauma, spinal trauma, brain edema, disorders of sensory function, abnormality of sensory function, disorders of autonomic nervous function, abnormality of autonomic nervous function, whiplash injury etc.), memory disorders (e.g., senile dementia, amnesia, cerebrovascular dementia etc.), cerebrovascular disorders (e.g., disorders and aftereffect and/or complication from intracerebral hemorrhage, brain infarction, etc, asymptomatic cerebro-vascular accident, transient cerebral ischemic attack, hypertensive encephalopathia, blood-brain barrier disorder etc.), recurrence and aftereffect of cerebro-vascular accident (neural symptoms, mental symptoms, subjective symptoms, disorders of daily living activities etc.), post-cerebrovascular occlusion central hypofunction; disorder or abnormality of cerebral circulation and/or autoregulation of renal circulation], sleep disorder,
(9) Circulatory diseases [for example, acute coronary artery syndromes (e.g., acute cardiac infarction, unstable angina etc.), peripheral arterial obstruction, Raynaud's disease; Buerger disease; restenosis after coronary-artery intervention (percutaneous transluminal coronary angioplasty (PTCA), directional coronary atherectomy (DCA), stenting etc.), restenosis after coronary-artery bypass operation, restenosis after intervention (angioplasty, atherectomy, stenting etc.) or bypass operation in other peripheral artery, ischemic cardiac diseases (e.g., cardiac infarction, angina etc.), myocarditis, intermittent claudication, lacunar infarction, arteriosclerosis (e.g., atherosclerosis etc.), cardiac failure (acute cardiac failure, chronic cardiac failure accompanied by congestion), arrhythmia, progress of atherosclerotic plaque, thrombosis, hypertension, hypertensive tinnitus; hypotension etc.],
(10) pain (for example, headache, migraine, neuralgia and pelvic visceral pain including cystalgia etc.],
(11) Autoimmune diseases [for example, collagen disease, systemic lupus erythematosus, scleroderma, polyarteritis, myasthenia gravis, multiple sclerosis, Sjogren's syndrome, Behcet's disease etc.],
(12) Hepatic diseases [e.g., hepatitis (including chronic hepatitis), cirrhosis, interstitial hepatic diseases etc.],
(13) Pancreatic diseases [e.g., pancreatitis (including chronic pancreatitis) etc.],
(14) Renal diseases [e.g., nephritis, glomerulonephritis, glomerulosclerosis, renal failure, thrombotic microangiopathy, dialysis complications, organ disorders including nephropathia by radiation, diabetic nephropathia etc.],
(15) Metabolic diseases [e.g., diabetic diseases (insulin-dependent diabetes, diabetic complications, diabetic retinopathy, diabetic microangiopathy, diabetic neuropathy etc.); glucose tolerance abnormality, obesity, prostatomegaly, sexual dysfunction etc.],
(16) Endocrine diseases [e.g., Addison's disease, Cushing's syndrome, melanocytoma, primary aldosteronism etc.],
(17) Other diseases
(a) Transplant rejection [e.g., posttransplantational rejection, posttransplantational polycythemia, hypertension, organ disorder and/or vascular hypertrophy, graft-versus-host disease etc.],
(b) Abnormality in characteristic of blood and/or blood components [e.g., enhancement in platelet aggregation, abnormality of erythrocyte deformability, enhancement in leukocyte adhesiveness, increase in blood viscosity, polycythemia, vascular peliosis, autoimmune hemolytic anemia, disseminated intravascular coagulation syndrome (DIC), multiple myelopathy etc.],
(c) Gynecologic diseases [e.g., climacteric disorder, gestational toxicosis, endometriosis, hysteromyoma, ovarian disease, mammary disease, premenstrual syndrome etc.],
(d) Dermatic diseases [e.g., keloid, angioma, psoriasis, pruritus etc.],
(e) Ophthalmic diseases [e.g., glaucoma, ocular hypertension disease etc.],
(f) Otolaryngological diseases [e.g., Menuel syndrome, tinnitus, gustation disorder, dizziness, disequilibrium, dysphagia etc.],
(g) Diseases due to environmental and/or occupational factors (e.g., radiation disorder, disorders by ultraviolet ray-infrared ray-laser ray, altitude sickness etc.),
(h) ataxia, rigidity, tremor, motion impairment, akinesia,
(i) chronic fatigue syndrome,
(j) sudden infant death syndrome,
(k) hiccup,
(l) diseases causing palpitation, vertigo, heartburn and the like.

Of these diseases, the compound of the present invention is particularly useful as a tachykinin receptor antagonist, an agent for improving lower urinary tract symptoms such as frequent urination, incontinence and the like, a therapeutic drug for these lower urinary tract symptoms, an agent for the prophylaxis or treatment of gastrointestinal diseases, or an agent for the prophylaxis or treatment of central neurological disease. Specifically, the compound of the present invention is useful as an agent for the prophylaxis or treatment of overactive bladder, irritable bowel syndrome, inflammatory bowel disease, vomiting, nausea, depression, anxiety neurosis, anxiety, pelvic visceral pain or interstitial cystitis.

A pharmaceutical preparation containing the compound of the present invention may be in any solid preparation such as powder, granule, tablet, capsule, suppository, orally-disintegrating film etc., or in any liquid form of syrup, emulsion, injection, suspension etc.

A pharmaceutical preparation containing the compound of the present invention can be produced by any conventional method, for example, blending, kneading, granulation, tabletting, coating, sterilization, emulsification etc., in accordance with the form of the preparation to be produced. For the production of such pharmaceutical preparations, for example, reference can be made to each of the items in General principles for pharmaceutical preparations in the Japanese Pharmacopeia. In addition, the pharmaceutical preparation of the present invention may be formulated into a sustained release preparation containing an active ingredient and a biodegradable polymer compound. The sustained release preparation can be produced according to the method described in JP-A-9-263545.

In the pharmaceutical preparations of the present invention, the content of the compound or a salt thereof in the present invention varies depending on the forms of the preparations, but is generally about 0.01 to 100% by weight, preferably about 0.1 to 50% by weight, more preferably 0.5 to 20% by weight, relative to the total weight of each preparation.

When the compound of the present invention is used in the above-mentioned pharmaceutical preparations, it may be used alone, or in admixture with a suitable, pharmaceutically acceptable carrier, for example, excipients (e.g., starch, lactose, sucrose, calcium carbonate, calcium phosphate etc.), binders (e.g., starch, arabic gum, carboxymethyl cellulose, hydroxypropyl cellulose, crystalline cellulose, alginic acid, gelatin, polyvinyl pyrrolidone etc.), lubricants (e.g., stearic acid, magnesium stearate, calcium stearate, talc etc.), disintegrants (e.g., calcium carboxymethylcellulose, talc etc.), diluents (e.g., water for injection, physiological saline etc.) and if desired, with the additives (e.g., a stabilizer, a preservative, a colorant, a fragrance, a dissolution aid, an emulsifier, a buffer, an isotonic agent etc.) and the like, by ordinary methods. It can be formulated into the solid preparations such as powders, fine granules, granules, tablets, capsules, orally-disintegrating films etc., or into the liquid preparations such as injections etc., and can be administered orally or parenterally.

The dose of the pharmaceutical preparation of the present invention varies depending on the kind of the compound of the present invention or a pharmaceutically acceptable salt thereof, the administration route, the condition and the age of patients etc. For example, the dose for oral administration of the pharmaceutical preparation to an adult patient suffering from abnormal urination is generally from about 0.005 to 50 mg/kg body/day, preferably from about 0.05 to 10 mg/kg body/day, more preferably from about 0.2 to 4 mg/kg body/day, based on the compound of the present invention, which may be administered once a day or in two or three divided portions a day.

The dose when the pharmaceutical composition of the present invention is a sustained release preparation varies depending on the kinds and the content of the compound of the present invention, the formulation, the duration time of drug release, the animals to be administered (e.g., mammals such as humans, rats, mice, cats, dogs, rabbits, bovines, swines etc.), and the object of administration. For example, when it is parenterally administered, preferably about 0.1 to about 100 mg of the compound of the present invention is released from the preparation for 1 week.

The compound of the present invention can be used in a mixture or combination with other pharmaceutically active ingredients at a suitable ratio.

Combination of the compound of the present invention with other pharmaceutically active ingredients can give the following excellent effects:
(1) a dose can be reduced as compared with separate administration of the compound of the present invention or other pharmaceutically active ingredients. More specifically, when the compound of the present invention is combined with anticholinergic agents or NK-2 receptor antagonists, the dose can be reduced as compared with separate administration of anticholinergic agents or NK-2 receptor antagonists, and therefore, side effects such as dry mouth can be reduced;
(2) according to symptoms of patient (mild symptoms, severe symptoms etc.), a drug to be combined with the compound of the present invention can be selected;
(3) by choosing other pharmaceutically active ingredients which have different mechanism of action from that of the compound of the present invention, the therapeutic period can be designed longer;
(4) by choosing other pharmaceutically active ingredients which have different mechanism of action from that of the compound of the present invention, continuation of therapeutic effects can be obtained; and
(5) by combining the compound of the present invention and other pharmaceutically active ingredients, synergic effects can be obtained.

A drug which is mixed or combined with the compound of the present invention (hereinafter, briefly referred to as combination drugs) includes the following:

### (1) Agent for Treating Diabetes

Insulin preparations (e.g., animal insulin preparations extracted from the bovine or swine pancreas; human insulin preparations synthesized by a genetic engineering technique using Escherichia coli or a yeast; insulin zinc; protamine zinc insulin; a fragment or a derivative of insulin (e.g., INS-1 etc.), and the like), agents for potentiating insulin sensitivity (e.g., pioglitazone hydrochloride, troglitazone, rosiglitazone or its maleate, JTT-501, MCC-555, YM-440, GI-262570, KRP-297, FK-614, CS-011 etc.), α-glucosidase inhibitors (e.g., voglibose, acarbose, miglitol, emiglitate etc.), biguanides (e.g., phenformin, metformin, buformin etc.), sulfonylureas (e.g., tolbutamide, glibenclamide, gliclazide, chlorpropamide, tolazamide, acetohexamide, glyclopyramide, glimepiride etc.) and other insulin secretagogues (e.g., repaglinide, senaglinide, mitiglinide or its calcium salt hydrate, GLP-1, nateglinide etc.), dipeptidyl peptidase IV inhibitor (e.g., Vildagliptin, sitagliptin, saxagliptin, alogliptin, NVP-DPP-728, PT-100, P32/98 etc.), amylin agonists (e.g., pramlintide etc.), phosphotyrosine phosphatase inhibitors (e.g., vanadic acid etc.), gluconeogenesis inhibitors (e.g., glycogen phosphorylase inhibitors, glucose-6-phosphatase inhibitors, glucagon antagonists etc.), SGLT (sodium-glucose cotransporter) inhibitors (e.g., T-1095 etc.) and the like.

### (2) Agent for Treating Diabetic Complications

Aldose reductase inhibitors (e.g., tolrestat, epalrestat, zenarestat, zopolrestat, fidarestat (SNK-860), minalrestat (ARI-509), CT-112 etc.), neurotrophic factors (e.g., NGF, NT-3 etc.), AGE inhibitors (e.g., ALT-945, pimagedine, pyratoxathine, N-phenacylthiazolium bromide (ALT-766), EXO-226 etc.), active oxygen scavengers (e.g., thioctic acid etc.), cerebral vasodilators (e.g., tiapuride etc.) and the like.

### (3) Antihyperlipidemic Agent

Statin compounds inhibiting cholesterol synthesis (e.g., pravastatin, simvastatin, lovastatin, atorvastatin, fluvastatin, cerivastatin or their salt (e.g., sodium salt etc.) and the like), squalene synthase inhibitors or fibrate compounds having triglyceride lowering action (e.g., bezafibrate, clofibrate, simfibrate, clinofibrate etc.) and the like.

### (4) Hypotensive Agent

Angiotensin converting enzyme inhibitors (e.g., captopril, enalapril, delapril etc.), angiotensin II antagonists (e.g., losartan, candesartan cilexetil etc.), calcium antagonists (e.g., manidipine, nifedipine, amlodipine, efonidipine, nicardipine etc.), clonidine, and the like.

### (5) Antiobesity Agent

Antiobesity drugs acting on the central nervous system (e.g. dexfenfluramine, fenfluramine, phentermine, sibutramine, anfepramone, dexamphetamine, mazindol, phenylpropanolamine, clobenzorex etc.), pancreatic lipase inhibitors (e.g. orlistat etc.), β₃ agonists (e.g. CL-316243, SR-58611-A, UL-TG-307, AJ-9677, AZ40140, YM-178, KRP-204, KUC-7483, KUC-7322, KUL-7211, MN-246, L-796568, talibegron, solabegron etc.), anorectic peptides (e.g. leptin, CNTF (Ciliary Neurotrophic Factor) etc.), cholecystokinin agonists (e.g. lintitript, FPL-15849 etc.), serotonin 2C receptor agonists (e.g., APD-356, SCA-136, ATHX-105, WAY-163909, YM-348), and the like.

### (6) Diuretic Agent

Xanthine derivatives (e.g., theobromine sodium salicylate, theobromine calcium salicylate etc.), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide etc.), antialdosterone preparations (e.g., spironolactone, triamterene etc.), carbonic anhydrase inhibitors (e.g., acetazolamide etc.), chlorobenzenesulfonamide preparations (e.g., chlorthalidone, mefruside, indapamide etc.), azosemide, isosorbide, ethacrynic acid, piretanide, bumetanide, furosemide etc.

### (7) Chemotherapeutic Agent

Alkylating agents (e.g., cyclophosphamide, ifosfamide etc.), metabolic antagonists (e.g., methotrexate, 5-fluorouracil etc.), antitumor antibiotics (e.g., mitomycin, adriamycin etc.), plant-derived antitumor agents (e.g., vincristine, vindesine, taxol etc.), cisplatin, carboplatin, etoposide etc. Among these, 5-fluorouracil derivatives such as Furtulon and Neo-Furtulon are preferred.

### (8) Immunotherapeutic Agent

Microorganism- or bacterium-derived components (e.g., muramyl dipeptide derivatives, Picibanil etc.), immunopotentiator polysaccharides (e.g., lentinan, schizophyllan, krestin etc.), genetically engineered cytokines (e.g., interferons, interleukins (IL) etc.), colony stimulating factors (e.g., granulocyte colony stimulating factor, erythropoietin etc.) and the like. Among these, IL-1, IL-2, IL-12 etc. are preferred.

### (9) Therapeutic Agent Recognized to Ameliorate Cachexia in Animal Models or Clinical Practice

Progesterone derivatives (e.g., megestrol acetate) [Journal of Clinical Oncology, vol. 12, pp. 213-225, 1994], metoclopramide pharmaceuticals, tetrahydrocannabinol pharmaceuticals (the above reference is applied to both), fat metabolism ameliorating agents (e.g., eicosapentanoic acid) [British Journal of Cancer, vol. 68, pp. 314-318, 1993], growth hormones, IGF-1, and antibodies to the cachexia-inducing factors such as TNF-60 , LIF, IL-6 and oncostatin M.

### (10) Antiinflammatory Agent

Steroids (e.g., dexamethasone etc.), sodium hyaluronate, cyclooxygenase inhibitors (e.g., indomethacin, ketoprofen, loxoprofen, meloxicam, ampiroxicam, celecoxib, rofecoxib etc.) and the like.

### (11) Miscellaneous

Glycosylation inhibitors (e.g., ALT-711 etc.), nerve regeneration promoting drugs (e.g., Y-128, VX853, prosaptide etc.), drugs acting on the central nervous system (e.g., antidepressants such as desipramine, amitriptyline, imipramine, fluoxetine, paroxetine, doxepin, duloxetine, venlafaxine etc.), anticonvulsants (e.g., lamotrigine, carbamazepine, gabapentin), antiarrhythmic drugs (e.g., mexiletine), acetylcholine receptor ligands (e.g., ABT-594), endothelin receptor antagonists (e.g., ABT-627), monoamine uptake inhibitors (e.g., tramadol), indoleamine uptake inhibitors (e.g., fluoxetine, paroxetine), narcotic analgesics (e.g., morphine), nonnarcotic analgesics (e.g., buprenorphine, axomadol), GABA receptor agonists, GABA uptake inhibitors (e.g., tiagabine), α2 receptor agonists (e.g., clonidine), local analgesics (e.g., capsaicin), protein kinase C inhibitors (e.g., LY-333531), antianxiety drugs (e.g., benzodiazepines), phosphodiesterase inhibitors (e.g., sildenafil), dopamine receptor agonists (e.g., apomorphine), dopamine receptor antagonists (e.g., haloperidol), serotonin receptor agonists (e.g., tandospirone citrate, sumatryptan, tegaserod), serotonin receptor antagonists (e.g., cyproheptadine hydrochloride, ondansetron), serotonin uptake inhibitors (e.g., fluvoxamine maleate, fluoxetine, paroxetine), sleep-inducing drugs (e.g., triazolam, zolpidem), anticholinergic agents, α₁ receptor blocking agents (e.g., tamsulosin, urapidil, naftopidil, silodosin), muscle relaxants (e.g., baclofen etc.), potassium channel openers (e.g., nicorandil), calcium channel blocking agents (e.g., nifedipine), chloride channel openers (e.g., lubiprostone), agents for preventing and/or treating Alzheimer's disease (e.g., donepezil, rivastigmine, galanthamine), agents for treating Parkinson's disease (e.g., L-dopa), agents for preventing and/or treating multiple sclerosis (e.g., interferon β-1a), histamine H₁ receptor inhibitors (e.g., promethazine hydrochloride), proton pump inhibitors (e.g., lansoprazole, omeprazole), antithrombotic agents (e.g., aspirin, cilostazol), NK-2 receptor antagonists, NK-3 receptor antagonists (e.g., talnetant), agents of treating HIV infection (saquinavir, zidovudine, lamivudine, nevirapine), agents of treating chronic obstructive pulmonary diseases (salmeterol, thiotropium bromide, cilomilast), diuretics (e.g., furosemide), antidiuretics (e.g., vasopressin V2 receptor agonist) and the like.

Anticholinergic agents include, for example, atropine, scopolamine, homatropine, tropicamide, cyclopentolate, butyl scopolamine bromide, propantheline bromide, methylbenactyzium bromide, mepenzolate bromide, flavoxate, pirenzepine, ipratropium bromide, trihexyphenidyl, oxybutynin, propiverine, darifenacin, tolterodine, solifenacin, temiverine, trospium chloride or a salt thereof (e.g., atropine sulfate, scopolamine hydrobromide, homatropine hydrobromide, cyclopentolate hydrochloride, flavoxate hydrochloride, pirenzepine hydrochloride, trihexyphenidyl hydrochloride, oxybutynin chloride, tolterodine tartrate, solifenacin succinate etc.) and the like, preferably, oxybutynin, propiverine, darifenacin, tolterodine, solifenacin, temiverine, trospium chloride or a salt thereof (e.g., oxybutynin chloride, tolterodine tartrate, solifenacin succinate etc.). In addition, acetylcholine esterase inhibitors (e.g., distigmine etc.) and the like can be used.

NK-2 receptor antagonists include, for example, a piperidine derivative such as GR159897, GR149861, SR48968 (saredutant), SR144190, YM35375, YM38336, ZD7944, L-743986, MDL105212A, ZD6021, MDL105172A, SCH205528, SCH62373, R-113281 etc., a perhydroisoindole derivative such as RPR-106145 etc., a quinoline derivative such as SB-414240 etc., a pyrrolopyrimidine derivative such as ZM-253270 etc., a pseudopeptide derivative such as MEN11420 (nepadutant), SCH217048, L-659877, PD-147714 (CAM-2291), MEN10376, S16474 etc., and others such as GR100679, DNK333, GR94800, UK-224671, MEN10376, MEN10627, or a salt thereof, and the like.

The pharmaceutical composition comprising a mixture or combination of the compound of the present invention and a concomitant drug may be formulated into
(1) a single formulation as a pharmaceutical composition containing the compound of the present invention and the concomitant drug, or
(2) a formulation comprising the compound of the present invention and the concomitant drug which are separately formulated. Hereinafter, they are generally abbreviated as the combination drug of the present invention.

The combination drug of the present invention can be formulated by mixing the compound of the present invention and the active ingredient of the concomitant drug separately or simultaneously as they are or together with a pharmaceutically acceptable carrier etc. in the same manner as in the above-mentioned pharmaceutical preparation comprising the compound of the present invention.

A daily dose of the combination drug of the present invention varies depending on severity of the symptoms, age, sex, weight and sensitivity of the subject to be administered, time and interval of administration, property, formulation and kinds of pharmaceutical preparation, kinds of active ingredients, etc., and is not particularly limited. The daily dose in terms of the compound of the present invention is not particularly limited if it causes no problems of side effects. In the case of oral administration, a daily dosage is generally in a range of about 0.005 to 100 mg, preferably about 0.05 to 50 mg, and more preferably about 0.2 to 30 mg, per 1 kg body weight of mammals, which may be administered once a day or in two or three divided portions a day.

The dose of the compound or the combination drug of the present invention may be set within the range such that it causes no problems of side effects. The daily dose as the compound or the combination drug of the present invention varies depending on severity of symptoms, age, sex, weight and sensitivity of the subject to be administered, time and interval of administration, property, formulation and kinds of pharmaceutical preparation, kinds of active ingredients, etc., and is not particularly limited. In the case of oral administration, a daily dosage in terms of active ingredients is generally in the order of about 0.001 to 2000 mg, preferably about 0.01 to 500 mg, and more preferably about 0.1 to 100 mg, per 1 kg body weight of mammals, which may be administered once a day or in two to four divided portions a day.

In administering the combination drug of the present invention, the compound of the present invention and the combination drugs may be administered at the same time or, the combination drugs may be administered before administering the compound of the present invention, and vice versa. In case of staggered administration, the time interval varies depending on the active ingredients to be administered, a formulation and an administration route. For example, if the combination drugs are administered first, the compound of the present invention may be administered 1 minute to 3 days, preferably 10 min to 1 day, more preferably 15 min to 1 hr. after administering the combination drugs. If the compound of the present invention is administered first, the combination drugs may be administered 1 minute to 1 day, preferably 10 min to 6 hr, more preferably 15 min to 1 hr. after administering the compound of the present invention.

In a preferred administration method, about 0.001 to 200 mg/kg of the combination drugs formulated as an oral preparation is administered orally and then after about 15 minutes, about 0.005 to 100 mg/kg of the compound of the present invention formulated as an oral preparation is administered orally as a daily dose.

In the combination drug of the present invention, the content of the compound of the present invention varies depending on the forms of the preparation, but generally in the order of 0.01 to 100 wt %, preferably 0.1 to 50 wt %, and further preferably 0.5 to 20 wt %, relative to the total preparation.

In addition, the compounds described in Reference Examples of the present specification also have a superior tachykinin receptor antagonistic action as does compound (I) and the like.

### EXAMPLES

The present invention is further described in detail in with reference to Reference Examples, Examples, Preparative Examples and Experimental Examples which are not intended to restrict the invention and may be modified without departing from the scope of the invention.

Elution in the column chromatography in the following Reference Examples and Examples was conducted under observation by TLC (thin layer chromatography), unless otherwise specifically indicated. In the TLC observation, 60F254, TLC plates, produced by Merck & Co., Inc. was used, and the solvent employed as an elution solvent in the column chromatography was used as an eluent. For the detection, a UV detector was used. As silica gel for the column chromatography, Silica Gel 60 (70 to 230 mesh) produced by Merck & Co., Inc. was used. The “room temperature” referred herein means temperature generally from about 10° C. to 35° C. For drying extract, sodium sulfate or magnesium sulfate was used. The abbreviations in Examples and Reference Examples mean the following.

LC: liquid chromatography

MS: mass spectrometry spectrum

ESI: electrospray ionization method

FAB: fast atom bombardment method

M: molecular weight of the compound

NMR: nuclear magnetic resonance spectrum

Hz: hertz

J: coupling constant

m: multiplet

q: quartet

t: triplet

d: doublet

s: singlet

br: broad

dt: double triplet

ddd: double double doublet

brs: broad singlet

^{t}Bu: tert-butyl group

Boc: tert-butyloxycarbonyl group

N: normal concentration

MPa: mega pascal

MeOH: methanol

EtOH: ethanol

DMF: N,N-dimethylformamide

THF: tetrahydrofuran

DMSO: dimethyl sulfoxide

IPE: diisopropyl ether

DME: 1,2-dimethoxyethane

HOBt-H₂O: 1-hydroxybenzotriazole 1 hydrate

HOBt-NH₃: 1-hydroxybenzotriazole ammonia complex

WSC HCl: 1-ethyl-3-(3-dimethylaminopropyl)carbodiimide hydrochloride

NaBH(OAc)₃: sodium triacetoxyborohydride

Pd(PPh₃)₄: tetrakis(triphenylphosphine)palladium (0)

Et₃N: triethylamine

TFA: trifluoroacetic acid

LC-MS in Examples and Reference Examples were measured under the following conditions.
Analysis by LC-MS
Instrument: Waters LC-MS system
HPLC: Agilent HP1100
MS: Micromass ZMD
HPLC conditions
Column: CAPCELL PAK C18UG120, S-3 μm, 1.5×35 mm (Shiseido)

Solvents: Solution A; water containing 0.05% trifluoroacetic acid, Solution B; acetonitrile containing 0.05% trifluoroacetic acid

Gradient cycles: 0.00 min. (Solution A/Solution B=90/10), 2.00 min. (Solution A/Solution B=5/95), 2.75 min. (Solution A/Solution B=5/95), 2.76 min. (Solution A/Solution B=90/10), 3.60 min. (Solution A/Solution B=90/10)

Injection volume: 2 μL, Flow rate: 0.5 mL/min, Detection method: UV 220 nm

MS conditions

ionization method: ESI

In the description of mass spectometry for the compounds as exemplified below, molecular weight of the corresponding compounds is represented by M.

Purification by preparative HPLC in Examples and Reference Examples was carried out under the following conditions.

Instrument: High Throughput Purification System, Gilson Company, Inc.

Column: CombiPrep ODS-A S-5 μm, 50×20 mm (YMC)

Solvents: Solution A; 0.1% trifluoroacetic acid-containing water, Solution B; 0.1% trifluoroacetic acid-containing acetonitrile

Gradient cycle: 0.00 minute (Solution A/Solution B=95/5), 1.00 minute (Solution A/Solution B=95/5), 5.20 min. (Solution A/Solution B=5/95), 6.40 min. (Solution A/Solution B=5/95), 6.50 min. (Solution A/Solution B=95/5), 6.60 min. (Solution A/Solution B=95/5)

Flow rate: 25 ml/min, Detection method: UV 220 nm

Chiral HPLC conditions (measurement of diastereomer, enantiomer excess of Reference Example 1)

Column: CHIRALCEL OD-RH 4.6 mm ID×150 mm

Solvent: 50 mM potassium dihydrogen phosphate (pH 8.0)/acetonitrile=85/15

Injection volume: 20 μL

Flow rate: 0.3 mL/min

Temperature: 40° C.

Detection method: UV 220 nm

The reaction by microwave in the Examples was performed using the following apparatus.
Instrument: Emrys Optimizer, Biotage Japan Ltd.

### Reference Example 1

### (3R*,4S*)-1-[amino(oxo)acetyl]-N-[3,5-bis(trifluoromethyl)benzyl]-N-methyl-3-phenylpiperidine-4-carboxamide

To a solution of (3R*,4S*)—N-[3,5-bis(trifluoromethyl)benzyl]-N-methyl-3-phenylpiperidine-4-carboxamide monohydrochloride (400 mg) synthesized by a known method (WO2005/068427), oxamic acid (113 mg) and Et₃N (348 μL) in THF (10 mL) were added WSC HCl (244 mg) and HOBt.H₂O (193 mg), and the mixture was stirred at room temperature for 19 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (184 mg, 43%) as a white amorphous solid.
MS (ESI+):516 (M+H)

### Reference Example 2

### (3R*,4R*)-1-[amino(oxo)acetyl]-N-[3,5-bis(trifluoromethyl)benzyl]-N-methyl-3-phenylpiperidine-4-carboxamide

The title compound was obtained by reaction and purification in the same manner as in Reference Example 1 and using (3R*,4R*)—N-[3,5-bis(trifluoromethyl)benzyl]-N-methyl-3-phenylpiperidine-4-carboxamide monohydrochloride synthesized by a known method (WO2005/068427) and oxamic acid.
MS (ESI+):516 (M+H)

### Reference Example 3

### Methyl 3-[((3R*,4S*)-4-{[[3,5-bis(trifluoromethyl)benzyl](methyl)amino]carbonyl}-3-phenylpiperidin-1-yl)carbonyl]azetidine-1-carboxylate

To a solution of the compound (300 mg) obtained in Example 6 and Et₃N (185 μL) in THF (6 mL) were added methyl chloroformate (50.2 μL) at 0° C., and the mixture was stirred for 1.5 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (122 mg, 39%) as a white amorphous solid.
MS (ESI+):586 (M+H)

### Reference Example 4

### (3R*,4S*)-1-[(1-acetylazetidin-3-yl)carbonyl]-N-(3,5-bis(trifluoromethyl)benzyl)-N-methyl-3-phenylpiperidine-4-carboxamide

The title compound was obtained by reaction and purification in the same manner as in Reference Example 3 and using acetyl chloride.
MS (ESI+):570 (M+H)

### Reference Example 5

### tert-butyl 4-[((3R*,4S*)-4-{[[3,5-bis(trifluoromethyl)benzyl](methyl)amino]carbonyl}-3-phenylpiperidin-1-yl)carbonyl]piperidine-1-carboxylate

The title compound was obtained by reaction and purification in the same manner as in Reference Example 1 and using N-Boc-isonipecotic acid.
MS (ESI+):656 (M+H)

### Reference Example 6

### (3R*,4S*)—N-[3,5-bis(trifluoromethyl)benzyl]-N-methyl-3-phenyl-1-(piperidin-4-ylcarbonyl)piperidine-4-carboxamide monohydrochloride

To a mixed solution of the compound (1.98 g) obtained in Reference Example 5 in ethyl acetate (20 mL)-EtOH (5 mL) was added a 4N hydrogen chloride/ethyl acetate (2.27 mL) solution, and the mixture was stirred with heating at 60° C. for 2 hr. The reaction mixture was concentrated under reduced pressure, and the precipitate was collected by filtration with IPE and hexane to give the title compound (1.79 g, 100%) as a white amorphous solid.
MS (ESI+):556 (M−HCl+H)

### Reference Example 7

### (3R*,4R*)—N-[3,5-bis(trifluoromethyl)benzyl]-N-methyl-1-(methylsulfonyl)-3-phenylpiperidine-4-carboxamide

### Reference Example 8

### (3R*,4R*)—N-[3,5-bis(trifluoromethyl)benzyl]-N-methyl-1-[2-(methylsulfonyl)ethyl]-3-phenylpiperidine-4-carboxamide monohydrochloride

To a solution of 2-(methylsulfonyl)ethanol (204 mg) in THF (10 mL) were added methanesulfonyl chloride (124 μL) and Et₃N (218 μL) at room temperature. The mixture was stirred at room temperature for 2 hr, and the precipitate was collected by filtration. To the filtrate was added a solution of (3R*,4R*)—N-[3,5-bis(trifluoromethyl)benzyl]-N-methyl-3-phenylpiperidine-4-carboxamide monohydrochloride (300 mg) and Et₃N (218 μL) in THF (20 mL) at room temperature, and the mixture was stirred for 26 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The compound (118 mg, 36%) of Reference Example 7 was obtained as a white amorphous solid from a fraction with a short retention time. A fraction with a long retention time was concentrated under reduced pressure and treated with 1 equivalent amount of 4N hydrogen chloride/ethyl acetate to give the compound (148 mg, 43%) of Reference Example 8 as a white amorphous solid.

### Compound of Reference Example 7

MS (ESI+):523 (M+H)

### Compound of Reference Example 8

MS (ESI+):551 (M−HCl+H)

### Reference Example 9

### (3R*,4S*)—N-[3,5-bis(trifluoromethyl)benzyl]-N-methyl-3-phenyl-1-pyrimidin-2-ylpiperidine-4-carboxamide monohydrochloride

To a solution of (3R*,4S*)—N-[3,5-bis(trifluoromethyl)benzyl]-N-methyl-3-phenylpiperidine-4-carboxamide monohydrochloride (400 mg) in THF (8 mL) was added Et₃N (128 μL), and the mixture was stirred at room temperature for 20 min. The precipitate was filtered off, and the filtrate was concentrated under reduced pressure. The residue was dissolved in EtOH (2 mL), and 2-chloropyrimidine (98.2 mg) was added. The reaction container was irradiated in a microwave reaction apparatus at 100° C., 30 min×2 times. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (106.2 mg, 23%) as a white amorphous solid.
MS (ESI+):523 (M−HCl+H)

### Reference Example 10

### (3R*,4S*)—N-[3,5-bis(trifluoromethyl)benzyl]-N-methyl-3-phenyl-1-pyrazin-2-ylpiperidine-4-carboxamide monohydrochloride

The title compound was obtained by reaction and purification in the same manner as in Reference Example 9 and using 2-chloropyrazine.
MS (ESI+):523 (M−HCl+H)

The compounds described in Reference Examples 1-10 are as follows (Table 1).

TABLE 1

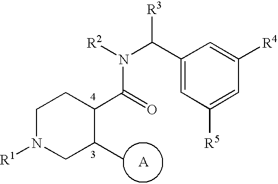

| Ref.Ex. No. | stereo-chemistry | 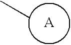 | R¹ | R² | R³ | R⁴ | R⁵ | ^{additives} | MS(ESI) |
|---|---|---|---|---|---|---|---|---|---|
| 1 | (3R*, 4S*) | 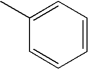 | 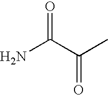 | CH₃ | H | CF₃ | CF₃ | | 516(M + H) |
| 2 | (3R*, 4R*) | 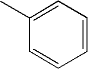 | 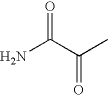 | CH₃ | H | CF₃ | CF₃ | | 516(M + H) |
| 3 | (3R*, 4S*) | 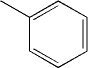 | 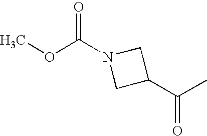 | CH₃ | H | CF₃ | CF₃ | | 586(M + H) |
| 4 | (3R*, 4S*) | 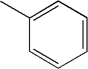 | 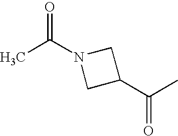 | CH₃ | H | CF₃ | CF₃ | | 570(M + H) |
| 5 | (3R*, 4S*) | 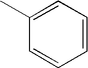 | 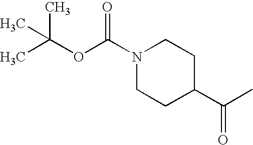 | CH₃ | H | CF₃ | CF₃ | | 656(M + H) |
| 6 | (3R*, 4S*) | 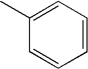 | 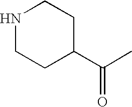 | CH₃ | H | CF₃ | CF₃ | HCl | 556(M −HCl + H) |
| 7 | (3R*, 4R*) | 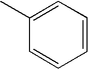 | 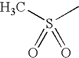 | CH₃ | H | CF₃ | CF₃ | | 523(M + H) |
| 8 | (3R*, 4R*) | 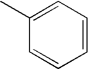 | 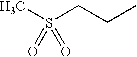 | CH₃ | H | CF₃ | CF₃ | HCl | 551(M −HCl + H) |
| 9 | (3R*, 4S*) | 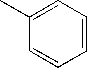 | 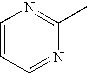 | CH₃ | H | CF₃ | CF₃ | HCl | 523(M −HCl + H) |
| 10 | (3R*, 4S*) | 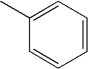 | 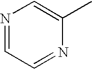 | CH₃ | H | CF₃ | CF₃ | HCl | 523(M −HCl + H) |

### Reference Example 11

### 1-glycoloylpiperidine-4-carboxylic acid

### (Step 1)

To a solution of N-Boc-isonipecotic acid (2.3 g) and DMF (about 50 μL) in THF (15 mL) was added oxalyl chloride (0.95 mL) at 0° C., and the mixture was stirred for 0° C. for 1 hr. The mixture was concentrated under reduced pressure at 0° C., and THF (15 mL) was added to the residue. THF solution was cooled to 0° C., benzyl alcohol (3.12 mL), Et₃N (2.1 mL) and 4-dimethylaminopyridine (0.24 g) were added, and the mixture was stirred at room temperature for 24 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent; 10% ethyl acetate/hexane) to give crude 4-benzyl 1-tert-butyl piperidine-1,4-dicarboxylate (5.0 g) as a colorless oil.

### (Step 2)

To a solution of the compound (5.0 g) obtained in step 1 in ethyl acetate (10 mL) was added 4N hydrogen chloride/ethyl acetate (10 mL) solution, and the mixture was stirred for 50° C. for 1 hr. The reaction mixture was concentrated under reduced pressure to give benzyl piperidine-4-carboxylate monohydrochloride (2.5 g, 97%) as a white powder.

### (Step 3)

To a solution of the compound (10.0 g) obtained in step 2, glycolic acid (4.46 g) and Et₃N (5.46 mL) in CH₃CN (100 mL) were added WSC.HCl (15.0 g) and HOBt.H₂O (8.98 g), and the mixture was stirred at room temperature for 2 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 50→100% ethyl acetate/hexane) to give benzyl 1-glycoloylpiperidine-4-carboxylate (6.25 g, 57%) as a colorless oil.
MS (ESI+):278 (M+H)

### (Step 4)

A solution of the compound (6.25 g) obtained in step 3 and 10% Pd-carbon (2.5 g) in EtOH (200 mL) was stirred under 1 atm hydrogen atmosphere at room temperature for 14 hr. The catalyst was filtered off, and the filtrate was concentrated under reduced pressure to give the title compound (3.90 g, 93%) as a white powder.
elemental analysis value: C₈H₁₃NO₄
Found C, 51.33; H, 7.00; N, 7.48
Calculated C, 51.06; H, 7.01; N, 7.42

The compounds described in Reference Example 11 are as follows (Table 2).

TABLE 2

| Ref. | Structural |
|---|---|
| Ex. No. | formula |
| 11 | 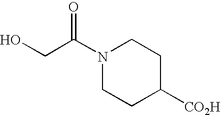 |

### Reference Example 12

### (3R,4S*)-1-[amino(oxo)acetyl]-N-[3,5-bis(trifluoromethyl)benzyl]-3-(4-fluorophenyl)-N-methylpiperidine-4-carboxamide

The title compound was obtained by reaction and purification in the same manner as in Reference Example 1 and using (3R*,4S*)—N-[3,5-bis(trifluoromethyl)benzyl]-N-methyl-3-(4-fluorophenyl)piperidine-4-carboxamide monohydrochloride synthesized by a known method (WO2005/068427).
MS (ESI+):534 (M+H)

### Reference Example 13

### (3R,4R)-1-[amino(oxo)acetyl]-N-[3,5-bis(trifluoromethyl)benzyl]-3-(4-fluorophenyl)-N-methylpiperidine-4-carboxamide

The title compound was obtained by reaction and purification in the same manner as in Reference Example 1 and using (3R*,4R*)—N-[3,5-bis(trifluoromethyl)benzyl]-N-methyl-3-(4-fluorophenyl)piperidine-4-carboxamide monohydrochloride synthesized by a known method (WO2005/068427).
MS (ESI+):534 (M+H)

### Reference Example 14

### tert-butyl 4-{[(3R*,4S*)-4-{[[3,5-bis(trifluoromethyl)benzyl](methyl)amino]carbonyl}-3-(4-fluorophenyl)piperidin-1-yl]carbonyl}piperidine-1-carboxylate

The title compound was obtained by reaction and purification in the same manner as in Reference Example 1 and using (3R*,4S*)—N-[3,5-bis(trifluoromethyl)benzyl]-N-methyl-3-(4-fluorophenyl)piperidine-4-carboxamide monohydrochloride and N-Boc-isonipecotic acid.
MS (ESI+):674 (M+H)

### Reference Example 15

### (3R*,4S*)—N-[3,5-bis(trifluoromethyl)benzyl]-3-(4-fluorophenyl)-N-methyl-1-(piperidin-4-ylcarbonyl)piperidine-4-carboxamide monohydrochloride

The title compound was obtained by reaction and purification in the same manner as in Reference Example 6 and using the compound obtained in Reference Example 14.
MS (ESI+):574 (M−HCl+H)

### Reference Example 16

### Methyl 4-{[(3R*,4S *)-4-{[[3,5-bis(trifluoromethyl)benzyl](methyl)amino]carbonyl}-3-(4-fluorophenyl)piperidin-1-yl]carbonyl}piperidine-1-carboxylate

The title compound was obtained by reaction and purification in the same manner as in Reference Example 3 and using the compound obtained in Reference Example 15.
MS (ESI+):632 (M+H)

### Reference Example 17

### (3R*,4S*)—N-[3,5-bis(trifluoromethyl)benzyl]-N-cyclopropyl-3-(4-fluorophenyl)piperidine-4-carboxamide monohydrochloride

### (Step 1)

A solution of 1-benzyl-5-(4-fluorophenyl)-1,2,3,6-tetrahydropyridine-4-carboxylic acid (10.0 g) synthesized by a known method (WO2003/014121) and 10% Pd-carbon (2.50 g) in EtOH (250 mL) was stirred under 5 atm hydrogen atmosphere at 80° C. for 5.5 hr. The catalyst was filtered off, and the filtrate was concentrated under reduced pressure. To a solution of the obtained residue in CH₃CN (50 mL) were added Boc₂O (9.70 g) and Et₃N (6.01 mL) at room temperature, and the mixture was stirred for 2 days. The reaction mixture was poured into water, and the resultant product was extracted with ethyl acetate. The organic layer was washed with aqueous citric acid solution and water and dried, and the solvent was evaporated under reduced pressure to give (3R*,4S*)-1-(tert butoxycarbonyl)-3-(4-fluorophenyl)piperidine-4-carboxylic acid (3.63 g, 35%) as a white powder.
MS (ESI+):324 (M+H)

### (Step 2)

To a solution of the compound (2.00 g) obtained in step 1 and cyclopropylamine (515 μL) in CH₃CN (5 mL) were added WSC HCl (1.42 g) and HOBt H₂O (1.14 g), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 50→100% ethyl acetate/hexane) to give tert-butyl (3R*,4S*)-4-[(cyclopropylamino)carbonyl]-3-(4-fluorophenyl)piperidine-1-carboxylate (2.09 g, 93%) as a white amorphous solid.
MS (ESI+):307 (M−^{t}Bu)

### (Step 3)

To a solution of the compound (2.08 g) obtained in step 2 in DMF (15 mL) was added sodium hydride (60% in oil, 298 mg) at 0° C., and the mixture was stirred for 1 min. A solution of 3,5-bis(trifluoromethyl)benzyl bromide (2.29 g) in THF (15 mL) was added to the reaction mixture at 0° C. over 30 min, and the mixture was stirred at room temperature for 3 days. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 0→33% ethyl acetate/hexane) to give tert-butyl (3R*,4S*)-4-{[[3,5-bis(trifluoromethyl)benzyl]{cyclopropyl}amino]carbonyl}-3-(4-fluorophenyl)piperidine-1-carboxylate (2.34 g, 69%) as a white amorphous solid.
MS (ESI+):589 (M+H)

### (Step 4)

To a solution of the compound (2.34 g) obtained in step 3 in ethyl acetate (35 mL) was added 4N hydrogen chloride/ethyl acetate (15 mL) solution, and the mixture was stirred at room temperature for 18 hr. The reaction mixture was concentrated under reduced pressure, and the residue was crystallized from EtOH-IPE to give the title compound (1.93 g, 92%) as a white powder.
MS (ESI+):489 (M−HCl+H)

### Reference Example 18

### (3R*,4S*)-1-[amino(oxo)acetyl]-N-[3,5-bis(trifluoromethyl)benzyl]-N-cyclopropyl-3-(4-fluorophenyl)piperidine-4-carboxamide

The title compound was obtained by reaction and purification in the same manner as in Reference Example 1 and using the compound obtained in Reference Example 17.
MS (ESI+):560 (M+H)

### Reference Example 19

### (3R*,4S*)-1-(2-amino-2-oxoethyl)-N-[3,5-bis(trifluoromethyl)benzyl]-N-cyclopropyl-3-(4-fluorophenyl)piperidine-4-carboxamide monohydrochloride

To a solution of the compound (210 mg) obtained in Reference Example 17 and 2-iodoacetamide (104 mg) in DMF (3 mL) was added Et₃N (139 μL) at room temperature, and the mixture was stirred at room temperature for 8 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 0→100% ethyl acetate/hexane). The thus-obtained colorless oil was treated with 1 equivalent amount of 4N hydrogen chloride/ethyl acetate to give the title compound (170 mg) as a white amorphous solid.
MS (ESI+):546 (M−HCl+H)

### Reference Example 20

### tert-butyl (3R*,4R*)-4-{[[3,5-bis(trifluoromethyl)benzyl](methyl)amino]carbonyl}-3-(2-methylphenyl)piperidine-1-carboxylate

### (Step 1)

To a solution of ethyl 1-methyl-1,2,3,6-tetrahydropyridine-4-carboxylate (8.77 g) and copper (I) iodide (2.14 g) in Et₂O (80 mL) was added 1M 2-methylphenylmagnesium bromide (100 mL/THF solution) at −30° C. over 1 hr, and the mixture was further stirred at −10° C. for 1 hr. Saturated aqueous ammonium chloride solution and ethyl acetate were added to the reaction mixture, and insoluble material was filtered off. The filtrate was washed with aqueous ammonium chloride solution and saturated brine and dried, and the solvent was evaporated under reduced pressure. The obtained residue was purified by silica gel column chromatography (NH Chromatorex) (solvent gradient; 33→66% ethyl acetate/hexane) to give crude ethyl 1-methyl-3-(2-methylphenyl)piperidine-4-carboxylate (11.85 g, 88%) as a colorless oil.

### (Step 2)

To a solution of the compound (11.85 g) obtained in step 1 in CH₃CN (150 mL) was added 1-chloroethyl chloroformate (5.87 mL) at 0° C., and the mixture was heated under reflux for 2 hr. The reaction mixture was concentrated under reduced pressure, methanol (150 mL) was added to the residue, and the mixture was heated under reflux for 3 hr. The reaction mixture was concentrated under reduced pressure, a solution of Et₃N (8.21 mL) and Boc₂O (12.9 g) in CH₃CN (20 mL) was added to a solution of the residue in CH₃CN (150 mL) at 0° C., and the mixture was stirred at room temperature for 14 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 14→20% ethyl acetate/hexane) to give crude 1-tert-butyl 4-ethyl 3-(2-methylphenyl)piperidine-1,4-dicarboxylate (8.34 g, 53%) as a colorless oil.

### (Step 3)

To a solution of the compound (8.34 g) obtained in step 2 in MeOH (50 mL) was added 28% sodium methoxide-MeOH (6.95 g) at room temperature, and the mixture was heated under reflux for 3 hr. A 2N aqueous sodium hydroxide solution (18 mL) and THF (25 mL) were added to the reaction mixture, and the mixture was stirred at 50° C. for 3 hr. The reaction mixture was concentrated under reduced pressure, and the residue was partitioned between 1M KHSO₄ aqueous solution and ethyl acetate. The organic layer was washed with water and saturated brine and dried, and the solvent was evaporated under reduced pressure. The obtained residue was purified by silica gel column chromatography (solvent gradient; 14→20% ethyl acetate/hexane), and crystallized from ethyl acetate-IPE-hexane to give (3R,4R*)-1-(tert-butoxycarbonyl)-3-(2-methylphenyl)piperidine-4-carboxylic acid (2.40 g, 31%) as a white powder.
MS (ESI+):246 (M−^{t}BuO)

### (Step 4)

To a solution of the compound (2.40 g) obtained in step 3, 1-[3,5-bis(trifluoromethyl)phenyl]-N-methylmethanamine monohydrochloride (2.52 g) and Et₃N (1.26 ml) in CH₃CN (30 mL) were added WSC.HCl (1.73 g) and HOBt-H₂O (0.69 g), and the mixture was stirred at room temperature for 18 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→50% ethyl acetate/hexane). The residue was crystallized from ethyl acetate-IPE-hexane to give the title compound (2.87 g, 68%) as a white powder.
MS (ESI+):485 (M−^{t}BuO)

### Reference Example 21

### (3R*,4R*)—N-[3,5-bis(trifluoromethyl)benzyl]-N-methyl-3-(2-methylphenyl)piperidine-4-carboxamide monohydrochloride

The title compound was obtained by reaction and purification in the same manner as in Reference Example 17, step 4 and using the compound obtained in Reference Example 20.
MS (ESI+):459 (M−HCl+H)

### Reference Example 22

### (3R*,4R*)-1-[(1-acetylpiperidin-4-yl)carbonyl]-N-[3,5-bis(trifluoromethyl)benzyl]-N-methyl-3-(2-methylphenyl)piperidine-4-carboxamide

The title compound was obtained by reaction and purification in the same manner as in Reference Example 1 and using the compound obtained in Reference Example 21 and 1-acetylpiperidine-4-carboxylic acid.
MS (ESI+):612 (M+H)

### Reference Example 23

### Methyl 4-{((3R*,4R*)-4-{[[3,5-bis(trifluoromethyl)benzyl](methyl)amino]carbonyl}-3-(2-methylphenyl)piperidine-1-yl]carbonyl}piperidine-1-carboxylate

The title compound was obtained by reaction and purification in the same manner as in Reference Example 3 and using the compound obtained in Example 19, step 2.
MS (ESI+):628 (M+H)

### Reference Example 24

### Methyl 4-{[(3R*,4R*)-4-{[[3,5-bis(trifluoromethyl)benzyl](cyclopropyl)amino]carbonyl}-3-(2-methylphenyl)piperidin-1-yl]carbonyl}piperidine-1-carboxylate

### (Step 1)

tert-Butyl 4-{[(3R*,4R*)-4-{[[3,5-bis(trifluoromethyl)benzyl](cyclopropyl)amino]carbonyl}-3-(2-methylphenyl)piperidin-1-yl]carbonyl}piperidine-1-carboxylate was obtained by reaction and purification in the same manner as in Reference Example 17 and Reference Example 5 and using the compound obtained in Reference Example 20, step 3.
MS (ESI+):696 (M+H)

### (Step 2)

(3R*,4R*)—N-[3,5-bis(Trifluoromethyl)benzyl]-N-cyclopropyl-3-(2-methylphenyl)-1-(piperidin-4-ylcarbonyl)piperidine-4-carboxamide monohydrochloride was obtained by reaction and purification in the same manner as in Reference Example 6 and using the compound obtained in step 1.
MS (ESI+):596 (M−HCl+H)

### (Step 3)

The title compound was obtained by reaction and purification in the same manner as in Reference Example 3 and using the compound obtained in step 2.
MS (ESI+):654 (M+H)

### Reference Example 25

### tert-butyl (3R*,4R*)-4-{[[3,5-bis(trifluoromethyl)benzyl](methyl)amino]carbonyl}-3-(2-ethylphenyl)piperidine-1-carboxylate

The title compound was obtained by reaction and purification in the same manner as in Reference Example 20 and using 2-ethylphenylmagnesium bromide.
MS (ESI+):573 (M+H)

### Reference Example 26

### (3R*,4R*)—N-[3,5-bis(trifluoromethyl)benzyl]-3-(2-ethylphenyl)-N-methylpiperidine-4-carboxamide monohydrochloride

The title compound was obtained by reaction and purification in the same manner as in Reference Example 17, step 4 and using the compound obtained in Reference Example 25.
MS (ESI+):473 (M−HCl+H)

### Reference Example 27

### (3R*,4R*)-1-[(1-acetylpiperidin-4-yl)carbonyl]-N-[3,5-bis(trifluoromethyl)benzyl]-3-(2-ethylphenyl)-N-methylpiperidine-4-carboxamide

The title compound was obtained by reaction and purification in the same manner as in Reference Example 1 and using the compound obtained in Reference Example 26 and 1-acetylpiperidine-4-carboxylic acid.
MS (ESI+):626 (M+H)

### Reference Example 28

### (3R,4R*)-1-[amino(oxo)acetyl]-N-[3,5-bis(trifluoromethyl)benzyl]-3-(2-ethylphenyl)-N-methylpiperidine-4-carboxamide

The title compound was obtained by reaction and purification in the same manner as in Reference Example 1 and using the compound obtained in Reference Example 26.
MS (ESI+):544 (M+H)

### Reference Example 29

### tert-butyl (3R*,4R*)-4-{[[3,5-bis(trifluoromethyl)benzyl](methyl)amino]carbonyl}-3-(2-isopropylphenyl)piperidine-1-carboxylate

### (Step 1)

To a solution of sodium hydride (60% in oil,2.50 g) in DMF (100 mL) was added ethyl 1-benzyl-3-oxopiperidine-4-carboxylate monohydrochloride (7.44 g) at 0° C., and the mixture was stirred for 5 min. N-Phenylbis(trifluoromethanesulfonimide) (10.0 g) was added, and the mixture was stirred for 0° C. for 1 hr. The reaction mixture was poured into ice water, and the resultant product was extracted with ethyl acetate. The organic layer was washed with water and saturated brine and dried, and the solvent was evaporated under reduced pressure. To a mixture of the obtained residue in toluene (100 mL) and water (6 mL) were added (2-isopropylphenyl)boronic acid (6.15 g), potassium carbonate (3.45 g) and tetrakis(triphenylphosphine)palladium (0) (2.89 g), and the mixture was stirred under an argon atmosphere at 100° C. for 14 hr. The reaction mixture was poured into water, and the resultant product was extracted with ethyl acetate. The organic layer was washed with saturated aqueous sodium hydrogen carbonate and saturated brine and dried, and the solvent was evaporated under reduced pressure. The obtained residue was purified by silica gel column chromatography (solvent gradient; 5→25% ethyl acetate/hexane) to give ethyl 1-benzyl-5-(2-isopropylphenyl)-1,2,3,6-tetrahydropyridine-4-carboxylate (9.09 g, 100%) as a pale-yellow oil.
MS (ESI+):364 (M+H)

### (Step 2)

To a solution of the compound (9.80 g) obtained in step 1 in CH₃CN (50 mL) was added 1-chloroethyl chloroformate (3.77 mL) at 0° C., and the mixture was heated under reflux at 100° C. for 1 hr. The reaction mixture was concentrated under reduced pressure, methanol (50 mL) was added to the residue, and the mixture was heated under reflux for 1 hr. The reaction mixture was concentrated under reduced pressure. To a solution of the residue in CH₃CN (40 mL) were added a solution of Et₃N (4.51 mL) and Boc₂O (7.05 g) in CH₃CN (10 mL) at 0° C., and the mixture was stirred at room temperature for 14 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 10→50% ethyl acetate/hexane) to give crude 1-tert-butyl 4-ethyl 3-(2-isopropylphenyl)piperidine-1,4-dicarboxylate (6.72 g, 66%) as a colorless oil. To a solution of the oil (3.91 g) obtained above in MeOH (50 mL) was added magnesium (2.3 g), and the mixture was stirred at room temperature for 14 hr. A saturated aqueous ammonium chloride solution (50 mL) was added to the reaction mixture, and the resultant product was extracted with ethyl acetate. The organic layer was washed with water and saturated brine and dried, and the solvent was evaporated under reduced pressure. To a solution of the obtained residue in EtOH (50 mL) was added sodium hydride (60% in oil, 0.83 g), and the mixture was stirred for 80° C. for 2 hr. The reaction mixture was poured into an aqueous ammonium chloride solution, and the resultant product was extracted with ethyl acetate. The organic layer was washed with water and saturated brine and dried, and the solvent was evaporated under reduced pressure. The obtained residue was purified by silica gel column chromatography (solvent gradient; 20→50% ethyl acetate/hexane) to give 1-tert-butyl 4-ethyl (3R*,4R*)-3-(2-isopropylphenyl)piperidine-1,4-dicarboxylate (3.39 g, 87%) as a colorless oil.
MS (ESI+):376 (M+H)

### (Step 3)

To a solution of the compound (3.29 g) obtained in step 2 in a mixture of THF (30 mL) and EtOH (10 mL) was added 8N aqueous sodium hydroxide solution (50 mL), and the mixture was stirred at 80° C. for 2 days. The reaction mixture was weakly acidified with an aqueous citric acid solution, and the resultant product was extracted with ethyl acetate. The organic layer was washed with water and saturated brine and dried, and the solvent was evaporated under reduced pressure. The obtained residue was purified by silica gel column chromatography (solvent gradient; 10→67% ethyl acetate/hexane), and crystallized from ethyl acetate-hexane to give (3R*,4R*)-1-(tert-butoxycarbonyl)-3-(2-isopropylphenyl)piperidine-4-carboxylic acid (1.02 g, 34%) as a white powder. elemental analysis value: C₂₀H₂₉NO₄
Found C, 69.14; H, 8.41; N, 4.03
Calculated C, 68.93; H, 8.37; N, 3.91

### (Step 4)

To a solution of the compound (0.95 g) obtained in step 3 and DMF (about 50 μL) in THF (10 mL) was added oxalyl chloride (0.29 mL) at 0° C. The mixture was stirred at 0° C. for 1 hr, and the reaction mixture was concentrated under reduced pressure under ice-cooling. A solution of the obtained residue in THF (10 mL) was added to a solution of 1-[3,5-bis(trifluoromethyl)phenyl]-N-methylmethanamine monohydrochloride (0.92 g) and Et₃N (0.96 mL) in THF (10 mL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→40% ethyl acetate/hexane), and crystallized from ethyl acetate-hexane to give the title compound (1.36 g, 85%) as a white powder.
MS (ESI+):587 (M+H)

### Reference Example 30

### (3R*,4R*)—N-[3,5-bis(trifluoromethyl)benzyl]-3-(2-isopropylphenyl)-N-methylpiperidine-4-carboxamide monohydrochloride

To a solution of the compound (1.26 g) obtained in Reference Example 29 in ethyl acetate (10 mL) was added 4N hydrogen chloride/ethyl acetate (1.26 mL) solution, and the mixture was stirred with heating at 50° C. for 2 hr. The reaction mixture was concentrated under reduced pressure, and the residue was crystallized from ethyl acetate-IPE to give the title compound (0.935 g, 83%) as a white powder.
MS (ESI+):487 (M−HCl+H)

### Reference Example 31

### (3R,4R*)-1-[amino(oxo)acetyl]-N-[3,5-bis(trifluoromethyl)benzyl]-3-(2-isopropylphenyl)-N-methylpiperidine-4-carboxamide

The title compound was obtained by reaction and purification in the same manner as in Reference Example 1 and using the compound obtained in Reference Example 30.
MS (ESI+):558 (M+H)

### Reference Example 32

### (3R*,4R*)-1-[(1-acetylpiperidin-4-yl)carbonyl]-N-[3,5-bis(trifluoromethyl)benzyl]-3-(2-isopropylphenyl)-N-methylpiperidine-4-carboxamide

The title compound was obtained by reaction and purification in the same manner as in Reference Example 1 and using the compound obtained in Reference Example 30 and 1-acetylpiperidine-4-carboxylic acid.
MS (ESI+):640 (M+H)

### Reference Example 33

### tert-butyl (3R*,4S*)-4-{([3,5-bis(trifluoromethyl)benzyl](methyl)amino]carbonyl}-3-(3-methylphenyl)piperidine-1-carboxylate

### Reference Example 34

### tert-butyl (3R*,4R*)-4-{[[3,5-bis(trifluoromethyl)benzyl](methyl)amino]carbonyl}-3-(3-methylphenyl)piperidine-1-carboxylate

### (Step 1)

To a solution of ethyl 1-methyl-1,2,3,6-tetrahydropyridine-4-carboxylate (15.0 g) and copper (I) iodide (2.76 g) in Et₂O (50 mL) was added 1M 3-methylphenylmagnesium bromide-Et₂O (132 mL) solution at −30° C. over 1 hr, and the mixture was further stirred at −10° C. for 1 hr. Saturated aqueous ammonium chloride solution and ethyl acetate were added to the reaction mixture, and insoluble material was filtered off. The filtrate was washed with aqueous ammonium chloride solution and saturated brine and dried, and the solvent was evaporated under reduced pressure. The obtained residue was purified by distillation under reduced pressure to give ethyl 1-methyl-3-(3-methylphenyl)piperidine-4-carboxylate (12.7 g, 54.8%) as a colorless oil.
boiling point: 110-125° C. (7 mmHg)

### (Step 2)

To a solution of the compound (12.5 g) obtained in step 1 in CH₃CN (50 mL) was added 1-chloroethyl chloroformate (8.21 mL) at 0° C., and the mixture was heated under reflux for 2 hr. The reaction mixture was concentrated under reduced pressure, methanol (50 mL) was added to the residue, and the mixture was heated-under reflux for 1 hr. The reaction mixture was concentrated under reduced pressure. To a solution of the residue in CH₃CN (100 mL) were added Et₃N (10.0 mL) and Boc₂O (11.5 g) at 0° C., and the mixture was stirred at room temperature for 14 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 14→20% ethyl acetate/hexane) to give 1-tert-butyl 4-ethyl 3-(3-methylphenyl)piperidine-1,4-dicarboxylate (8.34 g, 53%) as a colorless oil.

### (Step 3)

To a solution of the compound (15.1 g) obtained in step 2 in THF (70 mL) was added 2N aqueous potassium hydroxide solution (144 mL), and the mixture was stirred for 50° C. for 48 hr. The reaction mixture was weakly acidified with an aqueous citric acid solution, and the resultant product was extracted with ethyl acetate. The organic layer was washed with water and saturated brine and dried, and the solvent was evaporated under reduced pressure. The obtained residue was purified by silica gel column chromatography (solvent gradient; 10→100% ethyl acetate/hexane), and crystallized from ethyl acetate-hexane to give 1-(tert-butoxycarbonyl)-3-(3-methylphenyl)piperidine-4-carboxylic acid (1.69 g, 12%) as a white powder.
melting point: 121-123° C.

### (Step 4)

To a solution of the compound (1.00 g) obtained in step 3, 1-[3,5-bis(trifluoromethyl)phenyl]-N-methylmethanamine monohydrochloride (1.05 g) and Et₃N (0.53 mL) in CH₃CN (15 mL) were added WSC.HCl (0.90 g) and HOBt H₂O (0.58 g), and the mixture was stirred at room temperature for 24 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→20% ethyl acetate/hexane).

The compound (0.371 g, 21%) of Reference Example 33 was obtained as a white powder from a fraction with a short retention time. The compound (0.265 g, 15%) of Reference Example 34 was obtained as a white powder from a fraction with a long retention time.

### Compound of Reference Example 33

MS (ESI+):559 (M+H)

### Compound of Reference Example 34

MS (ESI+):559 (M+H)

### Reference Example 35

### (3R*,4S*)—N-[3,5-bis(trifluoromethyl)benzyl]-N-methyl-3-(3-methylphenyl)piperidine-4-carboxamide monohydrochloride

The title compound was obtained by reaction and purification in the same manner as in Reference Example 6 and using the compound obtained in Reference Example 33.
MS (ESI+):459 (M−HCl+H)

### Reference Example 36

(3R*,4R*)—N-[3,5-bis(trifluoromethyl)benzyl]-N-methyl-3-(3-methylphenyl)piperidine-4-carboxamide monohydrochloride

The title compound was obtained by reaction and purification in the same manner as in Reference Example 6 and using the compound obtained in Reference Example 34.
MS (ESI+):459 (M−HCl+H)

### Reference Example 37

### (3R*,4S*)-1-[amino(oxo)acetyl]-N-[3,5-bis(trifluoromethyl)benzyl]-N-methyl-3-(3-methylphenyl)piperidine-4-carboxamide

The title compound was obtained by reaction and purification in the same manner as in Reference Example 1 and using the compound obtained in Reference Example 35.
MS (ESI+):530 (M+H)

### Reference Example 38

### (3R*,4R*)-1-[amino(oxo)acetyl]-N-[3,5-bis(trifluoromethyl)benzyl]-N-methyl-3-(3-methylphenyl)piperidine-4-carboxamide

The title compound was obtained by reaction and purification in the same manner as in Reference Example 1 and using the compound obtained in Reference Example 36.
MS (ESI+):530 (M+H)

### Reference Example 39

### (3R*,4R*)-1-[(1-acetylpiperidin-4-yl) carbonyl]-N-[3,5-bis(trifluoromethyl)benzyl]-N-methyl-3-(3-methylphenyl)piperidine-4-carboxamide

The title compound was obtained by reaction and purification in the same manner as in Reference Example 1 and using the compound obtained in Reference Example 36 and 1-acetylpiperidine-4-carboxylic acid.
MS (ESI+):612 (M+H)

### Reference Example 40

### tert-butyl (3R*,4S*)-4-[[3,5-bis(trifluoromethyl)benzyl](methyl)amino]carbonyl}-3-(4-methylphenyl)piperidine-1-carboxylate

### Reference Example 41

### tert-butyl (3R*,4R*)-4-{[[3,5-bis(trifluoromethyl)benzyl](methyl)amino]carbonyl}-3-(4-methylphenyl)piperidine-1-carboxylate

The title compound was obtained by reaction and purification in the same manner as in Reference Examples 33-34 and using 4-methylphenylmagnesium bromide.

The compound of Reference Example 40 was obtained as a white powder from a fraction with a short retention time. The compound of Reference Example 41 was obtained as a white powder from a fraction with a long retention time.

### Compound of Reference Example 40

MS (ESI+):559 (M+H)

### Compound of Reference Example 41

MS (ESI+):559 (M+H)

### Reference Example 42

### (3R*,4S*)—N-[3,5-bis(trifluoromethyl)benzyl]-N-methyl-3-(4-methylphenyl)piperidine-4-carboxamide monohydrochloride

The title compound was obtained by reaction and purification in the same manner as in Reference Example 6 and using the compound obtained in Reference Example 40.
MS (ESI+):459 (M−HCl+H)

### Reference Example 43

### (3R*,4R*)—N-[3,5-bis(trifluoromethyl)benzyl]-N-methyl-3-(4-methylphenyl)piperidine-4-carboxamide monohydrochloride

The title compound was obtained by reaction and purification in the same manner as in Reference Example 6 and using the compound obtained in Reference Example 41.
MS (ESI+):459 (M−HCl+H)

### Reference Example 44

### (3R*,4R*)-1-[amino(oxo)acetyl]-N-[3,5-bis(trifluoromethyl)benzyl]-N-methyl-3-(4-methylphenyl)piperidine-4-carboxamide

The title compound was obtained by reaction and purification in the same manner as in Reference Example 1 and using the compound obtained in Reference Example 43.
MS (ESI+):530 (M+H)

### Reference Example 45

### (3R*,4R*)-1-[(1-acetylpiperidin-4-yl)carbonyl]-N-[3,5-bis(trifluoromethyl)benzyl]-N-methyl-3-(4-methylphenyl)piperidine-4-carboxamide

The title compound was obtained by reaction and purification in the same manner as in Reference Example 1 and using the compound obtained in Reference Example 43 and 1-acetylpiperidine-4-carboxylic acid.
MS (ESI+):612 (M+H)

### Reference Example 46

### (3R*,4S*)-1-[amino(oxo)acetyl]-N-[3,5-bis(trifluoromethyl)benzyl]-N-methyl-3-(4-methylphenyl)piperidine-4-carboxamide

The title compound was obtained by reaction and purification in the same manner as in Reference Example 1 and using the compound obtained in Reference Example 42.
MS (ESI+):530 (M+H)

### Reference Example 47

### (3R*,4S*)-1-[amino(oxo)acetyl]-N-[3,5-bis(trifluoromethyl)benzyl]-3-(4-fluoro-2-methylphenyl)-N-methylpiperidine-4-carboxamide

The title compound was obtained by reaction and purification in the same manner as in Reference Example 1 and using (3R*,4S)—N-[3,5-bis(trifluoromethyl)benzyl]-3-(4-fluoro-2-methylphenyl)-N-methylpiperidine-4-carboxamide monohydrochloride synthesized by a known method (WO2005/068427).
MS (ESI+):548

### Reference Example 48

### tert-butyl 4-{[(3R*,4R*)-4-{[[3,5-bis(trifluoromethyl)benzyl](methyl)amino]carbonyl}-3-(4-fluoro-2-methylphenyl)piperidin-1-yl]carbonyl}piperidine-1-carboxylate

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride synthesized by a known method (WO2005/068427) and N-Boc-isonipecotic acid.
MS (ESI+):688 (M+H)

### Reference Example 49

### (3R*4R*)—N-[3,5-bis(trifluoromethyl)benzyl]-3-(4-fluoro-2-methylphenyl)-N-methyl-1-(piperidin-4-ylcarbonyl)piperidine-4-carboxamide monohydrochloride

The title compound was obtained by reaction and purification in the same manner as in Reference Example 6 and using the compound obtained in Reference Example 48.
MS (ESI+):588 (M−HCl+H)

### Reference Example 50

### (3R*,4R*)—N-[3,5-bis(trifluoromethyl)benzyl]-3-(4-fluoro-2-methylphenyl)-1-glycoloyl-N-methylpiperidine-4-carboxamide

The title compound was obtained by reaction and purification in the same manner as in Reference Example 1 and using (3R*,4R)—N-[3,5-bis(trifluoromethyl)benzyl]-3-(4-fluoro-2-methylphenyl)-N-methylpiperidine-4-carboxamide monohydrochloride and glycolic acid.
MS (ESI+):535 (M+H)

### Reference Example 51

### (3R*,4R*)—N-[3,5-bis(trifluoromethyl)benzyl]-3-(4-fluoro-2-methylphenyl)-1-(3-hydroxy-3-methylbutanoyl)-N-methylpiperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and β-hydroxyisovaleric acid.
MS (ESI+):577 (M+H)

### Reference Example 52

### (3R*,4R*)—N-[3,5-bis(trifluoromethyl)benzyl]-3-(4-fluoro-2-methylphenyl)-N-methyl-1-[(methylsulfonyl)acetyl]piperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and methanesulfonylacetic acid.
MS (ESI+):597 (M+H)

### Reference Example 53

### (3R*,4R*)—N-[3,5-bis(trifluoromethyl)benzyl]-3-(4-fluoro-2-methylphenyl)-1-(3-hydroxybutanoyl)-N-methylpiperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and 3-hydroxybutyric acid.
MS (ESI+):563 (M+H)

### Reference Example 54

### Methyl 4-{[(3R*,4R*)-4-{[[3,5-bis(trifluoromethyl)benzyl](methyl)amino]carbonyl}-3-(4-fluoro-2-methylphenyl)piperidin-1-yl]carbonyl}piperidine-1-carboxylate

The title compound was obtained by reaction and purification in the same manner as in Reference Example 3 and using the compound obtained in Reference Example 49.
MS (ESI+):646 (M+H)

### Reference Example 55

### (3R*,4R*)—N-[3,5-bis(trifluoromethyl)benzyl]-3-(4-fluoro-2-methylphenyl)-N-methyl-1-{[1-(methylsulfonyl)piperidin-4-yl]carbonyl}piperidine-4-carboxamide

The title compound was obtained by reaction and purification in the same manner as in Reference Example 3 and using the compound obtained in Reference Example 49 and methanesulfonyl chloride.
MS (ESI+):666 (M+H)

### Reference Example 56

### (3R*,4R*)—N-[3,5-bis(trifluoromethyl)benzyl] -3-(4-fluoro-2-methylphenyl)-N-methyl-1-(5-oxo-D-prolyl)piperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and D-pyroglutamic acid.
MS (ESI+):588 (M+H)

### Reference Example 57

### (3R*,4R*)—N-[3,5-bis(trifluoromethyl)benzyl]-3-(4-fluoro-2-methylphenyl)-N-methyl-1-(1-methyl-5-oxo-D-prolyl)piperidine-4-carboxamide

To a solution of the compound (100 mg) obtained in Reference Example 56 in THF (5 mL) was added a suspension of sodium hydride (60% in oil, 8.2 mg) in THF (5 mL) at 0° C. After stirring at 0° C. for 5 min, methyl iodide (28 mg) was added, and the mixture was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound.
MS (ESI+):602 (M+H)

### Reference Example 58

### Methyl 3-{[(3R*,4R*)-4-{[[3,5-bis(trifluoromethyl)benzyl](methyl)amino]carbonyl}-3-(4-fluoro-2-methylphenyl)piperidin-1-yl]carbonyl}azetidine-1-carboxylate

### (Step 1)

tert-Butyl 3-{[(3R*,4R*)-4-{[[3,5-bis(trifluoromethyl)benzyl](methyl)amino]carbonyl}-3-(4-fluoro-2-methylphenyl)piperidin-1-yl]carbonyl}azetidine-1-carboxylate was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and Boc-azetidine-3-carboxylic acid.
MS (ESI+):660 (M+H)

### (Step 2)

(3R*,4R*)-1-(Azetidin-3-ylcarbonyl)-N-[3,5-bis(trifluoromethyl)benzyl]-3-(4-fluoro-2-methylphenyl)-N-methylpiperidine-4-carboxamide monohydrochloride was obtained by reaction and purification in the same manner as in Reference Example 6 and using the compound obtained in step 1.
MS (ESI+):560 (M−HCl+H)

### (Step 3)

The title compound was obtained by reaction and purification in the same manner as in Reference Example 3 and using the compound obtained in step 2.
MS (ESI+):618 (M+H)

### Reference Example 59

### (3R*,4R*)—N-[3,5-bis(trifluoromethyl)benzyl]-3-(4-fluoro-2-methylphenyl)-1-(2-hydroxy-2-methylpropanoyl)-N-methylpiperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and 2-hydroxyisobutyric acid.
MS (ESI+):563 (M+H)

### Reference Example 60

### (3R*,4R*)—N-[3,5-bis(trifluoromethyl)benzyl]-3-(4-fluoro-2-methylphenyl)-1-[(1-hydroxycyclopropyl)carbonyl]-N-methylpiperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and 1-hydroxy-1-cyclopropanecarboxylic acid.
MS (ESI+):561 (M+H)

### Reference Example 61

### (3R*,4R*)—N-[3,5-bis(trifluoromethyl)benzyl]-3-(4-fluoro-2-methylphenyl)-1-lactoyl-N-methylpiperidine-4-carboxamide

The title compound was obtained by reaction and purification in the same manner as in Reference Example 1 and using (3R*,4R*)—N-[3,5-bis(trifluoromethyl)benzyl]-3-(4-fluoro-2-methylphenyl)-N-methylpiperidine-4-carboxamide monohydrochloride and lactic acid.
MS (ESI+):549 (M+H)

### Reference Example 62

### (3R*,4R*)—N-[3,5-bis(trifluoromethyl)benzyl]-1-[2-(dimethylamino)-2-oxoethyl]-3-(4-fluoro-2-methylphenyl)-N-methylpiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (256 mg), 2-chloro-N,N-dimethylacetamide (77 μL) and NaI (60 mg) in DMF (10 mL) was added Et₃N (174 μL), and the mixture was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent; 100% ethyl acetate) to give a colorless oil. The obtained oil was treated with 1 equivalent amount of 0.4N hydrogen chloride/ethyl acetate to give the title compound (217 mg, 73%) as a white powder.
MS (ESI+):562 (M−HCl+H)

### Reference Example 63

### (3R*,4R*)—N-[3,5-bis(trifluoromethyl)benzyl]-3-(4-fluoro-2-methylphenyl)-N-methyl-1-(tetrahydro-2H-pyran-4-yl)piperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (205 mg), tetrahydro-4H-pyran-4-one (48 mg) in acetic acid (0.4 mL)-dichloromethane (5 mL) was added NaBH(OAc)₃ (254 mg), and the mixture was stirred at room temperature for one day. Tetrahydro-4H-pyran-4-one (160 mg) and TiCl₄ (catalytic amount) were further added, and the mixture was stirred for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give a colorless oil (116 mg). The oil was dissolved in ethyl acetate (2 mL), and treated with 1 equivalent amount of 0.4N hydrogen chloride/ethyl acetate to give the title compound (105 mg, 44%) as a white powder.
MS (ESI+):561 (M−HCl+H)

### Reference Example 64

### (3R*,4R*)-1′-acetyl-N-[3,5-bis(trifluoromethyl)benzyl]-3-(4-fluoro-2-methylphenyl)-N-methyl-1,4′-bipiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (205 mg), 1-acetyl-4-piperidone (339 mg) in acetic acid (0.4 mL)-dichloromethane (5 mL) were added NaBH(OAc)₃ (254 mg) and TiCl₄ (catalytic amount), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give a colorless oil (125 mg). The oil was dissolved in ethyl acetate (2 mL), and treated with 1 equivalent amount of 0.4N hydrogen chloride/ethyl acetate to give the title compound (107 mg, 42%) as a white powder.
MS (ESI+):602 (M−HCl+H)

### Reference Example 65

### (3R*,4R*)—N-[3,5-bis(trifluoromethyl)benzyl]-3-(4-fluoro-2-methylphenyl)-N-methyl-1′-(methylsulfonyl)-1,4′-bipiperidine-4-carboxamide monohydrochloride

A colorless oil was obtained by reaction and purification in the same manner as in Reference Example 3 and using the compound obtained in Example 106 and methanesulfonyl chloride. The obtained oil was treated with 1 equivalent amount of 4N hydrogen chloride/ethyl acetate to give the title compound.
MS (ESI+):638 (M−HCl+H)

### Reference Example 66

### tert-butyl [(3R*,4R*)-4-{[[3,5-bis(trifluoromethyl)benzyl](methyl)amino]carbonyl}-3-(4-fluoro-2-methylphenyl)piperidin-1-yl]acetate

To a solution of (3R*,4R*)—N-[3,5-bis(trifluoromethyl)benzyl]-3-(4-fluoro-2-methylphenyl)-N-methylpiperidine-4-carboxamide monohydrochloride (256 mg), tert-butyl bromoacetate (146 mg) and NaI (15 mg) in DMF (4 mL) was added Et₃N (174 μL) and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→50% ethyl acetate/hexane) to give the title compound (295 mg, 100%) as a colorless amorphous solid.
MS (ESI+):592 (M+H)

### Reference Example 67

### (3R*,4R*)—N-[3,5-bis(trifluoromethyl)benzyl]-3-(4-fluoro-2-methylphenyl)-N-methyl-1-(2-morpholin-4-yl-2-oxoethyl)piperidine-4-carboxamide monohydrochloride

A solution of the compound (295 mg) obtained in Reference Example 66 in TFA (3.5 mL) was stirred at room temperature for 14 hr. The reaction solution was concentrated under reduced pressure, and CH₃CN (3.0 mL) was added to the residue. Then, WSC.HCl (115 mg), HOBt-H₂O (91.9 mg), morpholine (52.5 μL) and Et₃N (83.6 μL) were added, and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→100% ethyl acetate/hexane) to give a colorless oil. The obtained oil was treated with 1 equivalent amount of 4N hydrogen chloride/ethyl acetate to give the title compound (219 mg, 68%) as a white powder.
MS (ESI+):604 (M−HCl+H)

### Reference Example 68

### (3R*,4R*)—N-[3,5-bis(trifluoromethyl)benzyl]-3-(4-fluoro-2-methylphenyl)-N-methyl-1-[2-(methylsulfonyl)ethyl]piperidine-4 4-carboxamide monohydrochloride

To a solution of 2-(methylsulfonyl)ethanol (95.6 mg) in THF (5 mL) were added methylsulfonyl chloride (75.4 μL) and Et₃N (122 μL), and the mixture was stirred at room temperature for 20 min. Insoluble material was filtered off, and the filtrate was added to a solution of (3R*,4R*)—N-[3,5-bis(trifluoromethyl)benzyl]-3-(4-fluoro-2-methylphenyl)-N-methylpiperidine-4-carboxamide monohydrochloride (150 mg) and Et₃N (122 μL) in THF (5 mL), and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→100% ethyl acetate/hexane) to give a colorless oil. The obtained oil was treated with 1 equivalent amount of 4N hydrogen chloride/ethyl acetate to give the title compound (36.8 mg, 55%) as a white powder.
MS (ESI+):583 (M−HCl+H)

### Reference Example 69

### (3R*,4R*)-1-[2-(acetylamino)ethyl]-N-[3,5-bis(trifluoromethyl)benzyl]-3-(4-fluoro-2-methylphenyl)-N-methylpiperidine-4-carboxamide monohydrochloride

A solution of (3R*,4R*)—N-[3,5-bis(trifluoromethyl)benzyl]-3-(4-fluoro-2-methylphenyl)-N-methylpiperidine-4-carboxamide monohydrochloride (200 mg), N-(2-chloroethyl)acetamide (62.9 mg), K₂CO₃ (70.4 mg) and NaI (76.4 mg) in CH₃CN (5 mL) was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→50% ethyl acetate/hexane) to give a colorless oil. The obtained oil was treated with 1 equivalent amount of 4N hydrogen chloride/ethyl acetate to give the title compound (101.7 mg, 44%) as a white powder.
MS (ESI+):562 (M−HCl+H)

### Reference Example 70

### (3R*,4R*)—N-[3,5-bis(trifluoromethyl)benzyl]-3-(4-fluoro-2-methylphenyl)-1-(2-hydroxyethyl)-N-methylpiperidine-4-carboxamide monohydrochloride

A solution of (3R*,4R*)—N-[3,5-bis(trifluoromethyl)benzyl]-3-(4-fluoro-2-methylphenyl)-N-methylpiperidine-4-carboxamide monohydrochloride (205 mg), 2-iodoethanol (62.3 μL) and K₂CO₃ (83 mg) in DMF (3 mL) was stirred at room temperature for 2 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 30→100% ethyl acetate/hexane) to give a colorless oil, which was treated with 1 equivalent amount of 4N hydrogen chloride/ethyl acetate to give the title compound (117 mg, 53%) as a white powder.
MS (ESI+):521 (M−HCl+H)

### Reference Example 71

### (3R*,4R*)—N-[3,5-bis(trifluoromethyl)benzyl]-3-(4-fluoro-2-methylphenyl)-1-(3-hydroxypropyl)-N-methylpiperidine-4-carboxamide monohydrochloride

A solution of (3R*,4R*)—N-[3,5-bis(trifluoromethyl)benzyl]-3-(4-fluoro-2-methylphenyl)-N-methylpiperidine-4-carboxamide monohydrochloride (170 mg),3-bromopropanol (70.5 mg) and Et₃N (115 μL) in THF (5 mL) was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give a colorless oil, which was treated with 1 equivalent amount of 4N hydrogen chloride/ethyl acetate to give the title compound (107 mg, 57%) as a white powder.
MS (ESI+):535 (M−HCl+H)

### Reference Example 72

### Methyl (3R*,4R*)-4-{[[3,5-bis(trifluoromethyl)benzyl](methyl)amino]carbonyl}-3-(4-fluoro-2-methylphenyl)piperidine-1-carboxylate

The title compound was obtained by reaction and purification in the same manner as in Reference Example 3 and using (3R*,4R*)—N-[3,5-bis(trifluoromethyl)benzyl]-3-(4-fluoro-2-methylphenyl)-N-methylpiperidine-4-carboxamide monohydrochloride and methyl chloroformate.
MS (ESI+):535 (M+H)

### Reference Example 73

### (3R*,4R*)—N-[3,5-bis(trifluoromethyl)benzyl]-3-(4-fluoro-2-methylphenyl)-1-(isopropylsulfonyl)-N-methylpiperidine-4-carboxamide

The title compound was obtained by reaction and purification in the same manner as in Reference Example 3 and using (3R*,4R*)—N-[3,5-bis(trifluoromethyl)benzyl]-3-(4-fluoro-2-methylphenyl)-N-methylpiperidine-4-carboxamide monohydrochloride and isopropylsulfonyl chloride.
MS (ESI+):583 (M+H)

### Reference Example 74

### (3S,4S)-1-[amino(oxo)acetyl)-N-[3,5-bis(trifluoromethyl)benzyl]-3-(4-fluoro-2-methylphenyl)-N-methylpiperidine-4-carboxamide

To a solution of (3S,4S)—N-[3,5-bis(trifluoromethyl)benzyl]-3-(4-fluoro-2-methylphenyl)-N-methylpiperidine-4-carboxamide monohydrochloride (108.6 g) synthesized by a known method (WO2006/004195), oxamic acid (22.6 g) and Et₃N (35.4 mL) in CH₃CN (1060 mL) were added WSC HCl (48.7 g) and HOBt-H₂O (32.5 g), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 50→100% ethyl acetate/hexane). The obtained resultant product was dissolved in MeOH (600 mL), activated carbon (5 g) was added and the mixture was stirred at room temperature for 1 hr. Activated carbon was filtered off, and the filtrate was concentrated under reduced pressure to give a pale-pink amorphous solid (119.7 g). The obtained amorphous solid was dissolved in MeOH (424 mL) at 50° C., and water (352 mL) was added. A seed crystal was added, and the mixture was allowed to cool to room temperature by stirring. After stirring for 3 more hours, water (490 mL) was added. After stirring at room temperature for 3 more hours, the precipitate was collected by filtration. The precipitate was washed with water, mixed with water (1400 mL), and the mixture was stirred at 90° C. for 14 hr. After cooling to room temperature, the title compound (110.9 g, 96%) was obtained as a white powder by filtration.
MS (ESI+):548 (M+H)
[α]_{D}²⁵−19.1° (c 1.0, MeOH)
melting point: 124° C.
elemental analysis value: C₂₅H₂₄N₃O₃F₇
Found C, 54.85; H, 4.42; N, 7.68
Calculated C, 54.64; H, 4.39; N, 7.66

### Reference Example 75

### (3R,4R)-1-[amino(oxo)acetyl]-N-[3,5-bis(trifluoromethyl)benzyl]-3-(4-fluoro-2-methylphenyl)-N-methylpiperidine-4-carboxamide

### (Step 1)

(3R*,4R*)-1-(tert-Butoxycarbonyl)-3-(4-fluoro-2-methylphenyl)piperidine-4-carboxylic acid (42.9 g) synthesized by a known method (WO2006/004195) was dissolved in ethyl acetate (1270 mL), a solution of (R)-(−)-1-phenylethylamine (7.70 g) in ethyl acetate (635 mL) was added at room temperature over 2 hr, and the mixture was stirred for one more hour. The precipitate was filtrated and washed with ethyl acetate to give a white powder (24.3 g). The obtained white powder was dissolved in aqueous citric acid solution (citric acid 14 g/water 200 mL) and ethyl acetate (200 mL), and the organic layer was separated. The organic layer was washed with water (twice) and saturated brine and dried, and the solvent was evaporated under reduced pressure to give (3R,4R)-1-(tert-butoxycarbonyl)-3-(4-fluoro-2-methylphenyl)piperidine-4-carboxylic acid (17.8 g, 39%) as a white powder.
MS (ESI+):264 (M+H)

### (Step 2)

To a solution of the compound (2.0 g) obtained in step 1 and DMF (31 μL) in THF (18 mL) was added oxalyl chloride (0.61 ML ) at 0° C. The mixture was stirred at 0° C. for 1 hr, and the reaction mixture was concentrated under reduced pressure under ice-cooling. A solution of the obtained residue in THF (40 mL) was added to a solution of 1-[3,5-bis(trifluoromethyl)phenyl]-N-methylmethanamine monohydrochloride (1.91 g) and ⁱPr₂NEt (2.28 mL) in THF (40 mL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→40% ethyl acetate/hexane), and crystallized from ethyl acetate-IPE to give tert-butyl (3R,4R)-4-{[[3,5-bis(trifluoromethyl)benzyl](methyl)amino]carbonyl}-3-(4-fluoro-2-methylphenyl)piperidine-1-carboxylate (3.03 g, 89%) as a white powder.
MS (ESI+):577 (M+H)

### (Step 3)

To a solution of the compound (2.93 g) obtained in step 2 in ethyl acetate (10 mL) was added 4N hydrogen chloride/ethyl acetate (20 mL) solution, and the mixture was stirred with heating at room temperature for 14 hr. The reaction mixture was concentrated under reduced pressure, and the precipitate was filtered with IPE and hexane to give (3R,4R)—N-[3,5-bis(trifluoromethyl)benzyl]-3-(4-fluoro-2-methylphenyl)-N-methylpiperidine-4-carboxamide monohydrochloride (2.55 g, 98%) as a white powder.
MS (ESI+):477 (M−HCl+H)

### (Step 4)

To a solution of the compound (2.45 g) obtained in step 3, oxamic acid (0.64 g) and Et₃N (1.0 mL) in CH₃CN (24 mL) were added WSC HCl (1.37 g) and HOBt.H₂O (1.10 g), and the mixture was stirred at room temperature for 3 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained resultant product was dissolved in MeOH (20 mL) at 50° C., and water (4 mL) was added. The mixture was stirred for 2 hr with cooling to room temperature, and the precipitate was collected by filtration. The precipitate was washed with water, mixed with water (20 mL), and the mixture was stirred at 85° C. for 8 hr. After cooling to room temperature, the mixture was filtrated to give the title compound (2.34 g, 90%) as a white powder.
MS (ESI+):548 (M+H)
[α]_{D}²⁵+19.5° (c 1.0, MeOH)
elemental analysis value: C₂₅H₂₄N₃O₃F₇
Found C, 54.85; H, 4.42; N, 7.68
Calculated C, 54.62; H, 4.44; N, 7.72

### Reference Example 76

### (3S,4S)-1-[amino(oxo)acetyl]-N-{(1R)-1-[3,5-bis(trifluoromethyl)phenyl]ethyl}-3-(4-fluoro-2-methylphenyl)-N-methylpiperidine-4-carboxamide

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (210.8 mg) synthesized by a known method (WO2006/004195), oxamic acid (46.3 mg) and Et₃N (61 μL) in CH₃CN (4 mL) were added WSC.HCl (99.7 mg) and HOBt.H₂O (79.6 mg), and the mixture was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 40→100% ethyl acetate/hexane) to give the title compound (157 mg, 70%) as a white powder.
MS (ESI+):562 (M+H)
[α]_{D}²⁵+85.4° (c 1.01, MeOH)
elemental analysis value: C₂₆H₂₆N₃O₃F₇
Found C, 55.62; H, 4.67; N, 7.48
Calculated C, 55.59; H, 4.72; N, 7.51

### Reference Example 77

### (3S,4S)—N-{(1R)-1-[3,5-bis(trifluoromethyl)phenyl]ethyl}-3-(4-fluoro-2-methylphenyl)-N-methyl-1-(methylsulfonyl)piperidine-4-carboxamide

To a solution of 2-(methylsulfonyl)ethanol (186 mg) in THF (10 mL) were added methanesulfonyl chloride (113 μL) and Et₃N (198 μL) at room temperature. After stirring at room temperature for 2.5 hr, the precipitate was filtered off. To the filtrate was added a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (300 mg) synthesized by a known method (WO2006/004195) and Et₃N (198 μL) in THF (20 mL) at room temperature, and the mixture was stirred for 20.5 hr. The reaction mixture was poured into water, and the resultant product was extracted with ethyl acetate. The organic layer was washed with saturated aqueous sodium hydrogen carbonate solution and saturated brine and dried, and the solvent was evaporated-under reduced pressure. The obtained residue was purified by silica gel column chromatography (NH Chromatorex) (solvent gradient; 50→100% ethyl acetate/hexane) to give the title compound (69.7 mg, 22%) as a white amorphous solid.
MS (ESI+):569 (M+H)
[α]_{D}²⁵+100.3° (c 1.01, MeOH)

### Reference Example 78

### tert-butyl (3R*,4R*)-4-{[[3,5-bis(trifluoromethyl)benzyl](cyclopropyl)amino]carbonyl}-3-(4-fluoro-2-methylphenyl)piperidine-1-carboxylate

To a solution of (3R*,4R*)-1-(tert-butoxycarbonyl)-3-(4-fluoro-2-methylphenyl)piperidine-4-carboxylic acid (3.37 g) synthesized by a known method (WO2006/004195) and cyclopropylamine (831 μL) in CH₃CN (50 mL) were added WSC.HCl (2.30 g) and HOBt.H₂O (1.84 g), and the mixture was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure to give tert-butyl (3R*,4R*)-4-[(cyclopropylamino)carbonyl]-3-(4-fluoro-2-methylphenyl)piperidine-1-carboxylate (3.05 g, 81%) as a white powder.
MS (ESI+):321 (M−^{t}Bu+2H)

### (Step 2)

To a solution of the compound (753 mg) obtained in step 1 in DMF (8 mL) was added sodium hydride (60% in oil, 96 mg) at 0° C., and the mixture was stirred for 15 min. 3,5-bis(Trifluoromethyl)benzyl bromide (550 μL) was added to the reaction mixture and the mixture was stirred at room temperature for one day. Sodium hydride (60% in oil, 96 mg) and 3,5-bis(trifluoromethyl)benzyl bromide (550 μL) were further added, and the mixture was stirred at 60° C. for 2 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 25→50% ethyl acetate/hexane) to give the title compound (608 mg, 50%) as a white amorphous solid.
MS (ESI+):547 (M−^{t}Bu+2H)

### Reference Example 79

### (3R*,4R*)—N-[3,5-bis(trifluoromethyl)benzyl]-N-cyclopropyl-3-(4-fluoro-2-methylphenyl)piperidine-4-carboxamide monohydrochloride

To a solution of the compound (2.33 g) obtained in Reference Example 78 in ethyl acetate (4 mL) was added a 4N hydrogen chloride/ethyl acetate (16 mL) solution, and the mixture was stirred at room temperature for 3 hr. The reaction mixture was concentrated under reduced pressure, and the residue was crystallized from EtOH-IPE to give the title compound (2.09 g, 100%) as a white powder.
MS (ESI+):503 (M−HCl+H)

### Reference Example 80

### (3R*,4R*)—N⁴-(3,5-bis(trifluoromethyl)benzyl]-N⁴-cyclopropyl-3-(4-fluoro-2-methylphenyl)piperidine-1,4-dicarboxamide

To a solution of the compound (150 mg) obtained in Reference Example 79 and triphosgene (113 mg) in THF (5 mL) was added Et₃N (116 μL) at 0° C., and the mixture was stirred at 0° C. for 30 min. A 28% aqueous ammonia solution (928 μL) was added at 0° C., and the mixture was stirred at room temperature for 19 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (99.1 mg, 65%) as a white powder.
MS (ESI+):546 (M+H)

### Reference Example 81

### (3R*,4R*)-1-[amino(oxo)acetyl]-N-[3,5-bis(trifluoromethyl)benzyl]-N-cyclopropyl-3-(4-fluoro-2-methylphenyl)piperidine-4-carboxamide

The title compound was obtained by reaction and purification in the same manner as in Reference Example 1 and using the compound obtained in Reference Example 79.
MS (ESI+):574 (M+H)

### Reference Example 82

### (3R*,4R*)—N-[3,5-bis(trifluoromethyl)benzyl]-N-cyclopropyl-1-[(2,6-dioxopiperidin-4-yl)carbonyl]-3-(4-fluoro-2-methylphenyl)piperidine-4-carboxamide

The title compound was obtained by reaction and purification in the same manner as in Reference Example 1 and using the compound obtained in Reference Example 79 and 2,6-dioxopiperidine-4-carboxylic acid.
MS (ESI+):642 (M+H)

### Reference Example 83

### (3R*,4R*)—N-[3,5-bis(trifluoromethyl)benzyl]-N-cyclopropyl-3-(4-fluoro-2-methylphenyl)-1-glycoloylpiperidine-4-carboxamide

The title compound was obtained by reaction and purification in the same manner as in Reference Example 1 and using the compound obtained in Reference Example 79 and glycolic acid.
MS (ESI+):561 (M+H)

### Reference Example 84

### (3R*,4R*)—N-[3,5-bis(trifluoromethyl)benzyl]-N-cyclopropyl-3-(4-fluoro-2-methylphenyl)-1-(3-hydroxy-3-methylbutanoyl)piperidine-4-carboxamide

The title compound was obtained by reaction and purification in the same manner as in Reference Example 1 and using the compound obtained in Reference Example 79 and β-hydroxyisovaleric acid.
MS (ESI+):603 (M+H)

### Reference Example 85

### (3R*,4R*)-1-(N-acetylglycyl)-N-[3,5-bis(trifluoromethyl)benzyl]-N-cyclopropyl-3-(4-fluoro-2-methylphenyl)piperidine-4-carboxamide

The title compound was obtained by reaction and purification in the same manner as in Reference Example 1 and using the compound obtained in Reference Example 79 and N-acetylglycine.
MS (ESI+): 602 (M+H)

### Reference Example 86

### (3R*,4R*)—N-[3,5-bis(trifluoromethyl)benzyl]-N-cyclopropyl-3-(4-fluoro-2-methylphenyl)-1-(methylsulfonyl)piperidine-4-carboxamide

### Reference Example 87

### (3R*,4R*)—N-[3,5-bis(trifluoromethyl)benzyl]-N-cyclopropyl-3-(4-fluoro-2-methylphenyl)-1-[2-(methylsulfonyl)ethyl]piperidine-4-carboxamide monohydrochloride

To a solution of 2-(methylsulfonyl)ethanol (97 mg) in THF (4 mL) were added methanesulfonyl chloride (73.6 μL) and Et₃N (129 μL) at room temperature. The mixture was stirred at room temperature for 1 hr, and the precipitate was filtered off. To the filtrate was added a solution of the compound (200 mg) obtained in Reference Example 79 and Et₃N (129 μL) in THF (4 mL) at room temperature, and the mixture was stirred for 24 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The compound (55.2 mg, 26%) of Reference Example 86 was obtained as a white amorphous solid from a fraction with a short retention time. A fraction with a long retention time was concentrated under reduced pressure and treated with 1 equivalent amount of 4N hydrogen chloride/ethyl acetate to give the compound (54.3 mg, 23%) of

### Reference Example 87 as a white powder.

### Compound of Reference Example 86

MS (ESI+):581 (M+H)

### Compound of Reference Example 87

MS (ESI+):609 (M−HCl+H)

### Reference Example 88

### (3R*,4R*)-1′-acetyl-N-[3,5-bis(trifluoromethyl)benzyl]-N-cyclopropyl-3-(4-fluoro-2-methylphenyl)-1,4′-bipiperidine-4-carboxamide monohydrochloride

To a solution of the compound (269 mg) obtained in Reference Example 79 and 1-acetyl-4-piperidone (91.8 mg) in EtOH (4 mL) was added TiCl₄ (192 μL), and the mixture was stirred at room temperature for 1 hr. NaBH(OAc)₃ (212 mg) was added, and the mixture was stirred at room temperature for one day. 1-Acetyl-4-piperidone (307 mg), Et₃N (84 μL) and NaBH(OAc)₃ (106 mg) were added to the reaction mixture, and the mixture was stirred at room temperature for 3 days. The reaction mixture was poured into an aqueous sodium hydrogen carbonate solution, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 50→100% ethyl acetate/hexane) to give a colorless oil (116 mg). The oil was treated with 1 equivalent amount of 4N hydrogen chloride/ethyl acetate to give the title compound (99.0 mg, 30%) as a white powder.
MS (ESI+):628 (M−HCl+H)

### Reference Example 89

### tert-butyl (3R*,4R*)-4-{[{1-[3,5-bis(trifluoromethyl)phenyl]ethyl}(cyclopropyl)amino]carbonyl}-3-(4-fluoro-2-methylphenyl)-piperidine-1-carboxylate (less polar)

### Reference Example 90

### tert-butyl (3R*,4R*)-4-{[{1-[3,5-bis(trifluoromethyl)phenyl]ethyl}(cyclopropyl)amino]carbonyl}-3-(4-fluoro-2-methylphenyl)piperidine-1-carboxylate (more polar)

### (Step 1)

To a solution of 3′,5′-bis(trifluoromethyl)acetophenone (37.1 g) in MeOH (100 mL) was added a solution of cyclopropylamine (20.7 g) in MeOH (45 mL) at 0° C., and the mixture was stirred at room temperature for one day. The reaction mixture was cooled to 0° C., NaBH₄ (5.48 g) was added, and the mixture was stirred at room temperature for 2 hr. Water was added to the reaction mixture, and the mixture was concentrated under reduced pressure. A 2N aqueous sodium hydroxide solution was poured onto the residue, and the resultant product was extracted with ethyl acetate. The organic layer was washed with water and saturated brine and dried, and the solvent was evaporated under reduced pressure. The obtained residue was treated with 1 equivalent amount of 4N hydrogen chloride/ethyl acetate to give N-{1-[3,5-bis(trifluoromethyl)phenyl]ethyl}cyclopropanamine monohydrochloride (37.0 g, 76%) as a white powder.

¹H-NMR (300 MHz, DMSO-d₆):δ 0.50-1.00 (4H, m), 1.67 (3H, d, J=6.9 Hz), 4.75 (1H, br), 8.17 (1H, s), 8.42 (2H, s), 9.79 (1H, br), 10.17 (1H, br)

### (Step 2)

To a solution of (3R*,4R)-1-(tert-butoxycarbonyl)-3-(4-fluoro-2-methylphenyl)piperidine-4-carboxylic acid (0.67 g) synthesized by a known method (WO2006/004195) and DMF (10 μL) in THF (15 mL) was added oxalyl chloride (0.21 mL) at.0° C. The mixture was stirred at 0° C. for 1 hr, and concentrated under reduced pressure under ice-cooling. A solution of the obtained residue in THF (10 mL) was added to a solution of the compound (0.80 g) obtained in step 1 and Et₃N (0.51 mL) in THF (5 mL) at. 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→40% ethyl acetate/hexane). The compound (0.36 g, 29%) of Reference Example 89 was obtained as a colorless oil from a fraction with a short retention time. The compound (0.45 g, 37%) of Reference Example 90 was obtained as a colorless oil from a fraction with a long retention time.

### Compound of Reference Example 89

MS (ESI+):561 (M−^{t}Bu+2H)

### Compound of Reference Example 90

MS (ESI+):561 (M−^{t}Bu+2H)

### Reference Example 91

### (3R*,4R*)—N-{1-[3,5-bis(trifluoromethyl)phenyl]ethyl}-N-cyclopropyl-3-(4-fluoro-2-methylphenyl)piperidine-4-carboxamide monohydrochloride (less polar)

The title compound was obtained by reaction and purification in the same manner as in Reference Example 6 and using the compound obtained in Reference Example 89.
MS (ESI+):517 (M−HCl+H)

### Reference Example 92

### (3R*,4R*)—N-{1-[3,5-bis(trifluoromethyl)phenyl]ethyl}-N-cyclopropyl-3-(4-fluoro-2-methylphenyl)piperidine-4-carboxamide monohydrochloride (more polar)

The title compound was obtained by reaction and purification in the same manner as in Reference Example 6 and using the compound obtained in Reference Example 90.
MS (ESI+):517 (M−HCl+H)

### Reference Example 93

### (3R,4R)-1-[amino(oxo)acetyl]-N-1-[3,5-bis(trifluoromethyl)phenyl]ethyl}-N-cyclopropyl-3-(4-fluoro-2-methylphenyl)piperidine-4-carboxamide (less polar)

The title compound was obtained by reaction and purification in the same manner as in Reference Example 1 and using the compound obtained in Reference Example 91.

¹H-NMR (300 MHz, CDCl₃):δ 0.015-0.030 (2H, m), 0.50-0.75 (2H, m), 1.32 (3H, d, J=7.0 Hz), 1.90-2.20 (2H, m), 2.30-2.50 (4H, m), 2.69-2.85 (1H, m), 3.05-3.30 (1H, m), 3.35-3.60 (1H, m), 3.65-3.80 (1H, m), 4.55-5.20 (2H, m), 5.30 (1H, q, J=7.2 Hz), 5.52-5.59 (1H, m), 6.78-6.90 (2H, m), 6.95-7.05 (1H, m), 7.10-7.20 (1H, m), 7.52 (2H, s), 7.69 (1H, s)
MS (ESI+):588 (M+H)

### Reference Example 94

### (3R*,4R*)-1-[amino(oxo)acetyl]-N-{1-[3,5-bis(trifluoromethyl)phenyl]ethyl}-N-cyclopropyl-3-(4-fluoro-2-methylphenyl)piperidine-4-carboxamide (more polar)

The title compound was obtained by reaction and purification in the same manner as in Reference Example 1 and using the compound obtained in Reference Example 92.

¹H-NMR (300 MHz, CDCl₃):δ 0.45-0.65 (2H, m), 0.80-0.95 (2H, m)), 1.51 (3H, d, J=7.2 Hz), 1.80-2.10 (2H, m), 2.40-2.50 (4H, m), 2.71-2.86 (1H, m), 3.02-3.26 (1H, m), 3.45-3.70 (1H, m), 3.70-3.85 (1H, m), 4.54-5.25 (2H, m), 5.30-5.40 (1H, m), 5.50-5.60 (1H, m), 6.75-6.90 (2H, m), 6.90-7.08 (1H, m), 7.08-7.18 (1H, m), 7.26 (2H, s), 7.66 (1H, s)
MS (ESI+):588 (M+H)

### Reference Example 95

### tert-butyl (3R*,4R*)-4-{[[3-chloro-5-(trifluoromethyl)benzyl](methyl)amino]carbonyl}-3-(4-fluoro-2-methylphenyl)piperidine-1-carboxylate

### (Step 1)

To a solution of 3-trifluoromethylbenzoic acid (203.4 g) in concentrated sulfuric acid (880 mL) was added 90% fuming nitric acid (210 mL) at 0° C. over 1 hr. The mixture was stirred at 35° C. for 3 hr, and slowly poured onto ice (about 1 kg). The precipitate was filtrated with water (500 mL), and dissolved in ethyl acetate (500 mL). The ethyl acetate solution was washed with water and dried, and the solvent was evaporated under reduced pressure to give 3-nitro-5-(trifluoromethyl)benzoic acid (232.5 g, 92%) as a white powder.
¹H-NMR (300 MHz, CDCl₃):δ 8.69 (1H, s), 8.74 (1H, s), 9.1 (1H, s)

### (Step 2)

A suspension of the compound (103 g×2 batch) obtained in step 1 and 10% palladium-carbon (5.15 g×2 batch) in EtOH (1500 mL×2 batch) was stirred under 1 atm hydrogen atmosphere, at room temperature for 7 hr. The catalyst was filtered off, and the filtrate was concentrated under reduced pressure to give 3-amino-5-(trifluoromethyl)benzoic acid (180 g, 100%) as a pale-yellow powder.

¹H-NMR (300 MHz, CDCl₃):δ 4.00-6.00 (2H, br), 7.10 (1H, s), 7.53 (1H, s), 7.72 (1H, s)

### (Step 3)

A solution of ^{t}BuONO (35.2 g) and CuCl (38.6 g) in CH₃CN (375 mL) was warmed to 45-50° C. The compound (50.0 g) obtained in step 2 was added slowly while cooling the reaction mixture to maintain 45-50° C. After stirring at 50° C. for 30 min, the mixture was cooled to 10° C. 6N Hydrochloric acid (250 mL) was added and the mixture was stirred for 10 min. The resultant product was extracted twice with ethyl acetate. The organic layer was-washed with 10% aqueous citric acid solution and water and dried, and the solvent was evaporated under reduced pressure. The obtained residue was purified by silica gel column chromatography (solvent gradient; 10→25% ethyl acetate/hexane) to give 3-chloro-5-(trifluoromethyl)benzoic acid (33.1 g, 60%) as a white powder.

¹H-NMR (300 MHz, CDCl₃):δ 4.00-6.00 (1H, br), 7.86 (1H, s), 8.26 (2H, s)

### (Step 4)

A solution of the compound (21.6 g) obtained in step 3, MF (100 μL) and thionyl chloride (21.0 mL) in toluene (63 mL) was stirred at 85° C. for 1 hr. The reaction mixture was concentrated under reduced pressure, and the residue was dissolved in DME (50 mL). After cooling the DME solution to −78° C., NaBH₄ (5.83 g) was added slowly. The reaction mixture was stirred at room temperature for 1 hr, and poured into 6N hydrochloric acid and ice. The resultant product was extracted twice with ethyl acetate, and the organic layer was washed with a saturated aqueous ammonium chloride solution and water and dried, and the solvent was evaporated under reduced pressure. However, the compound obtained in step 3 remained unreactive in the obtained residue. The residue was dissolved in THF (50 mL), 1.1M borane-THF complex (280 mL/THF solution) was added at 0° C. The mixture was stirred at 90° C. for 2 hr and, after cooling, 6N hydrochloric acid (50 mL) was added. The resultant product was extracted twice with ethyl acetate, and the organic layer was washed with a saturated aqueous ammonium chloride solution and water and dried, and the solvent was evaporated under reduced pressure. The obtained residue was purified by silica gel column chromatography (solvent gradient; 5→10% ethyl acetate/hexane) to give [3-chloro-5-(trifluoromethyl)phenyl]methanol (10.7 g, 53%) as a colorless oil.

¹H-NMR (300 MHz, CDCl₃):δ 1.92 (1H, t, J=5.9 Hz), 4.76 (2H, d, J=6.0 Hz), 7.52 (2H, s), 7.55 (1H, s)

### (Step 5)

A solution of the compound (1.00 g) obtained in step 4 and MnO₂ (2.06 g) in toluene (30 mL) was stirred at 50° C. for 12 hr. MnO₂ (2.06 g) was further added, and the mixture was stirred at 50° C. for 2 hr. Insoluble material was filtered off, and the filtrate was concentrated under reduced pressure. The obtained residue was purified by silica gel column chromatography (solvent; 5% ethyl acetate/hexane) to give 3-chloro-5-(trifluoromethyl)benzaldehyde (0.609 g, 62%) as a pale-yellow oil.

¹H-NMR (300 MHz, CDCl₃):δ 7.86 (1H, s), 8.02 (1H, s), 8.04 (1H, s), 10.00 (1H, s)

### (Step 6)

To a solution of the compound (7.57 g) obtained in step 5 in MeOH (49mL) was added 38% methylamine (9.8 mL/MeOH solution), and the mixture was stirred at room temperature for 14 hr. The reaction mixture was cooled to 0° C., NaBH₄ (1.40 g) was added, and the mixture was stirred at room temperature for 2 hr. Water was added to the reaction mixture, and the mixture was concentrated under reduced pressure. A 2N aqueous sodium hydroxide solution was poured onto the residue, and the resultant product was extracted with ethyl acetate. The organic layer was washed with water and saturated brine and dried, and the solvent was evaporated under reduced pressure. The obtained residue was treated with 1 equivalent amount of 4N hydrogen chloride/ethyl acetate to give 1-[3-chloro-5-(trifluoromethyl)phenyl]-N-methylmethanamine monohydrochloride (7.10 g, 75%) as a white powder.

¹H-NMR (300 MHz, CDCl₃):δ 2.60 (3H, s), 4.12 (2H, s), 7.64 (1H, s), 7.80 (1H, s), 7.89 (1H, s)

### (Step 7)

To a solution of (3R*,4R*)-1-(tert-butoxycarbonyl)-3-(4-fluoro-2-methylphenyl)piperidine-4-carboxylic acid (1.26 g) synthesized by a known method (WO2006/004195) and DMF (50 μL) in THF (25 mL) was added oxalyl chloride (0.39 mL) at 0° C. The mixture was stirred at 0° C. for 1 hr, and the reaction mixture was concentrated under reduced pressure under ice-cooling. A solution of the obtained residue in CH₃CN (10 mL) was added to a solution of the compound (1.17 g) obtained in step 6 and Et₃N (1.18 mL) in THF (25 mL) at 0° C., and the mixture was stirred at room temperature for 2 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→50% ethyl acetate/hexane) to give the title compound (1.43 g, 71%) as a white powder.
MS (ESI+):487 (M−^{t}Bu+2H)

### Reference Example 96

### (3R*,4R*)—N-[3-chloro-5-(trifluoromethyl)benzyl]-3-(4-fluoro-2-methylphenyl)-N-methylpiperidine-4-carboxamide monohydrochloride

The title compound was obtained by reaction and purification in the same manner as in Reference Example 6 and using the compound obtained in Reference Example 95.
MS (ESI+):443 (M−HCl+H)

### Reference Example 97

### (3R*,4R*)-1-[amino(oxo)acetyl]-N-[3-chloro-5-(trifluoromethyl)benzyl]-3-(4-fluoro-2-methylphenyl)-N-methylpiperidine-4-carboxamide

The title compound was obtained by reaction and purification in the same manner as in Reference Example 1 and using the compound obtained in Reference Example 96.
MS (ESI+):514 (M+H)

### Reference Example 98

### tert-butyl (3R*,4R*)-4-{[{1-[3-chloro-5-(trifluoromethyl)phenyl]ethyl}[methyl]amino]carbonyl}-3-(4-fluoro-2-methylphenyl)piperidine-1-carboxylate (less polar)

### Reference Example 99

### tert-butyl (3R*,4R*)-4-{[{1-[3-chloro-5-(trifluoromethyl)phenyl]ethyl}[methyl]amino]carbonyl}-3-(4-fluoro-2-methylphenyl)piperidine-1-carboxylate (more polar)

### (Step 1)

To a solution of the compound (13.8 g) obtained in Reference Example 95, step 3, N,O-dimethylhydroxylamine hydrochloride (7.88 g) and Et₃N (11.3 mL) in CH₃CN (100 mL) were added WSC.HCl (25.8 g) and HOBt.H₂O (15.5 g), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→20% ethyl acetate/hexane) to give 3-chloro-N-methoxy-N-methyl-5-(trifluoromethyl)benzamide (15.09 g, 84%) as a pale-yellow oil.

¹H-NMR (300 MHz, CDCl₃):δ 3.38 (3H, s), 3.56 (3H, s), 7.69 (1H, s), 7.86 (2H, m)

### (Step 2)

To a solution of the compound (15.0 g) obtained in step 1 in THF (150 mL) was added 1M methylmagnesium bromide (80 mL/THF solution) at −30° C., and the mixture was stirred at room temperature for 2 hr. The reaction mixture was poured into an aqueous ammonium chloride solution, and the resultant product was extracted with ethyl acetate. The organic layer was washed with water and saturated brine and dried, and the solvent was evaporated under reduced pressure. The obtained residue was purified by silica gel column chromatography (solvent gradient; 5→10% ethyl acetate/hexane) to give 1-[3-chloro-5-(trifluoromethyl)phenyl]ethanone (12.37 g, 99%) as a colorless oil.

¹H-NMR (300 MHz, CDCl₃):δ 2.65 (3H, s), 7.80 (1H, s), 8.07 (1H, s), 8.09 (1H, s)

### (Step 3)

To a solution of the compound (12.0 g) obtained in step 2 in MeOH (23 mL) was added 38% methylamine (14.9 mL/MeOH solution), and the mixture was stirred at room temperature for 2 days. The reaction mixture was cooled to 0° C., NaBH₄ (2.10 g) was added, and the mixture was stirred at room temperature for 2 hr. Water was added to the reaction mixture, and the mixture was concentrated under reduced pressure. A 2N aqueous sodium hydroxide solution was poured onto the residue, and the resultant product was extracted with ethyl acetate. The organic layer was washed with water and saturated brine and dried, and the solvent was evaporated under reduced pressure to give crude 1-[3-chloro-5-(trifluoromethyl)phenyl]-N-methylethanamine (12.26 g, 96%) as a colorless oil.

¹H-NMR (300 MHz, CDCl₃):δ 1.34 (3H, d, J=6.6 Hz), 2.30 (3H, s), 3.69 (1H, q, J=6.6 Hz), 7.46-7.48 (2H, m), 7.50 (1H, s)

### (Step 4)

To a solution of (3R*,4R)-1-(tert-butoxycarbonyl)-3-(4-fluoro-2-methylphenyl)piperidine-4-carboxylic acid (2.00 g) synthesized by a known method (WO2006/004195) and DMF (30 μL) in THF (15 mL) was added oxalyl chloride (0.62 mL) at 0° C. The mixture was stirred at 0° C. for 1 hr, and the reaction mixture was concentrated under reduced pressure under ice-cooling. A solution of the obtained residue in THF (10 mL) was added to a solution of the compound (1.69 g) obtained in step 3 and Et₃N (1.24 mL) in CH₃CN (25 mL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→40% ethyl acetate/hexane). The compound (0.90 g, 27%) of Reference Example 98 was obtained as a colorless oil from a fraction with a short retention time. The compound (2.15 g, 65%) of Reference Example 99 was obtained as a white powder from a fraction with a long retention time.

### Compound of Reference Example 98

MS (ESI+): 501 (M−^{t}Bu+2H)

### Compound of Reference Example 99

MS (ESI+):501 (M−^{t}Bu+2H)

### Reference Example 100

### (3R*,4R*)—N-{1-[3-chloro-5-(trifluoromethyl)phenyl]ethyl}-3-(4-fluoro-2-methylphenyl)-N-methylpiperidine-4-carboxamide monohydrochloride (less polar)

The title compound was obtained by reaction and purification in the same manner as in Reference Example 6 and using the compound obtained in Reference Example 98.
MS (ESI+):457 (M−HCl+H)

### Reference Example 101

### (3R*,4R*)—N-{1-[3-chloro-5-(trifluoromethyl)phenyl]ethyl}-3-(4-fluoro-2-methylphenyl)-N-methylpiperidine-4-carboxamide monohydrochloride (more polar)

The title compound was obtained by reaction and purification in the same manner as in Reference Example 6 and using the compound obtained in Reference Example 99.
MS (ESI+):457 (M−HCl+H)

### Reference Example 102

### (3R*,4R*)-1-[amino(oxo)acetyl]-N-{1-[3-chloro-5-(trifluoromethyl)phenyl]ethyl)-3-(4-fluoro-2-methylphenyl)-N-methylpiperidine-4-carboxamide (less polar)

The title compound was obtained by reaction and purification in the same manner as in Reference Example 1 and using the compound obtained in Reference Example 100.
MS (ESI+):528 (M+H)

### Reference Example 103

### (3R*,4R*)-1-[amino(oxo)acetyl]-N-{1-[3-chloro-5-(trifluoromethyl)phenyl]ethyl}-3-(4-fluoro-2-methylphenyl)-N-methylpiperidine-4-carboxamide (more polar)

The title compound was obtained by reaction and purification in the same manner as in Reference Example 1 and using the compound obtained in Reference Example 101.
MS (ESI+):528 (M+H)

### Reference Example 110

### tert-butyl (3R*,4R*)-4-{[[3,5-bis(trifluoromethyl)benzyl](methyl)amino]carbonyl}-3-(3,4-dichlorophenyl)piperidine-1-carboxylate

### (Step 1)

A solution of ethyl 1-benzyl-5-(3,4-dichlorophenyl)-1,2,3,6-tetrahydropyridine-4-carboxylate (19.7 g) synthesized by a known method (WO2005/068427) and 1-chloroethyl chloroformate (9.85 mL) in CH₃CN (100 mL) was added at 0° C., and the mixture was heated under reflux for 2 hr. The reaction mixture was concentrated under reduced pressure, methanol (150 mL) was added to the residue, and the mixture was heated under reflux for 1 hr. The reaction mixture was concentrated under reduced pressure. To a solution of the residue in CH₃CN (150 mL) were added. Et₃N (7.7 mL) and: Boc₂O (12.1 g), and the mixture was stirred at room temperature for 14 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 5→10% ethyl acetate/hexane) to give 1-tert-butyl 4-ethyl 5-(3,4-dichlorophenyl)-3,6-dihydropyridine-1,4(2H)-dicarboxylate (10.2 g, 51%) as a white powder.

¹H-NMR (300 MHz, CDCl₃):δ 0.97 (3H, t, J=7.2 Hz), 1.48 (9H, s), 2.51-2.56 (2H, m), 3.58 (2H, t, J=5.7 Hz), 3.97 (2H, q, J=7.2 Hz), 4.05-4.15 (2H, m), 7.02 (1H, dd, J=8.4,2.1 Hz), 7.28 (1H, d, J=2.1 Hz), 7.41 (1H, d, J=8.4 Hz)

### (Step 2)

To a solution of the compound (7.10 g) obtained in step 1 in MeOH (144 mL) was added magnesium (4.31 g), and the mixture was stirred at room temperature for 14 hr. A saturated aqueous ammonium chloride solution (300 mL) was added to the reaction mixture, and the resultant product was extracted with ethyl acetate. The organic layer was washed with water and saturated brine and dried, and the solvent was evaporated under reduced pressure. To a solution of the obtained residue in EtOH (116 mL) was added sodium ethoxide (sodium 0.82 g/EtOH 66 mL), and the mixture was stirred at 90° C. for 2 hr. The reaction mixture was poured into an aqueous ammonium chloride solution, and the resultant product was extracted with ethyl acetate. The organic layer was washed with water and saturated brine and dried, and the solvent was evaporated under reduced pressure. The obtained residue was purified by silica gel column chromatography (solvent gradient; 5→50% ethyl acetate/hexane) to give ethyl (3R*,4R*)-1-(tert-butoxycarbonyl)-3-(3,4-dichlorophenyl)piperidine-4-carboxylate (7.2 g, 100%) as a pale-yellow oil.

¹H-NMR (300 MHz, CDCl₃):δ 1.05 (3H, t, J=7.2 Hz), 1.47 (9H, s), 1.65-1.85 (1H, m), 1.90-2.05 (1H, m), 2.60-3.00 (4H, m), 3.96 (2H, q, J=7.2 Hz), 3.80-4.40 (2H, br), 7.04-7.37 (3H, m)

### (Step 3)

To a solution of the compound (7.2 g) obtained in step 2 in EtOH (11 mL) was added 8N aqueous sodium hydroxide solution (11 mL), and the mixture was stirred at 90° C. for 1 hr. The reaction mixture was weakly acidified with an aqueous citric acid solution, and the resultant product was extracted with ethyl acetate. The organic layer was washed with water and saturated brine and dried, and the solvent was evaporated under reduced pressure to give (3R*,4R*)-1-(tert-butoxycarbonyl)-3-(3,4-dichlorophenyl)piperidine-4-carboxylic acid (5.89 g, 89%) as a white powder.

¹H-NMR (300 MHz, CDCl₃):δ 1.45 (9H, s), 1.65-2.20 (2H, m), 2.60-3.00 (4H, m), 3.20-4.40 (2H, m), 7.02-7.36 (3H, m)

### (Step 4)

To a solution of the compound (0.74 g) obtained in step 3 and DMF (about 20 μL) in THF (10 mL) was added oxalyl chloride (0.26 mL) at 0° C. The mixture was stirred at 0° C. for 1 hr, and the reaction mixture was concentrated under reduced pressure under ice-cooling. A solution of the obtained residue in THF (10 mL) was added to a solution of 1-[3,5-bis(trifluoromethyl)phenyl]-N-methylmethanamine monohydrochloride (0.70 g) and Et₃N (0.69 mL) in CH₃CN (15 mL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→50% ethyl acetate/hexane) to give the title compound (1.12 g, 92%) as a white powder.

¹H-NMR (300 MHz, CDCl₃):δ 1.47 (9H, s), 1.75-2.00 (2H, m), 2.60-3.30 (7H, m), 4.00-4.80 (4H, m), 6.96-7.83 (6H, m)
MS (ESI+):558 (M−^{t}Bu+2H)

### Reference Example 111

### (3R*,4R*)—N-[3,5-bis(trifluoromethyl)benzyl]-3-(3,4-dichlorophenyl)-N-methylpiperidine-4-carboxamide monohydrochloride

The title compound was obtained by reaction and purification in the same manner as in Reference Example 6 and using the compound obtained in Reference Example 110.
MS (ESI+):514 (M−HCl+H)

### Reference Example 112

### (3R*,4R*)-1-[amino(oxo)acetyl]-N-[3,5-bis(trifluoromethyl)benzyl]-3-(3,4-dichlorophenyl)-N-methylpiperidine-4-carboxamide

The title compound was obtained by reaction and purification in the same manner as in Reference Example 1 and using the compound obtained in Reference Example 111.
MS (ESI+):585 (M+H)

### Reference Example 113

### (3R ,4R)-1-[(1-acetylpiperidin-4-yl)carbonyl]-N-[3,5-bis(trifluoromethyl)benzyl]-3-(3,4-dichlorophenyl)-N-methylpiperidine-4-carboxamide

The title compound was obtained by reaction and purification in the same manner as in Reference Example 1 and using the compound obtained in Reference Example 111 and 1-acetylpiperidine-4-carboxylic acid.
MS (ESI+):667 (M+H)

### Reference Example 114

### tert-butyl (3R*,4S*)-4-{[[3,5-bis(trifluoromethyl)benzyl](methyl)amino]carbonyl}-3-(3-methyl-2-thienyl)piperidine-1-carboxylate

### (Step 1)

To a solution of ethyl 1-benzyl-3-oxopiperidine-4-carboxylate hydrochloride (8.74 g) in DMF (40 mL) was added sodium hydride (60% in oil, 2.93 g) at 0° C., and the mixture was stirred for 5 min. N-phenylbis(trifluoromethanesulfonimide) (11.5 g) was added, and the mixture was stirred at 0° C. for 1 hr. The reaction mixture was poured into ice water, and the resultant product was extracted with ethyl acetate. The organic layer was washed with saturated aqueous sodium hydrogen carbonate solution and saturated brine and dried, and the solvent was evaporated under reduced pressure to give a colorless oil. To a solution of the oil obtained above in toluene (118 mL)-H₂O (7 mL) were added 3-methylthiophene-2-boronic acid (5.00 g), potassium carbonate (4.05 g) and tetrakis(triphenylphosphine)palladium (0) (3.39 g), and the mixture was stirred under an argon atmosphere at 100° C. for 14 hr. The reaction mixture was poured into water, and the resultant product was extracted with ethyl acetate. The organic layer was washed with aqueous ammonium chloride solution and saturated brine and dried, and the solvent was evaporated under reduced pressure. The obtained residue was purified by silica gel column chromatography (solvent gradient; 5→20% ethyl acetate/hexane) to give ethyl 1-benzyl-5-(3-methyl-2-thienyl)-1,2,3,6-tetrahydropyridine-4-carboxylate (7.13 g, 71%) as a colorless oil.

¹H-NMR (300 MHz, CDCl₃):δ 0.96 (3H, t, J=7.2 Hz), 2.05 (3H, s), 2.55-2.60 (2H, m), 2.67 (2H, t, J=7.6 Hz), 3.20 (2H, t, J=2.6 Hz), 3.64 (2H, s), 3.95 (2H, q, J=7.2 Hz), 6.76 (1H, d, J=5.4 Hz), 7.14 (1H, d, J=5.4 Hz), 7.24-7.36 (5H, m)

### (Step 2)

A solution of the compound (7.00 g) obtained in step 1 and 1-chloroethyl chloroformate (4.0 mL) in CH₃CN (50 mL) were added at 0° C., and the mixture was heated under reflux for 2 hr. The reaction mixture was concentrated under reduced pressure, methanol (50 mL) was added to the residue, and the mixture was heated under reflux for 1 hr. The reaction mixture was concentrated under reduced pressure, Et₃N (3.43 mL) and Boc₂O (5.37 g) were added to a solution of the residue in CH₃CN (50 mL), and the mixture was stirred at room temperature for 14 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 5→20% ethyl acetate/hexane) to give 1-tert-butyl 4-ethyl 5-(3-methyl-2-thienyl)-3,6-dihydropyridine-1,4(2H)-dicarboxylate (6.02 g, 84%) as a colorless oil.

¹H-NMR (300 MHz, CDCl₃):δ 0.97 (3H, t, J=7.2 Hz), 1.48 (9H, s), 2.09 (3H, s), 2.54-2.59 (2H, m), 3.59 (2H, t, J=5.9 Hz), 3.97 (2H, q, J=7.2 Hz), 4.08 (2H, s), 6.80 (1H, d, J=5.1 Hz), 7.19 (1H, d, J=5.1 Hz)

### (Step 3)

To a solution of the compound (5.98 g) obtained in step 2 in MeOH (70 mL) was added magnesium (4.13 g) and the mixture was stirred at room temperature for 14 hr. A saturated aqueous ammonium chloride solution (300 mL) was added to the reaction mixture, and the resultant product was extracted with ethyl acetate. The organic layer was washed with water and saturated brine and dried, and the solvent was evaporated under reduced pressure. Sodium hydride (60% in oil, 1.36 g) was added to a solution of the obtained residue in EtOH (100 mL), and the mixture was stirred at 90° C. for 2 hr. The reaction mixture was poured into an aqueous ammonium chloride solution, and the resultant product was extracted with ethyl acetate. The organic layer was washed with water and saturated brine and dried, and the solvent was evaporated under reduced pressure. The obtained residue was purified by silica gel column chromatography (solvent gradient; 5→20% ethyl acetate/hexane) to give 1-tert-butyl 4-ethyl (3R*,4S*)-3-(3-methyl-2-thienyl)piperidine-1,4-dicarboxylate (3.84 g, 64%) as a pale-yellow oil.

¹H-NMR (300 MHz, CDCl₃):δ 1.06 (3H, t, J=7.2 Hz), 1.47 (9H, s), 1.70-1.85 (1H, m), 1.90-2.00 (1H, m), 2.22 (3H, s), 2.55-2.90 (3H, m), 3.32 (1H, dt, J=11.1, 4.2 Hz), 3.90-4.40 (4H, m), 6.77 (1H, d, J=4.8 Hz), 7.08 (1H, d, J=4.8 Hz)

### (Step 4)

To a solution of the compound (3.74 g) obtained in step 3 in EtOH (50 mL) was added 12N aqueous sodium hydroxide solution (30 mL), and the mixture was stirred for 90° C. for 14 hr. The reaction mixture was weakly acidified with an aqueous citric acid solution, and the resultant product was extracted with ethyl acetate. The organic layer was washed with water and saturated brine and dried, and the solvent was evaporated under reduced pressure to give (3R*,4S*)-1-(tert-butoxycarbonyl)-3-(3-methyl-2-thienyl)piperidine-4-carboxylic acid (2.38 g, 69%) as a white powder.

¹H-NMR (300 MHz, CDCl₃):δ 1.46 (9H, s), 1.65-1.85 (1H, m), 1.96-2.10 (1H, m), 2.18 (3H, s), 2.62-2.90 (3H, m), 3.29 (1H, dt, J=11.1, 4.5 Hz), 4.10-4.30 (2H, m), 6.77 (1H, d, J=5.1 Hz)

### (Step 5)

To a solution of the compound (1.00 g) obtained in step 4 and DMF (about 30 μL) in THF (10 mL) was added oxalyl chloride (0.32 mL) at 0° C. The mixture was stirred at 0° C. for 1 hr, and the reaction mixture was concentrated under reduced pressure under ice-cooling. A solution of the obtained residue in THF (10 mL) was added to a solution of 1-[3,5-bis(trifluoromethyl)phenyl]-N-methylmethanamine monohydrochloride (1.08 g) and Et₃N (1.07 mL) in CH₃CN (20 mL) at 0° C., and the mixture was stirred at room temperature for 14 hr. The reaction mixture was poured into water, and the resultant product was extracted with ethyl acetate. The organic layer was washed with saturated aqueous sodium hydrogen carbonate-solution and saturated brine and dried, and the solvent was evaporated under reduced pressure. The obtained residue was purified by silica gel column chromatography (solvent gradient; 5→50% ethyl acetate/hexane) to give the title compound (1.61 g, 93%) as a white amorphous solid.

¹H-NMR (300 MHz, CDCl₃):δ 1.46-1.55 (9H, m), 1.80-1.90 (2H, m), 2.20-2.30 (3H, m), 2.60-3.10 (6H, m), 3.54 (1H, dt, J=11.4, 4.5 Hz), 4.10-5.00 (4H, m), 6.74-6.81 (1H, m), 6.99-7.04 (1H, m), 7.43-7.49 (2H, m), 7.72-7.81 (1H, m)
MS (ESI+):509 (M−^{t}Bu+2H)

### Reference Example 115

### (3R*,4S*)—N-[3,5-bis(trifluoromethyl)benzyl]-N-methyl-3-(3-methyl-2-thienyl)piperidine-4-carboxamide monohydrochloride

The title compound was obtained by reaction and purification in the same manner as in Reference Example 6 and using the compound obtained in Reference Example 114.
MS (ESI+):465 (M−HCl+H)

### Reference Example 116

### (3R*,4S*)-1-[amino(oxo)acetyl]-N-[3,5-bis(trifluoromethyl)benzyl]-N-methyl-3-(3-methyl-2-thienyl)piperidine-4-carboxamide

The title compound was obtained by reaction and purification in the same manner as in Reference Example 1 and using the compound obtained in Reference Example 115.
MS (ESI+):536 (M+H)

### Reference Example 117

### (3R*,4S*)-1-[(1-acetylpiperidin-4-yl)carbonyl]-N-[3,5-bis(trifluoromethyl)benzyl]-N-methyl-3-(3-methyl-2-thienyl)piperidine-4-carboxamide

The title compound was obtained by reaction and purification in the same manner as in Reference Example 1 and using the compound obtained in Reference Example 115 and 1-acetylpiperidine-4-carboxylic acid.
MS (ESI+):618 (M+H)

### Reference Example 118

### tert-butyl (3R*,4S*)-4-{[[3-chloro-5-(trifluoromethyl)benzyl](methyl)amino]carbonyl}-3-(3-methyl-2-thienyl)piperidine-1-carboxylate

To a solution of the compound (0.80 g) obtained in Reference Example 114, step 4 and DMF (about 30 μL) in THF (10 mL) was added oxalyl chloride (0.32 mL) at 0° C. The mixture was stirred at 0° C. for 1 hr, and the reaction mixture was concentrated under reduced pressure under ice-cooling. A solution of the obtained residue in CH₃CN (20 mL) was added to a solution of the compound (0.78 g) obtained in Reference Example 95, step 6 and Et₃N (0.86 mL) in THF (10 mL) at 0° C., and the mixture was stirred at room temperature for 2 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→50% ethyl acetate/hexane) to give the title compound (1.23 g, 94%) as a colorless oil.
MS (ESI+):475 (M−^{t}Bu+2H)

### Reference Example 119

### (3R*,4S*)—N-[3-chloro-5-(trifluoromethyl)benzyl]-N-methyl-3-(3-methyl-2-thienyl)piperidine-4-carboxamide monohydrochloride

The title compound was obtained by reaction and purification in the same manner as in Reference Example 6 and using the compound obtained in Reference Example 118.
MS (ESI+):431 (M−HCl+H)

### Reference Example 120

### (3R*,4S*)-1-[amino(oxo)acetyl]-N-[3-chloro-5-(trifluoromethyl)benzyl]-N-methyl-3-(3-methyl-2-thienyl)piperidine-4-carboxamide

The title compound was obtained by reaction and purification in the same manner as in Reference Example 1 and using the compound obtained in Reference Example 119.
MS (ESI+):501 (M+H)

The compounds described in Reference Examples 12-103 and 110-120 are as shown in (Table 3)-(Table 13).

TABLE 3

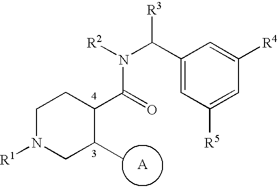

| Ref.Ex. No. | stereo-chemistry | 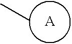 | R¹ | R² | R³ | R⁴ | R⁵ | ^{additives} | MS(ESI) |
|---|---|---|---|---|---|---|---|---|---|
| 12 | (3R*, 4S*) | 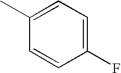 | 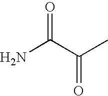 | CH₃ | H | CF₃ | CF₃ | | 534(M + H) |
| 13 | (3S*, 4S*) | 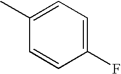 | 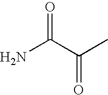 | CH₃ | H | CF₃ | CF₃ | | 534(M + H) |
| 14 | (3R*, 4S*) | 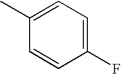 | 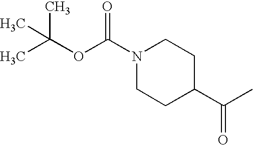 | CH₃ | H | CF₃ | CF₃ | | 674(M + H) |
| 15 | (3R*, 4S*) | 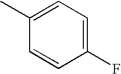 | 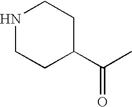 | CH₃ | H | CF₃ | CF₃ | HCl | 574(M −HCl + H) |
| 16 | (3R*, 4S*) | 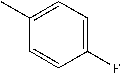 | 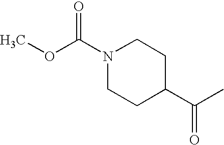 | CH₃ | H | CF₃ | CF₃ | | 632(M + H) |
| 17 | (3R*, 4S*) | 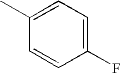 | H | 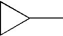 | H | CF₃ | CF₃ | HCl | 489(M −HCl + H) |
| 18 | (3R*, 4S*) | 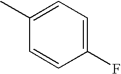 | 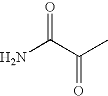 | 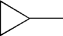 | H | CF₃ | CF₃ | | 560(M + H) |
| 19 | (3R*, 4S*) | 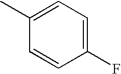 | 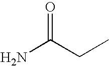 | 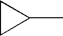 | H | CF₃ | CF₃ | HCl | 546(M −HCl + H) |
| 20 | (3R*, 4R*) | 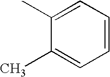 | 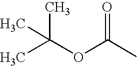 | CH₃ | H | CF₃ | CF₃ | | 485(M − ^{t}BuO) |
| 21 | (3R*, 4R*) | 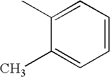 | H | CH₃ | H | CF₃ | CF₃ | HCl | 459(M −HCl + H) |

TABLE 4

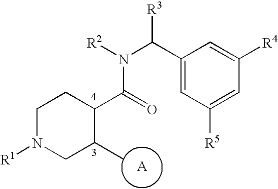

| Ref.Ex. No. | stereo-chemistry | 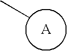 | R¹ | R² | R³ | R⁴ | R⁵ | additives | MS(ESI) |
|---|---|---|---|---|---|---|---|---|---|
| 22 | (3R*, 4R*) | 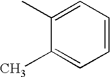 | 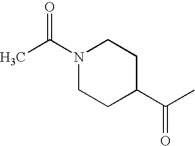 | CH₃ | H | CF₃ | CF₃ | | 612(M + H) |
| 23 | (3R*, 4R*) | 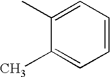 | 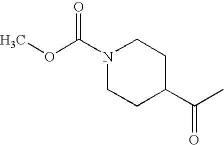 | CH₃ | H | CF₃ | CF₃ | | 628(M + H) |
| 24 | (3R*, 4R*) | 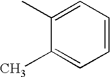 | 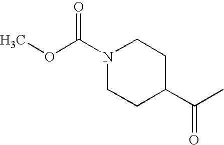 | 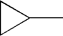 | H | CF₃ | CF₃ | | 654(M + H) |
| 25 | (3R*, 4R*) | 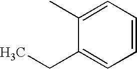 | 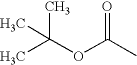 | CH₃ | H | CF₃ | CF₃ | | 573(M + H) |
| 26 | (3R*, 4R*) | 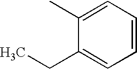 | H | CH₃ | H | CF₃ | CF₃ | HCl | 473(M − HCl + H) |
| 27 | (3R*, 4R*) | 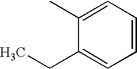 | 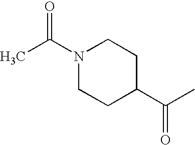 | CH₃ | H | CF₃ | CF₃ | | 626(M + H) |
| 28 | (3R*, 4R*) | 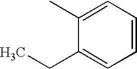 | 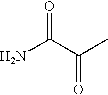 | CH₃ | H | CF₃ | CF₃ | | 544(M + H) |
| 29 | (3R*, 4R*) | 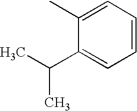 | 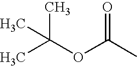 | CH₃ | H | CF₃ | CF₃ | | 587(M + H) |
| 30 | (3R*, 4R*) | 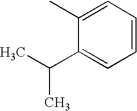 | H | CH₃ | H | CF₃ | CF₃ | HCl | 487(M − HCl + H) |
| 31 | (3R*, 4R*) | 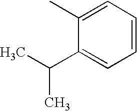 | 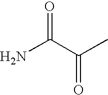 | CH₃ | H | CF₃ | CF₃ | | 558(M + H) |

TABLE 5

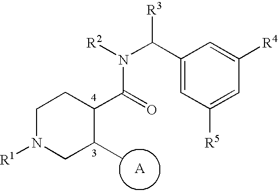

| Ref.Ex. No. | stereo-chemistry | 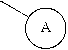 | R¹ | R² | R³ | R⁴ | R⁵ | additives | MS(ESI) |
|---|---|---|---|---|---|---|---|---|---|
| 32 | (3R*, 4R*) | 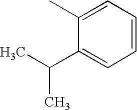 | 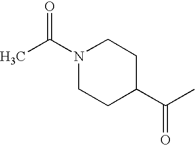 | CH₃ | H | CF₃ | CF₃ | | 640(M + H) |
| 33 | (3R*, 4S*) | 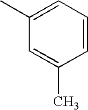 | 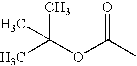 | CH₃ | H | CF₃ | CF₃ | | 559(M + H) |
| 34 | (3R*, 4R*) | 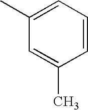 | 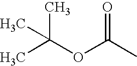 | CH₃ | H | CF₃ | CF₃ | | 559(M + H) |
| 35 | (3R*, 4S*) | 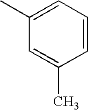 | H | CH₃ | H | CF₃ | CF₃ | HCl | 459(M − HCl + H) |
| 36 | (3R*, 4R*) | 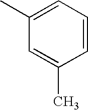 | H | CH₃ | H | CF₃ | CF₃ | HCl | 459(M − HCl + H) |
| 37 | (3R*, 4S*) | 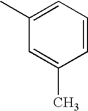 | 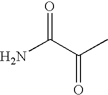 | CH₃ | H | CF₃ | CF₃ | | 530(M + H) |
| 38 | (3R*, 4R*) | 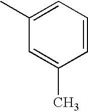 | 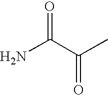 | CH₃ | H | CF₃ | CF₃ | | 530(M + H) |
| 39 | (3R*, 4R*) | 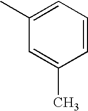 | 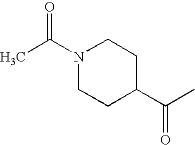 | CH₃ | H | CF₃ | CF₃ | | 612(M + H) |
| 40 | (3R*, 4R*) | 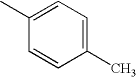 | 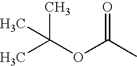 | CH₃ | H | CF₃ | CF₃ | | 559(M + H) |
| 41 | (3R*, 4S*) | 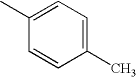 | 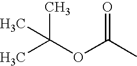 | CH₃ | H | CF₃ | CF₃ | | 559(M + H) |

TABLE 6

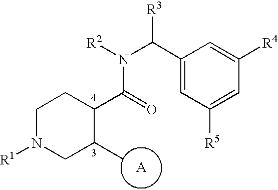

| Ref.Ex. No. | stereo-chemistry | 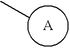 | R¹ | R² | R³ | R⁴ | R⁵ | additives | MS(ESI) |
|---|---|---|---|---|---|---|---|---|---|
| 42 | (3R*, 4R*) | 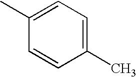 | H | CH₃ | H | CF₃ | CF₃ | HCl | 459(M − HCl + H) |
| 43 | (3R*, 4S*) | 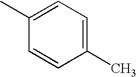 | H | CH₃ | H | CF₃ | CF₃ | HCl | 459(M − HCl + H) |
| 44 | (3R*, 4R*) | 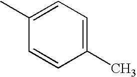 | 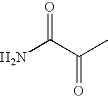 | CH₃ | H | CF₃ | CF₃ | | 530(M + H) |
| 45 | (3R*, 4R*) | 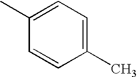 | 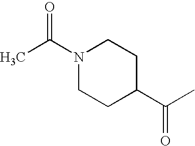 | CH₃ | H | CF₃ | CF₃ | | 612(M + H) |
| 46 | (3R*, 4S*) | 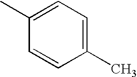 | 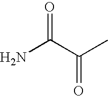 | CH₃ | H | CF₃ | CF₃ | | 530(M + H) |
| 47 | (3R*, 4S*) | 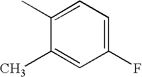 | 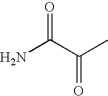 | CH₃ | H | CF₃ | CF₃ | | 548(M + H) |
| 48 | (3R*, 4R*) | 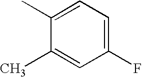 | 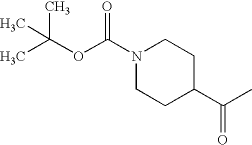 | CH₃ | H | CF₃ | CF₃ | | 688(M + H) |
| 49 | (3R*, 4R*) | 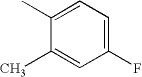 | 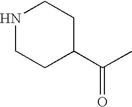 | CH₃ | H | CF₃ | CF₃ | HCl | 588(M − HCl + H) |
| 50 | (3R*, 4R*) | 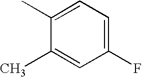 | 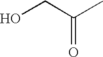 | CH₃ | H | CF₃ | CF₃ | | 535(M + H) |
| 51 | (3R*, 4S*) | 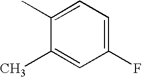 | 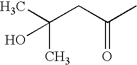 | CH₃ | H | CF₃ | CF₃ | | 577(M + H) |

TABLE 7

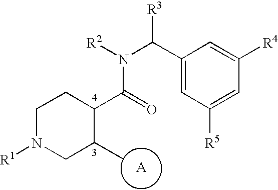

| Ref.Ex. No. | stereo-chemistry | 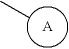 | R¹ | R² | R³ | R⁴ | R⁵ | additives | MS(ESI) |
|---|---|---|---|---|---|---|---|---|---|
| 52 | (3R*, 4R*) | 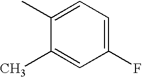 | 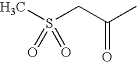 | CH₃ | H | CF₃ | CF₃ | | 597(M + H) |
| 53 | (3R*, 4R*) | 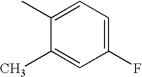 | 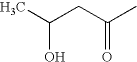 | CH₃ | H | CF₃ | CF₃ | | 563(M + H) |
| 54 | (3R*, 4R*) | 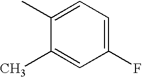 | 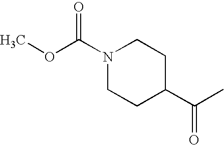 | CH₃ | H | CF₃ | CF₃ | | 646(M + H) |
| 55 | (3R*, 4R*) | 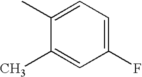 | 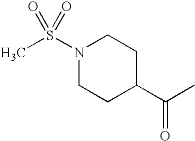 | CH₃ | H | CF₃ | CF₃ | | 666(M + H) |
| 56 | (3R*, 4R*) | 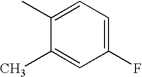 | 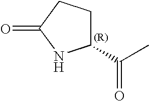 | CH₃ | H | CF₃ | CF₃ | | 588(M + H) |
| 57 | (3R*, 4R*) | 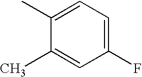 | 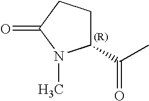 | CH₃ | H | CF₃ | CF₃ | | 602(M + H) |
| 58 | (3R*, 4R*) | 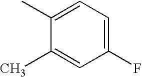 | 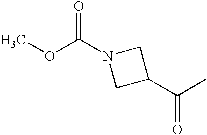 | CH₃ | H | CF₃ | CF₃ | | 618(M + H) |
| 59 | (3R*, 4R*) | 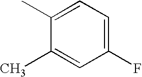 | 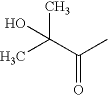 | CH₃ | H | CF₃ | CF₃ | | 563(M + H) |
| 60 | (3R*, 4R*) | 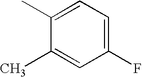 | 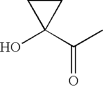 | CH₃ | H | CF₃ | CF₃ | | 561(M + H) |
| 61 | (3R*, 4R*) | 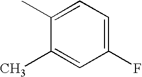 | 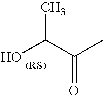 | CH₃ | H | CF₃ | CF₃ | | 549(M + H) |

TABLE 8

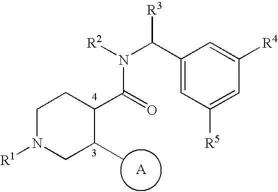

| Ref.Ex. No. | stereo-chemistry | 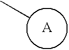 | R¹ | R² | R³ | R⁴ | R⁵ | additives | MS(ESI) |
|---|---|---|---|---|---|---|---|---|---|
| 62 | (3R*, 4R*) | 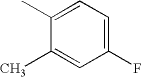 | 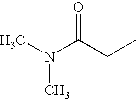 | CH₃ | H | CF₃ | CF₃ | HCl | 562(M − HCl + H) |
| 63 | (3R*, 4R*) | 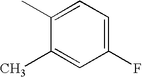 | 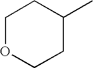 | CH₃ | H | CF₃ | CF₃ | HCl | 561(M − HCl + H) |
| 64 | (3R*, 4R*) | 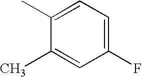 | 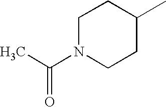 | CH₃ | H | CF₃ | CF₃ | HCl | 602(M − HCl + H) |
| 65 | (3R*, 4R*) | 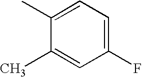 | 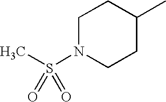 | CH₃ | H | CF₃ | CF₃ | HCl | 638(M − HCl + H) |
| 66 | (3R*, 4R*) | 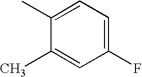 | 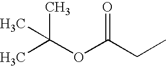 | CH₃ | H | CF₃ | CF₃ | | 592(M + H) |
| 67 | (3R*, 4R*) | 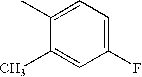 | 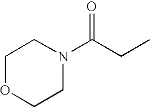 | CH₃ | H | CF₃ | CF₃ | HCl | 604(M − HCl + H) |
| 68 | (3R*, 4R*) | 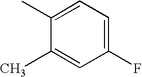 | 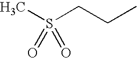 | CH₃ | H | CF₃ | CF₃ | HCl | 583(M − HCl + H) |
| 69 | (3R*, 4R*) | 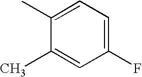 | 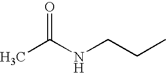 | CH₃ | H | CF₃ | CF₃ | HCl | 562(M − HCl + H) |
| 70 | (3R*, 4R*) | 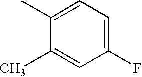 | 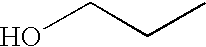 | CH₃ | H | CF₃ | CF₃ | HCl | 521(M − HCl + H) |
| 71 | (3R*, 4R*) | 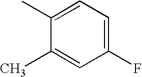 | 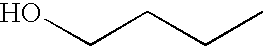 | CH₃ | H | CF₃ | CF₃ | HCl | 535(M − HCl + H) |
| 72 | (3R*, 4R*) | 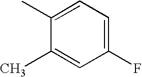 | 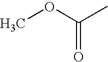 | CH₃ | H | CF₃ | CF₃ | | 535(M + H) |

TABLE 9

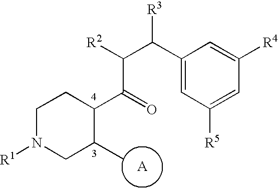

| Ref.Ex. No. | stereo-chemistry | 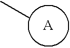 | R¹ | R² | R³ | R⁴ | R⁵ | additives | MS(ESI) |
|---|---|---|---|---|---|---|---|---|---|
| 73 | (3R*, 4R*) | 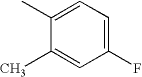 | 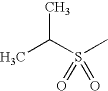 | CH₃ | H | CF₃ | CF₃ | | 583(M + H) |
| 74 | (3S, 4S) | 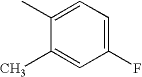 | 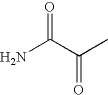 | CH₃ | H | CF₃ | CF₃ | | 548(M + H) |
| 75 | (3R, 4R) | 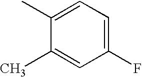 | 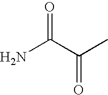 | CH₃ | H | CF₃ | CF₃ | | 548(M + H) |
| 76 | (3S, 4S) | 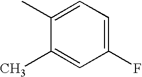 | 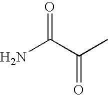 | CH₃ | (R)-CH₃ | CF₃ | CF₃ | | 562(M + H) |
| 77 | (3S, 4S) | 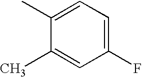 | 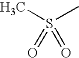 | CH₃ | (R)-CH₃ | CF₃ | CF₃ | | 569(M + H) |
| 78 | (3R*, 4R*) | 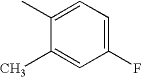 | 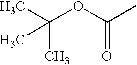 | 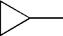 | H | CF₃ | CF₃ | | 547(M − ^{t}Bu + 2 H) |
| 79 | (3R*, 4R*) | 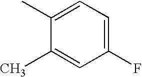 | H | 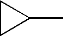 | H | CF₃ | CF₃ | HCl | 503(M − HCl + H) |
| 80 | (3R*, 4R*) | 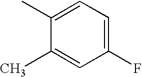 | 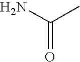 | 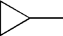 | H | CF₃ | CF₃ | | 546(M + H) |
| 81 | (3R*, 4R*) | 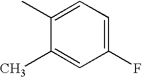 | 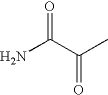 | 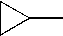 | H | CF₃ | CF₃ | | 574(M + H) |
| 82 | (3R*, 4R*) | 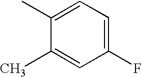 | 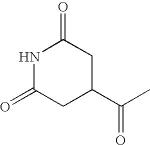 | 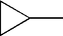 | H | CF₃ | CF₃ | | 642(M + H) |

TABLE 10

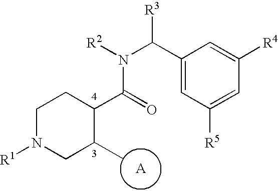

| Ref.Ex. No. | stereo-chemistry | 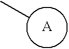 | R¹ | R² | R³ | R⁴ | R⁵ | additives | MS(ESI) |
|---|---|---|---|---|---|---|---|---|---|
| 83 | (3R*, 4R*) | 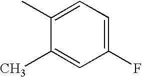 | 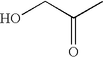 | 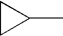 | H | CF₃ | CF₃ | | 561(M + H) |
| 84 | (3R*, 4R*) | 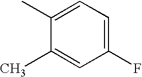 | 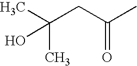 | 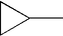 | H | CF₃ | CF₃ | | 603(M + H) |
| 85 | (3R*, 4R*) | 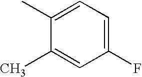 | 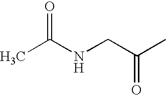 | 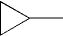 | H | CF₃ | CF₃ | | 602(M + H) |
| 86 | (3R*, 4R*) | 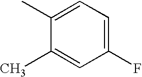 | 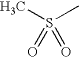 | 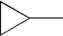 | H | CF₃ | CF₃ | | 581(M + H) |
| 87 | (3R*, 4R*) | 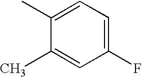 | 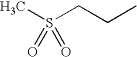 | 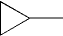 | H | CF₃ | CF₃ | HCl | 609(M − HCl + H) |
| 88 | (3R*, 4R*) | 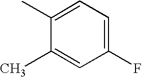 | 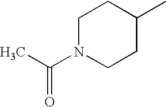 | 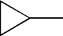 | H | CF₃ | CF₃ | HCl | 628(M − HCl + H) |
| 89 | (3R*, 4R*)(less polar) | 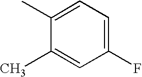 | 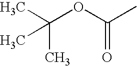 | 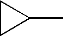 | CH₃ | CF₃ | CF₃ | | 561 (M − ^{t}Bu + 2 H) |
| 90 | (3R*, 4R*)(more polar) | 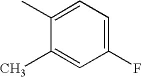 | 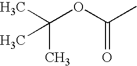 | 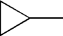 | CH₃ | CF₃ | CF₃ | | 561 (M − ^{t}Bu + 2 H) |
| 91 | (3R*, 4R*)(less polar) | 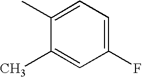 | H | 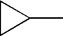 | CH₃ | CF₃ | CF₃ | HCl | 517(M − HCl + H) |
| 92 | (3R*, 4R*)(more polar) | 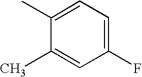 | H | 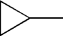 | CH₃ | CF₃ | CF₃ | HCl | 517(M − HCl + H) |

TABLE 11

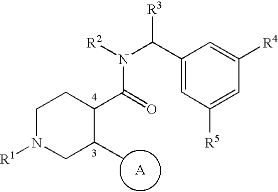

| Ref.Ex. No. | stereo-chemistry | 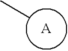 | R¹ | R² | R³ | R⁴ | R⁵ | additives | MS(ESI) |
|---|---|---|---|---|---|---|---|---|---|
| 93 | (3R*, 4R*)(less polar) | 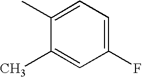 | 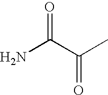 | 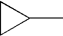 | CH₃ | CF₃ | CF₃ | | 588(M + H) |
| 94 | (3R*, 4R*)(more polar) | 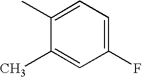 | 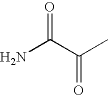 | 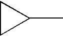 | CH₃ | CF₃ | CF₃ | | 588(M + H) |
| 95 | (3R*, 4R*) | 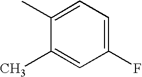 | 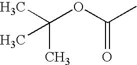 | CH₃ | H | Cl | CF₃ | | 487(M − ^{t}Bu + 2 H) |
| 96 | (3R*, 4R*) | 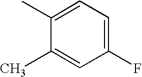 | H | CH₃ | H | Cl | CF₃ | HCl | 443(M − HCl + H) |
| 97 | (3R*, 4R*) | 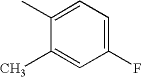 | 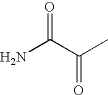 | CH₃ | H | Cl | CF₃ | | 514(M + H) |
| 98 | (3R*, 4R*)(less polar) | 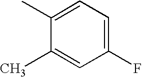 | 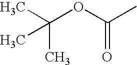 | CH₃ | CH₃ | Cl | CF₃ | | 501(M − ^{t}Bu + 2 H) |
| 99 | (3R*, 4R*)(more polar) | 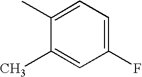 | 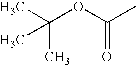 | CH₃ | CH₃ | Cl | CF₃ | | 501(M − ^{t}Bu + 2 H) |
| 100 | (3R*, 4R*)(less polar) | 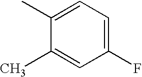 | H | CH₃ | CH₃ | Cl | CF₃ | HCl | 457(M − HCl + H) |
| 101 | (3R*, 4R*)(more polar) | 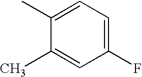 | H | CH₃ | CH₃ | Cl | CF₃ | HCl | 457(M − HCl + H) |
| 102 | (3R*, 4R*)(less polar) | 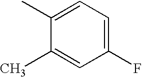 | 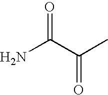 | CH₃ | CH₃ | Cl | CF₃ | | 528(M + H) |

TABLE 12

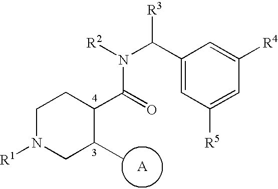

| Ref.Ex. No. | stereo-chemistry | 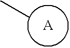 | R¹ | R² | R³ | R⁴ | R⁵ | additives | MS(ESI) |
|---|---|---|---|---|---|---|---|---|---|
| 103 | (3R*, 4R*)(more polar) | 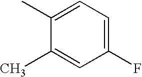 | 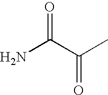 | CH₃ | CH₃ | Cl | CF₃ | | 528(M + H) |
| 110 | (3R*, 4R*) | 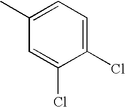 | 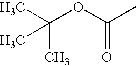 | CH₃ | H | CF₃ | CF₃ | | 558(M − ^{t}Bu + 2 H) |
| 111 | (3R*, 4R*) | 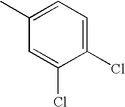 | H | CH₃ | H | CF₃ | CF₃ | HCl | 514(M − HCl + H) |
| 112 | (3R*, 4R*) | 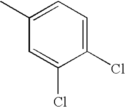 | 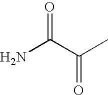 | CH₃ | H | CF₃ | CF₃ | | 585(M + H) |

TABLE 13

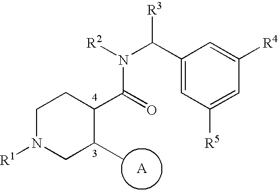

| Ref.Ex. No. | stereo-chemistry | 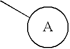 | R¹ | R² | R³ | R⁴ | R⁵ | additives | MS(ESI) |
|---|---|---|---|---|---|---|---|---|---|
| 113 | (3R*, 4R*) | 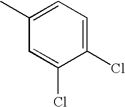 | 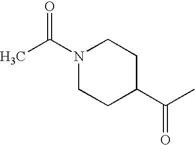 | CH₃ | H | CF₃ | CF₃ | | 667(M + H) |
| 114 | (3R*, 4S*) | 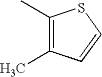 | 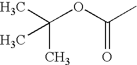 | CH₃ | H | CF₃ | CF₃ | | 509(M − ^{t}Bu + 2 H) |
| 115 | (3R*, 4S*) | 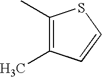 | H | CH₃ | H | CF₃ | CF₃ | HCl | 465(M − HCl + H) |
| 116 | (3R*, 4S*) | 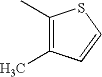 | 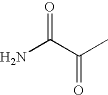 | CH₃ | H | CF₃ | CF₃ | | 536(M + H) |
| 117 | (3R*, 4S*) | 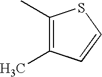 | 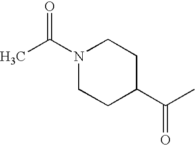 | CH₃ | H | CF₃ | CF₃ | | 618(M + H) |
| 118 | (3R*, 4S*) | 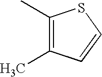 | 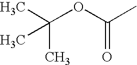 | CH₃ | H | Cl | CF₃ | | 475(M − ^{t}Bu + 2 H) |
| 119 | (3R*, 4S*) | 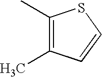 | H | CH₃ | H | Cl | CF₃ | HCl | 431(M − HCl + H) |
| 120 | (3R*, 4S*) | 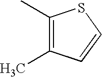 | 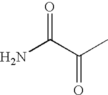 | CH₃ | H | Cl | CF₃ | | 501(M + H) |

### Reference Example 127

### 1-lactoylpiperidine-4-carboxylic acid

The title compound was obtained by reaction and purification in the same manner as in Reference Example 11, steps 3-4, and using the compound obtained in Reference Example 11, step 2, and lactic acid.

elemental analysis value: C₉H₁₅NO₄

Found C, 53.72; H, 7.51; N, 6.96

Calculated C, 53.52; H, 7.56; N, 6.88

### Reference Example 128

### 1-[(2S)-2-hydroxypropanoyl]piperidine-4-carboxylic acid

The title compound was obtained by reaction and purification in the same manner as in Reference Example 11, steps 3-4, and using the compound obtained in Reference Example 11, step 2, and L-lactic acid.

elemental analysis value: C₉H₁₅NO₄

Found C, 53.72; H, 7.51; N, 6.96

Calculated C, 53.50; H, 7.62; N, 6.79

### Reference Example 129

### 1-[(2R)-2-hydroxypropanoyl]piperidine-4-carboxylic acid

The title compound was obtained by reaction and purification in the same manner as in Reference Example 11, steps 3-4, and using the compound obtained in Reference Example 11, step 2, and D-lactic acid.

elemental analysis value: C₉H₁₅NO₄

Found C, 53.72; H, 7.51; N, 6.96

Calculated C, 53.55; H, 7.60; N, 6.82

The compounds described in Reference Examples 127-129 are as shown in (Table 14).

TABLE 14

| Ref. | Structural |
|---|---|
| Ex. No. | formula |
| 127 | 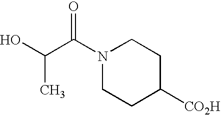 |
| 128 | 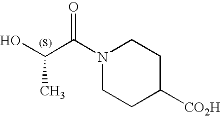 |
| 129 | 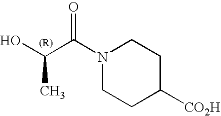 |

### Example 1

### (3R*,4S*)—N-[3,5-bis(trifluoromethyl)benzyl]-N-methyl-1-[3-(methylsulfonyl)propanoyl]-3-phenylpiperidine-4-carboxamide

To a solution of (3R*,4S*)—N-[3,5-bis(trifluoromethyl)benzyl]-N-methyl-3-phenylpiperidine-4-carboxamide monohydrochloride (206 mg) synthesized by a known method (WO2005/068427), 3-(methylsulfonyl)propanoic acid (94.9 mg) and Et₃N (174 μL) in THF (5 mL) were added WSC.HCl (122 mg) and HOBt.H₂O (96.5 mg), and the mixture was stirred at room temperature for 19 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (327 mg, 68%) as a white amorphous solid.

MS (ESI+):579 (M+H)

### Example 2

### (3R*,4S*)—N-[3,5-bis(trifluoromethyl)benzyl]-N-methyl-3-phenyl-1-(pyridin-2-ylcarbonyl)piperidine-4-carboxamide

The title compound was obtained by reaction and purification in the same manner as in Example 1 and using picolinic acid.

MS (ESI+):550 (M+H)

### Example 3

### (3R*,4S*)—N-[3,5-bis(trifluoromethyl)benzyl]-N-methyl-3-phenyl-1-(pyrazin-2-ylcarbonyl)piperidine-4-carboxamide

The title compound was obtained by reaction and purification in the same manner as in Example 1 and using 2-pyrazinecarboxylic acid.

MS (ESI+):551 (M+H)

### Example 4

### (3R*,4S*)—N-[3,5-bis(trifluoromethyl)benzyl]-N-methyl-3-phenyl-1-(pyridin-3-ylcarbonyl)piperidine-4-carboxamide

The title compound was obtained by reaction and purification in the same manner as in Example 1 and using nicotinic acid.

MS (ESI+):550 (M+H)

### Example 5

### tert-butyl 3-[((3R*,4S*)-{[[3,5-bis(trifluoromethyl)benzyl](methyl)amino]carbonyl}-3-phenylpiperidin-1-yl)carbonyl]azetidine-1-carboxylate

The title compound was obtained by reaction and purification in the same manner as in Example 1 and using Boc-azetidine-3-carboxylic acid.

MS (ESI+):628 (M+H)

### Example 6

### (3R*,4S*)-1-(azetidin-3-ylcarbonyl)-N-[3,5-bis(trifluoromethyl)benzyl]-N-methyl-3-phenylpiperidine-4-carboxamide monohydrochloride

To a solution of the compound (1.67 g) obtained in Example 5 in a mixture of ethyl acetate (15 mL)-EtOH (5 mL) was added 4N hydrogen chloride/ethyl acetate (2.0 mL) solution, and the mixture was stirred with heating at 60° C. for 2 hr. The reaction mixture was concentrated under reduced pressure, and ethyl acetate was added to the residue, and the mixture was basified with an aqueous sodium hydrogen carbonate solution. The organic layer was separated and washed with water, and the solvent was evaporated under reduced pressure. A part of the obtained residue was purified by silica gel column chromatography (NH Chromatorex) (solvent gradient; 50→100% ethyl acetate/hexane) and preparative HPLC. The thus-obtained colorless oil (80.6 mg) was treated with 1 equivalent amount of 4N hydrogen chloride/ethyl acetate to give the title compound (53.9 mg) as a white amorphous solid.

MS (ESI+):528 (M−HCl+H)

### Example 7

### (3R*,4S*)—N-[3,5-bis(trifluoromethyl)benzyl]-1-[(1-glycoloylpiperidin-4-yl)carbonyl]-N-methyl-3-phenylpiperidine-4-carboxamide

The title compound was obtained by reaction and purification in the same manner as in Example 1 and using the compound obtained in Reference Example 6 and glycolic acid.

MS (ESI+):614 (M+H)

### Example 8

### (3R*,4R*)—N-[3,5-bis(trifluoromethyl)benzyl]-1-[(1-glycoloylpiperidin-4-yl)carbonyl]-N-methyl-3-phenylpiperidine-4-carboxamide

The title compound was obtained by reaction and purification in the same manner as in Example 1 and using (3R*,4R*)—N-[3,5-bis(trifluoromethyl)benzyl]-N-methyl-3-phenylpiperidine-4-carboxamide monohydrochloride synthesized by a known method (WO2005/068427) and the compound obtained in Reference Example 11.

MS (ESI+):614 (M+H)

### Example 9

### (3R*,4S*)-1-[4-(acetylamino)benzoyl]-N-[3,5-bis(trifluoromethyl)benzyl]-N-methyl-3-phenylpiperidine-4-carboxamide

The title compound was obtained by reaction and purification in the same manner as in Example 1 and using 4-acetamidobenzoic acid.

MS (ESI+):606 (M+H)

### Example 10

### (3R*,4S*)—N-[3,5-bis(trifluoromethyl)benzyl]-1-(4-cyanobenzoyl)-N-methyl-3-phenylpiperidine-4-carboxamide

The title compound was obtained by reaction and purification in the same manner as in Example 1 and using 4-cyanobenzoic acid.

MS (ESI+):574 (M+H)

### Example 11

### (3R*,4R*)—N-[3,5-bis(trifluoromethyl)benzyl]-1-({[(1R,4S)-7,7-dimethyl-2-oxobicyclo[2.2.1]hept-1-yl]methyl}sulfonyl)-3-(4-fluoro-2-methylphenyl)-N-methylpiperidine-4-carboxamide

To a solution of (3R*,4R*)—N-[3,5-bis(trifluoromethyl)benzyl]-N-methyl-3-phenylpiperidine-4-carboxamide monohydrochloride (150 mg) and Et₃N (102 μL) in THF (6 mL) was added (−)-10-camphorsulfonylchloride (89.8 mg) at 0° C., and the mixture was stirred at room temperature for 2 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (174 mg, 86%) as white crystals.

MS (ESI+):691 (M+H)

### Example 12

### (3R*,4S*)—N-[3,5-bis(trifluoromethyl)benzyl]-1(6-chloropyridazin-3-yl)-N-methyl-3-phenylpiperidine-4-carboxamide monohydrochloride

To a solution of (3R*,4S*)—N-[3,5-bis(trifluoromethyl)benzyl]-N-methyl-3-phenylpiperidine-4-carboxamide monohydrochloride (400 mg) in THF (8 mL) was added Et₃N (128 μL), and the mixture was stirred at room temperature for 20 min. The precipitate was filtered off, and the filtrate was concentrated under reduced pressure. The residue was dissolved in EtOH (4 mL), 3,6-dichloropyridazine (105 mg) was added, and the mixture was heated under reflux for 6 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (53.6 mg, 62%) as white crystals.

MS (ESI+):557 (M−HCl+H)

### Example 13

### (3R*,4S*)—N-[3,5-bis(trifluoromethyl)benzyl]-N-methyl-3-phenyl-1-pyridin-2-ylpiperidine-4-carboxamide monohydrochloride

The title compound was obtained by reaction and purification in the same manner as in Example 12 and using 2-chloropyridine.

MS (ESI+):522 (M−HCl+H)

### Example 14

### (3R*,4S*)—N-[3,5-bis(trifluoromethyl)benzyl]-3-(4-fluorophenyl)-1-[(1-glycoloylpiperidin-4-yl)carbonyl]-N-methylpiperidine-4-carboxamide

The title compound was obtained by reaction and purification in the same manner as in Example 1 and using (3R*,4S*)—N-[3,5-bis(trifluoromethyl)benzyl]-N-methyl-3-(4-fluorophenyl)piperidine-4-carboxamide monohydrochloride synthesized by a known method (WO2005/068427) and the compound obtained in Reference Example 11.

MS (ESI+):632 (M+H)

### Example 15

### (3R*,4R*)—N-[3,5-bis(trifluoromethyl)benzyl]-3-(4-fluorophenyl)-1-[(1-glycoloylpiperidin-4-yl)carbonyl]-N-methylpiperidine-4-carboxamide

The title compound was obtained by reaction and purification in the same manner as in Example 1 and using (3R*,4R*)—N-[3,5-bis(trifluoromethyl)benzyl]-N-methyl-3-(4-fluorophenyl)piperidine-4-carboxamide monohydrochloride synthesized by a known method (WO2005/068427) and the compound obtained in Reference Example 11.

MS (ESI+):632 (M+H)

### Example 16

### (3R*,4R*)—N-[3,5-bis(trifluoromethyl)benzyl]-1-[(1-glycoloylpiperidin-4-yl)carbonyl]-N-methyl-3-(2-methylphenyl)piperidine-4-carboxamide

To a solution of the compound (198 mg) obtained in Reference Example 21, the compound (97.3 mg) obtained in Reference Example 11 and Et₃N (61 μL) in CH₃CN (4 mL) were added WSC.HCl (100 mg) and HOBt.H₂O (79.6 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) and crystallized from ethyl acetate-hexane to give the title compound (223 mg, 89%) as a white powder.

MS (ESI+):628 (M+H)

melting point: 173-174° C.

### Example 17

### (3R,4R)—N-[3,5-bis(trifluoromethyl)benzyl]-1-[[1-glycoloylpiperidin-4-yl]carbonyl]-N-methyl-3-(2-methylphenyl)piperidine-4-carboxamide

### Example 18

### (3S,4S)—N-[3,5-bis(trifluoromethyl)benzyl]-1-[[1-glycoloylpiperidin-4-yl]carbonyl]-N-methyl-3-(2-methylphenyl)piperidine-4-carboxamide

The compound (1.16 g) obtained in Example 16 was optically resolved by chiral column chromatography. The compound (0.509 g) of Example 17 was obtained as a white amorphous solid by concentrating a fraction with a short retention time. The compound (0.491 g) of Example 18 was obtained as a white amorphous solid by concentrating a fraction with a long retention time.

Purification conditions by chiral column chromatography

column: CHIRALPAK AD 50 mm ID×500 mm L

solvent: hexane/2-propanol=50/50

flow rate: 75 mL/min

temperature: 30° C.

detection method: UV 230 nm

Compound of Example 17

MS (ESI+): 628 (M+H)

[α]_{D}²⁵+3.8° (c 0.94, MeOH)

elemental analysis value: C₃₁H₃₅N₃O₄F₆

Found C, 59.38; H, 5.68; N, 6.63

Calculated C, 59.32; H, 5.62; N, 6.70

¹H-NMR (300 MHz, CDCl₃):δ 1.64-2.20 (5H, m), 2.38 (3H, d, J=13.5 Hz), 2.45-2.85 (3H, m), 2.92 (3H, d, J=8.7 Hz), 2.99-3.18 (2H, m), 3.19-3.31 (1H, m), 3.32-3.70 (3H, m), 3.80-3.95 (1H, m), 4.01-4.35 (4H, m), 4.40-4.59 (1H, m), 4.62-4.76 (1H, m), 4.79-4.94 (1H, m), 7.04-7.17 (4H, m), 7.36 (2H, d, J=11.7 Hz), 7.72 (1H, s)

Compound of Example 18

MS (ESI+):628 (M+H)

[α]_{D}²⁵−5.4° (c 1.00, MeOH)

elemental analysis value: C₃₁H₃₅N₃O₄F₆

Found C, 59.25; H, 5.83; N, 6.53

Calculated C, 59.32; H, 5.62; N, 6.70

¹H-NMR (300 MHz, CDCl₃):δ 1.64-2.20 (5H, m), 2.38 (3H, d, J=13.5 Hz), 2.45-2.85 (3H, m), 2.92 (3H, d, J=8.7 Hz), 2.99-3.18 (2H, m), 3.19-3.31 (1H, m), 3.32-3.70 (3H, m), 3.80-3.95 (1H, m), 4.01-4.35 (4H, m), 4.40-4.59 (1H, m), 4.62-4.76 (1H, m), 4.79-4.94 (1H, m), 7.04-7.17 (4H, m), 7.36 (2H, d, J=11.7 Hz), 7.72 (1H, s)

### Example 19

### (3R*,4R*)—N-[3,5-bis(trifluoromethyl)benzyl]-1-[(1-lactoylpiperidin-4-yl)carbonyl]-N-methyl-3-(2-methylphenyl)piperidine-4-carboxamide

(step 1)

tert-Butyl 4-{[(3R*,4R*)-4-{[[3,5-bis(trifluoromethyl)benzyl](methyl)amino]carbonyl}-3-(2-methylphenyl)piperidin-1-yl]carbonyl}piperidine-1-carboxylate was obtained as a white amorphous solid by reaction and purification in the same manner as in Example 1 and using the compound obtained in Reference Example 21 and N-Boc-isonipecotic acid.

MS (ESI+):670 (M+H)

(step 2)

(3R*,4R*)—N-[3,5-bis(Trifluoromethyl)benzyl]-N-methyl-3-(2-methylphenyl)-1-(piperidin-4-ylcarbonyl)piperidine-4-carboxamide monohydrochloride was obtained as a white amorphous solid by reaction and purification in the same manner as in Example 6 and using the compound obtained in step 1.

MS (ESI+):570 (M−HCl+H)

(step 3)

The title compound was obtained as a white amorphous solid by reaction and purification in the same manner as in Example 1 and using the compound obtained in step 2 and lactic acid.

MS (ESI+):642 (M+H)

### Example 20

### (3R*,4R*)—N-[3,5-bis(trifluoromethyl)benzyl]-1-({1-[(1-hydroxycyclopropyl)carbonyl]piperidin-4-yl}carbonyl)-N-methyl-3-(2-methylphenyl)piperidine-4-carboxamide

The title compound was obtained as a white amorphous solid by reaction and purification in the same manner as in Example 1 and using the compound obtained in Example 19, step 2 and 1-hydroxy-1-cyclopropanecarboxylic acid.

MS (ESI+):654 (M+H)

### Example 21

### Ethyl 4-{[(3R*,4R*)-4-{[[3,5-bis(trifluoromethyl)benzyl](methyl)amino]carbonyl}-3-(2-methylphenyl)piperidin-1-yl]carbonyl}piperidine-1-carboxylate

To a solution of the compound (200 mg) obtained in Example 19, step 2 and Et₃N (115 μL) in THF (5 mL) was added ethyl chloroformate (115 μL) at 0° C., and the mixture was stirred at room temperature for 6.5 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (66.9 mg, 32%) as a white amorphous solid.

MS (ESI+):642 (M+H)

### Example 22

### Isopropyl 4-{[(3R*,4R*)-4-{[[3,5-bis(trifluoromethyl)benzyl](methyl)amino]carbonyl}-3-(2-methylphenyl)piperidin-1-yl]carbonyl}piperidine-1-carboxylate

The title compound was obtained as a white amorphous solid by reaction and purification in the same manner as in Example 21 and using isopropyl chloroformate.

MS (ESI+):656 (M+H)

### Example 23

### (3R*,4R*)—N-[3,5-bis(trifluoromethyl)benzyl]-1-{[1-(cyclopropylsulfonyl)piperidin-4-yl]carbonyl}-N-methyl-3-(2-methylphenyl)piperidine-4-carboxamide

The title compound was obtained as a white amorphous solid by reaction and purification in the same manner as in Example 21 and using cyclopropanesulfonyl chloride.

MS (ESI+):674 (M+H)

### Example 24

### (3R*,4R*)—N-3,5-bis(trifluoromethyl)benzyl]-1-[(5,5-dimethyl-2,4-dioxo-1,3-oxazolidin-3-yl)acetyl]-N-methyl-3-(2-methylphenyl)piperidine-4-carboxamide

The title compound was obtained as a white amorphous solid by reaction and purification in the same manner as in Example 1 and using the compound obtained in Reference Example 21 and (5,5-dimethyl-2,4-dioxo-1,3-oxazolidin-3-yl)acetic acid synthesized by a known method (Journal of the American Chemical Society (1948), 70 1021-1022).

MS (ESI+):628 (M+H)

### Example 25

### (3R*,4R*)-1-(2-amino-2-oxoethyl)-N-[3,5-bis(trifluoromethyl)benzyl]-N-methyl-3-(2-methylphenyl)piperidine-4-carboxamide monohydrochloride

To a solution of the compound (198 mg) obtained in Reference Example 21 and 2-iodoacetamide (104 mg) in DMF (3 mL) was added Et₃N (139 μL) at room temperature, and the mixture was stirred at room temperature for 8 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 0→100% ethyl acetate/hexane). The thus-obtained colorless oil was treated with 1 equivalent amount of 4N hydrogen chloride/ethyl acetate to give the title compound (151 mg, 73%) as a white amorphous solid.

MS (ESI+):516 (M−HCl+H)

### Example 26

### (3S,4S)—N-[3,5-bis(trifluoromethyl)benzyl]-1-[(1-lactoylpiperidin-4-yl)carbonyl]-N-methyl-3-(2-methylphenyl)piperidine-4-carboxamide

(step 1)

To a solution of (3R*,4R*)-1-(tert-butoxycarbonyl)-3-(2-methylphenyl)piperidine-4-carboxylic acid (7.00 g) obtained in Reference Example 20, step 3 in acetone (87.6 mL) was added a solution of (S)-1-phenylethylamine (1.35 g) in acetone (21.9 mL), and the mixture was stirred at room temperature for 4 hr. The precipitate was filtrated using acetone (18.2 mL). To the precipitate obtained above in water (30 mL) and ethyl acetate (30 mL) was added citric acid (1.33 g). The organic layer was separated, washed with water and dried. The solvent was evaporated under reduced pressure to give (3S,4S)-1-(tert-butoxycarbonyl)-3-(2-methylphenyl)piperidine-4-carboxylic acid (1.51 g) as a white powder.

MS (ESI+):246 (M−^{t}BuO)

(step 2)

To a solution of the compound (1.30 g) obtained in step 1 and 1-[3,5-bis(trifluoromethyl)phenyl]-N-methylmethanamine monohydrochloride (1.43 g) in CH₃CN (20 mL) were added WSC.HCl (1.43 g) and HOBt.H₂O (1.19 g), and the mixture was stirred at room temperature for 16 hr. The reaction mixture was poured into water, and the resultant product was extracted with ethyl acetate. The organic layer was washed with saturated aqueous sodium hydrogen-carbonate solution and saturated brine and dried, and the solvent was evaporated under reduced pressure. The obtained residue was purified by silica gel column chromatography (solvent gradient; 10→25% ethyl acetate/hexane) to give tert-butyl (3S,4S)-4-{[[3,5-bis(trifluoromethyl)benzyl](methyl)amino]carbonyl}-3-(2-methylphenyl)piperidine-1-carboxylate (1.97 g, 87%) as a white powder.

MS (ESI+):559 (M+H)

(step 3)

To a solution of the compound (2.00 g) obtained in step 2 in a mixture of ethyl acetate (36 mL)-EtOH (3.6 mL) was added 4N hydrogen chloride/ethyl acetate (2.69 mL) solution, and the mixture was stirred with heating at 60° C. for 4 hr. The reaction mixture was concentrated under reduced pressure to give (3S,4S)—N-[3,5-bis(trifluoromethyl)benzyl]-N-methyl-3-(2-methylphenyl)piperidine-4-carboxamide monohydrochloride (1.54 g, 87%) as a white powder.

MS (ESI+):459 (M−HCl+H)

(step 4)

To a solution of the compound (200 mg) obtained in step 3, the compound (106 mg) obtained in Reference Example 127 and Et₃N (169 μL) in THF (10 mL) were added WSC.HCl (119 mg) and HOBt.H₂O (93.8 mg), and the mixture was stirred at room temperature for 22 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (119.5 mg, 46%) as a white amorphous solid.

MS (ESI+):642 (M+H)

### Example 27

### (3R*,4R*)—N-[3,5-bis(trifluoromethyl)benzyl]-1-{[1-(3-hydroxy-3-methylbutanoyl)piperidin-4-yl]carbonyl}-N-methyl-3-(2-methylphenyl)piperidine-4-carboxamide

The title compound was obtained as a white amorphous solid by reaction and purification in the same manner as in Example 1 and using the compound obtained in Example 19, step 2 and β-hydroxyisovaleric acid.

MS (ESI+):670 (M+H)

### Example 28

### (3S,4S)—N-(3,5-bis(trifluoromethyl)benzyl]-1-({1-[(2S)-2-hydroxypropanoyl]piperidin-4-yl}carbonyl)-N-methyl-3-(2-methylphenyl)piperidine-4-carboxamide

To a solution of the compound (200 mg) obtained in Example 26, step 3, the compound (106 mg) obtained in Reference Example 128 and Et₃N (169 μL) in THF (10 mL) were added WSC.HCl (119 mg) and HOBt.H₂O (93.8 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (164.4 mg, 63%) as a white amorphous solid.

MS (ESI+):642 (M+H)

[α]_{D}²⁵−7.2° (c 1.0, MeOH)

elemental analysis value: C₃₂H₃₇N₃O₄F₆

Found C, 59.52; H, 5.93; N, 6.36

Calculated C, 59.90; H, 5.81; N, 6.55

¹H-NMR (300 MHz, CDCl₃):δ 1.23-1.38 (4H, m), 1.66-2.01 (3H, m), 2.35 (3H, d, J=13.8 Hz), 2.50-2.88 (4H, m), 2.93 (3H, d, J=8.1 Hz), 3.01-3.19 (2H, m), 3.26 (1H, dt, J=10.7, 4.4 Hz), 3.34-3.54 (1H, m), 3.71-3.94 (3H, m), 4.05-4.37 (2H, m), 4.39-4.61 (2H, m), 4.70 (1H, t, J=16.3 Hz), 4.80-4.95 (1H, m), 7.06-7.17 (4H, m), 7.36 (2H, d, J=12.3 Hz), 7.72 (1H, s)

### Example 29

### (3S,4S)—N-[3,5-bis(trifluoromethyl)benzyl]-1-({1-[(2R)-2-hydroxypropanoyl]piperidin-4-yl}carbonyl)-N-methyl-3-(2-methylphenyl)piperidine-4-carboxamide

To a solution of the compound (200 mg) obtained in Example 26, step 3, the compound (106 mg) obtained in Reference Example 129 and Et₃N (169 μL) in THF (10 mL) were added WSC.HCl (119 mg) and HOBt.H₂O (93.8 mg), and the mixture was stirred at room temperature for 18 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (130.6 mg, 50%) as a white amorphous solid.

MS (ESI+):642 (M+H)

[α]_{D}²⁵−4.2° (c 1.0, MeOH)

elemental analysis value: C₃₂H₃₇N₃O₄F₆.0.3H₂O

Found C, 59.41; H, 5.93; N, 6.42

Calculated C, 59.40; H, 5.86; N, 6.49

¹H-NMR (300 MHz, CDCl₃):δ 1.28-1.39 (4H, m), 1.67-2.01 (3H, m), 2.38 (3H, d, J=14.7 Hz), 2.51-2.88 (4H, m), 2.93 (3H, d, J=8.4 Hz), 3.02-3.19 (2H, m), 3.20-3.32 (1H, m), 3.34-3.57 (1H, m), 3.71-3.94 (3H, m), 4.04-4.35 (2H, m), 4.40-4.55 (2H, m), 4.64-4.76 (1H, m), 4.81-4.96 (1H, m), 7.04-7.19 (4H, m), 7.36 (2H, d, J=12.9 Hz), 7.72 (1H, s)

### Example 30

### (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-N-methyl-3-(2-methylphenyl)piperidine-4-carboxamide

(step 1)

To a solution of the compound (5.00 g) obtained in Example 26, step 1 and DMF (about 50 μL) in THF (50 mL) was added oxalyl chloride (1.56 mL) at 0° C. After stirring at 0° C. for 10 min, a solution of (1S)-1-[3,5-bis(trifluoromethyl)phenyl]-N-methylethanamine monohydrochloride (4.67 g) and Et₃N (6.55 mL) in THF (50 mL) was added at 0° C., and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→30% ethyl acetate/hexane), and crystallized from IPE-hexane to give tert-butyl (3S,4S)-4-{[{(1S)-1-[3,5-bis(trifluoromethyl)phenyl]ethyl)(methyl)amino]carbonyl}-3-(2-methylphenyl)piperidine-1-carboxylate (2.84 g, 32%) as a white powder.

MS (ESI+):573 (M+H)

(step 2)

To a solution of the compound (2.63 g) obtained in step 1 in a mixture of ethyl acetate (46 mL)-EtOH (4.6 mL) was added 4N hydrogen chloride/ethyl acetate (3.4 mL) solution, and the mixture was stirred with heating at 60° C. for 10 hr. The reaction mixture was concentrated under reduced pressure, and crystallized from Et₂O to give (3S,4S)—N-{(1S)-1-[3,5-bis(trifluoromethyl)phenyl]ethyl}-N-methyl-3-(2-methylphenyl)piperidine-4-carboxamide monohydrochloride (2.29 g, 98%) as a white powder.

MS (ESI+):473 (M−HCl+H)

(step 3)

To a solution of the compound (200 mg) obtained in step 2, (5,5-dimethyl-2,4-dioxo-1,3-oxazolidin-3-yl)acetic acid (95.6 mg) and Et₃N (169 μL) in THF (10 mL) were added WSC.HCl (119 mg) and HOBt.H₂O (93.8 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (161.0 mg, 64%) as a white amorphous solid.

MS (ESI+):642 (M+H)

[α]_{D}²⁵−56.7° (c 0.96, MeOH)

elemental analysis value: C₃₁H₃₃N₃O₃F₆

Found C, 57.71; H, 5.17; N, 6.40

Calculated C, 58.03; H, 5.18; N, 6.55

¹H-NMR (300 MHz, CDCl₃):δ 1.45 (3H, d, J=7.2 Hz), 1.65 (6H, s), 1.85-2.03 (2H, m), 2.43 (3H, d, J=5.1 Hz), 2.65 (3H, d, J=5.1 Hz), 2.69-2.85 (1H, m), 3.09-3.34 (2H, m), 3.41-3.65 (1H, m), 3.68-3.99 (1H, m), 4.25-4.40 (2H, m), 4.56-4.83 (1H, m), 5.88 (1H, q, J=5.8 Hz), 6.99-7.19 (4H, m), 7.26 (2H, s), 7.70 (1H, s)

### Example 31

### (3R*,4R*)—N-[3,5-bis(trifluoromethyl)benzyl]-1-[1(1-glycoloylpiperidin-4-yl)carbonyl]-3-(2-isopropylphenyl)-N-methylpiperidine-4-carboxamide

The title compound was obtained by reaction and purification in the same manner as in Example 1 and using the compound obtained in Reference Example 30 and the compound obtained in Reference Example 11.

MS (ESI+):656 (M+H)

### Example 32

### (3R*,4R*)—N-[3,5-bis(trifluoromethyl)benzyl]-1-[(1-glycoloylpiperidin-4-yl)carbonyl]-N-methyl-3-(3-methylphenyl)piperidine-4-carboxamide

The title compound was obtained by reaction and purification in the same manner as in Example 1 and using the compound obtained in Reference Example 36 and the compound obtained in Reference Example 11.

MS (ESI+):628 (M+H)

### Example 33

### (3R*,4S*)—N-[3,5-bis(trifluoromethyl)benzyl]-1-[(1-glycoloylpiperidin-4-yl)carbonyl]-N-methyl-3-(3-methylphenyl)piperidine-4-carboxamide

The title compound was obtained by reaction and purification in the same manner as in Example 1 and using the compound obtained in Reference Example 35 and the compound obtained in Reference Example 11.

MS (ESI+):628 (M+H)

### Example 34

### (3R*,4R*)—N-[3,5-bis(trifluoromethyl)benzyl]-1-[(1-glycoloylpiperidin-4-yl)carbonyl]-N-methyl-3-(4-methylphenyl)piperidine-4-carboxamide

The title compound was obtained by reaction and purification in the same manner as in Example 1 and using the compound obtained in Reference Example 42 and the compound obtained in Reference Example 11.

MS (ESI+):628 (M+H)

### Example 35

### (3R*,4S*)—N-[3,5-bis(trifluoromethyl)benzyl]-3-(4-fluoro-2-methylphenyl)-1-[(1-glycoloylpiperidin-4-yl)carbonyl]-N-methylpiperidine-4-carboxamide

The title compound was obtained by reaction and purification in the same manner as in Example 1 and using (3R*,4S*)—N-[3,5-bis(trifluoromethyl)benzyl]-3-(4-fluoro-2-methylphenyl)-N-methylpiperidine-4-carboxamide monohydrochloride synthesized by a known method (WO2005/068427) and the compound obtained in Reference Example 11.

MS (ESI+):646 (M+H)

### Example 36

### (3R*,4R*)—N-(3,5-bis(trifluoromethyl)benzyl)-3-(4-fluoro-2-methylphenyl)-N-methyl-1-(1H-tetrazol-5-ylacetyl)piperidine-4-carboxamide

The title compound was obtained by reaction and purification in the same manner as in Example 1 and using (3R*,4R*)—N-[3,5-bis(trifluoromethyl)benzyl-3-(4-fluoro-2-methylphenyl)-N-methylpiperidine-4-carboxamide monohydrochloride synthesized by a known method (WO2005/068427) and 1H-tetrazol-5-ylacetic acid.

MS (ESI+):587 (M+H)

### Example 37

### (3R*,4R*)—N-(3,5-bis(trifluoromethyl)benzyl)-3(4-fluoro-2-methylphenyl)-N-methyl-1-{[1-(1H-tetrazol-1-ylacetyl)piperidin-4-yl]carbonyl}piperidine-4-carboxamide

The title compound was obtained by reaction and purification in the same manner as in Example 1 and using the compound obtained in Reference Example 49 and 1H-tetrazole-1-acetic acid.

MS (ESI+):698 (M+H)

### Example 38

### (3R*,4R*)-1-{[1-(N-acetylglycyl)piperidin-4-yl]carbonyl}-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using the compound obtained in Reference Example 49 and N-acetylglycine.

MS (ESI+):687 (M+H)

### Example 39

### (3R*,4R*)—N-[3,5-bis(trifluoromethyl)benzyl]-3-(4-fluoro-2-methylphenyl)-1-(3-hydroxypropanoyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 3-hydroxypropionic acid.

MS (ESI+):549 (M+H)

### Example 40

### (3R*,4R*)—N-[3,5-bis(trifluoromethyl)benzyl]-3-(4-fluoro-2-methylphenyl)-N-methyl-1-[3-(methylsulfonyl)propanoyl]piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride.

MS (ESI+):611 (M+H)

### Example 41

### (3R*,4R*)-1-[4-(aminosulfonyl)butanoyl]-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 3-carboxypropanesulfonamide.

MS (ESI+):626 (M+H)

### Example 42

### (3R*,4R*)—N-[3,5-bis(trifluoromethyl)benzyl]-3-(4-fluoro-2-methylphenyl)-N-methyl-1-[3-(phenylsulfonyl)propanoyl]piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 3-(phenylsulfonyl)propionic acid.

MS (ESI+):673 (M+H)

### Example 43

### (3R*,4R*)—N-[3,5-bis(trifluoromethyl)benzyl]-1-[(1,1-dioxidothiomorpholin-4-yl)acetyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 4-thiomorpholine acetic acid 1,1-dioxide monohydrate.

MS (ESI+):652 (M+H)

### Example 44

### (3R*,4R*)—N-[3,5-bis(trifluoromethyl)benzyl]-3-(4-fluoro-2-methylphenyl)-1-[(1-glycoloylpiperidin-4-yl)carbonyl]-N-methylpiperidine-4-carboxamide

The title compound was obtained by reaction and purification in the same manner as in Example 1 and using the compound obtained in Reference Example 49 and glycolic acid.

MS (ESI+):646 (M+H)

### Example 45

### (3S,4S)—N-[3,5-bis(trifluoromethyl)benzyl]-3-(4-fluoro-2-methylphenyl)-1-[(1-lactoylpiperidin-4-yl)carbonyl]-N-methylpiperidine-4-carboxamide

The title compound was obtained by reaction and purification in the same manner as in Example 1 and using (3S,4S)—N-[3,5-bis(trifluoromethyl)benzyl]-3-(4-fluoro-2-methylphenyl)-N-methylpiperidine-4-carboxamide monohydrochloride synthesized by a known method (WO2006/004195) and lactic acid.

MS (ESI+):660 (M+H)

### Example 46

### (3R*,4R*)—N-[3,5-bis(trifluoromethyl)benzyl]-3-(4-fluoro-2-methylphenyl)-1-{[1-(2-hydroxy-2-methylpropanoyl)piperidin-4-yl]carbonyl}-N-methylpiperidine-4-carboxamide

The title compound was obtained by reaction and purification in the same manner as in Example 1 and using the compound obtained in Reference Example 49 and 2-hydroxyisobutyric acid.

MS (ESI+):674 (M+H)

### Example 47

### (3R*,4R*)—N-[3,5-bis(trifluoromethyl)benzyl]-3-(4-fluoro-2-methylphenyl)-1-{[1-(methoxyacetyl)piperidin-4-yl]carbonyl}-N-methylpiperidine-4-carboxamide

The title compound was obtained by reaction and purification in the same manner as in Example 1 and using the compound obtained in Reference Example 49 and glycolic acid.

MS (ESI+):660 (M+H)

### Example 48

### 4-{[(3R*,4R*)-4-{[[3,5-bis(trifluoromethyl)benzyl](methyl)amino]carbonyl}-3-(4-fluoro-2-methylphenyl)piperidin-1-yl]carbonyl}piperidine-1-carboxamide

To a solution of the compound (250 mg) obtained in Reference Example 49 and triphosgene (245 mg) in THF (5 mL) was added Et₃N (112 μL) at 0° C., and the mixture was stirred at 0° C. for 20 min. A 28% aqueous ammonia solution (1.33 mL) was added at 0° C., and the mixture was stirred at room temperature for 22 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (157 mg, 62%) as a white amorphous solid.

MS (ESI+):631 (M+H)

### Example 49

### (3R*,4R*)-1-({1-[amino(oxo)acetyl]piperidin-4-yl}carbonyl)-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using the compound obtained in Reference Example 49 and oxamic acid.

MS (ESI+):659 (M+H)

### Example 50

### (3R*,4R*)—N-[3,5-bis(trifluoromethyl)benzyl]-3-(4-fluoro-2-methylphenyl)-1-[(1-isopropylpiperidin-4-yl)carbonyl]-N-methylpiperidine-4-carboxamide monohydrochloride

To a solution of the compound (102 mg) obtained in Reference Example 49 and acetone (59.5 μL) in ethyl acetate (7 mL) was added Et₃N (22.8 μL), and the mixture was stirred at room temperature for 5 min. NaBH(OAc)₃ (239 mg) and acetic acid (9.4 μL) were added, and the mixture was stirred at room temperature for 19 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was treated with 1 equivalent amount of 4N hydrogen chloride/ethyl acetate to give the title compound (122 mg, 88%) as a white amorphous solid.

MS (ESI+):630 (M−HCl+H)

### Example 51

### tert-butyl 3-{[(3R*,4R*)-4-{[[3,5-bis(trifluoromethyl)benzyl](methyl)amino]carbonyl}-3-(4-fluoro-2-methylphenyl)piperidin-1-yl]carbonyl}piperidine-1-carboxylate

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 1-Boc-piperidine-3-carboxylic acid.

MS (ESI+):688 (M+H)

### Example 52

### (3R*,4R*)—N-[3,5-bis(trifluoromethyl)benzyl]-3-(4-fluoro-2-methylphenyl)-N-methyl-1-(piperidin-3-ylcarbonyl)piperidine-4-carboxamide monohydrochloride

The title compound was obtained by reaction and purification in the same manner as in Example 6 and using the compound obtained in Example 51.

MS (ESI+):588 (M−HCl+H)

### Example 53

### (3R*,4R*)-1-[(1-acetylpiperidin-3-yl)carbonyl]-N-[3,5-bis(trifluoromethyl)benzyl]-3-(4-fluoro-2-methylphenyl)-N-methylpiperidine-4-carboxamide

To a solution of the compound (187 mg) obtained in Example 52 and ⁱPr₂NEt (125 μL) in CH₃CN (4 mL) was added acetyl chloride (25.6 μL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (86 mg, 46%) as a white amorphous solid.

MS (ESI+):630 (M+H)

### Example 54

### (3R*,4R*)—N-[3,5-bis(trifluoromethyl)benzyl]-3-(4-fluoro-2-methylphenyl)-1-[(1-glycoloylpiperidin-3-yl)carbonyl]-N-methylpiperidine-4-carboxamide

The title compound was obtained by reaction and purification in the same manner as in Example 1 and using the compound obtained in Example 52 and glycolic acid.

MS (ESI+):646 (M+H)

### Example 55

### (3R*,4R*)—N-[3,5-bis(trifluoromethyl)benzyl]-3-(4-fluoro-2-methylphenyl)-1-{[1-(2-hydroxy-2-methylpropanoyl)piperidin-3-yl]carbonyl}-N-methylpiperidine-4-carboxamide

The title compound was obtained by reaction and purification in the same manner as in Example 1 and using the compound obtained in Example 52 and 2-hydroxyisobutyric acid.

MS (ESI+):674 (M+H)

### Example 56

### (3R*,4R*)—N-[3,5-bis(trifluoromethyl)benzyl]-3-(4-fluoro-2-methylphenyl)-1-({1-[1-hydroxy-2-(methylsulfonyl)ethyl]piperidin-3-yl}carbonyl)-N-methylpiperidine-4-carboxamide

The title compound was obtained by reaction and purification in the same manner as in Example 1 and using the compound obtained in Example 52 and methanesulfonylacetic acid.

MS (ESI+):708 (M+H)

### Example 57

### Methyl 3-{[(3R*,4R*)-4-{[(3,5-bis(trifluoromethyl)benzyl](methyl)amino]carbonyl}-3-(4-fluoro-2-methylphenyl)piperidin-1-yl]carbonyl}piperidine-1-carboxylate

To a solution of the compound (250 mg) obtained in Example 52 and Et₃N (134 μL) in CH₃CN (4 mL) was added methyl chloroformate (37.1 μL) at 0° C., and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) and crystallized from ethyl acetate-IPE to give the title compound (206 mg, 80%) as a white powder.

MS (ESI+):646 (M+H)

### Example 58

### (3R*,4R*)-1-({1-[amino(oxo)acetyl]piperidin-3-yl}carbonyl)-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using the compound obtained in Example 52 and oxamic acid.

MS (ESI+):659 (M+H)

### Example 59

### (3R*,4R*)—N-[3,5-bis(trifluoromethyl)benzyl]-3-(4-fluoro-2-methylphenyl)-N-methyl-1-{[1-(methylsulfonyl)piperidin-3-yl]carbonyl}piperidine-4-carboxamide

The title compound was obtained by reaction and purification in the same manner as in Example 57 and using the compound obtained in Example 52 and methanesulfonyl chloride.

MS (ESI+):666 (M+H)

### Example 60

### (3R*,4R*)—N-[3,5-bis(trifluoromethyl)benzyl]-3-(4-fluoro-2-methylphenyl)-1-{[trans-4-(glycoloylamino)cyclohexyl]carbonyl}-N-methylpiperidine-4-carboxamide

(step 1)

tert-Butyl (trans-4-{[(3R*,4R*){[[3,5-bis(trifluoromethyl)benzyl](methyl)amino]carbonyl}-3-(4-fluoro-2-methylphenyl)piperidin-1-yl]carbonyl}cyclohexyl)carbamate was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and trans-4-[(tert-butoxycarbonyl)amino]cyclohexanecarboxylic acid.

MS (ESI+):702 (M+H)

(step 2)

(3R*,4R*)-1-[(trans-4-Aminocyclohexyl)carbonyl]-N-[3,5-bis(trifluoromethyl)benzyl]-3-(4-fluoro-2-methylphenyl)-N-methylpiperidine-4-carboxamide monohydrochloride was obtained by reaction and purification in the same manner as in Example 6 and using the compound obtained in step 1.

MS (ESI+):602 (M−HCl+H)

(step 3)

The title compound was obtained by reaction and purification in the same manner as in Example 1 and using the compound obtained in step 2 and glycolic acid.

MS (ESI+):660 (M+H)

### Example 61

### (3R*,4R*)—N-[3,5-bis(trifluoromethyl)benzyl]-3-(4-fluoro-2-methylphenyl)-1-({trans-4-[(methoxyacetyl)amino]cyclohexyl}carbonyl)-N-methylpiperidine-4-carboxamide

The title compound was obtained by reaction and purification in the same manner as in Example 1 and using the compound obtained in Example 60, step 2 and methoxyacetic acid.

MS (ESI+):674 (M+H)

### Example 62

### (3R*,4R*)-1-({trans-4-[(aminocarbonyl)amino]cyclohexyl}carbonyl)-N-[3,5-bis(trifluoromethyl)benzyl]-3-(4-fluoro-2-methylphenyl)-N-methylpiperidine-4-carboxamide

The title compound was obtained by reaction and purification in the same manner as in Example 48 and using the compound obtained in Example 60, step 2.

MS (ESI+):645 (M+H)

### Example 63

### N-(trans-4-[(3R*,4R*)-4-{[[3,5-bis(trifluoromethyl)benzyl](methyl)amino]carbonyl}-3-(4-fluoro-2-methylphenyl)piperidin-1-yl]carbonyl}cyclohexyl)ethanediamide

The title compound was obtained by reaction and purification in the same manner as in Example 1 and using the compound obtained in Example 60, step 2 and oxamic acid.

MS (ESI+):673 (M+H)

### Example 64

### (3R*,4R*)—N-[3,5-bis(trifluoromethyl)benzyl]-3-(4-fluoro-2-methylphenyl)-N-methyl-1-({trans-4-[(methylsulfonyl)amino]cyclohexyl}carbonyl)piperidine-4-carboxamide

The title compound was obtained by reaction and purification in the same manner as in Example 57 and using the compound obtained in Example 60, step 2 and methanesulfonyl chloride.

MS (ESI+):680 (M+H)

### Example 65

### (3R*,4R*)—N-[3,5-bis(trifluoromethyl)benzyl]-3-(4-fluoro-2-methylphenyl)-N-methyl-1-(tetrahydrofuran-3-ylcarbonyl)piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and tetrahydro-3-furoic acid.

MS (ESI+):575 (M+H)

### Example 66

### (3R*,4R*)—N-[3,5-bis(trifluoromethyl)benzyl]-3-(4-fluoro-2-methylphenyl)-N-methyl-1-{[(4R)-2-oxo-1,3-thiazolidin-4-yl]carbonyl}piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and L-2-oxothiazolidine-4-carboxylic acid.

MS (ESI+):606 (M+H)

### Example 67

### tert-butyl 3-{[(3R*,4R*)-4-{[[3,5-bis(trifluoromethyl)benzyl](methyl)amino]carbonyl}-3-(4-fluoro-2-methylphenyl)piperidin-1-yl]carbonyl}pyrrolidine-1-carboxylate

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 1-tert-butyl pyrrolidine-1,3-dicarboxylate ester.

MS (ESI+):674 (M+H)

### Example 68

### Methyl 3-{[(3R*,4R*)-4-{[[3,5-bis(trifluoromethyl)benzyl](methyl)amino]carbonyl}-3-(4-fluoro-2-methylphenyl)piperidin-1-yl]carbonyl}pyrrolidine-1-carboxylate

(step 1)

(3R*,4R*)—N-[3,5-bis(Trifluoromethyl)benzyl]-3-(4-fluoro-2-methylphenyl)-N-methyl-1-(pyrrolidin-3-ylcarbonyl)piperidine-4-carboxamide monohydrochloride was obtained by reaction and purification in the same manner as in Example 6 and using the compound obtained in Example 67.

MS (ESI+):574 (M−HCl+H)

(step 2)

The title compound was obtained by reaction and purification in the same manner as in Example 57 and using the compound obtained in step 1.

MS (ESI+):632 (M+H)

### Example 69

### (3R*,4R*)—N-[3,5-bis(trifluoromethyl)benzyl]-3-(4-fluoro-2-methylphenyl)-1-[(1-glycoloylpyrrolidin-3-yl)carbonyl]-N-methylpiperidine-4-carboxamide

The title compound was obtained by reaction and purification in the same manner as in Example 1 and using the compound obtained in Example 68, step 1 and glycolic acid.

MS (ESI+):632 (M+H)

### Example 70

### (3R*,4R*)—N-[3,5-bis(trifluoromethyl)benzyl]-3-(4-fluoro-2-methylphenyl)-1-{[1-(2-hydroxy-2-methylpropanoyl)azetidin-3-yl]carbonyl}-N-methylpiperidine-4-carboxamide

The title compound was obtained by reaction and purification in the same manner as in Example 1 and using the compound obtained in Reference Example 58, step 2 and 2-hydroxyisobutyric acid.

MS (ESI+):646 (M+H)

### Example 71

### (3R*,4R*)—N-[3,5-bis(trifluoromethyl)benzyl]-3-(4-fluoro-2-methylphenyl)-1-[(1-glycoloylazetidin-3-yl)carbonyl]-N-methylpiperidine-4-carboxamide

The title compound was obtained by reaction and purification in the same manner as in Example 1 and using the compound obtained in Reference Example 58, step 2 and glycolic acid.

MS (ESI+):618 (M+H)

### Example 72

### (3R*,4R*)-1-(1-adamantylcarbonyl)-N-(3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 1-adamantanecarboxylic acid.

MS (ESI+):639 (M+H)

### Example 73

### (3R*,4R*)-1-(3-amino-2,2-dimethyl-3-oxopropanoyl)-N-[3,5-bis(trifluoromethyl)benzyl]-3-(4-fluoro-2-methylphenyl)-N-methylpiperidine-4-carboxamide

(step 1)

To a solution of dimethylmalonic acid (79.3 mg) in a mixture of DMF (10 μL) and THF (10 mL) was added oxalyl chloride (51.5 μL) at 0° C., and the mixture was stirred for 0° C. for 2 hr. (3R*,4R*)—N-[3,5-bis(Trifluoromethyl)benzyl]-3-(4-fluoro-2-methylphenyl)-N-methylpiperidine-4-carboxamide monohydrochloride (154 mg) and ⁱPr₂NEt (261 μL) were added to the reaction mixture, and the mixture was stirred at room temperature for 3 days. The reaction mixture was poured into 0.1N hydrochloric acid, and the resultant product was extracted with ethyl acetate. The organic layer was washed with saturated brine and dried, and the solvent was evaporated under reduced pressure to give crude 3-[(3R*,4R*)-4-{[[3,5-bis(trifluoromethyl)benzyl](methyl)amino]carbonyl}-3-(4-fluoro-2-methylphenyl)piperidin-1-yl]-2,2-dimethyl-3-oxopropanoic acid (206 mg) as a colorless oil.

(step 2)

To a solution of the compound (206 mg) obtained in step 1 in CH₃CN (4 mL) were added WSC.HCl (115 mg) and HOBt.NH₃ (91.3 mg), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 40→100% ethyl acetate/hexane) to give the title compound (80 mg, 45%) as a white amorphous solid.

MS (ESI+):590 (M+H)

### Example 74

### (3R*,4R*)—N-(3,5-bis(trifluoromethyl)benzyl)-1-(N,N-dimethylglycyl)-3-(4-fluoro-2-methylphenyl)-N-methylpiperidine-4-carboxamide monohydrochloride

A colorless amorphous solid was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and N,N-dimethylglycine. The obtained amorphous solid was treated with 1 equivalent amount of 4N hydrogen chloride/ethyl acetate to give the title compound.

MS (ESI+):562 (M−HCl+H)

### Example 75

### (3R*,4R*)—N-[3,5-bis(trifluoromethyl)benzyl]-1-[(2,4-dioxo-3,4-dihydropyrimidin-1(2H)-yl)acetyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and (2,4-dioxo-3,4-dihydropyrimidin-1(2H)-yl)acetic acid.

MS (ESI+):629 (M+H)

### Example 76

### (3R*,4R*)—N-[3,5-bis(trifluoromethyl)benzyl]-1-[(5,5-dimethyl-2,4-dioxo-1,3-oxazolidin-3-yl)acetyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and (5,5-dimethyl-2,4-dioxo-1,3-oxazolidin-3-yl)acetic acid.

MS (ESI+):646 (M+H)

### Example 77

### (3R*,4R*)—N-[3,5-bis(trifluoromethyl)benzyl]-1-[(2,4-dioxo-1,3-oxazolidin-3-yl)acetyl]-3-(4-fluoro-2-methylphenyl)-N-methylpiperidine-4-carboxamide

(step 1)

Crude (2,4-dioxo-1,3-oxazolidin-3-yl)acetic acid was obtained by reaction and purification in the same manner as in Example 78, step 1 and using 2,4-oxazolidinedione.

(step 2)

The title compound was obtained by reaction and purification in the same manner as in Example 1 and using the compound obtained in step 1 and (3R*,4R*)—N-[3,5-bis(trifluoromethyl)benzyl]-3-(4-fluoro-2-methylphenyl)-N-methylpiperidine-4-carboxamide monohydrochloride.

MS (ESI+):618 (M+H)

### Example 78

### (3R*,4R*)—N-[3,5-bis(trifluoromethyl)benzyl]-3-(4-fluoro-2-methylphenyl)-N-methyl-1-[(3,4,4-trimethyl-2,5-dioxoimidazolidin-1-yl)acetyl]piperidine-4-carboxamide

(step 1)

To a solution of 1,5,5-trimethylhydantoin (4.98 g) in DMF (30 mL) was added 60% NaH (1.68 g) at 0° C., and the mixture was stirred for 30 min. A solution of benzyl bromoacetate (8.0 g) in DMF (10 mL) was added to the reaction mixture, and the mixture was stirred at room temperature for 3 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 50→100% ethyl acetate/hexane) to give a colorless oil (8.5 g). A suspension of the obtained oil (7.3 g) and 10% palladium-carbon (0.73 g) in EtOH (130 mL) was stirred under a hydrogen atmosphere (0.1 MPa) at room temperature for 5 hr. The catalyst was filtered off, and the filtrate was concentrated under reduced pressure to give (3,4,4-trimethyl-2,5-dioxoimidazolidin-1-yl)acetic acid (4.6 g) as white crystals.

melting point: 130-132° C.

(step 2)

The title compound was obtained by reaction and purification in the same manner as in Example 1 and using the compound obtained in step 1 and (3R*,4R*)—N-[3,5-bis(trifluoromethyl)benzyl]-3-(4-fluoro-2-methylphenyl)-N-methylpiperidine-4-carboxamide monohydrochloride.

MS (ESI+):659 (M+H)

### Example 79

### (3R*,4R*)—N-[3,5-bis(trifluoromethyl)benzyl]-1-[(4,4-dimethyl-2,6-dioxopiperidin-1-yl)acetyl]-3-(4-fluoro-2-methylphenyl)-N-methylpiperidine-4-carboxamide

(step 1)

(4,4-Dimethyl-2,6-dioxopiperidin-1-yl)acetic acid was obtained by reaction and purification in the same manner as in Example 78, step 1 and using 3,3-dimethylglutarimide.

melting point: 125° C.

(step 2)

The title compound was obtained by reaction and purification in the same manner as in Example 1 and using the compound obtained in step 1 and (3R*,4R*)—N-[3,5-bis(trifluoromethyl)benzyl]-3-(4-fluoro-2-methylphenyl)-N-methylpiperidine-4-carboxamide monohydrochloride.

MS (ESI+):658 (M+H)

### Example 80

### (3R*,4R*)—N-[3,5-bis(trifluoromethyl)benzyl]-1-[(3,5-dioxomorpholin-4-yl)acetyl]-3-(4-fluoro-2-methylphenyl)-N-methylpiperidine-4-carboxamide

(step 1)

(3,5-Dioxomorpholin-4-yl)acetic acid was obtained by reaction and purification in the same manner as in Example 78, step 1 and using 3,5-dioxomorpholine.

melting point: 101° C.

(step 2)

The title compound was obtained by reaction and purification in the same manner as in Example 1 and using the compound obtained in step 1 and (3R*,4R*)—N-[3,5-bis(trifluoromethyl)benzyl]-3-(4-fluoro-2-methylphenyl)-N-methylpiperidine-4-carboxamide monohydrochloride.

MS (ESI+):632 (M+H)

### Example 81

### (3S,4S)—N-[3,5-bis(trifluoromethyl)benzyl]-1-[3-(5,5-dimethyl-2,4-dioxo-1,3-oxazolidin-3-yl)propanoyl]-3-(4-fluoro-2-methylphenyl)-N-methylpiperidine-4-carboxamide

(step 1)

A solution of 5,5-dimethyloxazolidine-2,4-dione (1.0 g) and benzyl acrylate (5.18 g) in a mixture of pyridine (38.8 mL) and water (7.8 mL) was refluxed under heating for 19 hr, and the mixture was stirred at room temperature for 3 days. The reaction mixture was poured into water, and the resultant product was extracted with ethyl acetate. The organic layer was washed with aqueous citric acid solution and water and dried, and the solvent was evaporated under reduced pressure. The obtained residue was purified by silica gel column chromatography (solvent gradient; 10% ethyl acetate/hexane) to give benzyl 3-(5,5-dimethyl-2,4-dioxo-1,3-oxazolidin-3-yl)propanoate (2.84 g, 100%) as a colorless oil.

¹H-NMR (300 MHz, CDCl₃):δ 1.51 (6H, s), 2.75 (2H, t, J=6.8 Hz), 3.85 (2H, d, J=6.8 Hz), 5.10 (2H, s), 7.31-7.39 (5H, m)

(step 2)

A suspension of the compound (2.39 g) obtained in step 1 and 10% palladium-carbon (800 mg) in EtOH (24 mL) was stirred under 1 atm of hydrogen atmosphere, at room temperature for 12 hr. The catalyst was filtered off, and the filtrate was concentrated under reduced pressure to give 3-(5,5-dimethyl-2,4-dioxo-1,3-oxazolidin-3-yl)propanoic acid (1.45 g, 88%) as a white powder.

¹H-NMR (300 MHz, CDCl₃):δ 1.57 (6H, s), 2.78 (2H, t, J=7.0 Hz), 3.84 (2H, t, J=7.0 Hz)

(step 3)

The title compound was obtained by reaction and purification in the same manner as in Example 1 and using the compound obtained in step 2 and (3R*,4R*)—N-[3,5-bis(trifluoromethyl)benzyl]-3-(4-fluoro-2-methylphenyl)-N-methylpiperidine-4-carboxamide monohydrochloride.

MS (ESI+):660 (M+H)

### Example 82

### (3R*,4R*)—N-[3,5-bis(trifluoromethyl)benzyl]-1-[(2,6-dioxo-1,2,3,6-tetrahydropyrimidin-4-yl)carbonyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and ortho acid monohydrate.

MS (ESI+):615 (M+H)

### Example 83

### (3R*,4R*)—N-[3,5-bis(trifluoromethyl)benzyl]-3-(4-fluoro-2-methylphenyl)-N-methyl-1-(pyridin-2-ylcarbonyl)piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and picolinic acid.

MS (ESI+):582 (M+H)

### Example 84

### Methyl 6-{[(3R*,4R*)-4-{[[3,5-bis(trifluoromethyl)benzyl](methyl)amino]carbonyl}-3-(4-fluoro-2-methylphenyl)piperidin-1-yl]carbonyl}nicotinate

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 5-(methoxycarbonyl)pyridine-2-carboxylic acid.

MS (ESI+):640 (M+H)

### Example 85

### (3R*,4R*)—N-[3,5-bis(trifluoromethyl)benzyl]-3-(4-fluoro-2-methylphenyl)-1-[(5-hydroxypyridin-2-yl)carbonyl]-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 5-hydroxypicolinic acid.

MS (ESI+):598 (M+H)

### Example 86

### (3R*,4R*)—N-[3,5-bis(trifluoromethyl)benzyl]-3-(4-fluoro-2-methylphenyl)-1-[(6-hydroxypyridin-2-yl)carbonyl]-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 6-hydroxypicolinic acid.

MS (ESI+):598 (M+H)

### Example 87

### (3R*,4R*)—N-[3,5-bis(trifluoromethyl)benzyl]-1-[(5-bromopyridin-2-yl)carbonyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 5-bromopyridine-2-carboxylic acid.

MS (ESI+):660, 662 (M+H)

### Example 88

### (3R*,4R*)—N-[3,5-bis(trifluoromethyl)benzyl]-3-(4-fluoro-2-methylphenyl)-N-methyl-1-[(6-methylpyridin-2-yl)carbonyl]piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 6-methylpicolinic acid.

MS (ESI+):596 (M+H)

### Example 89

### 6-{[(3R*,4R*)-4-{[[3,5-bis(trifluoromethyl)benzyl](methyl)amino]carbonyl}-3-(4-fluoro-2-methylphenyl)piperidin-1-yl]carbonyl}nicotinamide

A solution of the compound (150 mg) obtained in Example 84 in a mixture of 28% aqueous ammonia solution (0.5 mL) and MeOH (1.5 mL) was irradiated in a microwave reaction apparatus at 100° C. for 30 min. The reaction mixture was concentrated under reduced pressure. WSC.HCl (55.1 mg), HOBt.NH₃ (42.8 mg), Et₃N (39.2 μL) and THF (5 mL) were added to the obtained residue, and the mixture was stirred at room temperature for 18 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (48 mg, 33%) as a white powder.

MS (ESI+):625 (M+H)

### Example 90

### (3R*,4R*)-1-[(6-aminopyridin-3-yl)carbonyl]-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 6-aminonicotinic acid.

MS (ESI+):597 (M+H)

### Example 91

### (3R*,4R*)-1-{[6-(acetylamino)pyridin-3-yl]carbonyl}-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 2-acetamido-5-pyridinecarboxylic acid.

MS (ESI+):639 (M+H)

### Example 92

### (3R*,4R*)—N-[3,5-bis(trifluoromethyl)benzyl]-3-(4-fluoro-2-methylphenyl)-N-methyl-1-(pyrimidin-5-ylcarbonyl)piperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and pyrimidine-5-carboxylic acid.

MS (ESI+):583 (M+H)

### Example 93

### (3R*,4R*)-1-[(2-aminopyrimidin-5-yl)carbonyl]-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 2-aminopyrimidine-5-carboxylic acid.

MS (ESI+):598 (M+H)

### Example 94

### (3R*,4R*)-1-{[2-(acetylamino)pyrimidin-5-yl]carbonyl}-N-[3,5-bis(trifluoromethyl)benzyl]-3-(4-fluoro-2-methylphenyl)-N-methylpiperidine-4-carboxamide

A solution of the compound (150 mg) obtained in Example 93 in acetic anhydride (2.5 mL) was stirred at 160° C. for 5 hr. The reaction mixture was poured into an aqueous sodium hydrogen carbonate solution, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 50→100% ethyl acetate/hexane) to give the title compound (61 mg, 38%) as a white amorphous solid.

MS (ESI+):640 (M+H)

### Example 95

### Methyl (5-{[(3R*,4R*)-4-{[[3,5-bis(trifluoromethyl)benzyl](methyl)amino]carbonyl}-3-(4-fluoro-2-methylphenyl)piperidin-1-yl]carbonyl}pyrimidin-2-yl)carbamate

To a solution of the compound (100 mg) obtained in Example 93 and sodium hydride (60% in oil, 8.0 mg) in THF (2 mL) was added methyl chloroformate (15.8 μL) at 0° C., and the mixture was stirred at room temperature for one day. The reaction mixture was poured into an aqueous sodium hydrogen carbonate solution, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 50→100% ethyl acetate/hexane) to give the title compound (35 mg, 32%) as a white powder.

MS (ESI+):656 (M+H)

### Example 96

### (3R*,4R*)-1-[4-(aminocarbonyl)benzoyl]-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and terephthalic acid monoamide.

MS (ESI+):624 (M+H)

### Example 97

### (3R*,4R*)—N-[3,5-bis(trifluoromethyl)benzyl]-3-(4-fluoro-2-methylphenyl)-1-(1H-imidazol-4-ylcarbonyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and imidazole-4-carboxylic acid.

MS (ESI+):571 (M+H)

### Example 98

### (3R*,4R*)—N-[3,5-bis(trifluoromethyl)benzyl]-3-(4-fluoro-2-methylphenyl)-1-(1H-indol-6-ylcarbonyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and indole-6-carboxylic acid.

MS (ESI+):620 (M+H)

### Example 99

### (3R*,4R)-1-(1H-benzoimidazol-6-ylcarbonyl)-N-[3,5-bis(trifluoromethyl)benzyl]-3-(4-fluoro-2-methylphenyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 5-benzoimidazolecarboxylic acid.

MS (ESI+):621 (M+H)

### Example 100

### (3R*,4R*)-1-(1,3-benzothiazol-2-ylcarbonyl)-N-[3,5-bis(trifluoromethyl)benzyl]-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 1,3-benzothiazole-2-carbonyl chloride.

MS (ESI+):638 (M+H)

### Example 101

### (3R*,4R)—N-[3,5-bis(trifluoromethyl)benzyl]-3-(4-fluoro-2-methylphenyl)-N-methyl-1-(1,3-thiazol-2-ylcarbonyl)piperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 1,3-thiazole-2-carbonyl chloride.

MS (ESI+):588 (M+H)

### Example 102

### (3R*,4R*)—N-[3,5-bis(trifluoromethyl)benzyl]-3-(4-fluoro-2-methylphenyl)-1-(1H-imidazol-2-ylcarbonyl)-N-methylpiperidine-4-carboxamide

The title compound was obtained by reaction and purification in the same manner as in Example 1 and using (3R*,4R*)—N-[3,5-bis(trifluoromethyl)benzyl]-3-(4-fluoro-2-methylphenyl)-N-methylpiperidine-4-carboxamide monohydrochloride and 1H-imidazole-2-carboxylic acid.

MS (ESI+):571 (M+H)

### Example 103

### (3R*,4R*)—N-[3,5-bis(trifluoromethyl)benzyl]-1-[2-(cyclopropylamino)-2-oxoethyl]-3-(4-fluoro-2-methylphenyl)-N-methylpiperidine-4-carboxamide monohydrochloride

(step 1)

A solution of the compound (236 mg) obtained in Reference Example 66 in TFA (3.5 mL) was stirred at room temperature for 14 hr. The reaction solution was concentrated under reduced pressure to give crude [(3R*,4R*)-4-{[[3,5-bis(trifluoromethyl)benzyl](methyl)amino]carbonyl}-3-(4-fluoro-2-methylphenyl)piperidin-1-yl]acetic acid as a colorless oil.

(step 2)

CH₃CN (3.5 mL) was added to the crude product obtained in step 1, WSC.HCl (92 mg), HOBt.H₂O (73.5 mg), cyclopropylamine (36 μL) and Et₃N (72.5 μL) were added, and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→100% ethyl acetate/hexane) to give a colorless oil. The obtained oil was treated with 1 equivalent amount of 4N hydrogen chloride/ethyl acetate to give the title compound (167 mg, 68%) as a white powder.

MS (ESI+):574 (M−HCl+H)

elemental analysis value: C₂₈H₃₁N₃O₂ClF₇

Found C, 55.18; H, 5.04; N, 6.40

Calculated C, 55.13; H, 5.12; N, 6.89

Melting point: 210-214° C.

¹H-NMR (300 MHz, CDCl₃):δ 0.51-0.56 (2H, m), 0.79-0.85 (2H, m), 1.83-2.02 (2H, m), 2.15-2.45 (5H, m), 2.72-3.01 (9H, m), 3.51-3.59 (1H, m), 4.24-4.80 (2H, m), 6.66-7.83 (7H, m) (NMR characterization was carried out as its free amine.)

### Example 104

### (3R*,4R*)—N-[3,5-bis(trifluoromethyl)benzyl]-3-(4-fluoro-2-methylphenyl)-1-[2-(4-hydroxypiperidin-1-yl)-2-oxoethyl]-N-methylpiperidine-4-carboxamide monohydrochloride

(step 1)

A solution of the compound (236 mg) obtained in Reference Example 66 in TFA (3.5 mL) was stirred at room temperature for 14 hr. The reaction solution was concentrated under reduced pressure to give crude [(3R*,4R*)-4-{[[3,5-bis(trifluoromethyl)benzyl](methyl)amino]carbonyl}-3-(4-fluoro-2-methylphenyl)piperidin-1-yl]acetic acid as a colorless oil.

(step 2)

CH₃CN (3.5 mL) was added to the crude product obtained in step 1, WSC.HCl (92 mg), HOBt.H₂O (73.5 mg), 4-hydroxypiperidine (52.8 mg) and Et₃N (72.5 μL) were added, and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 30→100% ethyl acetate/hexane) to give a colorless oil. The obtained oil was treated with 1 equivalent amount of 4N hydrogen chloride/ethyl acetate to give the title compound (188 mg, 72%) as a white powder.

MS (ESI+):618 (M−HCl+H)

elemental analysis value: C₃₀H₃₅N₃O₃ClF₇

Found C, 54.85; H, 5.42; N, 6.16

Calculated C, 55.09; H, 5.39; N, 6.42

Melting point: 220-224° C.

¹H-NMR (300 MHz, CDCl₃):δ 1.40-1.65 (2H, m), 1.83-2.45 (11H, m), 2.76-4.82 (15H, m), 6.69-7.84 (6H, m) (NMR characterization was carried out as its free amine.)

### Example 105

### tert-butyl (3R*,4R*)-4-{[[3,5-bis(trifluoromethyl)benzyl](methyl)amino]carbonyl}-3-(4-fluoro-2-methylphenyl)-1,4′-bipiperidine-1′-carboxylate

To a solution of (3R*,4R*)—N-[3,5-bis(trifluoromethyl)benzyl]-3-(4-fluoro-2-methylphenyl)-N-methylpiperidine-4-carboxamide monohydrochloride (527 mg) and 1-Boc-4-piperidone (1990 mg) in ethyl acetate (15 mL) was added Et₃N (181 μL), and the mixture was stirred at room temperature for 2 hr. Acetic acid (1 mL) and NaBH(OAc)₃ (1060 mg) were further added, and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 10→50% ethyl acetate/hexane), and successively by silica gel column chromatography (solvent gradient; 10→100% ethyl acetate/hexane) to give the title compound (547 mg, 83%) as a white powder.

MS (ESI+):660 (M+H)

### Example 106

### (3R*,4R*)—N-[3,5-bis(trifluoromethyl)benzyl]-3-(4-fluoro-2-methylphenyl)-N-methyl-1,4′-bipiperidine-4-carboxamide dihydrochloride

To a solution of the compound (470 mg) obtained in Example 105 in ethyl acetate (3 mL) was added 4N hydrogen chloride/ethyl acetate (3 mL) solution, and the mixture was stirred with heating at room temperature for 4 hr. The reaction mixture was concentrated under reduced pressure, and crystallized from MeOH-ethyl acetate to give the title compound (419 mg, 93%) as white crystals.

MS (ESI+):560 (M−2HCl+H)

### Example 107

### Isopropyl (3R*,4R*)-4-{[[3,5-bis(trifluoromethyl)benzyl](methyl)amino]carbonyl}-3-(4-fluoro-2-methylphenyl)-1,4′-bipiperidine-1′-carboxylate monohydrochloride

A colorless oil was obtained by reaction and purification in the same manner as in Example 57 and using the compound obtained in Example 106 and isopropyl chloroformate. The oil was treated with 1 equivalent amount of 4N hydrogen chloride/ethyl acetate to give the title compound.

MS (ESI+):646 (M−HCl+H)

### Example 108

### (3R*,4R*)-1′-[amino(oxo)acetyl]-N-[3,5-bis(trifluoromethyl)benzyl]-3-(4-fluoro-2-methylphenyl)-N-methyl-1,4′-bipiperidine-4-carboxamide monohydrochloride

A colorless oil was obtained by reaction and purification in the same manner as in Example 1 and using the compound obtained in Example 106 and oxamic acid. The oil was treated with 1 equivalent amount of 4N hydrogen chloride/ethyl acetate to give the title compound.

MS (ESI+):631 (M−HCl+H)

elemental analysis value: C₃₀H₃₄N₄O₃ClF₇.H₂O

Found C, 52.41; H, 5.43; N, 8.06

Calculated C, 52.60; H, 5.30; N, 8.18

Melting point: 175-176° C.

¹H-NMR (300 MHz, CDCl₃):δ 1.47-1.67 (2H, m), 1.83-2.03 (4H, m), 2.18-3.11 (14H, m), 3.49-3.57 (1H, m), 4.21-4.82 (4H, m), 5.91 (1H, br), 6.69-7.83 (7H, m) (NMR characterization was carried out as its free amine.)

### Example 109

### (3R*,4R*)—N⁴-[3,5-bis(trifluoromethyl)benzyl]-3-(4-fluoro-2-methylphenyl)-N⁴-methyl-1,4′-bipiperidine-1′,4-dicarboxamide monohydrochloride

A colorless oil was obtained by reaction and purification in the same manner as in Example 48 and using the compound obtained in Example 106. The oil was treated with 1 equivalent amount of 4N hydrogen chloride/ethyl acetate to give the title compound.

MS (ESI+):603 (M−HCl+H)

elemental analysis value: C₂₉H₃₄N₄O₂ClF₇.2.5H₂O

Found C, 50.71; H, 5.60; N, 8.36

Calculated C, 50.92; H, 5.75; N, 8.19

Melting point: 175-178° C.

¹H-NMR (300 MHz, CDCl₃):δ 1.43-1.57 (2H, m), 1.83-2.03 (4H, m), 2.18-3.10 (14H, m), 3.50-3.57 (1H, m), 3.95-5.15 (5H, m), 6.73-8.37 (7H, m) (NMR characterization was carried out as its free amine.)

### Example 110

### (3R*,4R*)—N-[3,5-bis(trifluoromethyl)benzyl]-3-(4-fluoro-2-methylphenyl)-1′-glycoloyl-N-methyl-1,4′-bipiperidine-4-carboxamide monohydrochloride

A colorless oil was obtained by reaction and purification in the same manner as in Example 1 and using the compound obtained in Example 106 and glycolic acid. The oil was treated with 1 equivalent amount of 4N hydrogen chloride/ethyl acetate to give the title compound.

MS (ESI+):618 (M−HCl+H)

### Example 111

### (3R,4R*)—N-[3,5-bis(trifluoromethyl)benzyl]-3-(4-fluoro-2-methylphenyl)-1′-(2-hydroxy-2-methylpropanoyl)-N-methyl-1,4′-bipiperidine-4-carboxamide monohydrochloride

A colorless oil was obtained by reaction and purification in the same manner as in Example 1 and using the compound obtained in Example 106 and 2-hydroxyisobutyric acid. The oil was treated with 1 equivalent amount of 4N hydrogen chloride/ethyl acetate to give the title compound.

MS (ESI+):646 (M−HCl+H)

### Example 112

### (3R*,4R*)-1-(2-amino-2-oxoethyl)-N-[3,5-bis(trifluoromethyl)benzyl]-3-(4-fluoro-2-methylphenyl)-N-methylpiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (205 mg), 2-bromoacetamide (82.8 mg) and NaI (12 mg) in DMF (3 mL) was added Et₃N (140 μL) at room temperature, and the mixture was stirred at room temperature for 4 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 0→100% ethyl acetate/hexane) to give a white amorphous solid. The thus-obtained amorphous solid was treated with 1 equivalent amount of 4N hydrogen chloride/ethyl acetate to give the title compound (215 mg, 94%) as a white powder.

MS (ESI+):534 (M−HCl+H)

elemental analysis value: C₂₅H₂₇N₃O₂ClF₇.H₂O.0.1IPE

Found C, 51.56; H, 5.01; N, 7.21

Calculated C, 51.40; H, 5.12; N, 7.02

Melting point: 175-177° C.

¹H-NMR (300 MHz, DMSO-d₆):δ 1.91-2.17 (2H, m), 2.32-2.41 (3H, m), 2.64-3.02 (3H, m), 3.20-3.35 (3H, m), 3.56-3.96 (5H, m), 4.18-5.09 (2H, m), 6.67-8.09 (8H, m) (NMR characterization was carried out as its free amine.)

### Example 113

### (3R*,4R*)-1-(2-amino-1,1-dimethyl-2-oxoethyl)-N-[3,5-bis(trifluoromethyl)benzyl]-3-(4-fluoro-2-methylphenyl)-N-methylpiperidine-4-carboxamide monohydrochloride

(step 1)

To a solution of 2-bromoisobutyryl bromide (4.58 g) in THF (10 mL) was added a solution of benzyl alcohol (2.05 g) and pyridine (1.62 mL) in THF (40 mL) at 0° C., and the mixture was stirred at room temperature for 4 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 0→20% ethyl acetate/hexane) to give benzyl 2-bromo-2-methylpropanoate (4.80 g, 98%) as a colorless oil.

¹H-NMR (300 MHz, CDCl₃):δ 1.95 (6H, s), 5.21 (2H, s), 7.33-7.39 (5H, m)

(step 2)

To a solution of the compound (206 mg) obtained in step 1, (3R*,4R*)—N-[3,5-bis(trifluoromethyl)benzyl]-3-(4-fluoro-2-methylphenyl)-N-methylpiperidine-4-carboxamide monohydrochloride (205 mg), and NaI (12 mg) in DMF (3 mL) was added ⁱPr₂NEt (418 μL) at room temperature, and the mixture was stirred for 50° C. for 1 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 0→33% ethyl acetate/hexane) to give benzyl 2-[(3R*,4R*)-4-{[[3,5-bis(trifluoromethyl)benzyl](methyl)amino]carbonyl}-3-(4-fluoro-2-methylphenyl)piperidin-1-yl]-2-methylpropanoate (172 mg, 66%) as a colorless oil.

MS (ESI+):653 (M+H)

(step 3)

A suspension of the compound (161 mg) obtained in step 2 and 10% palladium-carbon (10 mg) in a mixture of ethyl acetate (4 mL) and EtOH (2.5 mL) was stirred under 1 atm of hydrogen atmosphere and at room temperature for 12 hr. The catalyst was filtered off, and the filtrate was concentrated under reduced pressure to give 2-[(3R*,4R*)-4-{[[3,5-bis(trifluoromethyl)benzyl](methyl)amino]carbonyl}-3-(4-fluoro-2-methylphenyl)piperidin-1-yl]-2-methylpropanoic acid (137 mg, 99%) as a white powder.

MS (ESI+):563 (M+H)

(step 4)

To a solution of the compound (137 mg) obtained in step 3 in CH₃CN (6 mL) were added WSC.HCl (70.0 mg) and HOBt.NH₃ (55.5 mg), and the mixture was stirred at room temperature for 2 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 10→100% ethyl acetate/hexane) to give a colorless oil. The thus-obtained oil was treated with 1 equivalent amount of 4N hydrogen chloride/ethyl acetate to give the title compound (96 mg, 40%) as a white powder.

MS (ESI+):562 (M−HCl+H)

elemental analysis value: C₂₇H₃₁N₃O₂ClF₇

Found C, 53.96; H, 5.25; N, 6.92

Calculated C, 54.23; H, 5.23; N, 7.03

Melting point: 209-211° C.

¹H-NMR (300 MHz, CDCl₃):δ 1.17-1.28 (6H, m), 1.87-2.44 (8H, m), 2.78-3.06 (5H, m), 3.48-3.57 (1H, m), 4.25-4.80 (2H, m), 5.52 (1H, br), 6.74-7.84 (7H, m) (NMR characterization was carried out as its free amine.)

### Example 114

### (3R*,4R*)-1-{2-[4-(aminocarbonyl)piperidin-1-yl]-2-oxoethyl}-N-[3,5-bis(trifluoromethyl)benzyl]-3-(4-fluoro-2-methylphenyl)-N-methylpiperidine-4-carboxamide monohydrochloride

The title compound was obtained by reaction and purification in the same manner as in Example 104, step 2 and using the compound obtained in Example 104, step 1 and isonipecotamide.

MS (ESI+):645 (M−HCl+H)

### Example 115

### (3R*,4R*)—N-[3,5-bis(trifluoromethyl)benzyl]-3-(4-fluoro-2-methylphenyl)-N-methyl-1-[2-oxo-2-(3-oxopiperazin-1-yl)ethyl]piperidine-4-carboxamide monohydrochloride

The title compound was obtained by reaction and purification in the same manner as in Example 104, step 2 and using the compound obtained in Example 104, step 1 and piperazin-2-one.

MS (ESI+):617 (M−HCl+H)

### Example 116

### Methyl 3-[(3R*,4R*)-4-{[[3,5-bis(trifluoromethyl)benzyl](methyl)amino]carbonyl}-3-(4-fluoro-2-methylphenyl)piperidin-1-yl]propanoate monohydrochloride

The title compound was obtained by reaction and purification in the same manner as in Example 112 and using (3R*,4R*)—N-[3,5-bis(trifluoromethyl)benzyl]-3-(4-fluoro-2-methylphenyl)-N-methylpiperidine-4-carboxamide monohydrochloride and methyl 3-bromopropionate.

MS (ESI+):563 (M−HCl+H)

### Example 117

### (3R*,4R*)-1-(3-amino-3-oxopropyl)-N-[3,5-bis(trifluoromethyl)benzyl]-3-(4-fluoro-2-methylphenyl)-N-methylpiperidine-4-carboxamide monohydrochloride

A solution of the compound (222 mg) obtained in Example 116 and 28% aqueous ammonia (2.0 mL) in MeOH (1 mL) was stirred at 60° C. for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→5% ethyl acetate/hexane) to give a colorless oil. The thus-obtained oil was treated with 1 equivalent amount of 4N hydrogen chloride/ethyl acetate to give the title compound (127 mg, 55%) as a white powder.

MS (ESI+):548 (M−HCl+H)

### Example 118

### (3R*,4R*)—N-[3,5-bis(trifluoromethyl)benzyl]-3-(4-fluoro-2-methylphenyl)-N-methyl-1-(3-oxocyclopent-1-en-1-yl)piperidine-4-carboxamide

A solution of (3R*,4R*)—N-[3,5-bis(trifluoromethyl)benzyl]-3-(4-fluoro-2-methylphenyl)-N-methylpiperidine-4-carboxamide monohydrochloride (280 mg), 1,3-cyclopentanedione (64.3 mg) and p-toluenesulfonic acid monohydrate (10 mg) in toluene (5.5 mL) was stirred at 100° C. for 1.5 hr. The reaction mixture was poured into saturated aqueous sodium hydrogen carbonate solution, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→1% MeOH/ethyl acetate) to give the title compound (243 mg, 80%) as a white powder.

MS (ESI+):557 (M+H)

elemental analysis value: C₂₈H₂₇N₂O₂Cl₂F₇

Found C, 60.43; H, 5.09; N, 4.90

Calculated C, 60.43; H, 4.89; N, 5.03

Melting point: 192-193° C.

¹H-NMR (300 MHz, CDCl₃):δ 1.95-1.98 (2H, m), 2.39-2.64 (7H, m), 2.78-3.29 (6H, m), 3.48-4.23 (4H, m), 4.55-5.12 (2H, m), 6.78-7.85 (6H, m)

### Example 119

### (3R*,4R*)—N-[3,5-bis(trifluoromethyl)benzyl]-1-(4,4-dimethyl-3-oxocyclohex-1-en-1-yl)-3-(4-fluoro-2-methylphenyl)-N-methylpiperidine-4-carboxamide

The title compound was obtained by reaction and purification in the same manner as in Example 118 and using 4,4-dimethyl-1,3-cyclohexanedione.

MS (ESI+):599 (M+H)

elemental analysis value: C₃₁H₃₃N₂O₂F₇

Found C, 61.91; H, 5.47; N, 4.60

Calculated C, 62.20; H, 5.56; N, 4.68

Melting point: 137-138° C.

¹H-NMR (300 MHz, CDCl₃):δ 1.12 (6H, s), 1.83-1.98 (4H, m), 2.39-2.49 (5H, m), 2.78-3.06 (5H, m), 3.21-4.86 (6H, m), 5.16-5.19 (1H, m), 6.68-7.84 (6H, m)

### Example 120

### (3R*,4R*)—N-[3,5-bis(trifluoromethyl)benzyl]-3-(4-fluoro-2-methylphenyl)-1-(2-hydroxy-1-methylethyl)-N-methylpiperidine-4-carboxamide monohydrochloride

A solution of (3R*,4R*)—N-[3,5-bis(trifluoromethyl)benzyl]-3-(4-fluoro-2-methylphenyl)-N-methylpiperidine-4-carboxamide monohydrochloride (205 mg), hydroxyacetone (296 mg) and Et₃N (725 μL) in ethyl acetate (4 mL) was stirred at room temperature for 30 min. Acetic acid (0.2 mL) and NaBH(OAc)₃ (424 mg) were added, and the mixture was stirred at room temperature for 3 days. The reaction mixture was poured into saturated aqueous sodium hydrogen carbonate solution, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 0→100% ethyl acetate/hexane) to give a colorless oil. The thus-obtained oil was treated with 1 equivalent amount of 4N hydrogen chloride/ethyl acetate to give the title compound (136 mg, 60%) as a white powder.

MS (ESI+):535 (M−HCl+H)

elemental analysis value: C₂₆H₃₀N₂O₂ClF₇

Found C, 54.54; H, 5.30; N, 4.88

Calculated C, 54.69; H, 5.30; N, 4.91

Melting point: 234-236° C.

¹H-NMR (300 MHz, CDCl₃):δ 0.85-0.93 (3H, m), 1.83-3.64 (18H, m), 4.21-4.83 (2H, m), 6.70-7.84 (6H, m) (NMR characterization was carried out as its free amine.)

### Example 121

### (3R*,4R*)-1-(2-amino-1-methyl-2-oxoethyl)-N-[3,5-bis(trifluoromethyl)benzyl]-3-(4-fluoro-2-methylphenyl)-N-methylpiperidine-4-carboxamide monohydrochloride

(step 1)

Benzyl 2-bromopropanoate was obtained by reaction and purification in the same manner as in Example 113, step 1 and using 2-bromopropionyl bromide.

¹H-NMR (300 MHz, CDCl₃):δ 1.84 (3H, d, J=7.1 Hz), 4.41 (1H, q, J=7.1 Hz), 5.20 (2H, s), 7.33-7.41 (5H, m)

(step 2)

Benzyl 2-[(3R*,4R*)-4-{[[3,5-bis(trifluoromethyl)benzyl](methyl)amino]carbonyl}-3-(4-fluoro-2-methylphenyl)piperidin-1-yl]propanoate was obtained by reaction and purification in the same manner as in Example 113, step 2 and using the compound obtained in step 1.

MS (ESI+):639 (M+H)

(step 3)

2-[(3R*,4R*)-4-{[[3,5-bis(Trifluoromethyl)benzyl](methyl)amino]carbonyl}-3-(4-fluoro-2-methylphenyl)piperidin-1-yl]propanoic acid was obtained by reaction and purification in the same manner as in Example 113, step 3 and using the compound obtained in step 2.

MS (ESI+):549 (M+H)

(step 4)

The title compound was obtained by reaction and purification in the same manner as in Example 113, step 4 and using the compound obtained in step 3.

MS (ESI+):548 (M−HCl+H)

### Example 122

### (3R*,4R*)—N-[3,5-bis(trifluoromethyl)benzyl]-3-(4-fluoro-2-methylphenyl)-N-methyl-1-[l-methyl-2-(methylsulfonyl)ethyl]piperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide (315 mg), methylsulfonylacetone (180 mg) and acetic acid (38 μL) in ethyl acetate (30 mL) was added NaBH(OAc)₃ (443 mg), and the mixture was stirred at room temperature for one day. The reaction mixture was poured into saturated aqueous sodium hydrogen carbonate solution, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) and preparative HPLC. The thus-obtained colorless oil (149.6 mg) was treated with 1 equivalent amount of 4N hydrogen chloride/ethyl acetate to give the title compound (47.4 mg, 11%) as a white powder.

MS (ESI+):597 (M−HCl+H)

### Example 123

### (3R*,4R*)—N-[3,5-bis(trifluoromethyl)benzyl]-3-(4-fluoro-2-methylphenyl)-1-(3-hydroxy-3-methylbutyl)-N-methylpiperidine-4-carboxamide monohydrochloride

(step 1)

To a solution of 3-methyl-1,3-butanediol (1.0 g) in pyridine (10 mL) was added p-toluenesulfonyl chloride (2.26 g) at 0° C., and the mixture was stirred at room temperature for 27 hr. The reaction mixture was poured into ethyl acetate, organic layer was washed with 1N hydrochloric acid and water and dried, and the solvent was evaporated under reduced pressure. The obtained residue was purified by silica gel column chromatography (solvent gradient; 0→100% ethyl acetate/hexane) to give 3-hydroxy-3-methylbutyl 4-methylbenzenesulfonate (878 mg, 35%) as a colorless oil.

¹H-NMR (300 MHz, CDCl₃):δ 1.22 (6H, s), 1.34 (1H, s), 1.86 (2H, t, J=6.8 Hz), 2.45 (3H, s), 4.21 (2H, t, J=6.8 Hz), 7.35 (2H, d, J=8.0 Hz), 7.80 (2H, d, J=8.3 Hz)

(step 2)

A solution of the compound (151 mg) obtained in step 1, (3R*,4R*)—N-[3,5-bis(trifluoromethyl)benzyl]-3-(4-fluoro-2-methylphenyl)-N-methylpiperidine-4-carboxamide monohydrochloride (200 mg), potassium carbonate (65 mg) and NaI (88.1 mg) in CH₃CN (10 mL) was stirred at 80° C. for 18 hr. The reaction mixture was poured into saturated aqueous sodium hydrogen carbonate solution, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The thus-obtained colorless oil (202.4 mg) was treated with 1 equivalent amount of 4N hydrogen chloride/ethyl acetate to give the title compound (132.0 mg, 56%) as a white powder.

MS (ESI+):563 (M−HCl+H)

elemental analysis value: C₂₈H₃₃N₂O₂ClF₇.1.3H₂O

Found C, 54.04; H, 5.85; N, 4.43

Calculated C, 54.12; H, 5.77; N, 4.51

Melting point: 126-128° C.

¹H-NMR (300 MHz, DMSO-d₆):δ 1.12 (6H, s), 1.72-1.86 (2H, m), 1.87-2.06 (1H, m), 2.06-2.20 (1H, m), 2.33 (3H, s), 3.00 (3H, s), 3.03-3.21 (4H, m), 3.47-3.65 (2H, m), 3.66-3.79 (1H, m), 4.20 (1H, d, J=15.1 Hz), 4.54 (1H, s), 4.80 (1H, d, J=15.1 Hz), 6.83-6.96 (2H, m), 7.38 (1H, dd, J=8.1, 6.2 Hz)7.55 (2H, s), 7.93 (1H, s), 10.18 (1H, s)

### Example 124

### (3R*,4R*)—N-[3,5-bis(trifluoromethyl)benzyl]-1-[2-(5,5-dimethyl-2,4-dioxo-1,3-oxazolidin-3-yl)ethyl]-3-(4-fluoro-2-methylphenyl)-N-methylpiperidine-4-carboxamide monohydrochloride

(step 1)

To a solution of 5,5-dimethyl-1,3-oxazolidine-2,4-dione (500 mg) in DMF (10 mL) was added NaH (186 mg) at room temperature, and the mixture was stirred for 5 min. 1-Bromo-2-chloroethane (430 μL) was further added, and the mixture was stirred at room temperature for 4 days. The reaction mixture was concentrated under reduced pressure, and ethyl acetate was added to the residue. The organic layer was washed with aqueous citric acid solution and water and dried, and the solvent was evaporated under reduced pressure to give crude 3-(2-chloroethyl)-5,5-dimethyl-1,3-oxazolidine-2,4-dione as a colorless oil.

(step 2)

The title compound was obtained by reaction and purification in the same manner as in Example 123, step 2 and using the compound obtained in step 1.

MS (ESI+):632 (M−HCl+H)

### Example 125

### (3R*,4R*)—N-[3,5-bis(trifluoromethyl)benzyl]-1-[3-(5,5-dimethyl-2,4-dioxo-1,3-oxazolidin-3-yl)propyl]-3-(4-fluoro-2-methylphenyl)-N-methylpiperidine-4-carboxamide monohydrochloride

(step 1)

To a solution of 5,5-dimethyl-1,3-oxazolidine-2,4-dione (500 mg) in DMF (10 mL) was added NaH (186 mg) at room temperature, and the mixture was stirred for 5 min. 1-Bromo-3-chloropropane (508 μL) was further added, and the mixture was stirred at room temperature for 4 days. The reaction mixture was concentrated under reduced pressure, and ethyl acetate was added to the residue. The organic layer was washed with aqueous citric acid solution and water and dried, and the solvent was evaporated under reduced pressure to give crude 3-(3-chloropropyl)-5,5-dimethyl-1,3-oxazolidine-2,4-dione as a colorless oil.

(step 2)

The title compound was obtained by reaction and purification in the same manner as in Example 123, step 2 and using the compound obtained in step 1.

MS (ESI+):645 (M−HCl+H)

### Example 126

### (3R*,4R*)—N-[3,5-bis(trifluoromethyl)benzyl]-1-(6-chloropyridazin-3-yl)-3-(4-fluoro-2-methylphenyl)-N-methylpiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (200 mg) in THF (10 mL) was added Et₃N (59.8 μL), and the mixture was stirred at room temperature for 10 min. The precipitate was filtered off, and the filtrate was concentrated under reduced pressure. The residue was dissolved in DMF (2 mL), 3,6-dichloropyridazine (54.3 mg) was added, and the mixture was irradiated in a microwave reaction apparatus at 120° C. for 60 min. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (56.5 mg, 23%) as white crystals.

MS (ESI+):646 (M−HCl+H)

### Example 127

### (3R*,4R*)-1-[6-(acetylamino)pyridazin-3-yl]-N-[3,5-bis(trifluoromethyl)benzyl]-3-(4-fluoro-2-methylphenyl)-N-methylpiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (500 mg) in THF (10 mL) was added Et₃N (149 μL), and the mixture was stirred at room temperature for 20 min. The precipitate was filtered off, and the filtrate was concentrated under reduced pressure. The residue was dissolved in DMF (2 mL), N-(6-chloropyridazin-3-yl)acetamide (167 mg) synthesized by a known method (Journal of Organic Chemistry (1972), 37 (19), 2960-3) was added, and the mixture was irradiated in a microwave reaction apparatus at 100° C. for 60 min. The mixture was further irradiated in a microwave reaction apparatus at 150° C. for 60 min. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (39.6 mg, 6.3%) as white crystals.

MS (ESI+):612 (M−HCl+H)

### Example 128

### (3R*,4R*)—N-[3,5-bis(trifluoromethyl)benzyl]-3-(4-fluoro-2-methylphenyl)-N-methyl-1-(3-methyl-1,2,4-thiadiazol-5-yl)piperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (150 mg) in THF (5 mL) were added 5-chloro-3-methyl-1,2,4-thiadiazole (59 mg) and Et₃N (122 μL), and the mixture was stirred at room temperature for 19 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (49.9 mg, 28%) as white crystals.

MS (ESI+):575 (M−HCl+H)

### Example 129

### (3R*,4R*)-1-[5-(acetylamino)pyridin-2-yl]-N-[3,5-bis(trifluoromethyl)benzyl]-3-(4-fluoro-2-methylphenyl)-N-methylpiperidine-4-carboxamide monohydrochloride

To a solution of (3R*,4R*)—N-[3,5-bis(trifluoromethyl)benzyl]-3-(4-fluoro-2-methylphenyl)-N-methylpiperidine-4-carboxamide monohydrochloride (300 mg) in THF (10 mL) was added Et₃N (89.7 μL), and the mixture was stirred at room temperature for 30 min. The precipitate was filtered off, and the filtrate was concentrated under reduced pressure. 5-Acetamido-2-bromopyridine (132 mg) was added to the residue, and the mixture was stirred for 150° C. for 17 hr. After cooling, ethyl acetate was added to the reaction mixture. The organic layer was washed with saturated aqueous sodium hydrogen carbonate solution and water and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (65 mg, 17%) as white crystals.

MS (ESI+):611 (M−HCl+H)

elemental analysis value: C₃₀H₃₀N₂O₄ClF₇.1.1H₂O

Found C, 53.96; H, 4.73; N, 4.40

Calculated C, 54.04; H, 4.81; N, 8.40

Melting point: 213-215° C.

¹H-NMR (300 MHz, DMSO-d₆):δ 1.88-2.03 (2H, m), 2.03 (3H, s), 2.30 (3H, s), 3.03 (3H, s), 3.15-3.77 (4H, m), 4.00-4.16 (1H, m), 4.20 (1H, d, J=15.4 Hz), 4.28-4.40 (1H, m), 4.78 (1H, d, J=15.6 Hz), 6.83-6.96 (2H, m), 6.96-7.33 (2H, m), 7.36-7.50 (1H, m), 7.54 (2H, s), 7.77-7.90 (1H, m), 7.93 (1H, s), 8.33 (1H, s), 9.9.7 (1H, s)

### Example 130

### (3R*,4R*)—N-[3,5-bis(trifluoromethyl)benzyl]-3-(4-fluoro-2-methylphenyl)-N-methyl-1-(5-nitro-1,3-thiazol-2-yl)piperidine-4-carboxamide

To a solution of (3R*,4R*)—N-[3,5-bis(trifluoromethyl)benzyl]-3-(4-fluoro-2-methylphenyl)-N-methylpiperidine-4-carboxamide monohydrochloride (300 mg) in EtOH (10 mL) were added 2-bromo-5-nitrothiazole (208 mg) and sodium hydrogen carbonate (90.1 mg), and the mixture was heated under reflux for 17 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (481 mg, 82%) as white crystals.

MS (ESI+):605 (M+H)

### Example 131

### 2,2,2-trichloroethyl (3R*,4R*)-4-{[[3,5-bis(trifluoromethyl)benzyl](methyl)amino]carbonyl}-3-(4-fluoro-2-methylphenyl)piperidine-1-carboxylate

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 2,2,2-trichloroethyl chloroformate.

elemental analysis value: C₂₆H₂₄N₂O₃Cl₃F₇.H₂O

Found C, 47.78; H, 3.73; N, 4.29

Calculated C, 47.56; H, 3.84; N, 4.19

### Example 132

### (3R*,4R*)—N-[3,5-bis(trifluoromethyl)benzyl]-1-(ethylsulfonyl)-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and ethanesulfonyl chloride.

MS (ESI+):569 (M+H)

elemental analysis value: C₂₅H₂₇N₂O₃SF₇

Found C, 52.83; H, 4.74; N, 4.89

Calculated C, 52.81; H, 4.79; N, 4.93

Melting point: 170-171° C.

¹H-NMR (300 MHz, CDCl₃):δ 1.38 (3H, t, J=7 Hz), 1.91-2.08 (2H, m), 2.42-2.47 (3H, m), 2.73-3.15 (8H, m), 3.52-4.04 (3H, m), 4.17-4.87 (2H, m), 6.70-7.85 (6H, m)

### Example 133

### (3R*,4R*)—N-[3,5-bis(trifluoromethyl)benzyl]-1-(cyclopropylsulfonyl)-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and cyclopropanesulfonyl chloride.

MS (ESI+):581 (M+H)

### Example 134

### (3R*,4R*)—N-[3,5-bis(trifluoromethyl)benzyl]-3-(4-fluoro-2-methylphenyl)-1-(isobutylsulfonyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 2-methyl-1-propylsulfonyl chloride.

MS (ESI+):597 (M+H)

### Example 135

### Benzyl 4-{[(3R*,4R*)-4-{[[3,5-bis(trifluoromethyl)benzyl](methyl)amino]carbonyl}-3-(4-fluoro-2-methylphenyl)piperidin-1-yl]sulfonyl}piperidine-1-carboxylate

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and benzyl 4-(chlorosulfonyl)piperidine-1-carboxylate.

MS (ESI+):758 (M+H)

### Example 136

### (3R*,4R*)—N-[3,5-bis(trifluoromethyl)benzyl]-3-(4-fluoro-2-methylphenyl)-1-[(1-glycoloylpiperidin-4-yl)sulfonyl]-N-methylpiperidine-4-carboxamide

(step 1)

A suspension of the compound (616 mg) obtained in Example 135 and 10% palladium-carbon (120 mg) in EtOH (10 mL) was stirred under 1 atm of hydrogen atmosphere at room temperature for 30 hr. The catalyst was filtered off, and the filtrate was concentrated under reduced pressure. The obtained oil was treated with 1 equivalent amount of 4N hydrogen chloride/ethyl acetate to give (3R*,4R*)—N-[3,5-bis(trifluoromethyl)benzyl]-3-(4-fluoro-2-methylphenyl)-N-methyl-1-(piperidin-4-ylsulfonyl)piperidine-4-carboxamide monohydrochloride (438 mg, 82%) as a white powder.

MS (ESI+):624 (M−HCl+H)

(step 2)

The title compound was obtained by reaction and purification in the same manner as in Example 1 and using the compound obtained in step 1 and glycolic acid.

MS (ESI+):682 (M+H)

### Example 137

### (3R*,4R*)—N-[3,5-bis(trifluoromethyl)benzyl]-3-(4-fluoro-2-methylphenyl)-1-[(2-hydroxyethyl)sulfonyl]-N-methylpiperidine-4-carboxamide

To a solution of (3R*,4R*)—N-[3,5-bis(trifluoromethyl)benzyl]-3-(4-fluoro-2-methylphenyl)-N-methylpiperidine-4-carboxamide monohydrochloride (500 mg) and Et₃N (340 μL) in THF (10 mL) was added 2-chloroethanesulfonyl chloride (201 mg), and the mixture was stirred at room temperature for 18 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (solvent gradient; 0→100% ethyl acetate/hexane). A fraction with a short retention time was concentrated to give (3R*,4R*)—N-[3,5-bis(trifluoromethyl)benzyl]-3-(4-fluoro-2-methylphenyl)-N-methyl-1-(vinylsulfonyl)piperidine-4-carboxamide (138 mg, 24%) as a white powder, and a fraction with a long retention time was concentrated to give the title compound (103 mg, 18%) as a white amorphous solid.

Compound with a short retention time

MS (ESI+):567 (M+H)

Compound with a long retention time (compound of Example 137)

MS (ESI+):585 (M+H)

### Example 138

### (3R*,4R*)—N-[3,5-bis(trifluoromethyl)benzyl]-1-[(2-ethoxyethyl)sulfonyl]-3-(4-fluoro-2-methylphenyl)-N-methylpiperidine-4-carboxamide

To a solution of (3R*,4R*)—N-[3,5-bis(trifluoromethyl)benzyl]-3-(4-fluoro-2-methylphenyl)-N-methyl-1-(vinylsulfonyl)piperidine-4-carboxamide (411 mg) in a mixture of EtOH (4 mL) and THF (4 mL) was added 1N aqueous sodium hydroxide solution (3.4 mL), and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (92 mg, 22%) as a white powder.

MS (ESI+):613 (M+H)

elemental analysis value: C₂₇H₃₁N₂O₄SF₇

Found C, 52.74; H, 4.01; N, 4.48

Calculated C, 52.94; H, 5.10; N, 4.57

Melting point: 151-153° C.

¹H-NMR (300 MHz, CDCl₃):δ 1.22 (3H, t, J=7.0 Hz), 1.91-2.13 (2H, m), 2.42 (3H, s), 2.79 (1H, t, J=12.1 Hz), 2.90-3.00 (1H, m), 3.96 (3H, s), 3.06 (1H, dt, J=11.1, 4.4 Hz), 3.23 (2H, t, J=6.1 Hz), 3.52 (2H, q, J=7.2 Hz), 3.58 (1H, dt, J=11.1, 4.2 Hz), 3.73-3.79 (1H, m), 3.78 (2H, t, J=5.9 Hz), 3.99 (1H, d, J=12.5 Hz), 4.19 (1H, d, J=15.1 Hz), 4.84 (1H, d, J=15.1 Hz), 6.77 (1H, dt, J=8.2, 2.5 Hz), 6.83 (1H, dd, J=9.8, 2.7 Hz), 6.98 (1H, dd, J=8.5, 5.5 Hz), 7.36 (2H, s), 7.73 (1H, s)

### Example 139

### (3R*,4R*)—N-[3,5-bis(trifluoromethyl)benzyl]-3-(4-fluoro-2-methylphenyl)-N-methyl-1-[(2-morpholin-4-ylethyl)sulfonyl]piperidine-4-carboxamide monohydrochloride

(3R*,4R*)—N-[3,5-bis(Trifluoromethyl)benzyl]-3-(4-fluoro-2-methylphenyl)-N-methyl-1-(vinylsulfonyl)piperidine-4-carboxamide (238 mg) and morpholine (2 mL) were heated at 80° C. for 6 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The obtained oil was treated with 1 equivalent amount of 4N hydrogen chloride/ethyl acetate to give the title compound (151 mg, 52%) as a white powder.

MS (ESI+):654 (M−HCl+H)

Melting point: 196-198° C.

¹H-NMR (300 MHz, DMSO-d₆):δ 1.56-1.74 (1H, m), 1.97-2.10 (1H, m), 2.30 (3H, s), 2.44-2.57 (4H, m), 2.81-2.98 (2H, m), 3.02 (3H, s), 3.06-3.21 (3H, m), 3.23-3.83 (7H, m), 3.86-4.09 (2H, m), 4.20 (1H, d, J=14.9 Hz), 4.82 (1H, d, J=15.1 Hz), 6.82-6.94 (2H, m), 7.31-7.40 (1H, m), 7.55 (2H, s), 7.93 (1H, s)

### Example 140

### (3R*,4R*)—N-[3,5-bis(trifluoromethyl)benzyl]-1-[2-(5,5-dimethyl-2,4-dioxo-1,3-oxazolidin-3-yl)ethyl]sulfonyl}-3-(4-fluoro-2-methylphenyl)-N-methylpiperidine-4-carboxamide

A solution of (3R*,4R*)—N-[3,5-bis(trifluoromethyl)benzyl]-3-(4-fluoro-2-methylphenyl)-N-methyl-1-(vinylsulfonyl)piperidine-4-carboxamide (150 mg), 5,5-dimethyloxazolidine-2,4-dione (38.5. mg) and potassium carbonate (41.5 mg) in CH₃CN (10 mL) was refluxed under heating for 17 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (119 mg, 30%) as a white powder.

MS (ESI+):696 (M+H)

### Example 141

### (3R*,4R*)—N-[3,5-bis(trifluoromethyl)benzyl]-1-({[(1S,4R)-7,7-dimethyl-2-oxobicyclo[2.2.1]hept-1-yl]methyl}sulfonyl)-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride.

MS (ESI+):691 (M+H)

### Example 142

### (3R*,4R*)—N-[3,5-bis(trifluoromethyl)benzyl]-1-[(dimethylamino)sulfonyl]-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and dimethylsulfamoyl chloride.

MS (ESI+):584 (M+H)

elemental analysis value: C₂₅H₂₈N₃O₅SF₇

Found C, 51.56; H, 4.81; N, 7.27

Calculated C, 51.45; H, 4.84; N, 7.20

Melting point: 142-144° C.

¹H-NMR (300 MHz, CDCl₃):δ 1.91-2.11 (2H, m), 2.41 (3H, s), 2.77 (1H, t, J=12.0 Hz), 2.84 (6H, s), 2.87-3.00 (1H, m), 2.96 (3H, s), 3.07 (1H, dt, J=10.5, 5.4 Hz), 3.54 (1H, dt, J=11.1, 4.2 Hz), 3.64 (1H, dd, J=12.1, 2.8 Hz), 3.89 (1H, d, J=11.9 Hz), 4.21 (1H, d, J=15.1 Hz), 4.81 (1H, d, J=14.9 Hz), 6.74-6.87 (2H, m), 7.01 (1H, dd, J=8.3, 5.7 Hz), 7.37 (2H, s), 7.73 (1H, S)

### Example 143

### (3R*,4R*)-1-{[4-(acetylamino)phenyl]sulfonyl}-N-[3,5-bis(trifluoromethyl)benzyl]-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 4-acetamidobenzenesulfonyl chloride.

MS (ESI+):674 (M+H)

### Example 144

### (3R*,4R*)—N-[3,5-bis(trifluoromethyl)benzyl]-3-(4-fluoro-2-methylphenyl)-N-methyl-1-(pyridin-2-ylsulfonyl)piperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 2-chlorosulfonyl-pyridinium chloride.

MS (ESI+):618 (M+H)

elemental analysis value: C₂₈H₂₆N₃O₅SF₇

Found C, 54.42; H, 4.33; N, 6.67

Calculated C, 54.45; H, 4.24; N, 6.80

Melting point: 187-189° C.

¹H-NMR (300 MHz, CDCl₃):δ 1.82-2.09 (2H, m), 2.38 (3H, s), 2.74 (1H, t, J=12.3 Hz), 2.92 (3H, s), 2.94-3.09 (2H, m), 3.57 (1H, dt, J=11.4, 4.2 Hz), 3.97 (1H, dd, J=12.9, 2.7 Hz), 4.16 (1H, d, J=15.3 Hz), 4.23 (1H, d, J=13.3 Hz), 4.82 (1H, d, J=15.1 Hz), 6.73 (1H, dt, J=8.3, 2.7 Hz), 6.81 (1H, dd, J=9.8, 2.7 Hz), 6.93 (1H, dd, J=8.7, 5.7 Hz), 7.34 (2H, s), 7.51 (1H, ddd, J=7.0, 5.1, 1.1 Hz), 7.72 (1H, s), 7.87-8.00 (2H, m), 8.74 (1H, d, J=4.5 Hz)

### Example 145

### (3R*,4R*)—N-[3,5-bis(trifluoromethyl)benzyl]-3-(4-fluoro-2-methylphenyl)-N-methyl-1-(pyridin-3-ylsulfonyl)piperidine-4-carboxamide monohydrochloride

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and pyridine-3-sulfonyl chloride, and a treatment of the obtained resultant product with 1 equivalent amount of 4N hydrogen chloride/ethyl acetate.

MS (ESI+):618 (M−HCl+H)

### Example 146

### (3R*,4R*)—N-[3,5-bis(trifluoromethyl)benzyl]-1-[(6-chloropyridin-3-yl)sulfonyl]-3-(4-fluoro-2-methylphenyl)-N-methylpiperidine-4-carboxamide

The title compound was obtained by reaction and purification in the same manner as in Example 11 and using (3R*,4R*)—N-[3,5-bis(trifluoromethyl)benzyl]-3-(4-fluoro-2-methylphenyl)-N-methylpiperidine-4-carboxamide monohydrochloride and 6-chloro-pyridine-3-sulfonyl chloride.

MS (ESI+):652 (M+H)

### Example 147

### (3R*,4R*)—N-[3,5-bis(trifluoromethyl)benzyl]-3-(4-fluoro-2-methylphenyl)-N-methyl-1-[(6-morpholin-4-ylpyridin-3-yl)sulfonyl]piperidine-4-carboxamide monohydrochloride

A mixture of the compound (142 mg) obtained in Example 146 and morpholine (2 mL) was stirred at room temperature for 9 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give a colorless oil. The obtained oil was treated with 1 equivalent amount of 4N hydrogen chloride/ethyl acetate to give the title compound (86.6 mg, 54%) as a white powder.

MS (ESI+):703 (M−HCl+H)

### Example 148

### (3S,4S)—N-[3,5-bis(trifluoromethyl)benzyl]-1-[(5,5-dimethyl-2,4-dioxo-1,3-oxazolidin-3-yl)acetyl]-3-(4-fluoro-2-methylphenyl)-N-methylpiperidine-4-carboxamide

To a solution of (3S,4S)—N-[3,5-bis(trifluoromethyl)benzyl]-3-(4-fluoro-2-methylphenyl)-N-methylpiperidine-4-carboxamide monohydrochloride (615 mg) synthesized by a known method (WO2006/004195), (5,5-dimethyl-2,4-dioxo-1,3-oxazolidin-3-yl)acetic acid (292 mg) and Et₃N (217 μL) in CH₃CN (6 mL) were added WSC.HCl (299 mg) and HOBt.H₂O (184 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 10→67% ethyl-acetate/hexane) to give the title compound (671 mg, 87%) as a white powder.

MS (ESI+):646 (M+H)

[α]_{D}²⁵−12.9° (c 1.01, MeOH)

elemental analysis value: C₃₀H₃₀N₃O₅F₇.0.1IPE

Found C, 56.10; H, 5.02; N, 6.28

Calculated C, 56.04; H, 4.83; N, 6.41

¹H-NMR (300 MHz, CDCl₃):δ 1.65 (6H, s), 1.90-2.04 (2H, m), 2.36-4.91 (16H, m), 6.74-7.09 (3H, m), 7.36-7.43 (2H, m), 7.73-7.85 (1H, m)

### Example 149

### (3S,4S)—N-[3,5-bis(trifluoromethyl)benzyl]-3-(4-fluoro-2-methylphenyl)-1-[(1-glycoloylpiperidin-4-yl)carbonyl]-N-methylpiperidine-4-carboxamide

To a solution of (3S,4S)—N-[3,5-bis(trifluoromethyl)benzyl]-3-(4-fluoro-2-methylphenyl)-N-methylpiperidine-4-carboxamide monohydrochloride (200 mg) synthesized by a known method (WO2006/004195), the compound (94.9 mg) obtained in Reference Example 11 and Et₃N (163 μL) in THF (10 mL) were added WSC.HCl (114 mg) and HOBt.H₂O (90.5 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (25.1 mg, 10%) as a white powder.

MS (ESI+):646 (M+H)

¹H-NMR (300 MHz, CDCl₃): δ 1.65-2.02 (4H, m), 2.39 (3H, d, J=12.9 Hz), 2.43-2.90 (4H, m), 2.96 (3H, d, J=5.3 Hz), 3.00-3.14 (2H, m), 3.15-3.27 (1H, m), 3.29-3.52 (1H, m), 3.51-3.68 (2H, m), 3.73-3.91 (1H, m), 4.04-4.22 (3H, m), 4.24-4.61 (2H, m), 4.61-4.79 (1H, m), 4.88 (1H, d, J=15.1 Hz), 6.14-6.92 (2H, m), 7.00-7.13 (1H, m), 7.37 (2H, d, J=12.1 Hz), 7.74 (1H, s)

### Example 150

### (3S,4S)—N-[3,5-bis(trifluoromethyl)benzyl]-3-(4-fluoro-2-methylphenyl)-1-({1-[(2S)-2-hydroxypropanoyl]piperidin-4-yl}carbonyl)-N-methylpiperidine-4-carboxamide

To a solution of (3S,4S)—N-[3,5-bis(trifluoromethyl)benzyl]-3-(4-fluoro-2-methylphenyl)-N-methylpiperidine-4-carboxamide monohydrochloride (200 mg) synthesized by a known method (WO2006/004195), the compound (102 mg) obtained in Reference Example 128 and Et₃N (163 μL) in THF (10 mL) were added WSC.HCl (114 mg) and HOBt.H₂O (90.5 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (87.2 mg, 34%) as a white powder.

MS (ESI+):660 (M+H)

[α]_{D}²⁵−9.1° (c 1.00, MeOH)

elemental analysis value: C₃₂H₃₆N₃O₄F₇.0.3H₂O

Found C, 57.79; H, 5.61; N, 6.18

Calculated C, 57.79; H, 5.55; N, 6.32

¹H-NMR (300 MHz, CDCl₃):δ 1.22-1.42 (4H, m), 1.66-2.02 (3H, m), 2.40 (3H, d, J=12.3 Hz), 2.44-2.91 (4H, m), 2.93 (3H, s), 3.03-3.16 (2H, m), 3.15-3.29 (1H, m), 3.31-3.51 (1H, m), 3.71-3.96 (3H, m), 4.00-4.38 (2H, m), 4.40-4.60 (2H, m), 4.60-4.80 (1H, m), 4.83-4.95 (1H, m), 6.72-6.91 (2H, m), 7.01-7.13 (1H, m), 7.37 (2H, d, J=11.4 Hz), 7.74 (1H, s)

### Example 151

### (3S,4S)—N-[3,5-bis(trifluoromethyl)benzyl]-3-(4-fluoro-2-methylphenyl)-1-({1-[(2R)-2-hydroxypropanoyl]piperidin-4-yl}carbonyl)-N-methylpiperidine-4-carboxamide

To a solution of (3S,4S)—N-[3,5-bis(trifluoromethyl)benzyl]-3-(4-fluoro-2-methylphenyl)-N-methylpiperidine-4-carboxamide monohydrochloride (200 mg) synthesized by a known method (WO2006/004195), the compound (102 mg) obtained in Reference Example 129 and Et₃N (163 μL) in THF (10 mL) were added WSC.HCl (114 mg) and HOBt.H₂O (90.5 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (100.5 mg, 39%) as a white powder.

MS (ESI+):660 (M+H)

[α]_{D}²⁵−6.7° (c 1.00, MeOH)

elemental analysis value: C₃₂H₃₆N₃O₄F₇.0.3H₂O

Found C, 57.68; H, 5.57; N, 6.23

Calculated C, 57.79; H, 5.55; N, 6.32

¹H-NMR (300 MHz, CDCl₃):δ 1.28-1.46 (4H, m), 1.66-2.01 (3H, m), 2.39 (3H, d, J=12.5 Hz), 2.44-2.92 (4H, m), 2.97 (3H, s), 3.00-3.15 (2H, m), 3.16-3.30 (1H, m), 3.30-3.53 (1H, m), 3.72-3.92 (3H, m), 4.02-4.35 (2H, m), 4.40-4.55 (2H, m), 4.69 (1H, d, J=14.8 Hz), 4.88 (1H, d, J=14.8 Hz), 6.74-6.93 (2H, m), 7.00-7.14 (1H, m), 7.37 (2H, d, J=13.3 Hz), 7.74 (1H, s)

### Example 152

### (3S,4S)—N-{(1S)-1-[3,5-bis(trifluoromethyl)phenyl]ethyl}-3-(4-fluoro-2-methylphenyl)-1-[(1-glycoloylpiperidin-4-yl)carbonyl]-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (158 mg) synthesized by a known method (WO2006/004195), the compound (67.4 mg) obtained in Reference Example 11 and Et₃N (50.2 μL) in CH₃CN (3 mL) were added WSC.HCl (69.0 mg) and HOBt.H₂O (55.1 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 0→9% MeOH/ethyl acetate) to give the title compound (136 mg, 69%) as a white crystal powder.

MS (ESI+):660 (M+H)

[α]_{D}²⁵−53.9° (c 1.01, MeOH)

elemental analysis value: C₃₂H₃₆N₃O₄F₇.0.5H₂O

Found C, 57.22; H, 5.69; N, 6.26

Calculated C, 57.48; H, 5.58; N, 6.28

Melting point: 108-116° C.

¹H-NMR (300 MHz, CDCl₃):δ 1.47 (3H, d, J=7 Hz), 1.70-2.00 (7H, m), 2.40-4.90 (19H, m), 5.86-5.93 (1H, m), 6.75-7.84 (6H, m)

### Example 153

### (3S,4S)—N-{(1R)-1-[3,5-bis(trifluoromethyl)phenyl]ethyl}-3-(4-fluoro-2-methylphenyl)-1-[(1-glycoloylpiperidin-4-yl)carbonyl]-N-methylpiperidine-4-carboxamide

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (158 mg) synthesized by a known method (WO2006/004195), the compound (67.4 mg) obtained in Reference Example 11 and Et₃N (50.2 μL) in CH₃CN (3 mL) were added WSC.HCl (69.0 mg) and HOBt.H₂O (55.1 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 0→5% MeOH/ethyl acetate) to give the title compound (120 mg, 61%) as a white amorphous solid.

MS (ESI+):660 (M+H)

[α]_{D}²⁵+87.7° (c 1.00, MeOH)

elemental analysis value: C₃₂H₃₆N₃O₄F₇

Found C, 58.23; H, 5.75; N, 6.15

Calculated C, 58.27; H, 5.50; N, 6.37

¹H-NMR (300 MHz, CDCl₃):δ 1.17 (3H, d, J=7 Hz), 1.70-2.00 (7H, m), 2.43-5.29 (19H, m), 5.91 (1H, m), 6.63-7.81 (6H, m)

### Example 154

### (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (263 mg) synthesized by a known method (WO2006/004195), (5,5-dimethyl-2,4-dioxo-1,3-oxazolidin-3-yl)acetic acid (122 mg) and Et₃N (91 μL) in CH₃CN (4 mL) were added WSC.HCl (125 mg) and HOBt.H₂O (77 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 10→67% ethyl acetate/hexane) to give the title compound (300 mg, 91%) as a white powder.

MS (ESI+):660 (M+H)

[α]_{D}²⁵−54.7° (c 1.01, MeOH)

elemental analysis value: C₃₁H₃₂N₃O₅F₇

Found C, 56.48; H, 5.08; N, 6.20

Calculated C, 56.45; H, 4.89; N, 6.37

¹H-NMR (300 MHz, CDCl₃):δ 1.47 (3H, d, J=7 Hz), 1.65 (6H, s), 1.90-2.10 (2H, m), 2.40-4.79 (14H, m), 5.88-5.94 (1H, m), 6.71-7.84 (6H, m)

### Example 155

### (3S,4S)—N-{(1R)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (263 mg) synthesized by a known method (WO2006/004195), (5,5-dimethyl-2,4-dioxo-1,3-oxazolidin-3-yl)acetic acid (122 mg) and Et₃N (91 μL) in CH₃CN (4 mL) were added WSC.HCl (125 mg) and HOBt.H₂O (77 mg), and the mixture was stirred at room temperature for 3 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 10→67% ethyl acetate/hexane) to give the title compound (306 mg, 93%) as a white powder.

MS (ESI+):660 (M+H)

[α]_{D}²⁵+77.5° (c 1.01, MeOH)

elemental analysis value: C₃₁H₃₂N₃O₅F₇

Found C, 56.45; H, 5.05; N, 6.21

Calculated C, 56.45; H, 4.89; N, 6.37

¹H-NMR (300 MHz, CDCl₃):δ 1.15-1.18 (3H, m), 1.66 (6H, m), 1.90-2.10 (2H, m), 2.42-5.29 (14H, m), 5.87-5.94 (1H, m), 6.83-7.81 (6H, m)

### Example 156

### (3S,4S)-1-(2-amino-2-oxoethyl)-N-{(1S)-1-[3,5-bis(trifluoromethyl)phenyl]ethyl}-3-(4-fluoro-2-methylphenyl)-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (211 mg) synthesized by a known method (WO2006/004195) and 2-iodoacetamide (104 mg) in DMF (3 mL) was added Et₃N (139 μL) at room temperature, and the mixture was stirred at room temperature for 16 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 10→100% ethyl acetate/hexane) to give the title compound (212 mg, 97%) as a white crystal powder.

MS (ESI+):548 (M+H)

[α]_{D}²⁵−43.8° (c 1.00, MeOH)

elemental analysis value: C₂₆H₂₈N₃O₂F₇.0.1 hexane

Found C, 57.64; H, 5.60; N, 7.35

Calculated C, 57.45; H, 5.33; N, 7.56

Melting point: 161-164° C.

¹H-NMR (300 MHz, CDCl₃):δ 1.16-1.47 (3H, m), 1.87-2.08 (2H, m), 2.18-2.36 (2H, m), 2.42 (3H, s), 2.48-2.65 (3H, m), 2.88-3.11 (5H, m), 3.61 (1H, dt, J=11,4 Hz), 5.10-5.92 (2H, m), 6.72-7.03 (3H, m), 7.11 (1H, br), 7.28-7.83 (3H, m)

### Example 157

### (3S,4S)-1-(2-amino-2-oxoethyl)-N-{(1R)-1-[3,5-bis(trifluoromethyl)phenyl]ethyl}-3-(4-fluoro-2-methylphenyl)-N-methylpiperidine-4-carboxamide monohydrochloride

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (211 mg) synthesized by a known method (WO2006/004195) and 2-iodoacetamide (104 mg) in DMF (3 mL) was added Et₃N (139 μL) at room temperature, and the mixture was stirred at room temperature for 16 hr. The reaction mixture was poured into water, and the resultant product was extracted with ethyl acetate. The organic layer was washed with water and saturated brine and dried, and the solvent was evaporated under reduced pressure. The obtained residue was purified by silica gel column chromatography (NH Chromatorex) (solvent gradient; 10→100% ethyl acetate/hexane). The thus-obtained colorless oil was treated with 1 equivalent amount of 4N hydrogen chloride/ethyl acetate to give the title compound (216 mg, 92%) as a white crystal powder.

MS (ESI+):548 (M−HCl+H)

[α]_{D}²⁵+89.3° (c 1.00, MeOH)

elemental analysis value: C₂₆H₂₈N₃O₂F₇.0.5H₂O

Found C, 52.67; H, 5.37; N, 6.97

Calculated C, 52.66; H, 5.10; N, 7.09

Melting point: 133-139° C.

¹H-NMR (300 MHz, CDCl₃):δ 1.16-1.70 (3H, m), 1.85-3.74 (14H, m), 5.26-5.95 (2H, m), 6.60-7.80 (7H, m) (NMR characterization was carried out as its free amine.)

### Example 158

### (3S,4S)—N-{(1S)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (150 mg) synthesized by a known method (WO2006/004195), pyrimidine-5-carboxylic acid (47.1 mg) and Et₃N (119 μL) in THF (5 mL) were added WSC.HCl (83.5 mg) and HOBt.H₂O (66.7 mg), and the mixture was stirred at room temperature for 4 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (137 mg, 81%) as a white crystal powder.

MS (ESI+):597 (M+H)

[α]_{D}²⁵−73.7° (c 0.90, MeOH)

elemental analysis value: C₂₉H₂₇N₄O₂F₇.0.2H₂O

Found C, 58.08; H, 4.48; N, 9.20

Calculated C, 58.04; H, 4.60; N, 9.34

Melting point: 185-188° C.

¹H-NMR (300 MHz, CDCl₃):δ 1.47 (3H, d, J=6.4 Hz), 1.81-2.15 (2H, m), 2.39-2.56 (3H, m), 2.68 (3H, s), 2.85-3.02 (1H, m), 3.05-3.37 (2H, m), 3.46-4.01 (2H, m), 4.68-5.04 (1H, m), 5.91 (1H, q, J=6.6 Hz), 6.70-6.88 (2H, m), 6.94-7.09 (1H, m), 7.26 (2H, s), 7.73 (1H, s), 8.86 (2H, s), 9.28 (1H, s)

### Example 159

### (3S,4S)—N-{(1R)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (150 mg) synthesized by a known method (WO2006/004195), pyrimidine-5-carboxylic acid (47.1 mg) and Et₃N (119 μL) in THF (5 mL) were added WSC.HCl (83.5 mg) and HOBt.H₂O (66.7 mg), and the mixture was stirred at room temperature for 4 days. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (135 mg, 80%) as a white amorphous solid.

MS (ESI+):597 (M+H)

[α]_{D}²⁵+80.1° (c 0.95, MeOH)

elemental analysis value: C₂₉H₂₇N₄O₂F₇.0.3H₂O

Found C, 57.82; H, 4.65; N, 9.16

Calculated C, 57.87; H, 4.62; N, 9.31

¹H-NMR (300 MHz, CDCl₃):δ 1.46 (3H, d, J=7.0 Hz), 1.81-2.15 (2H, m), 2.27-2.53 (3H, m), 2.59 (3H, s), 2.73-3.02 (1H, m), 3.13-3.41 (1H, m), 3.20 (1H, dt, J=10.8, 4.1 Hz), 3.45-4.04 (2H, m), 4.72-5.09 (1H, m), 5.91 (1H, q, J=6.9 Hz), 6.78-6.89 (2H, m), 7.02-7.19 (1H, m), 7.58 (2H, s), 7.77 (1H, s), 8.86 (2H, s), 9.29 (1H, s)

### Example 160

### (3S,4S)—N-{(1S)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (120 mg) synthesized by a known method (WO2006/004195), 3-(methylsulfonyl)propanoic acid (45.1 mg) and Et₃N (95.2 μL) in THF (5 mL) were added WSC.HCl (66.8 mg) and HOBt.H₂O (52.8 mg), and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (98.1 mg, 69%) as a white amorphous solid.

MS (ESI+):625 (M+H)

[α]_{D}²⁵−46.4° (c 1.01, MeOH)

elemental analysis value: C₂₈H₃₁N₂O₄SF₇.0.3H₂O

Found C, 53.39; H, 5.04; N, 4.26

Calculated C, 53.38; H, 5.06; N, 4.45

¹H-NMR (300 MHz, CDCl₃):δ 1.46 (3H, d, J=7.2 Hz), 1.78-2.02 (2H, m), 2.42 (3H, d, J=11.4 Hz), 2.48-2.59 (1H, m), 2.67 (3H, d, J=2.7 Hz), 2.68-2.78 (1H, m), 2.89 (1H, t, J=7.2 Hz), 2.93-3.29 (3H, m), 2.99 (3H, d, J=3.4 Hz), 3.34-3.56 (2H, m), 3.77-4.11 (1H, m), 4.59-4.90 (1H, m), 5.90 (1H, q, J=6.4 Hz), 6.72-6.92 (2H, m), 6.96-7.07 (1H, m), 7.28 (2H, s), 7.72 (1H, s)

### Example 161

### (3S,4S)—N-{(1R)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide

To a solution of (3S,4S)—N-{(1R)-1-[3,5-bis(trifluoromethyl)phenyl]ethyl}-3-(4-fluoro-2-methylphenyl)-N-methylpiperidine-4-carboxamide monohydrochloride (120 mg) synthesized by a known method (WO2006/004195), 3-(methylsulfonyl)propanoic acid (45.1 mg) and Et₃N (95.2 μL) in THF (5 mL) were added WSC.HCl (66.8 mg) and HOBt.H₂O (52.8 mg), and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (85.2 mg, 60%) as a white amorphous solid.

MS (ESI+):625 (M+H)

[α]_{D}²⁵+95.1° (c 1.01, MeOH)

elemental analysis value: C₂₈H₃₁N₂O₄SF₇.0.2H₂O

Found C, 53.50; H, 5.18; N, 4.36

Calculated C, 53.53; H, 5.04; N, 4.46

¹H-NMR (300 MHz, CDCl₃):δ 1.36 (3H, d, J=8.1 Hz), 1.86-2.00 (2H, m), 2.44 (3H, d, J=10.6 Hz), 2.47-2.66 (1H, m), 2.58 (3H, s), 2.86-2.93 (1H, m), 2.93-3.02 (1H, m), 2.99 (3H, s), 3.08-3.29 (2H, m), 3.33-3.59 (3H, m), .3.80-4.11 (1H, m), 4.63-4.89 (1H, m), 5.91 (1H, q, J=6.6 Hz), 6.82-6.96 (2H, m), 7.06-7.15 (1H, m), 7.58 (2H, s), 7.77 (1H, s)

### Example 162

### (3S,4S)—N-{(1S)-1-[3,5-bis(trifluoromethyl)phenyl]ethyl}-1-(cyclopropylsulfonyl)-3-(4-fluoro-2-methylphenyl)-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (200 mg) synthesized by a known method (WO2006/004195) and Et₃N (132 μL) in THF (5 mL) was added cyclopropanesulfonyl chloride (64.0 mg) at 0° C., and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (137 mg, 61%) as a white crystal powder.

MS (ESI+):595 (M+H)

[α]_{D}²⁵−21.3° (c 1.03, MeOH)

Melting point: 154-156° C.

¹H-NMR (300 MHz, CDCl₃):δ 0.94-1.08 (2H, m), 1.16-1.27 (2H, m), 1.47 (3H, d, J=1.8 Hz), 1.93-2.13 (2H, m), 2.26-2.37 (1H, m), 2.45 (3H, s), 2.69 (3H, s), 2.79 (1H, t, J=12.1 Hz), 2.92-3.12 (2H, m), 3.57-3.66 (1H, m), 3.82 (1H, dd, J=12.5, 3.0 Hz), 4.04 (1H, d, J=12.1 Hz), 5.91 (1H, q, J=6.9 Hz), 6.77 (1H, dt, J=8.3, 2.7 Hz), 6.85 (1H, dd, J=9.8, 2.3 Hz), 7.00 (1H, dd, J=8.3, 5.7 Hz), 7.29 (2H, s), 7.72 (1H, s)

### Example 163

### (3S,4S)—N-[3,5-bis(trifluoromethyl)benzyl]-N-cyclopropyl-3-(4-fluoro-2-methylphenyl)-1-[(1-glycoloylpiperidin-4-yl)carbonyl]piperidine-4-carboxamide

The title compound was obtained by reaction and purification in the same manner as in Example 1 and using the compound obtained in Reference Example 79 and the compound obtained in Reference Example 11.

MS (ESI+):672 (M+H)

### Example 164

### (3R*,4R*)—N-[3,5-bis(trifluoromethyl)benzyl]-N-cyclopropyl-3-(4-fluoro-2-methylphenyl)-1-[3-(methylsulfonyl)propanoyl]piperidine-4-carboxamide

The title compound was obtained by reaction and purification in the same manner as in Example 1 and using the compound obtained in Reference Example 79.

MS (ESI+):637 (M+H)

### Example 165

### (3R*,4R*)—N-[3,5-bis(trifluoromethyl)benzyl]-N-cyclopropyl-3-(4-fluoro-2-methylphenyl)-1-(pyrimidin-5-ylcarbonyl)piperidine-4-carboxamide

The title compound was obtained by reaction and purification in the same manner as in Example 1 and using the compound obtained in Reference Example 79 and pyrimidine-5-carboxylic acid.

MS (ESI+):609 (M+H)

### Example 166

### (3R*,4R*)-1-{[4-(acetylamino)phenyl]sulfonyl}-N-[3,5-bis(trifluoromethyl)benzyl]-N-cyclopropyl-3-(4-fluoro-2-methylphenyl)piperidine-4-carboxamide

The title compound was obtained by reaction and purification in the same manner as in Example 11 and using the compound obtained in Reference Example 79 and 4-acetamidobenzenesulfonyl chloride.

MS (ESI+):700 (M+H)

### Example 167

### (3R*,4R*)—N-[3,5-bis(trifluoromethyl)benzyl]-N-cyclopropyl-3-(4-fluoro-2-methylphenyl)-1-(pyridin-3-ylsulfonyl)piperidine-4-carboxamide monohydrochloride

The title compound was obtained by reaction and purification in the same manner as in Example 11 and using the compound obtained in Reference Example 79 and pyridine-3-sulfonyl chloride, and a treatment of the obtained resultant product with 1 equivalent amount of 4N hydrogen chloride/ethyl acetate.

MS (ESI+):644 (M−HCl+H)

### Example 168

### (3R*,4R*)-1-[(1-acetylpiperidin-4-yl)methyl]-N-[3,5-bis(trifluoromethyl)benzyl]-N-cyclopropyl-3-(4-fluoro-2-methylphenyl)piperidine-4-carboxamide monohydrochloride

(step 1)

To a solution of the compound (500 mg) obtained in Reference Example 79 and Et₃N (129 μL) in ethyl acetate (30 mL) were added tert-butyl 4-formylpiperidine-1-carboxylate (297 mg) and acetic acid (53.3 μL), and the mixture was stirred at room temperature for 5 min. NaBH(OAc)₃ (517 mg) was added to the reaction mixture, and the mixture was stirred at room temperature for 13.5 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give tert-butyl 4-{[(3R*,4R*)-4-{[[3,5-bis(trifluoromethyl)benzyl](cyclopropyl)amino]carbonyl}-3-(4-fluoro-2-methylphenyl)piperidin-1-yl]methyl}piperidine-1-carboxylate (119.9 mg, 19%) as a white amorphous solid.

MS (ESI+):700 (M+H)

(step 2)

Crude (3R*,4R*)—N-[3,5-bis(trifluoromethyl)benzyl]-N-cyclopropyl-3-(4-fluoro-2-methylphenyl)-1-(piperidin-4-ylmethyl)piperidine-4-carboxamide dihydrochloride was obtained by reaction and purification in the same manner as in Example 6 and using the compound obtained in step 1.

MS (ESI+):600 (M−2HCl+H)

(step 3)

The title compound was obtained by reaction and purification in the same manner as in Example 11 and using the compound obtained in step 2 and acetyl chloride, and a treatment of the obtained resultant product with 1 equivalent amount of 4N hydrogen chloride/ethyl acetate.

MS (ESI+):642 (M−HCl+H)

### Example 169

### (3R*,4R*)-1-(2-amino-2-oxoethyl)-N-[3,5-bis(trifluoromethyl)benzyl]-N-cyclopropyl-3-(4-fluoro-2-methylphenyl)piperidine-4-carboxamide monohydrochloride

The title compound was obtained by reaction and purification in the same manner as in Example 25 and using the compound obtained in Reference Example 79.

MS (ESI+):560 (M−HCl+H)

¹H-NMR (300 MHz, CDCl₃):δ 0.60-1.10 (4H, m), 1.80-2.10 (2H, m), 2.20-2.50 (6H, m), 2.75-3.15 (4H, m), 3.35-3.65 (2H, m), 4.04-4.80 (2H, m), 5.94 (1H, s), 6.62-8.02 (7H, m) (NMR characterization was carried out as its free amine.)

### Example 170

### (3R*,4R*)—N-[3-chloro-5-(trifluoromethyl)benzyl]-3-(4-fluoro-2-methylphenyl)-1-[(1-glycoloylpiperidin-4-yl)carbonyl]-N-methylpiperidine-4-carboxamide

The title compound was obtained by reaction and purification in the same manner as in Example 1 and using the compound obtained in Reference Example 96 and the compound obtained in Reference Example 11.

MS (ESI+):612 (M+H)

### Example 171

### (3R*,4R*)—N-{1-[3-chloro-5-(trifluoromethyl)phenyl]ethyl}-3-(4-fluoro-2-methylphenyl)-1-[(1-glycoloylpiperidin-4-yl)carbonyl]-N-methylpiperidine-4-carboxamide (more polar)

The title compound was obtained by reaction and purification in the same manner as in Example 1 and using the compound obtained in Reference Example 101 and the compound obtained in Reference Example 11.

MS (ESI+):626 (M+H)

### Example 172

### (3R*,4R*)—N-[3,5-bis(trifluoromethyl)benzyl]-3-(3,4-dichlorophenyl)-1-[(1-glycoloylpiperidin-4-yl)carbonyl]-N-methylpiperidine-4-carboxamide

The title compound was obtained by reaction and purification in the same manner as in Example 1 and using the compound obtained in Reference Example 111 and the compound obtained in Reference Example 11.

MS (ESI+):682 (M+H)

### Example 173

### (3R*,4S*)—N-[3,5-bis(trifluoromethyl)benzyl]-1-[(1-glycoloylpiperidin-4-yl)carbonyl]-N-methyl-3-(3-methyl-2-thienyl)piperidine-4-carboxamide

The title compound was obtained by reaction and purification in the same manner as in Example 1 and using the compound obtained in Reference Example 115 and the compound obtained in Reference Example 11.

MS (ESI+):634 (M+H)

### Example 174

### (3R*,4S*)—N-[3-chloro-5-(trifluoromethyl)benzyl]-1-[(1-glycoloylpiperidin-4-yl)carbonyl]-N-methyl-3-(3-methyl-2-thienyl)piperidine-4-carboxamide

The title compound was obtained by reaction and purification in the same manner as in Example 1 and using the compound obtained in Reference Example 119 and the compound obtained in Reference Example 11.

MS (ESI+):600 (M+H)

### Example 175

### (3R*,4S*)—N-{(1S)-1-[3,5-bis(trifluoromethyl)phenyl]ethyl}-1-[(1-glycoloylpiperidin-4-yl)carbonyl]-N-methyl-3-(3-methyl-2-thienyl)piperidine-4-carboxamide (less polar)

To a solution of the compound (300 mg) obtained in Example 189, the compound (170 mg) obtained in Reference Example 11 and Et₃N (81 μL) in CH₃CN (5 mL) were added WSC.HCl (230 mg) and HOBt.H₂O (130 mg), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (215 mg, 57%) as a white amorphous solid.

MS (ESI+):648 (M+H)

[α]_{D}²⁵−75.4° (c 1.0, MeOH)

elemental analysis value: C₃₀H₃₅N₃O₄SF₆

Found C, 55.38; H, 5.38; N, 6.31

Calculated C, 55.63; H, 5.45; N, 6.49

¹H-NMR (300 MHz, CDCl₃):δ 1.27 (3H, d, J=8.4 Hz), 1.60-2.50 (9H, m), 2.50-5.40 (16H, m), 5.90-6.05 (1H, m), 6.78-6.88 (1H, m), 7.00-7.18 (1H, m), 7.59 (2H, s), 7.76 (1H, s)

### Example 176

### (3R*,4S*)—N-{(1S)-1-[3,5-bis(trifluoromethyl)phenyl]ethyl}-1-[(1-glycoloylpiperidin-4-yl)carbonyl]-N-methyl-3-(3-methyl-2-thienyl)piperidine-4-carboxamide (more polar)

To a solution of the compound (300 mg) obtained in Example 190, the compound (170 mg) obtained in Reference Example 11 and Et₃N (81 μL) in CH₃CN (5 mL) were added WSC.HCl (230 mg) and HOBt.H₂O (130 mg), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane) to give the title compound (364 mg, 97%) as a white amorphous solid.

MS (ESI+):648 (M+H)

[α]_{D}²⁵−82.6° (c 1.0, MeOH)

elemental analysis value: C₃₀H₃₅N₃O₄SF₆

Found C, 55.49; H, 5.52; N, 6.31

Calculated C, 55.63; H, 5.45; N, 6.49

¹H-NMR (300 MHz, CDCl₃):δ 1.49 (3H, d, J=7.2 Hz), 1.65-2.30 (9H, m), 2.50-5.20 (16H, m), 5.93 (1H, q, J=6.9 Hz), 6.75-6.81 (1H, m), 6.99-7.08 (1H, m), 7.36 (2H, s), 7.73 (1H, s)

### Example 177

### (3R*,4R*)—N-[3,5-bis(trifluoromethyl)benzyl]-3-(2-ethylphenyl)-1-[(1-glycoloylpiperidin-4-yl)carbonyl]-N-methylpiperidine-4-carboxamide

The title compound was obtained by reaction and purification in the same manner as in Example 1 and using the compound obtained in Reference Example 26 and the compound obtained in Reference Example 11.

MS (ESI+):642 (M+H)

### Example 178

### (3S,4S)-1-[amino(oxo)acetyl]-N-{(1S)-1-[3,5-bis(trifluoromethyl)phenyl]ethyl}-3-(4-fluoro-2-methylphenyl)-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (210.8 mg) synthesized by a known method (WO2006/004195), oxamic acid (46.3 mg) and Et₃N (61 μL) in CH₃CN (4 mL) were added WSC.HCl (99.7 mg) and HOBt.H₂O (79.6 mg), and the mixture was stirred at room temperature for 16 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 40→100% ethyl acetate/hexane) to give the title compound (198 mg, 88%) as a white crystal powder.

MS (ESI+):562 (M+H)

[α]_{D}²⁵−73.0° (c 1.00, MeOH)

elemental analysis value: C₂₆H₂₆N₃O₃F₇

Found C, 55.62; H, 4.67; N, 7.48

Calculated C, 55.72; H, 4.82; N, 7.43

Melting point: 97-109° C.

¹H-NMR (300 MHz, CDCl₃):δ 1.18-1.48 (3H, m), 1.90-2.02 (2H, m), 2.44-3.69 (10H, m), 4.55-5.23 (2H, m), 5.60-5.66 (1H, m), 5.87-5.94 (1H, m), 6.73-7.84 (7H, m)

### Example 179

### (3S,4S)—N-{(1S)-1-[3,5-bis(trifluoromethyl)phenyl]ethyl}-3-(4-fluoro-2-methylphenyl)-N-methyl-1-(methylsulfonyl)piperidine-4-carboxamide

### Example 180

### (3S,4S)—N-{(1S)-1-[3,5-bis(trifluoromethyl)phenyl]ethyl}-3-(4-fluoro-2-methylphenyl)-N-methyl-1-[2-(methylsulfonyl)ethyl]piperidine-4-carboxamide monohydrochloride

To a solution of 2-(methylsulfonyl)ethanol (186 mg) in THF (10 mL) were added methanesulfonyl chloride (113 μL) and Et₃N (198 μL) at room temperature. After stirring at room temperature for 2.5 hr, the precipitate was filtered off. To the filtrate was added a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (300 mg) synthesized by a known method (WO2006/004195) and Et₃N (198 μL) in THF (20 mL) at room temperature, and the mixture was stirred for 20.5 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (NH Chromatorex) (solvent gradient; 50→100% ethyl acetate/hexane). The compound (59.6 mg, 18%) of Example 179 was obtained as a white crystal powder from a fraction with a short retention time. A fraction with a long retention time was concentrated under reduced pressure and treated with 1 equivalent amount of 4N hydrogen chloride/ethyl acetate to give the compound (60.5 mg, 17%) of Example 180 as a white powder.

Compound of Example 179

MS (ESI+):569 (M+H)

[α]_{D}²⁵−27.9° (c 1.03, MeOH)

elemental analysis value: C₂₅H₂₇N₂O₃SF₇

Found C, 52.97; H, 4.64; N, 4.85

Calculated C, 52.81; H, 4.79; N, 4.93

Melting point: 168-172° C.

¹H-NMR (300 MHz, CDCl₃):δ 1.47 (3H, d, J=7.2 Hz), 1.95-2.14 (2H, m), 2.45 (3H, s), 2.66 (1H, t, J=12.0 Hz), 2.68 (3H, s), 2.85 (1H, dt, J=11.9, 3.8 Hz), 2.84 (3H, s), 3.02 (1H, dt, J=10.8, 4.5 Hz), 3.63 (1H, dt, J=11.2, 4.2 Hz), 3.82 (1H, ddd, J=12.5, 4.2, 1.5 Hz), 4.03 (1H, d, J=12.1), 5.91 (1H, q, J=7.2 Hz), 6.76 (1H, dt, J=8.3, 2.7 Hz), 6.86 (1H, dd, J=9.5, 2.7 Hz), 6.97 (1H, dd, J=8.5, 5.5 Hz), 7.28 (2H, s), 7.72 (1H, s)

Compound of Example 180

MS (ESI+):597 (M−HCl+H)

[α]_{D}²⁵−30.5° (c 1.01, MeOH)

elemental analysis value: C₂₇H₃₂N₂O₃SClF₇.1.2H₂O

Found C, 49.51; H, 5.06; N, 4.27

Calculated C, 49.54; H, 5.30; N, 4.28

¹H-NMR (300 MHz, DMSO-d₆):δ 1.44 (3H, d, J=7.0 Hz), 1.86-2.07 (1H, m), 2.09-2.24 (1H, m), 2.26-2.40 (1H, m), 2.33 (3H, s), 2.74 (3H, s), 3.00-3.27 (2H, m), 3.10 (3H, s), 3.38-3.56 (4H, m), 3.61-3.85 (3H, m), 5.69 (1H, q, J=6.6 Hz), 6.82-6.93 (2H, m), 7.34 (1H, dd, J=10.4, 5.8 Hz), 7.41 (2H, s), 7.94 (1H, s), 10.5 (1H, s)

### Example 181

### tert-butyl (3R*,4R*)-4-{[2-(3,5-dichlorophenyl)pyrrolidin-1-yl]carbonyl}-3-(4-fluoro-2-methylphenyl)piperidine-1-carboxylate (less polar)

### Example 182

### tert-butyl (3R*,4R*)-4-{[2-(3,5-dichlorophenyl)pyrrolidin-1-yl]carbonyl}-3-(4-fluoro-2-methylphenyl)piperidine-1-carboxylate (more polar)

(step 1)

To a solution of (3R*,4R*)-1-(tert-butoxycarbonyl)-3-(4-fluoro-2-methylphenyl)piperidine-4-carboxylic acid (1.2 g) synthesized by a known method (WO2006/004195), 2-(3,5-dichlorophenyl)pyrrolidine (1.0 g) synthesized in reference to a known method (WO2006/015150) and Et₃N (729 mg) in DMF (20 mL) were added WSC.HCl (1.03 g) and HOBt.H₂O (828 mg), and the mixture was stirred at room temperature for 14 hr. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→20% ethyl acetate/hexane). The compound (400 mg, 21%) of Example 181 was obtained as a white powder from a fraction with a short retention time. The compound (720 mg, 37%) of Example 182 was obtained as a white powder from a fraction with a long retention time.

Compound of Example 181

MS (ESI+):479 (M−^{t}Bu+2H)

Compound of Example 182

MS (ESI+):479 (M−^{t}Bu+2H)

### Example 183

### (3R*,4R*)-4-{[2-(3,5-dichlorophenyl)pyrrolidin-1-yl]carbonyl}-3-(4-fluoro-2-methylphenyl)piperidine monohydrochloride (less polar)

The title compound was obtained by reaction and purification in the same manner as in Reference Example 6 and using the compound obtained in Example 181.

MS (ESI+):435 (M−HCl+H)

### Example 184

### (3R*,4R*)-4-{[2-(3,5-dichlorophenyl)pyrrolidin-1-yl]carbonyl}-3-(4-fluoro-2-methylphenyl)piperidine monohydrochloride (more polar)

The title compound was obtained by reaction and purification in the same manner as in Reference Example 6 and using the compound obtained in Example 182.

MS (ESI+):435 (M−HCl+H)

### Example 185

### N-{2-[(3R*,4R*)-4-{[2-(3,5-dichlorophenyl)pyrrolidin-1-yl]carbonyl}-3-(4-fluoro-2-methylphenyl)piperidin-1-yl]-2-oxoethyl}acetamide (less polar)

The title compound was obtained by reaction and purification in the same manner as in Reference Example 1 and using the compound obtained in Example 183 and N-acetylglycine.

MS (ESI+):534 (M+H)

### Example 186

### N-{2-[(3R*,4R*)-4-{[2-(3,5-dichlorophenyl)pyrrolidin-1-yl]carbonyl}-3-(4-fluoro-2-methylphenyl)piperidin-1-yl]-2-oxoethyl}acetamide (more polar)

The title compound was obtained by reaction and purification in the same manner as in Reference Example 1 and using the compound obtained in Example 184 and N-acetylglycine.

MS (ESI+):534 (M+H)

### Example 187

### tert-butyl (3R*,4S*)-4-{[{(1S)-1-[3,5-bis(trifluoromethyl)phenyl]ethyl}[methyl]amino]carbonyl}-3-(3-methyl-2-thienyl)piperidine-1-carboxylate (less polar)

### Example 188

### tert-butyl (3R*,4S*)-4-{[{(1S)-1-[3,5-bis(trifluoromethyl)phenyl]ethyl}[methyl]amino]carbonyl}-3-(3-methyl-2-thienyl)piperidine-1-carboxylate (more polar)

To a solution of the compound (0.80 g) obtained in Reference Example 114, step 4 and DMF (15 μL) in THF (20 mL) was added oxalyl chloride (0.26 mL) at 0° C. The mixture was stirred at 0° C. for 1 hr, and the reaction mixture was concentrated under reduced pressure under ice-cooling. To a solution of the obtained residue in THF (10 mL) was added a solution of (1S)-1-[3,5-bis(trifluoromethyl)phenyl]-N-methylethanamine monohydrochloride (0.91 g) and ⁱPr₂NEt (1.03 mL) in THF (10 mL). at 0° C., and the mixture was stirred at room temperature for one day. The reaction mixture was poured into water, and the resultant product was extracted with ethyl acetate. The organic layer was washed with saturated aqueous sodium hydrogen carbonate solution and saturated brine and dried, and the solvent was evaporated under reduced pressure. The obtained residue was purified by silica gel column chromatography (solvent gradient; 5→40% ethyl acetate/hexane). The compound (0.71 g, 50%) of Example 187 was obtained as a white powder from a fraction with a short retention time. The compound (0.67 g, 47%) of Reference Example 188 was obtained as a white powder from a fraction with a long retention time.

Compound of Example 187

MS (ESI+):523 (M−^{t}Bu+2H)

[α]_{D}²⁵−81.7° (c 0.52, MeOH)

elemental analysis value: C₂₇H₃₂N₂O₃SF₆

Found C, 56.05; H, 5.57; N, 4.84

Calculated C, 56.11; H, 5.51; N, 4.81

Compound of Example 188

MS (ESI+):523 (M−^{t}Bu+2H)

[α]_{D}²⁵−86.9° (c 0.51, MeOH)

elemental analysis value: C₂₇H₃₂N₂O₃SF₆

Found C, 56.05; H, 5.57; N, 4.84

Calculated C, 55.84; H, 5.54; N, 4.70

### Example 189

### N-{(3R*,4S*)-(1S)-1-[3,5-bis(trifluoromethyl)phenyl]ethyl}-N-methyl-3-(3-methyl-2-thienyl)piperidine-4-carboxamide monohydrochloride (less polar)

The title compound was obtained by reaction and purification in the same manner as in Reference Example 6 and using the compound obtained in Example 187.

MS (ESI+):479 (M−HCl+H)

### Example 190

### (3R*,4S*)—N-{(1S)-1-[3,5-bis(trifluoromethyl)phenyl]ethyl}-N-methyl-3-(3-methyl-2-thienyl)piperidine-4-carboxamide monohydrochloride (more polar)

The title compound was obtained by reaction and purification in the same manner as in Reference Example 6 and using the compound obtained in Example 188.

MS (ESI+):479 (M−HCl+H)

### Example 191

### (3R*,4S*)-1-[amino(oxo)acetyl]-N-{(1S)-1-[3,5-bis(trifluoromethyl)phenyl]ethyl}-N-methyl-3-(3-methyl-2-thienyl)piperidine-4-carboxamide (less polar)

The title compound was obtained by reaction and purification in the same manner as in Reference Example 1 and using the compound obtained in Example 189.

MS (ESI+):550 (M+H)

### Example 192

### (3R*,4S*)-1-[amino(oxo)acetyl]-N-1(1S)-1-[3,5-bis(trifluoromethyl)phenyl]ethyl}-N-methyl-3-(3-methyl-2-thienyl)piperidine-4-carboxamide (more polar)

The title compound was obtained by reaction and purification in the same manner as in Reference Example 1 and using the compound obtained in Example 190.

MS (ESI+):550 (M+H)

### Example 193

### (3S,4S)-1-[amino(oxo)acetyl]-N-{(1S)-1-[3,5-bis(trifluoromethyl)phenyl]ethyl}-3-(4-fluoro-2-methylphenyl)-N-methylpiperidine-4-carboxamide

To a solution of (3S,4S)—N-{(1S)-1-[3,5-bis(trifluoromethyl)phenyl]ethyl}-3-(4-fluoro-2-methylphenyl)-N-methylpiperidine-4-carboxamide monohydrochloride (1.05 g) synthesized by a known method (WO2006/004195), oxamic acid (214 mg) and Et₃N (335 μL) in CH₃CN (10 mL) were added WSC.HCl (460 mg) and HOBt.H₂O (306 mg), and the mixture was stirred at room temperature for 3 hr. The reaction mixture was poured into water, and the resultant product was extracted with ethyl acetate. The organic layer was washed with saturated aqueous sodium hydrogen carbonate solution, water and saturated brine and dried, and the solvent was evaporated under reduced pressure. The obtained residue was purified by silica gel column chromatography (NH Chromatorex) (solvent gradient; 50→100% ethyl acetate/hexane) to give the title compound (833 mg, 74%) as a white crystal powder.

MS (ESI+):562 (M+H)

[α]_{D}²⁵−73.0° (c 1.00, MeOH)

elemental analysis value: C₂₆H₂₆N₃O₃F₇

Found C, 55.57; H, 4.61; N, 7.50

Calculated C, 55.72; H, 4.82; N, 7.43

Melting point: 121-126° C.

¹H-NMR (300 MHz, CDCl₃):δ 1.18-1.48 (3H, m), 1.90-2.02 (2H, m), 2.44-3.69 (10H, m), 4.55-5.23 (2H, m), 5.60-5.66 (1H, m), 5.87-5.94 (1H, m), 6.73-7.84 (7H, m)

The compounds described in Examples 1-192 are shown in (Table 15)-(Table 33).

TABLE 15

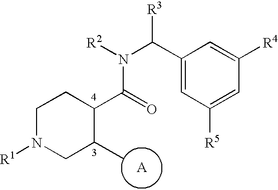

| Ex.No. | stereo-chemistry | 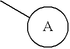 | R¹ | R² | R³ | R⁴ | R⁵ | additives | MS(ESI) |
|---|---|---|---|---|---|---|---|---|---|
| 1 | (3R*, 4S*) | 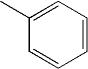 | 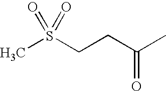 | CH₃ | H | CF₃ | CF₃ | | 579(M + H) |
| 2 | (3R*, 4S*) | 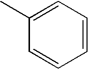 | 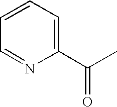 | CH₃ | H | CF₃ | CF₃ | | 550(M + H) |
| 3 | (3R*, 4S*) | 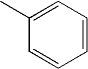 | 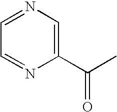 | CH₃ | H | CF₃ | CF₃ | | 551(M + H) |
| 4 | (3R*, 4S*) | 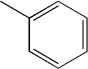 | 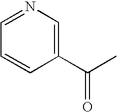 | CH₃ | H | CF₃ | CF₃ | | 550(M + H) |
| 5 | (3R*, 4S*) | 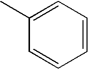 | 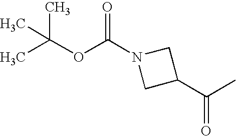 | CH₃ | H | CF₃ | CF₃ | | 628(M + H) |
| 6 | (3R*, 4S*) | 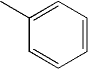 | 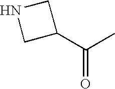 | CH₃ | H | CF₃ | CF₃ | HCl | 528(M − HCl + H) |
| 7 | (3R*, 4S*) | 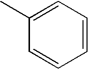 | 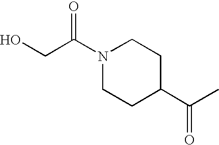 | CH₃ | H | CF₃ | CF₃ | | 614(M + H) |
| 8 | (3R*, 4R*) | 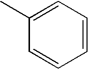 | 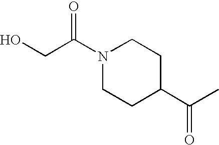 | CH₃ | H | CF₃ | CF₃ | | 614(M + H) |
| 9 | (3R*, 4S*) | 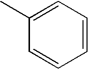 | 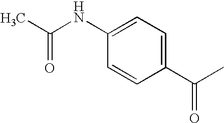 | CH₃ | H | CF₃ | CF₃ | | 606(M + H) |
| 10 | (3R*, 4S*) | 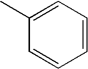 | 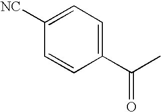 | CH₃ | H | CF₃ | CF₃ | | 574(M + H) |

TABLE 16

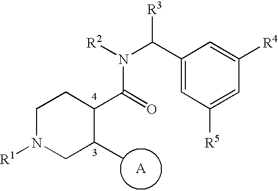

| Ex.No. | stereo-chemistry | 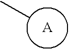 | R¹ | R² | R³ | R⁴ | R⁵ | additives | MS(ESI) |
|---|---|---|---|---|---|---|---|---|---|
| 11 | (3R*, 4R*) | 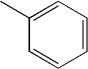 | 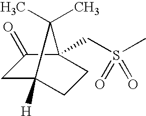 | CH₃ | H | CF₃ | CF₃ | | 691(M + H) |
| 12 | (3R*, 4S*) | 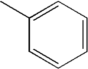 | 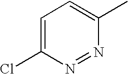 | CH₃ | H | CF₃ | CF₃ | HCl | 557(M − HCl + H) |
| 13 | (3R*, 4S*) | 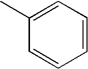 | 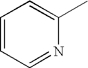 | CH₃ | H | CF₃ | CF₃ | HCl | 522(M − HCl + H) |
| 14 | (3R*, 4S*) | 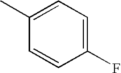 | 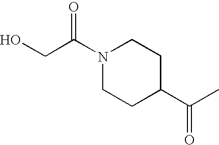 | CH₃ | H | CF₃ | CF₃ | | 632(M + H) |
| 15 | (3R*, 4R*) | 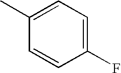 | 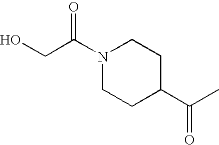 | CH₃ | H | CF₃ | CF₃ | | 632(M + H) |
| 16 | (3R*, 4R*) | 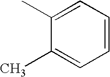 | 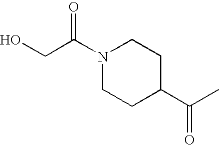 | CH₃ | H | CF₃ | CF₃ | | 628(M + H) |
| 17 | (3R, 4R) | 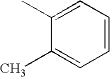 | 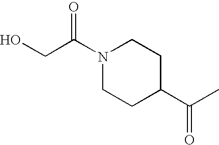 | CH₃ | H | CF₃ | CF₃ | | 628(M + H) |
| 18 | (3S, 4S) | 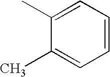 | 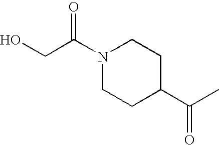 | CH₃ | H | CF₃ | CF₃ | | 628(M + H) |
| 19 | (3R*, 4R*) | 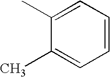 | 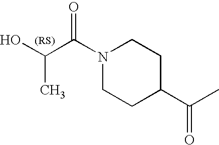 | CH₃ | H | CF₃ | CF₃ | | 642(M + H) |
| 20 | (3R*, 4R*) | 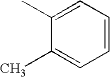 | 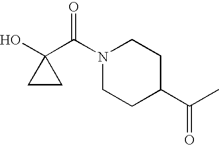 | CH₃ | H | CF₃ | CF₃ | | 654(M + H) |

TABLE 17

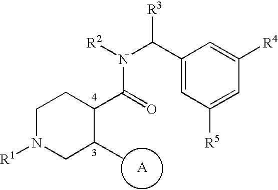

| Ex.No. | stereo-chemistry | 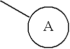 | R¹ | R² | R³ | R⁴ | R⁵ | additives | MS(ESI) |
|---|---|---|---|---|---|---|---|---|---|
| 21 | (3R*, 4R*) | 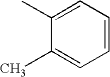 | 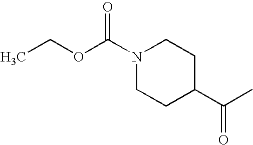 | CH₃ | H | CF₃ | CF₃ | | 642(M + H) |
| 22 | (3R*, 4R*) | 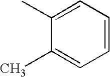 | 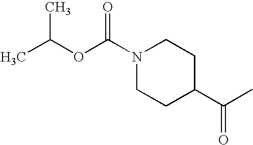 | CH₃ | H | CF₃ | CF₃ | | 656(M + H) |
| 23 | (3R*, 4R*) | 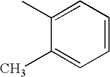 | 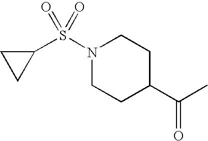 | CH₃ | H | CF₃ | CF₃ | | 674(M + H) |
| 24 | (3R*, 4R*) | 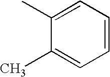 | 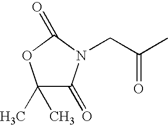 | CH₃ | H | CF₃ | CF₃ | | 628(M + H) |
| 25 | (3R*, 4R*) | 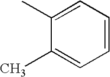 | 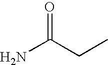 | CH₃ | H | CF₃ | CF₃ | HCl | 516(M − HCl + H) |
| 26 | (3S, 4S) | 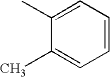 | 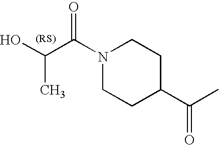 | CH₃ | H | CF₃ | CF₃ | | 642(M + H) |
| 27 | (3R*, 4R*) | 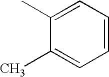 | 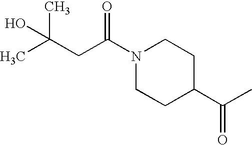 | CH₃ | H | CF₃ | CF₃ | | 670(M + H) |
| 28 | (3S, 4S) | 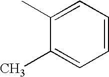 | 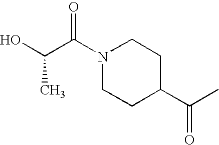 | CH₃ | H | CF₃ | CF₃ | | 642(M + H) |
| 29 | (3S, 4S) | 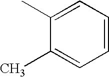 | 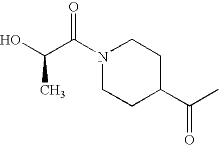 | CH₃ | H | CF₃ | CF₃ | | 642(M + H) |
| 30 | (3S, 4S) | 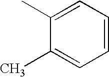 | 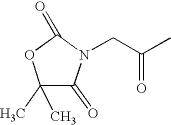 | CH₃ | (S)-CH₃ | CF₃ | CF₃ | | 642(M + H) |

TABLE 18

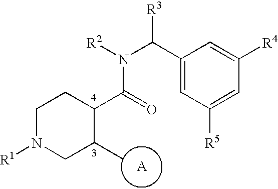

| | | | | | | | | | |
|---|---|---|---|---|---|---|---|---|---|
| Ex.No. | stereo-chemistry | 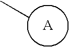 | R¹ | R² | R³ | R⁴ | R⁵ | additives | MS(ESI) |

| | | | | | | | | |
|---|---|---|---|---|---|---|---|---|
| 31 | (3R*, 4R*) | 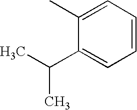 | 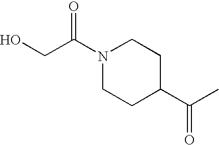 | CH₃ | H | CF₃ | CF₃ | 656(M + H) |
| 32 | (3R*, 4R*) | 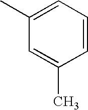 | 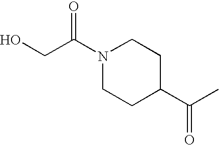 | CH₃ | H | CF₃ | CF₃ | 628(M + H) |
| 33 | (3R*, 4S*) | 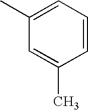 | 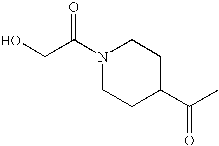 | CH₃ | H | CF₃ | CF₃ | 628(M + H) |
| 34 | (3R*, 4R*) | 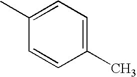 | 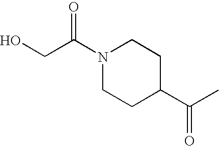 | CH₃ | H | CF₃ | CF₃ | 628(M + H) |
| 35 | (3R*, 4S*) | 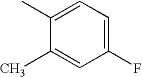 | 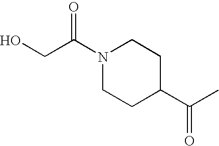 | CH₃ | H | CF₃ | CF₃ | 646(M + H) |
| 36 | (3R*, 4R*) | 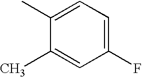 | 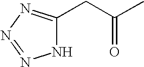 | CH₃ | H | CF₃ | CF₃ | 587(M + H) |
| 37 | (3R*, 4R*) | 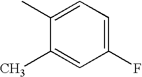 | 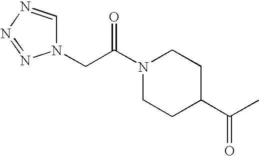 | CH₃ | H | CF₃ | CF₃ | 698(M + H) |
| 38 | (3R*, 4R*) | 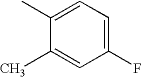 | 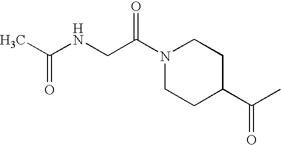 | CH₃ | H | CF₃ | CF₃ | 687(M + H) |
| 39 | (3R*, 4R*) | 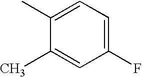 | 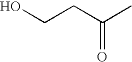 | CH₃ | H | CF₃ | CF₃ | 549(M + H) |
| 40 | (3R*, 4R*) | 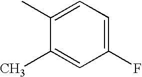 | 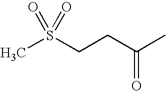 | CH₃ | H | CF₃ | CF₃ | 611(M + H) |

TABLE 19

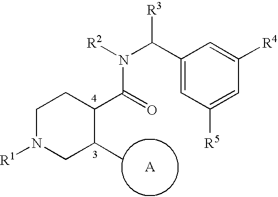

| Ex.No. | stereo-chemistry | 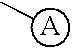 | R¹ | R² | R³ | R⁴ | R⁵ | additives | MS(ESI) |
|---|---|---|---|---|---|---|---|---|---|
| 41 | (3R*,4R*) | 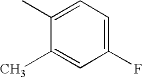 | 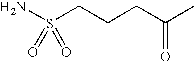 | CH₃ | H | CF₃ | CF₃ | | 626(M + H) |
| 42 | (3R*,4R*) | 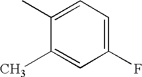 | 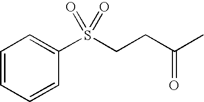 | CH₃ | H | CF₃ | CF₃ | | 673(M + H) |
| 43 | (3R*,4R*) | 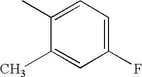 | 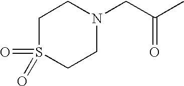 | CH₃ | H | CF₃ | CF₃ | | 652(M + H) |
| 44 | (3R*,4R*) | 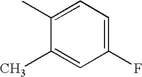 | 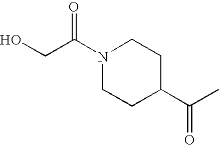 | CH₃ | H | CF₃ | CF₃ | | 646(M + H) |
| 45 | (3R*,4R*) | 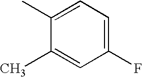 | 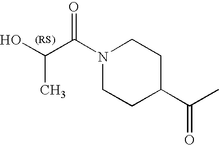 | CH₃ | H | CF₃ | CF₃ | | 660(M + H) |
| 46 | (3R*,4R*) | 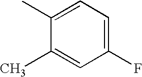 | 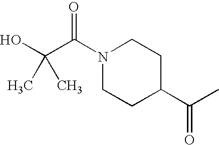 | CH₃ | H | CF₃ | CF₃ | | 674(M + H) |
| 47 | (3R*,4R*) | 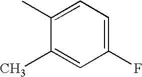 | 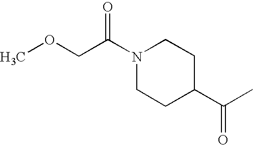 | CH₃ | H | CF₃ | CF₃ | | 660(M + H) |
| 48 | (3R*,4R*) | 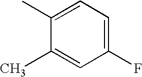 | 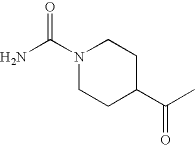 | CH₃ | H | CF₃ | CF₃ | | 631(M + H) |
| 49 | (3R*,4R*) | 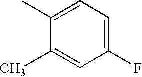 | 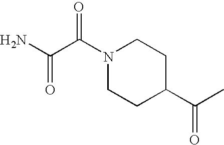 | CH₃ | H | CF₃ | CF₃ | | 659(M + H) |
| 50 | (3R*,4R*) | 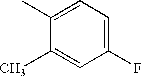 | 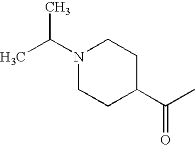 | CH₃ | H | CF₃ | CF₃ | HCl | 630(M − HCl + H) |

TABLE 20

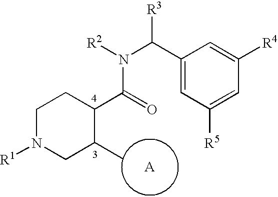

| Ex.No. | stereo-chemistry | 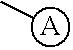 | R¹ | R² | R³ | R⁴ | R⁵ | additives | MS(ESI) |
|---|---|---|---|---|---|---|---|---|---|
| 51 | (3R*,4R*) | 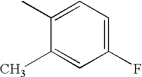 | 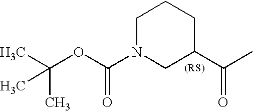 | CH₃ | H | CF₃ | CF₃ | | 688(M + H) |
| 52 | (3R*,4R*) | 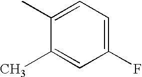 | 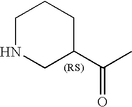 | CH₃ | H | CF₃ | CF₃ | HCl | 588(M − HCl + H) |
| 53 | (3R*,4R*) | 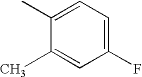 | 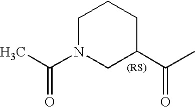 | CH₃ | H | CF₃ | CF₃ | | 630(M + H) |
| 54 | (3R*,4R*) | 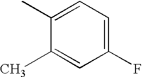 | 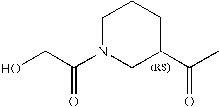 | CH₃ | H | CF₃ | CF₃ | | 646(M + H) |
| 55 | (3R*,4R*) | 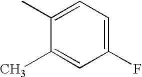 | 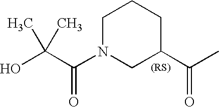 | CH₃ | H | CF₃ | CF₃ | | 674(M + H) |
| 56 | (3R*,4R*) | 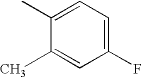 | 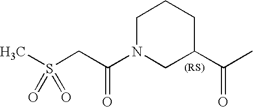 | CH₃ | H | CF₃ | CF₃ | | 708(M + H) |
| 57 | (3R*,4R*) | 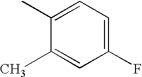 | 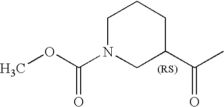 | CH₃ | H | CF₃ | CF₃ | | 646(M + H) |
| 58 | (3R*,4R*) | 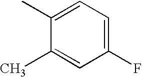 | 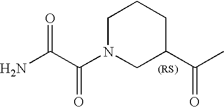 | CH₃ | H | CF₃ | CF₃ | | 659(M + H) |
| 59 | (3R*,4R*) | 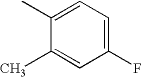 | 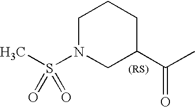 | CH₃ | H | CF₃ | CF₃ | | 666(M + H) |
| 60 | (3R*,4R*) | 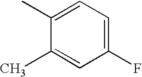 | 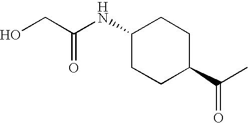 | CH₃ | H | CF₃ | CF₃ | | 660(M + H) |

TABLE 21

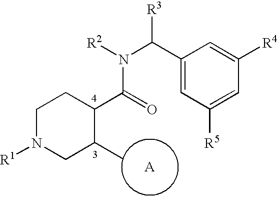

| Ex.No. | stereo-chemistry | 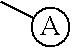 | R¹ | R² | R³ | R⁴ | R⁵ | additives | MS(ESI) |
|---|---|---|---|---|---|---|---|---|---|
| 61 | (3R*,4R*) | 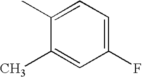 | 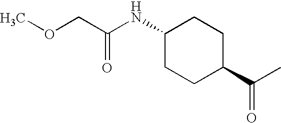 | CH₃ | H | CF₃ | CF₃ | | 674(M + H) |
| 62 | (3R*,4R*) | 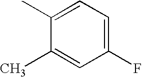 | 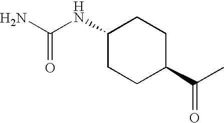 | CH₃ | H | CF₃ | CF₃ | | 645(M + H) |
| 63 | (3R*,4R*) | 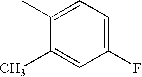 | 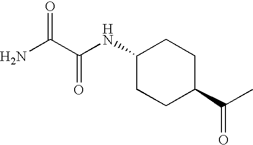 | CH₃ | H | CF₃ | CF₃ | | 673(M + H) |
| 64 | (3R*,4R*) | 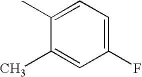 | 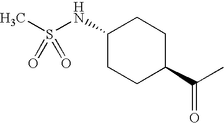 | CH₃ | H | CF₃ | CF₃ | | 680(M + H) |
| 65 | (3R*,4R*) | 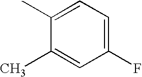 | 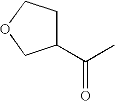 | CH₃ | H | CF₃ | CF₃ | | 575(M + H) |
| 66 | (3R*,4R*) | 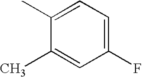 | 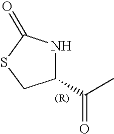 | CH₃ | H | CF₃ | CF₃ | | 606(M + H) |
| 67 | (3R*,4R*) | 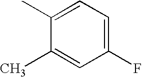 | 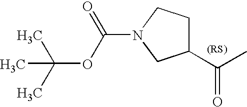 | CH₃ | H | CF₃ | CF₃ | | 674(M + H) |
| 68 | (3R*,4R*) | 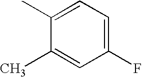 | 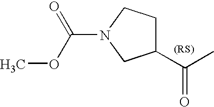 | CH₃ | H | CF₃ | CF₃ | | 632(M + H) |
| 69 | (3R*,4R*) | 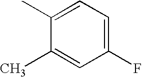 | 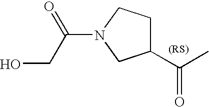 | CH₃ | H | CF₃ | CF₃ | | 632(M + H) |
| 70 | (3R*,4R*) | 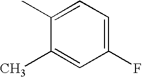 | 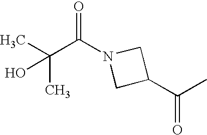 | CH₃ | H | CF₃ | CF₃ | | 646(M + H) |

TABLE 22

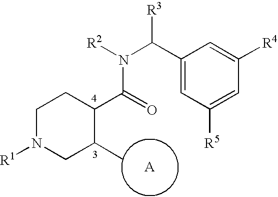

| Ex.No. | stereo-chemistry | 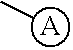 | R¹ | R² | R³ | R⁴ | R⁵ | additives | MS(ESI) |
|---|---|---|---|---|---|---|---|---|---|
| 71 | (3R*,4R*) | 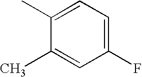 | 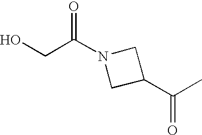 | CH₃ | H | CF₃ | CF₃ | | 618(M + H) |
| 72 | (3R*,4R*) | 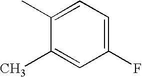 | 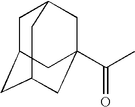 | CH₃ | H | CF₃ | CF₃ | | 639(M + H) |
| 73 | (3R*,4R*) | 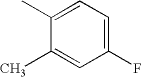 | 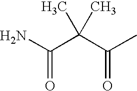 | CH₃ | H | CF₃ | CF₃ | | 590(M + H) |
| 74 | (3R*,4R*) | 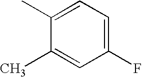 | 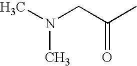 | CH₃ | H | CF₃ | CF₃ | HCl | 562(M − HCl + H) |
| 75 | (3R*,4R*) | 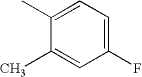 | 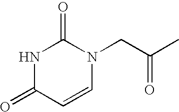 | CH₃ | H | CF₃ | CF₃ | | 629(M + H) |
| 76 | (3R*,4R*) | 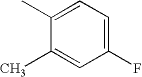 | 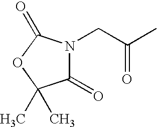 | CH₃ | H | CF₃ | CF₃ | | 646(M + H) |
| 77 | (3R*,4R*) | 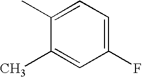 | 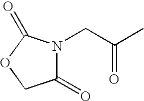 | CH₃ | H | CF₃ | CF₃ | | 618(M + H) |
| 78 | (3R*,4R*) | 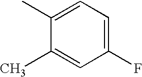 | 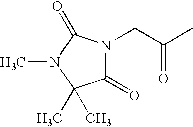 | CH₃ | H | CF₃ | CF₃ | | 659(M + H) |
| 79 | (3R*,4R*) | 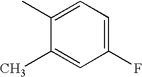 | 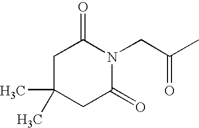 | CH₃ | H | CF₃ | CF₃ | | 658(M + H) |
| 80 | (3R*,4R*) | 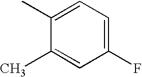 | 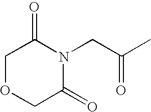 | CH₃ | H | CF₃ | CF₃ | | 632(M + H) |

TABLE 23

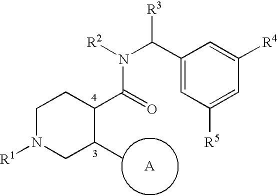

| Ex.No. | stereo-chemistry | 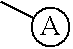 | R¹ | R² | R³ | R⁴ | R⁵ | additives | MS(ESI) |
|---|---|---|---|---|---|---|---|---|---|
| 81 | (3R*,4R*) | 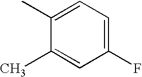 | 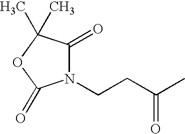 | CH₃ | H | CF₃ | CF₃ | | 660(M + H) |
| 82 | (3R*,4R*) | 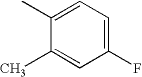 | 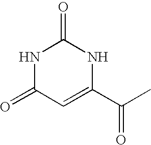 | CH₃ | H | CF₃ | CF₃ | | 615(M + H) |
| 83 | (3R*,4R*) | 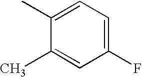 | 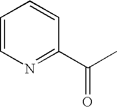 | CH₃ | H | CF₃ | CF₃ | | 582(M + H) |
| 84 | (3R*,4R*) | 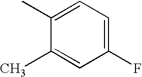 | 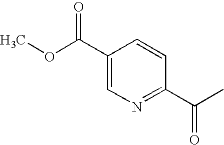 | CH₃ | H | CF₃ | CF₃ | | 640(M + H) |
| 85 | (3R*,4R*) | 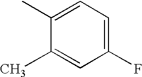 | 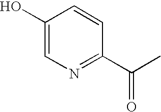 | CH₃ | H | CF₃ | CF₃ | | 598(M + H) |
| 86 | (3R*,4R*) | 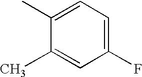 | 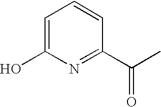 | CH₃ | H | CF₃ | CF₃ | | 598(M + H) |
| 87 | (3R*,4R*) | 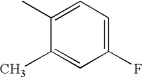 | 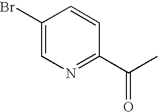 | CH₃ | H | CF₃ | CF₃ | | 660, 662(M + H) |
| 88 | (3R*,4R*) | 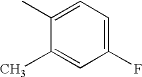 | 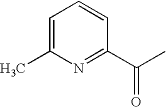 | CH₃ | H | CF₃ | CF₃ | | 596(M + H) |
| 89 | (3R*,4R*) | 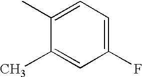 | 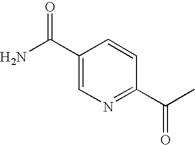 | CH₃ | H | CF₃ | CF₃ | | 625(M + H) |
| 90 | (3R*,4R*) | 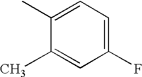 | 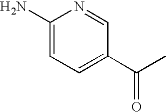 | CH₃ | H | CF₃ | CF₃ | | 597(M + H) |

TABLE 24

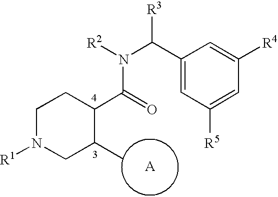

| Ex.No. | stereo-chemistry | 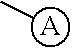 | R¹ | R² | R³ | R⁴ | R⁵ | additives | MS(ESI) |
|---|---|---|---|---|---|---|---|---|---|
| 91 | (3R*,4R*) | 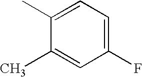 | 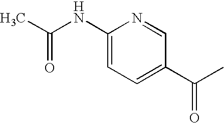 | CH₃ | H | CF₃ | CF₃ | | 639(M + H) |
| 92 | (3R*,4R*) | 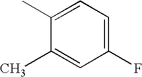 | 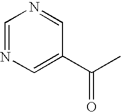 | CH₃ | H | CF₃ | CF₃ | | 583(M + H) |
| 93 | (3R*,4R*) | 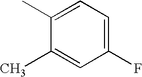 | 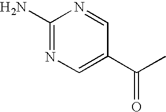 | CH₃ | H | CF₃ | CF₃ | | 598(M + H) |
| 94 | (3R*,4R*) | 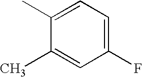 | 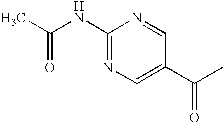 | CH₃ | H | CF₃ | CF₃ | | 640(M + H) |
| 95 | (3R*,4R*) | 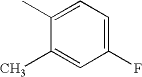 | 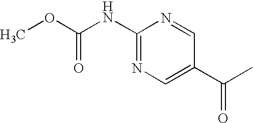 | CH₃ | H | CF₃ | CF₃ | | 656(M + H) |
| 96 | (3R*,4R*) | 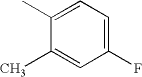 | 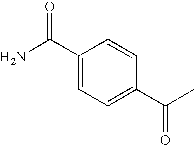 | CH₃ | H | CF₃ | CF₃ | | 624(M + H) |
| 97 | (3R*,4R*) | 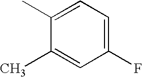 | 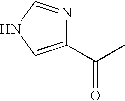 | CH₃ | H | CF₃ | CF₃ | | 571(M + H) |
| 98 | (3R*,4R*) | 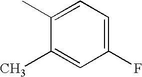 | 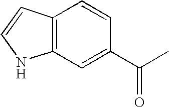 | CH₃ | H | CF₃ | CF₃ | | 620(M + H) |
| 99 | (3R*,4R*) | 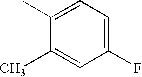 | 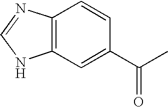 | CH₃ | H | CF₃ | CF₃ | | 621(M + H) |
| 100 | (3R*,4R*) | 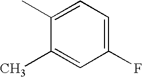 | 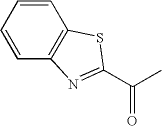 | CH₃ | H | CF₃ | CF₃ | | 638(M + H) |

TABLE 25

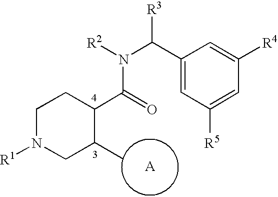

| Ex.No. | stereo-chemistry | 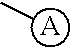 | R¹ | R² | R³ | R⁴ | R⁵ | additives | MS(ESI) |
|---|---|---|---|---|---|---|---|---|---|
| 101 | (3R*,4R*) | 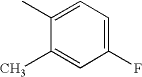 | 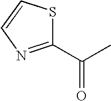 | CH₃ | H | CF₃ | CF₃ | | 588(M + H) |
| 102 | (3R*,4R*) | 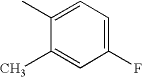 | 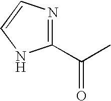 | CH₃ | H | CF₃ | CF₃ | | 571(M + H) |
| 103 | (3R*,4R*) | 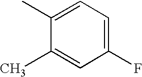 | 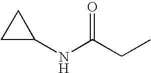 | CH₃ | H | CF₃ | CF₃ | HCl | 574(M − HCl + H) |
| 104 | (3R*,4R*) | 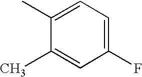 | 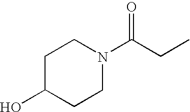 | CH₃ | H | CF₃ | CF₃ | HCl | 618(M − HCl + H) |
| 105 | (3R*,4R*) | 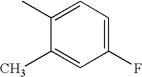 | 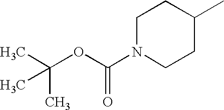 | CH₃ | H | CF₃ | CF₃ | | 660(M + H) |
| 106 | (3R*,4R*) | 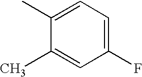 | 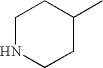 | CH₃ | H | CF₃ | CF₃ | 2HCl | 560(M − 2HCl + H) |
| 107 | (3R*,4R*) | 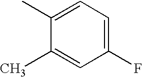 | 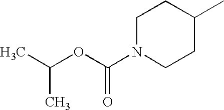 | CH₃ | H | CF₃ | CF₃ | HCl | 646(M − HCl + H) |
| 108 | (3R*,4R*) | 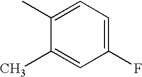 | 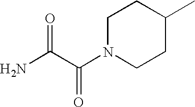 | CH₃ | H | CF₃ | CF₃ | HCl | 631(M − HCl + H) |
| 109 | (3R*,4R*) | 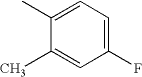 | 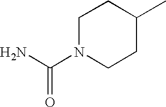 | CH₃ | H | CF₃ | CF₃ | HCl | 603(M − HCl + H) |
| 110 | (3R*,4R*) | 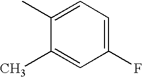 | 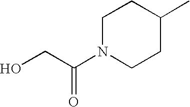 | CH₃ | H | CF₃ | CF₃ | HCl | 618(M − HCl + H) |

TABLE 26

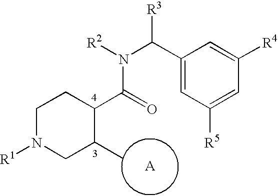

| Ex.No. | stereo-chemistry | 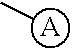 | R¹ | R² | R³ | R⁴ | R⁵ | additives | MS(ESI) |
|---|---|---|---|---|---|---|---|---|---|
| 111 | (3R*,4R*) | 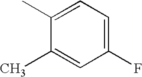 | 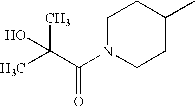 | CH₃ | H | CF₃ | CF₃ | HCl | 646(M − HCl + H) |
| 112 | (3R*,4R*) | 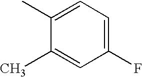 | 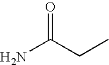 | CH₃ | H | CF₃ | CF₃ | HCl | 534(M − HCl + H) |
| 113 | (3R*,4R*) | 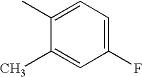 | 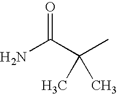 | CH₃ | H | CF₃ | CF₃ | HCl | 562(M − HCl + H) |
| 114 | (3R*,4R*) | 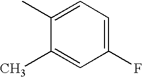 | 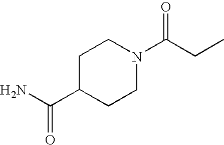 | CH₃ | H | CF₃ | CF₃ | HCl | 645(M − HCl + H) |
| 115 | (3R*,4R*) | 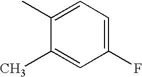 | 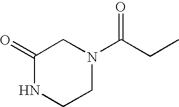 | CH₃ | H | CF₃ | CF₃ | HCl | 617(M − HCl + H) |
| 116 | (3R*,4R*) | 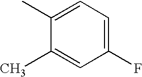 | 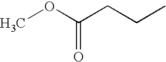 | CH₃ | H | CF₃ | CF₃ | HCl | 563(M − HCl + H) |
| 117 | (3R*,4R*) | 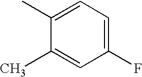 | 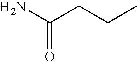 | CH₃ | H | CF₃ | CF₃ | HCl | 548(M − HCl + H) |
| 118 | (3R*,4R*) | 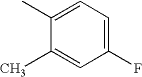 | 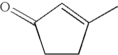 | CH₃ | H | CF₃ | CF₃ | | 557(M + H) |
| 119 | (3R*,4R*) | 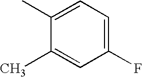 | 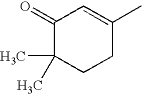 | CH₃ | H | CF₃ | CF₃ | | 599(M + H) |
| 120 | (3R*,4R*) | 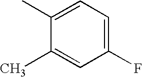 | 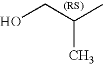 | CH₃ | H | CF₃ | CF₃ | HCl | 535(M − HCl + H) |

TABLE 27

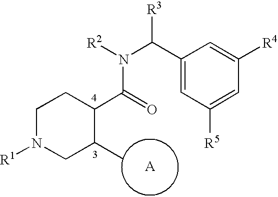

| Ex.No. | stereo-chemistry | 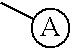 | R¹ | R² | R³ | R⁴ | R⁵ | additives | MS(ESI) |
|---|---|---|---|---|---|---|---|---|---|
| 121 | (3R*,4R*) | 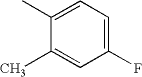 | 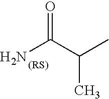 | CH₃ | H | CF₃ | CF₃ | HCl | 548(M − HCl + H) |
| 122 | (3R*,4R*) | 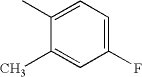 | 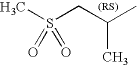 | CH₃ | H | CF₃ | CF₃ | HCl | 597(M − HCl + H) |
| 123 | (3R*,4R*) | 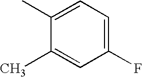 | 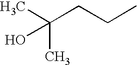 | CH₃ | H | CF₃ | CF₃ | HCl | 563(M − HCl + H) |
| 124 | (3R*,4R*) | 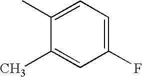 | 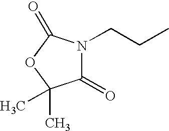 | CH₃ | H | CF₃ | CF₃ | HCl | 632(M − HCl + H) |
| 125 | (3R*,4R*) | 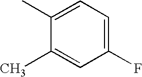 | 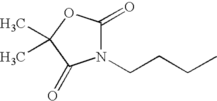 | CH₃ | H | CF₃ | CF₃ | HCl | 646(M − HCl + H) |
| 126 | (3R*,4R*) | 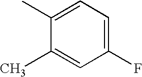 | 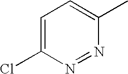 | CH₃ | H | CF₃ | CF₃ | HCl | 589(M − HCl + H) |
| 127 | (3R*,4R*) | 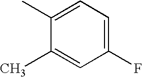 | 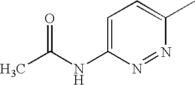 | CH₃ | H | CF₃ | CF₃ | HCl | 612(M − HCl + H) |
| 128 | (3R*,4R*) | 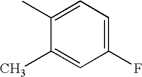 | 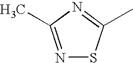 | CH₃ | H | CF₃ | CF₃ | HCl | 575(M − HCl + H) |
| 129 | (3R*,4R*) | 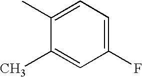 | 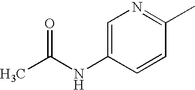 | CH₃ | H | CF₃ | CF₃ | HCl | 611(M − HCl + H) |
| 130 | (3R*,4R*) | 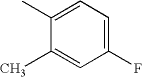 | 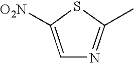 | CH₃ | H | CF₃ | CF₃ | | 605(M + H) |

TABLE 28

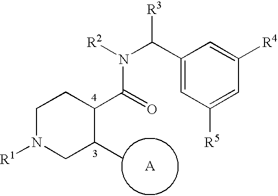

| Ex.No. | stereo-chemistry | 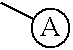 | R¹ | R² | R³ | R⁴ | R⁵ | additives | MS(ESI) |
|---|---|---|---|---|---|---|---|---|---|
| 131 | (3R*,4R*) | 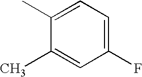 | 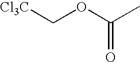 | CH₃ | H | CF₃ | CF₃ | | N.T. |
| 132 | (3R*,4R*) | 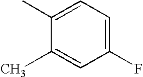 | 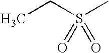 | CH₃ | H | CF₃ | CF₃ | | 569(M + H) |
| 133 | (3R*,4R*) | 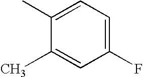 | 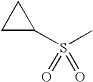 | CH₃ | H | CF₃ | CF₃ | | 581(M + H) |
| 134 | (3R*,4R*) | 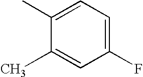 | 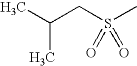 | CH₃ | H | CF₃ | CF₃ | | 597(M + H) |
| 135 | (3R*,4R*) | 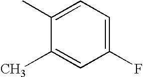 | 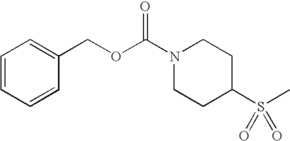 | CH₃ | H | CF₃ | CF₃ | | 758(M + H) |
| 136 | (3R*,4R*) | 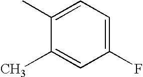 | 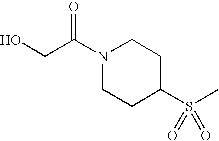 | CH₃ | H | CF₃ | CF₃ | | 682(M + H) |
| 137 | (3R*,4R*) | 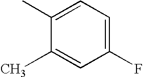 | 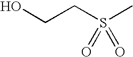 | CH₃ | H | CF₃ | CF₃ | | 585(M + H) |
| 138 | (3R*,4R*) | 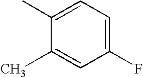 | 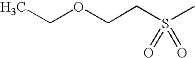 | CH₃ | H | CF₃ | CF₃ | | 613(M + H) |
| 139 | (3R*,4R*) | 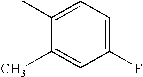 | 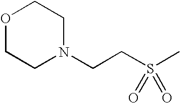 | CH₃ | H | CF₃ | CF₃ | HCl | 654(M − HCl + H) |
| 140 | (3R*,4R*) | 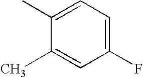 | 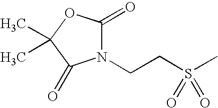 | CH₃ | H | CF₃ | CF₃ | | 696(M + H) |

TABLE 29

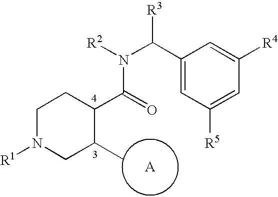

| Ex.No. | stereo-chemistry | 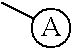 | R¹ | R² | R³ | R⁴ | R⁵ | additives | MS(ESI) |
|---|---|---|---|---|---|---|---|---|---|
| 141 | (3R*,4R*) | 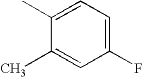 | 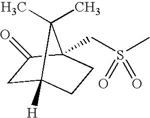 | CH₃ | H | CF₃ | CF₃ | | 691(M + H) |
| 142 | (3R*,4R*) | 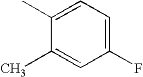 | 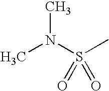 | CH₃ | H | CF₃ | CF₃ | | 584(M + H) |
| 143 | (3R*,4R*) | 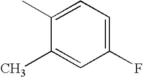 | 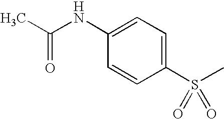 | CH₃ | H | CF₃ | CF₃ | | 674(M + H) |
| 144 | (3R*,4R*) | 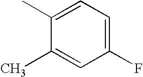 | 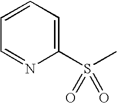 | CH₃ | H | CF₃ | CF₃ | | 618(M + H) |
| 145 | (3R*,4R*) | 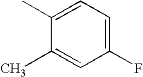 | 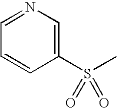 | CH₃ | H | CF₃ | CF₃ | HCl | 618(M − HCl + H) |
| 146 | (3R*,4R*) | 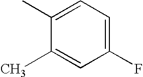 | 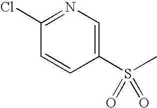 | CH₃ | H | CF₃ | CF₃ | | 652(M + H) |
| 147 | (3R*,4R*) | 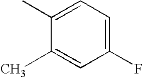 | 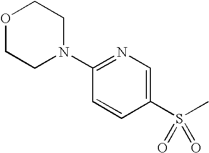 | CH₃ | H | CF₃ | CF₃ | HCl | 703(M − HCl + H) |
| 148 | (3S,4S) | 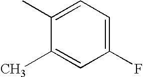 | 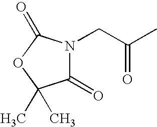 | CH₃ | H | CF₃ | CF₃ | | 646(M + H) |
| 149 | (3S,4S) | 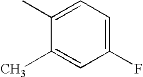 | 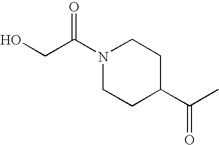 | CH₃ | H | CF₃ | CF₃ | | 646(M + H) |
| 150 | (3S,4S) | 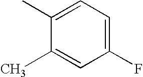 | 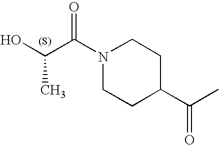 | CH₃ | H | CF₃ | CF₃ | | 660(M + H) |

TABLE 30

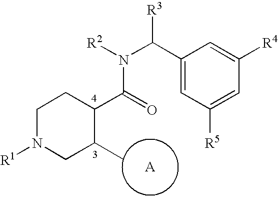

| Ex.No. | stereo-chemistry | 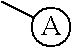 | R¹ | R² | R³ | R⁴ | R⁵ | additives | MS(ESI) |
|---|---|---|---|---|---|---|---|---|---|
| 151 | (3S,4S) | 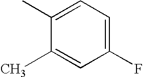 | 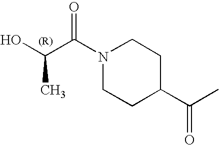 | CH₃ | H | CF₃ | CF₃ | | 660(M + H) |
| 152 | (3S,4S) | 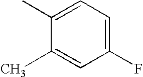 | 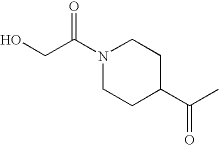 | CH₃ | (S)-CH₃ | CF₃ | CF₃ | | 660(M + H) |
| 153 | (3S,4S) | 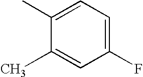 | 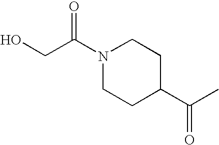 | CH₃ | (R)-CH₃ | CF₃ | CF₃ | | 660(M + H) |
| 154 | (3S,4S) | 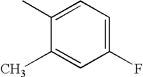 | 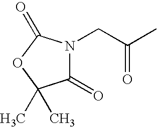 | CH₃ | (S)-CH₃ | CF₃ | CF₃ | | 660(M + H) |
| 155 | (3S,4S) | 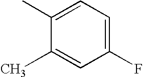 | 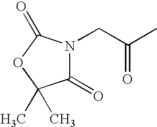 | CH₃ | (R)-CH₃ | CF₃ | CF₃ | | 660(M + H) |
| 156 | (3S,4S) | 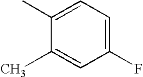 | 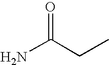 | CH₃ | (S)-CH₃ | CF₃ | CF₃ | | 548(M + H) |
| 157 | (3S,4S) | 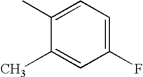 | 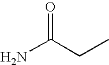 | CH₃ | (R)-CH₃ | CF₃ | CF₃ | HCl | 548(M − HCl + H) |
| 158 | (3S,4S) | 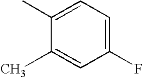 | 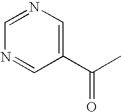 | CH₃ | (S)-CH₃ | CF₃ | CF₃ | | 597(M + H) |
| 159 | (3S,4S) | 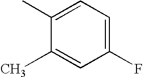 | 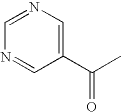 | CH₃ | (R)-CH₃ | CF₃ | CF₃ | | 597(M + H) |
| 160 | (3S,4S) | 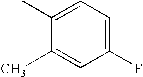 | 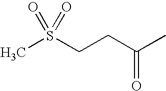 | CH₃ | (S)-CH₃ | CF₃ | CF₃ | | 625(M + H) |

TABLE 31

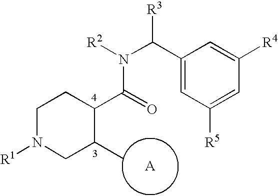

| Ex.No. | stereo-chemistry | 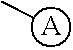 | R¹ | R² | R³ | R⁴ | R⁵ | additives | MS(ESI) |
|---|---|---|---|---|---|---|---|---|---|
| 161 | (3S,4S) | 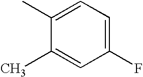 | 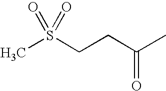 | CH₃ | (R)-CH₃ | CF₃ | CF₃ | | 625(M + H) |
| 162 | (3S,4S) | 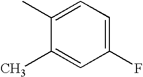 | 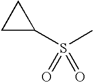 | CH₃ | (S)-CH₃ | CF₃ | CF₃ | | 595(M + H) |
| 163 | (3R*,4R*) | 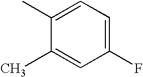 | 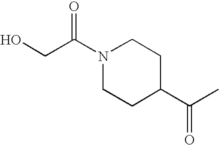 | 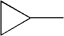 | H | CF₃ | CF₃ | | 672(M + H) |
| 164 | (3R*,4R*) | 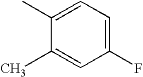 | 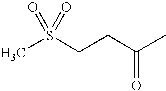 | 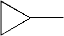 | H | CF₃ | CF₃ | | 637(M + H) |
| 165 | (3R*,4R*) | 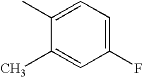 | 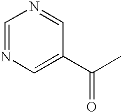 | 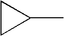 | H | CF₃ | CF₃ | | 609(M + H) |
| 166 | (3R*,4R*) | 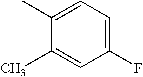 | 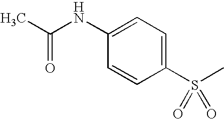 | 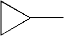 | H | CF₃ | CF₃ | | 700(M + H) |
| 167 | (3R*,4R*) | 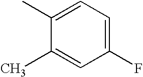 | 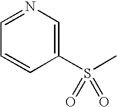 | 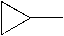 | H | CF₃ | CF₃ | HCl | 644(M − HCl + H) |
| 168 | (3R*,4R*) | 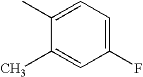 | 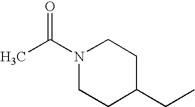 | 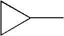 | H | CF₃ | CF₃ | HCl | 642(M − HCl + H) |
| 169 | (3R*,4R*) | 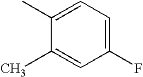 | 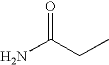 | 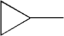 | H | CF₃ | CF₃ | HCl | 560(M − HCl + H) |
| 170 | (3R*,4R*) | 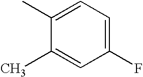 | 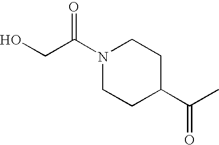 | CH₃ | H | Cl | CF₃ | | 612(M + H) |

TABLE 32

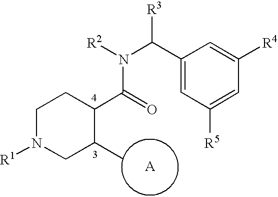

| Ex.No. | stereo-chemistry | 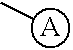 | R¹ | R² | R³ | R⁴ | R⁵ | additives | MS(ESI) |
|---|---|---|---|---|---|---|---|---|---|
| 171 | (3R*,4R*)(more polar) | 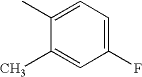 | 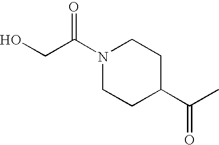 | CH₃ | CH₃ | Cl | CF₃ | | 626(M + H) |
| 172 | (3R*,4R*) | 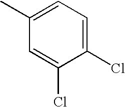 | 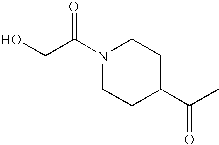 | CH₃ | H | CF₃ | CF₃ | | 682(M + H) |
| 173 | (3R*,4S*) | 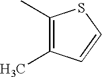 | 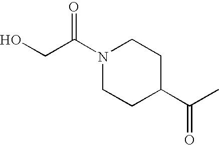 | CH₃ | H | CF₃ | CF₃ | | 634(M + H) |
| 174 | (3R*,4S*) | 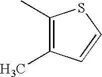 | 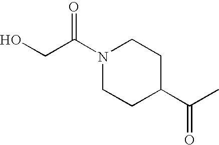 | CH₃ | H | Cl | CF₃ | | 600(M + H) |
| 175 | (3R*,4S*)(less polar) | 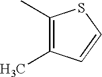 | 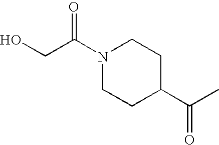 | CH₃ | (S)-CH₃ | CF₃ | CF₃ | | 648(M + H) |
| 176 | (3R*,4S*)(more polar) | 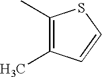 | 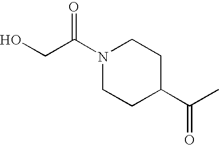 | CH₃ | (S)-CH₃ | CF₃ | CF₃ | | 648(M + H) |
| 177 | (3R*,4R*) | 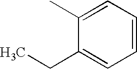 | 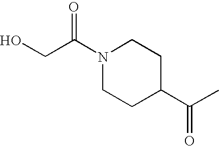 | CH₃ | H | CF₃ | CF₃ | | 642(M + H) |
| 178 | (3S,4S) | 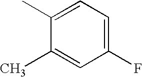 | 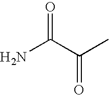 | CH₃ | (S)-CH₃ | CF₃ | CF₃ | | 562(M + H) |
| 179 | (3S,4S) | 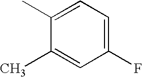 | 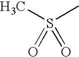 | CH₃ | (S)-CH₃ | CF₃ | CF₃ | | 569(M + H) |
| 180 | (3S,4S) | 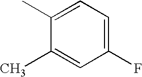 | 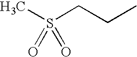 | CH₃ | (S)-CH₃ | CF₃ | CF₃ | HCl | 597(M − HCl + H) |

TABLE 33

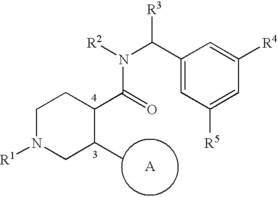

| Ex.No. | stereo-chemistry | 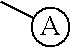 | R¹ | R² | R³ | R⁴ | R⁵ | additives | MS(ESI) |
|---|---|---|---|---|---|---|---|---|---|
| 181 | (3R*,4R*)(less polar) | 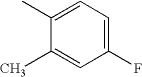 | 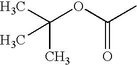 | —(CH₂)₃— | | Cl | Cl | | 479(M − ^{t}Bu + 2H) |
| 182 | (3R*,4R*)(more polar) | 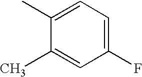 | 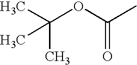 | —(CH₂)₃— | | Cl | Cl | | 479(M − ^{t}Bu + 2H) |
| 183 | (3R*,4R*)(less polar) | 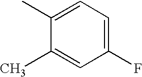 | H | —(CH₂)₃— | | Cl | Cl | HCl | 435(M − HCl + H) |
| 184 | (3R*,4R*)(more polar) | 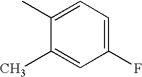 | H | —(CH₂)₃— | | Cl | Cl | HCl | 435(M − HCl + H) |
| 185 | (3R*,4R*)(less polar) | 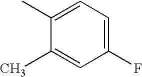 | 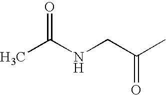 | —(CH₂)₃— | | Cl | Cl | | 534(M + H) |
| 186 | (3R*,4R*)(more polar) | 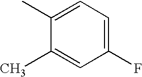 | 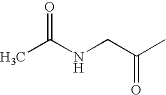 | —(CH₂)₃— | | Cl | Cl | | 534(M + H) |
| 187 | (3R*,4S*)(less polar) | 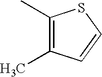 | 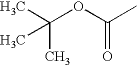 | CH₃ | (S)-CH₃ | CF₃ | CF₃ | | 523(M − ^{t}Bu + 2H) |
| 188 | (3R*,4S*)(more polar) | 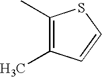 | 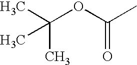 | CH₃ | (S)-CH₃ | CF₃ | CF₃ | | 523(M − ^{t}Bu + 2H) |
| 189 | (3R*,4S*)(less polar) | 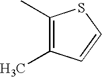 | H | CH₃ | (S)-CH₃ | CF₃ | CF₃ | HCl | 479(M − HCl + H) |
| 190 | (3R*,4S*)(more polar) | 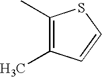 | H | CH₃ | (S)-CH₃ | CF₃ | CF₃ | HCl | 479(M − HCl + H) |
| 191 | (3R*,4S*)(less polar) | 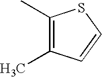 | 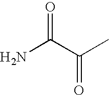 | CH₃ | (S)-CH₃ | CF₃ | CF₃ | | 550(M + H) |
| 192 | (3R*,4S*)(more polar) | 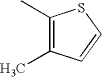 | 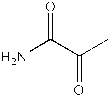 | CH₃ | (S)-CH₃ | CF₃ | CF₃ | | 550(M + H) |

### Preparative Example 1

| | |
|---|---|
| (1) Compound of Example 1 | 10 mg |
| (2) Lactose | 60 mg |
| (3) Corn starch | 35 mg |
| (4) Hydroxypropylmethylcellulose | 3 mg |
| (5) Magnesium stearate | 2 mg |

A mixture of the compound (10 mg) obtained in Example 1, lactose (60 mg) and corn starch (35 mg) is granulated using a 10 wt % aqueous solution (0.03 mL) of hydroxypropylmethylcellulose (3 mg as hydroxypropylmethylcellulose), and then dried at 40° C. and sieved. The obtained granules are mixed with magnesium stearate (2 mg) and compressed. The obtained uncoated tablets are sugar-coated with an aqueous suspension of sucrose, titanium dioxide, talc and gum Arabic. The thus-coated tablets are glazed with bees wax to obtain finally-coated tablets.

### Preparative Example 2

| | |
|---|---|
| (1) Compound of Example 1 | 10 mg |
| (2) Lactose | 70 mg |
| (3) Corn starch | 50 mg |
| (4) Soluble starch | 7 mg |
| (5) Magnesium stearate | 3 mg |

The compound obtained in Example 1 (10 mg) and magnesium stearate (3 mg) are granulated with an aqueous soluble starch solution (0.07 mL, 7 mg as soluble starch), dried, and mixed with lactose (70 mg) and corn starch (50 mg). The mixture is compressed to give tablets.

### Reference Preparative Example 1

| | | |
|---|---|---|
| (1) Rofecoxib | 5.0 | mg |
| (2) Sodium chloride | 20.0 | mg |
| (3) Distilled water | to 2 | mL of total volume |

Rofecoxib (5.0 mg) and sodium chloride (20.0 mg) are dissolved in distilled water, and water is added to make the total volume 2.0 mL. The solution is filtered, and filled into ampoule (2 mL) under sterile condition. The ampoule is sterilized, and then sealed to obtain a solution for injection.

### Reference Preparative Example 2

| | |
|---|---|
| (1) Rofecoxib | 50 mg |
| (2) Lactose | 34 mg |
| (3) Corn starch | 10.6 mg |
| (4) Corn starch (paste) | 5 mg |
| (5) Magnesium stearate | 0.4 mg |
| (6) Calcium carboxymethylcellulose | 20 mg |
| Total | 120 mg |

The above-mentioned (1) to (6) were mixed according to a conventional method and tableted by a tablet machine to obtain tablets.

### Preparative Example 3

The formulation prepared in Preparative Example 1 or 2, and the formulation prepared in Reference Preparative Example 1 or 2 are combined.

### Experimental Example 1

Radioligand Receptor Binding Inhibitory Activity (Binding Inhibitory Activity Using Receptor from Human Lymphoblast Cells (IM-9))

The method of M. A. Cascieri et al., “Molecular Pharmacology, vol. 42, p. 458 (1992)” was modified and used. The receptor was prepared from human lymphoblast cells (IM-9).

IM-9 cells (2×10⁵ cells/mL) were inoculated and incubated for 3 days, which was then subjected to centrifugation for 10 min. at 500×G to give cell pellets. The obtained pellets were washed with PBS (GIBCO), disrupted in buffer A (50 mM tris.hydrochloric acid buffer (pH 7.4) containing 120 mM sodium chloride, 5 mM potassium chloride, 2 μg/mL chymostatin, 40 μg/mL bacitracin, 40 μg/mL APMSF, 1 mM ethylenediamine tetra acetic acid) using a polytron.homogenizer [manufactured by Kinematika, Germany], and centrifuged at 100,000×G for 40 min. The precipitated fraction was suspended in buffer B (50 mM Tris-HCl (pH 7.4), 0.02% bovine serum albumin, 2 μg/mL chymostatin, 40 μg/mL bacitracin, 40 μg/mL APMSF, 3 mM MgCl₂), which was then cryopreserved (−80° C.) as a receptor specimen.

Buffer B (50 μL) was added to a 96 well microassay plate (CORNING). Thereto was added 50 μL of membrane preparation suspended in buffer B at 250 μg/mL. A measurement buffer (50 μL) containing 2% dimethyl sulfoxide was added to examine total binding; 4 μM unlabeled SP (50 μL) diluted with a measurement buffer containing 2% dimethyl sulfoxide was added to examine non-specific binding; and 50 μL of a test compound (containing 2% dimethyl sulfoxide) diluted with a measurement buffer was added to examine binding inhibitory activity of the test compound. In addition, 400 μM ¹²⁵I-BHSP solution (50 μL) was added to each well.

After reaction at 25° C. for 30 min, the cells were rapidly filtered through a GF/C filter plate (PerkinElmer) using a cell harvester (manufactured by PerkinElmer) to quench the reaction, and washed 10 times with 250 μL of 50 mM tris-HCl buffer (pH 7.4) containing 0.02% bovine serum albumin. The GF/C filter plate was dried, MicroScinti 0 (20 μL) was added, and the radioactivity was measured with TopCount (manufactured by PerkinElmer). The GF/C filter plate was immersed in 0.3% polyethylene imine for one day before use.

Specific binding is a value obtained by subtracting nonspecific binding from total binding. The binding inhibitory activity of a test compound is shown by a ratio of a value obtained by subtracting a measurement value with addition of a test compound from the total binding, relative to specific binding.

The antagonistic activity of the compounds obtained in the Examples was determined as a concentration necessary for causing 50% inhibition (IC₅₀ value) under the above-described conditions, and the results shown in Table 34 were obtained.

TABLE 34

| Example No. | IC₅₀ value (nM) |
|---|---|
| 18 | 0.024 |
| 28 | 0.022 |
| 29 | 0.023 |
| 30 | 0.057 |
| 103 | 0.096 |
| 104 | 0.042 |
| 108 | 0.026 |
| 109 | 0.023 |
| 112 | 0.038 |
| 113 | 0.061 |
| 118 | 0.060 |
| 119 | 0.060 |
| 120 | 0.043 |
| 123 | 0.025 |
| 129 | 0.095 |
| 132 | 0.040 |
| 138 | 0.078 |
| 139 | 0.037 |
| 142 | 0.055 |
| 144 | 0.045 |
| 148 | 0.060 |
| 149 | 0.015 |
| 150 | 0.033 |
| 151 | 0.020 |
| 152 | 0.015 |
| 154 | 0.097 |
| 156 | 0.024 |
| 158 | 0.033 |
| 160 | 0.032 |
| 162 | 0.081 |
| 169 | 0.077 |
| 176 | 0.051 |
| 178 | 0.017 |
| 179 | 0.049 |
| 180 | 0.023 |

The radio ligand means substance P labeled with [¹²⁵I].

From the Table, it has been clarified that the compounds of the present invention have superior antagonistic action for the substance P receptor.

### INDUSTRIAL APPLICABILITY

Compound (I), compound (II), a salt thereof and a prodrug thereof have a high tachykinin receptor antagonistic action, particularly, a high substance P receptor antagonistic action, superior drug efficacy sustainability (metabolic stability) and low toxicity (e.g. vascular toxicity), are safe as pharmaceutical agents, and least impact on other agents. Accordingly, compound (I), compound (II), a salt thereof and a prodrug thereof are useful as pharmaceutical agents, for example, tachykinin receptor antagonists, agents for the prophylaxis or treatment of lower urinary tract diseases and the like.

This application is based on a patent application No. 2007-114858 filed in Japan, the contents of which are incorporated in full herein by this reference.

1. A compound represented by the formula: 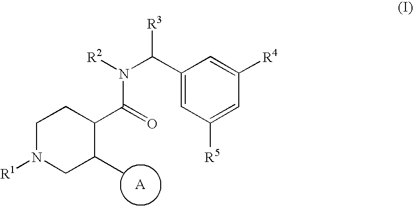
wherein
R¹ is
(1) carbamoylmethyl,
(2) methylsulfonylethylcarbonyl,
(3) aminosulfonylpropylcarbonyl,
(4) phenylsulfonylethylcarbonyl,
(5) pyridin-2-ylcarbonyl,
(6) 5-methoxycarbonylpyridin-2-ylcarbonyl,
(7) 5-hydroxypyridin-2-ylcarbonyl,
(8) 6-hydroxypyridin-2-ylcarbonyl,
(9) 5-bromopyridin-2-ylcarbonyl,
(10) 6-methylpyridin-2-ylcarbonyl,
(11) 5-carbamoylpyridin-2-ylcarbonyl,
(12) 2-aminopyridin-5-ylcarbonyl,
(13) 2-acetylaminopyridin-5-ylcarbonyl,
(14) pyridin-3-ylcarbonyl,
(15) pyrazin-2-ylcarbonyl,
(16) pyrimidin-5-ylcarbonyl,
(17) 2-aminopyrimidin-5-ylcarbonyl,
(18) 2-acetylaminopyrimidin-5-ylcarbonyl,
(19) 2-methoxycarbonylaminopyrimidin-5-ylcarbonyl,
(20) azetidin-3-ylcarbonyl,
(21) 1-tert-butoxycarbonylazetidin-3-ylcarbonyl,
(22) 1-(1-hydroxy-1-methylethylcarbonyl)azetidin-3-ylcarbonyl,
(23) 1-hydroxymethylcarbonylazetidin-3-ylcarbonyl,
(24) adamantan-1-ylcarbonyl,
(25) 1-carbamoyl-1-methylethylcarbonyl,
(26) dimethylaminomethylcarbonyl,
(27) 1,2,3,4-tetrahydropyrimidine-2,4-dion-1-ylmethylcarbonyl,
(28) 2,3,5,6-tetrahydropyrimidine-2,6-dion-4-ylmethylcarbonyl,
(29) 1-hydroxymethylcarbonylpiperidin-4-ylcarbonyl,
(30) 1-ethoxycarbonylpiperidin-4-ylcarbonyl,
(31) 1-isopropoxycarbonylpiperidin-4-ylcarbonyl,
(32) 1-cyclopropylsulfonylpiperidin-4-ylcarbonyl,
(33) 1-(1-hydroxyethylcarbonyl)piperidin-4-ylcarbonyl,
(34) 5,5-dimethyloxazolidine-2,4-dion-3-ylmethylcarbonyl,
(35) 5,5-dimethyloxazolidine-2,4-dion-3-ylethylcarbonyl,
(36) oxazolidine-2,4-dion-3-ylmethylcarbonyl,
(37) 1-(2-hydroxy-2-methylpropylcarbonyl)piperidin-4-ylcarbonyl,
(38) 3-methylimidazolidine-4,4-dimethyl-2,5-dion-1-ylmethylcarbonyl,
(39) 4,4-dimethylpiperidine-2,6-dion-1-ylmethylcarbonyl,
(40) morpholine-2,6-dion-1-ylmethylcarbonyl,
(41) imidazol-2-ylcarbonyl,
(42) imidazol-4-ylcarbonyl,
(43) 5,5-dimethyloxazolidine-2,4-dion-3-ylethyl,
(44) 5,5-dimethyloxazolidine-2,4-dion-3-ylpropyl,
(45) 6-chloropyridazin-3-yl,
(46) 6-acetylaminopyridazin-3-yl,
(47) 5-methyl-1,3,4-thiadiazol-2-yl,
(48) 5-acetylaminopyridin-2-yl,
(49) 4-nitrothiazol-2-yl,
(50) 2,2,2-trichloroethoxycarbonyl,
(51) ethylsulfonyl,
(52) cyclopropylsulfonyl,
(53) isobutylsulfonyl,
(54) 1-benzyloxycarbonylpiperidin-4-ylsulfonyl,
(55) 1-hydroxymethylcarbonylpiperidin-4-ylsulfonyl,
(56) ethoxyethylsulfonyl,
(57) hydroxyethylsulfonyl,
(58) morpholin-1-ylethylsulfonyl,
(59) 5,5-dimethyloxazolidine-2,4-dion-3-ylethylsulfonyl,
(60) indol-6-ylcarbonyl,
(61) benzimidazol-6-ylcarbonyl,
(62) benzthiazol-2-ylcarbonyl,
(63) thiazol-2-ylcarbonyl,
(64) cyclopropylaminocarbonylmethyl,
(65) 4-hydroxypiperidin-1-ylcarbonylmethyl,
(66) 1-tert-butoxycarbonylpiperidin-4-yl,
(67) piperidin-4-yl,
(68) 1-isopropoxycarbonylpiperidin-4-yl,
(69) 1-aminocarbonylcarbonylpiperidin-4-yl,
(70) 1-carbamoylpiperidin-4-yl,
(71) 1-hydroxymethylcarbonylpiperidin-4-yl,
(72) 1-(1-hydroxy-1-methylethylcarbonyl)piperidin-4-yl,
(73) 1-carbamoyl-1-methylethyl,
(74) 1-acetylpiperidin-4-ylmethyl,
(75) 4-carbamoylpiperidin-1-ylcarbonylmethyl,
(76) 3-oxopiperazin-1-ylcarbonylmethyl,
(77) methoxycarbonylethyl,
(78) carbamoylethyl,
(79) cyclopenten-3-on-1-yl,
(80) 4,4-dimethylcyclohexen-3-on-1-yl,
(81) 2-hydroxy-1-methylethyl,
(82) 1-carbamoylethyl,
(83) 2-methylsulfonyl-1-methylethyl,
(84) 3-hydroxy-3-methylbutyl,
(85) 4-acetylaminobenzoyl,
(86) 4-cyanobenzoyl,
(87) 4-carbamoylbenzoyl,
(88) 10-camphorsulfonyl,
(89) 6-chloropyridazin-3-yl,
(90) pyridin-2-yl,
(91) 1-(1-hydroxy-1-methylethylcarbonyl)piperidin-4-ylcarbonyl,
(92) 1-methoxymethylcarbonylpiperidin-4-ylcarbonyl,
(93) 1-aminocarbonylpiperidin-4-ylcarbonyl,
(94) 1-aminocarbonylcarbonylpiperidin-4-ylcarbonyl,
(95) 1-isopropylpiperidin-4-ylcarbonyl,
(96) 1-(1-hydroxycyclopropylcarbonyl)piperidin-4-ylcarbonyl,
(97) tetrazol-5-ylmethylcarbonyl,
(98) 1-(tetrazol-1-ylmethylcarbonyl)piperidin-4-ylcarbonyl,
(99) 1-(acetylaminomethylcarbonyl)piperidin-4-ylcarbonyl,
(100) 2-hydroxyethylcarbonyl,
(101) thiomorpholine-1,1-dioxido-4-ylmethylcarbonyl,
(102) dimethylaminosulfonyl,
(103) 4-acetylaminophenylsulfonyl,
(104) pyridin-2-ylsulfonyl,
(105) pyridin-3-ylsulfonyl,
(106) 6-chloropyridin-3-ylsulfonyl,
(107) 6-(morpholin-1-yl)pyridin-3-ylsulfonyl,
(108) piperidin-3-ylcarbonyl,
(109) 1-tert-butoxycarbonylpiperidin-3-ylcarbonyl,
(110) 1-methoxycarbonylpiperidin-3-ylcarbonyl,
(111) 1-acetylpiperidin-3-ylcarbonyl,
(112) 1-hydroxymethylcarbonylpiperidin-3-ylcarbonyl,
(113) 1-(1-hydroxy-1-methylethylcarbonyl)piperidin-3-ylcarbonyl,
(114) 1-methylsulfonylmethylcarbonylpiperidin-3-ylcarbonyl,
(115) 1-methylsulfonylpiperidin-3-ylcarbonyl,
(116) 1-aminocarbonylcarbonylpiperidin-3-ylcarbonyl,
(117) 4-hydroxymethylcarbonylaminocyclohexan-1-ylcarbonyl,
(118) 4-methoxymethylcarbonylaminocyclohexan-1-ylcarbonyl,
(119) 4-aminocarbonylaminocyclohexan-1-ylcarbonyl,
(120) 4-aminocarbonylcarbonylaminocyclohexan-1-ylcarbonyl,
(121) 4-methylsulfonylaminocyclohexan-1-ylcarbonyl,
(122) tetrahydrofuran-3-ylcarbonyl,
(123) thiazolidin-2-on-4-ylcarbonyl,
(124) 1-tert-butoxycarbonylpyrrolidin-3-ylcarbonyl,
(125) 1-methoxycarbonylpyrrolidin-3-ylcarbonyl, or
(126) 1-hydroxymethylcarbonylpyrrolidin-3-ylcarbonyl;
R² is methyl or cyclopropyl;
R³ is a hydrogen atom or methyl;
R⁴ is a chlorine atom or trifluoromethyl;
R⁵ is a chlorine atom or trifluoromethyl; and
a group represented by 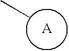
is a group represented by the formula: 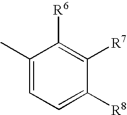
wherein
R⁶ is a hydrogen atom, methyl, ethyl or isopropyl;
R⁷ is a hydrogen atom, methyl or chlorine atom; and
R⁸ is a hydrogen atom, a fluorine atom, a chlorine atom or methyl; or
3-methylthiophen-2-yl;
or a salt thereof.

2. The compound of claim 1, wherein the partial structure:

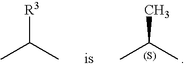

3. A compound represented by the formula: 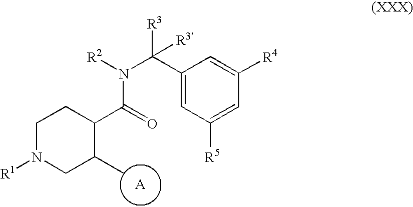
wherein
R¹ is a hydrogen atom, a hydrocarbon group optionally having substituent(s), an acyl group or a heterocyclic group optionally having substituent(s);
R² is an optionally halogenated C₁₋₆ alkyl group;
R³ and R^{3′} are each independently a hydrogen atom or methyl, or R³ and R^{3′} are optionally bonded to each other to form a ring together with a carbon atom bonded thereto;
R⁴ is a chlorine atom or trifluoromethyl;
R⁵ is a chlorine atom or trifluoromethyl; and a group represented by the formula: 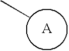
is a heterocyclic group optionally having substituent(s);
or a salt thereof.

4. A compound represented by the formula: 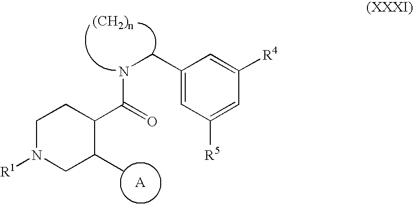
wherein
R¹ is a hydrogen atom, a hydrocarbon group optionally having substituent(s), an acyl group or a heterocyclic group optionally having substituent(s);
R⁴ is a chlorine atom or trifluoromethyl;
R⁵ is a chlorine atom or trifluoromethyl;
a group represented by the formula: 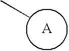
is a group represented by the formula: 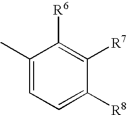
wherein
R⁶ is a hydrogen atom, methyl, ethyl or isopropyl;
R⁷ is a hydrogen atom, methyl or a chlorine atom; and
R⁸ is a hydrogen atom, a fluorine atom, a chlorine atom or methyl; and
n is an integer of 3 to 6;
or a salt thereof.

5. An optically active compound represented by the formula: 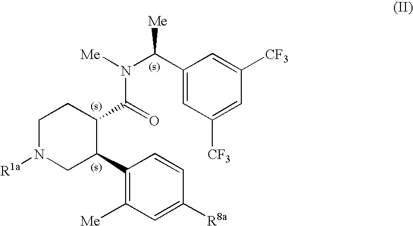
wherein
R^{1a} is
(1) 5,5-dimethyloxazolidine-2,4-dion-3-ylmethylcarbonyl,
(2) 1-hydroxymethylcarbonylpiperidin-4-ylcarbonyl,
(3) carbamoylmethyl,
(4) pyrimidin-5-ylcarbonyl,
(5) methylsulfonylethylcarbonyl,
(6) cyclopropylsulfonyl,
(7) aminocarbonylcarbonyl,
(8) methylsulfonyl, or
(9) methylsulfonylethyl;
R^{8a} is
(1) a hydrogen atom, or
(2) a fluorine atom; and
(s) shows that the steric configuration of an asymmetric carbon is an S-configuration;
or a salt thereof.

6. (3R,4R)—N-[3,5-bis(Trifluoromethyl)benzyl]-1-{[1-(hydroxyacetyl)piperidin-4-yl]carbonyl}-N-methyl-3-(2-methylphenyl)piperidine-4-carboxamide or a salt thereof.

7. (3S,4S)—N-[3,5-bis(trifluoromethyl)benzyl]-1-{[1-(hydroxyacetyl)piperidin-4-yl]carbonyl}-N-methyl-3-(2-methylphenyl)piperidine-4-carboxamide or a salt thereof.

8. (3S,4S)—N-[3,5-bis(trifluoromethyl)benzyl]-1-({1-[(2S)-2-hydroxypropanoyl]piperidin-4-yl}carbonyl)-N-methyl-3-(2-methylphenyl)piperidine-4-carboxamide or a salt thereof.

9. (3S,4S)—N-[3,5-bis(trifluoromethyl)benzyl]-1-({1-[(2R)-2-hydroxypropanoyl]piperidin-4-yl}carbonyl)-N-methyl-3-(2-methylphenyl)piperidine-4-carboxamide or a salt thereof.

10. (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-N-methyl-3-(2-methylphenyl)piperidine-4-carboxamide or a salt thereof.

11. (3S,4S)—N-[3,5-bis(trifluoromethyl)benzyl]-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.

12. (3R*,4R*)—N-[3,5-bis(trifluoromethyl)benzyl]-1-[2-(cyclopropylamino)-2-oxoethyl]-3-(4-fluoro-2-methylphenyl)-N-methylpiperidine-4-carboxamide or a salt thereof.

13. (3R*,4R*)—N-[3,5-bis(trifluoromethyl)benzyl]-3-(4-fluoro-2-methylphenyl)-1-[2-(4-hydroxypiperidin-1-yl)-2-oxoethyl]-N-methylpiperidine-4-carboxamide or a salt thereof.

14. (3R*,4R*)-1′-[amino(oxo)acetyl]-N-[3,5-bis(trifluoromethyl)benzyl]-3-(4-fluoro-2-methylphenyl)-N-methyl-1,4′-bipiperidine-4-carboxamide or a salt thereof.

15. (3S ,4S)—N⁴-[3,5-bis(trifluoromethyl)benzyl]-3-(4-fluoro-2-methylphenyl)-N⁴-methyl-1,4′-bipiperidine-1′,4-dicarboxamide or a salt thereof.

16. (3R*,4R*)-1-(2-amino-2-oxoethyl)-N-[3,5-bis(trifluoromethyl)benzyl]-3-(4-fluoro-2-methylphenyl)-N-methylpiperidine-4-carboxamide or a salt thereof.

17. (3R*,4R*)-1-(2-amino-1,1-dimethyl-2-oxoethyl)-N-[3,5-bis(trifluoromethyl)benzyl]-3-(4-fluoro-2-methylphenyl)-N-methylpiperidine-4-carboxamide or a salt thereof.

18. (3R*,4R*)—N-[3,5-bis(trifluoromethyl)benzyl]-3-(4-fluoro-2-methylphenyl)-N-methyl-1-(3-oxocyclopent-1-en-1-yl)piperidine-4-carboxamide or a salt thereof.

19. (3R*,4R*)—N-[3,5-bis(trifluoromethyl)benzyl]-1-(4,4-dimethyl-3-oxocyclohex-1-en-1-yl)-3-(4-fluoro-2-methylphenyl)-N-methylpiperidine-4-carboxamide or a salt thereof.

20. (3R*,4R*)—N-[3,5-bis(trifluoromethyl)benzyl]-3-(4-fluoro-2-methylphenyl)-1-(2-hydroxy-1-methylethyl)-N-methylpiperidine-4-carboxamide or a salt thereof.

21. (3R*,4R*)—N-[3,5-bis(trifluoromethyl)benzyl]-3-(4-fluoro-2-methylphenyl)-1-(3-hydroxy-3-methylbutyl)-N-methylpiperidine-4-carboxamide or a salt thereof.

22. (3R*,4R*)-1-[5-(acetylamino)pyridin-2-yl]-N-[3,5-bis(trifluoromethyl)benzyl]-3-(4-fluoro-2-methylphenyl)-N-methylpiperidine-4-carboxamide or a salt thereof.

23. (3R*,4R*)—N-[3,5-bis(trifluoromethyl)benzyl]-1-(ethylsulfonyl)-3-(4-fluoro-2-methylphenyl)-N-methylpiperidine-4-carboxamide or a salt thereof.

24. (3R*,4R*)—N-[3,5-bis(trifluoromethyl)benzyl]-1-[(2-ethoxyethyl)sulfonyl]-3-(4-fluoro-2-methylphenyl)-N-methylpiperidine-4-carboxamide or a salt thereof.

25. (3R*,4R*)—N-[3,5-bis(trifluoromethyl)benzyl]-3-(4-fluoro-2-methylphenyl)-N-methyl-1-[(2-morpholin-4-ylethyl)sulfonyl]piperidine-4-carboxamide or a salt thereof.

26. (3R*,4R*)—N-[3,5-bis(trifluoromethyl)benzyl]-1-(dimethylsulfamoyl)-3-(4-fluoro-2-methylphenyl)-N-methylpiperidine-4-carboxamide or a salt thereof.

27. (3R*,4R*)—N-[3,5-bis(trifluoromethyl)benzyl]-3-(4-fluoro-2-methylphenyl)-N-methyl-1-(pyridin-2-ylsulfonyl)piperidine-4-carboxamide or a salt thereof.

28. (3S,4S)—N-[3,5-bis(trifluoromethyl)benzyl]-3-(4-fluoro-2-methylphenyl)-1-{[1-(hydroxyacetyl)piperidin-4-yl]carbonyl}-N-methylpiperidine-4-carboxamide or a salt thereof.

29. (3S,4S)—N-[3,5-bis(trifluoromethyl)benzyl]-3-(4-fluoro-2-methylphenyl)-1-({1-[(2S)-2-hydroxypropanoyl]piperidin-4-yl}carbonyl)-N-methylpiperidine-4-carboxamide or a salt thereof.

30. (3S,4S)—N-[3,5-bis(trifluoromethyl)benzyl]-3-(4-fluoro-2-methylphenyl)-1-({1-[(2R)-2-hydroxypropanoyl]piperidin-4-yl}carbonyl)-N-methylpiperidine-4-carboxamide or a salt thereof.

31. (3S,4S)—N-{(1S)-1-[3,5-bis(trifluoromethyl)phenyl]ethyl}-3-(4-fluoro-2-methylphenyl)-1-{[1-(hydroxyacetyl)piperidin-4-yl]carbonyl}-N-methylpiperidine-4-carboxamide or a salt thereof.

32. (3S,4S)—N-{(1R)-1-[3,5-bis(trifluoromethyl)phenyl]ethyl}-3-(4-fluoro-2-methylphenyl)-1-{[1-(hydroxyacetyl)piperidin-4-yl]carbonyl}-N-methylpiperidine-4-carboxamide or a salt thereof.

33. (3S,4S)—N-{(1S)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.

34. (3S,4S)—N-{(1R)-1-[3,5-bis(trifluoromethyl)phenyl]ethyl}-1-[(5,5-dimethyl-2,4-dioxo-1,3-oxazolidin-3-yl)acetyl]-3-(4-fluoro-2-methylphenyl)-N-methylpiperidine-4-carboxamide or a salt thereof.

35. (3S,4S)-1-(2-amino-2-oxoethyl)-N-{(1S)-1-[3,5-bis(trifluoromethyl)phenyl]ethyl}-3-(4-fluoro-2-methylphenyl)-N-methylpiperidine-4-carboxamide or a salt thereof.

36. (3S,4S)-1-(2-amino-2-oxoethyl)-N-{(1R)-1-[3,5-bis(trifluoromethyl)phenyl]ethyl}-3-(4-fluoro-2-methylphenyl)-N-methylpiperidine-4-carboxamide or a salt thereof.

37. (3S,4S)—N-{(1S)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide or a salt thereof.

38. (3S,4S)—N-{(1R)-1-[3,5-bis(trifluoromethyl)phenyl]ethyl}-3-(4-fluoro-2-methylphenyl)-N-methyl-1-(pyrimidin-5-ylcarbonyl)piperidine-4-carboxamide or a salt thereof.

39. (3S,4S)—N-{(1S)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide or a salt thereof.

40. (3S,4S)—N-{(1R)-1-[3,5-bis(trifluoromethyl)phenyl]ethyl}-3-(4-fluoro-2-methylphenyl)-N-methyl-1-[3-(methylsulfonyl)propanoyl]piperidine-4-carboxamide or a salt thereof.

41. (3S,4S)—N-{(1S)-1-[3,5-bis(trifluoromethyl)phenyl]ethyl}-1-(cyclopropylsulfonyl)-3-(4-fluoro-2-methylphenyl)-N-methylpiperidine-4-carboxamide or a salt thereof.

42. (3R*,4R*)-1-(2-amino-2-oxoethyl)-N-[3,5-bis(trifluoromethyl)benzyl]-N-cyclopropyl-3-(4-fluoro-2-methylphenyl)piperidine-4-carboxamide or a salt thereof.

43. N-{(1S)-1-[3,5-bis(trifluoromethyl)phenyl]ethyl}-1-{[1-(hydroxyacetyl)piperidin-4-yl]carbonyl}-N-methyl-3-(3-methylthiophen-2-yl)piperidine-4-carboxamide or a salt thereof.

44. N-{(1S)-1-[3,5-bis(trifluoromethyl)phenyl]ethyl }-1-{[1-(hydroxyacetyl)piperidin-4-yl]carbonyl}-N-methyl-3-(3-methylthiophen-2-yl)piperidine-4-carboxamide or a salt thereof.

45. (3S,4S)-1-[amino(oxo)acetyl]-N-{(1S)-1-[3,5-bis(trifluoromethyl)phenyl]ethyl}-3-(4-fluoro-2-methylphenyl)-N-methylpiperidine-4-carboxamide or a salt thereof.

46. (3S,4S)—N-{(1S)-1-[3,5-bis(trifluoromethyl)phenyl]ethyl}-3-(4-fluoro-2-methylphenyl)-N-methyl-1-(methylsulfonyl)piperidine-4-carboxamide or a salt thereof.

47. (3S,4S)—N-{(1S)-1-[3,5-bis(trifluoromethyl)phenyl]ethyl}-3-(4-fluoro-2-methylphenyl)-N-methyl-1-[2-(methylsulfonyl)ethyl]piperidine-4-carboxamide or a salt thereof.

48. A prodrug of the compound of any one of claims 1 to 47.

49. A pharmaceutical composition comprising the compound of any one of claims 1 to 47, or a prodrug thereof.

50-52. (canceled)

53. A method for the prophylaxis or treatment of a lower urinary tract disease, a gastrointestinal disease or a central nervous system disease in a mammal, which comprises administering an effective amount of the compound of any one of claims 1 to 47, a salt thereof, or a prodrug thereof.

54. (canceled)
